(19) United States
(12) Patent Application Publication
(10) Pub. No.: US 2024/0083836 A1
(43) Pub. Date: Mar. 14, 2024

(54) MATERIALS FOR ELECTRONIC DEVICES

(71) Applicant: Merck Patent GmbH, Darmstadt (DE)

(72) Inventors: Teresa MUJICA-FERNAUD, Darmstadt (DE); Elvira MONTENEGRO, Darmstadt (DE); Christian WIRGES, Darmstadt (DE)

(21) Appl. No.: 18/266,827

(22) PCT Filed: Dec. 15, 2021
(86) PCT No.: PCT/EP2021/085804

§ 371 Date: Jun. 13, 2023

(30) Foreign Application Priority Data
Dec. 18, 2020 (EP) 20215338.3

(51) Int. Cl. C07C 211/57; C09K 11/06; H10K 50/11; H10K 50/15; H10K 85/60

(52) U.S. Cl. CPC C07C 211/57; C09K 11/06; H10K 50/11; H10K 50/15; H10K 85/626; H10K 85/633; H10K 85/636

(57) ABSTRACT

The present application relates to compounds of a formula (I), (II) or (III), to processes for preparing such compounds, and to electronic devices comprising one or more such compounds, and to the use of such compounds in electronic devices.

The present application relates to aromatic amines having particular aromatic or heteroaromatic ring systems on the amine nitrogen atom. The compounds are suitable for use in electronic devices.

Electronic devices in the context of this application are understood to mean what are called organic electronic devices, which comprise organic semiconductor materials as functional materials. More particularly, these are understood to mean OLEDs (organic electroluminescent devices). The term OLEDs is understood to mean electronic devices which have one or more layers comprising organic compounds and emit light on application of electrical voltage. The construction and general principle of function of OLEDs are known to those skilled in the art.

In electronic devices, especially OLEDs, there is great interest in an improvement in the performance data. In these aspects, it has not yet been possible to find any entirely satisfactory solution.

A great influence on the performance data of electronic devices is possessed by emission layers and layers having a hole-transporting function. Novel compounds are also being sought for use in these layers, especially hole-transporting compounds and compounds that can serve as hole-transporting matrix material, especially for phosphorescent emitters, in an emitting layer. For this purpose, there is a search especially for compounds that have a high glass transition temperature, high stability, and high conductivity for holes. A high stability of the compound is a prerequisite for achieving a long lifetime of the electronic device. There is moreover a need to find compounds whose use in electronic devices results in improvement of the performance data of the devices, especially in high efficiency, long lifetime and low operating voltage.

In the prior art, triarylamine compounds in particular, for example spirobifluoreneamines and fluoreneamines, are known as hole transport materials and hole-transporting matrix materials for electronic devices. However, there remains room for improvement in respect of the abovementioned properties.

It has now been found that aromatic amines of the formulae below which are characterized in that they have particular aromatic or heteroaromatic ring systems on the amine nitrogen atom are of excellent suitability for use in electronic devices. They are especially suitable for use in OLEDs, and even more particularly therein for use as hole transport materials and for use as hole-transporting matrix materials, especially for phosphorescent emitters. The compounds lead to high lifetime, high efficiency and low operating voltage of the devices. Further preferably, the compounds found have a high glass transition temperature, high stability, low sublimation temperature, good solubility, good synthetic accessibility and high conductivity for holes.

The present application thus provides a compound of one of the following formulae:

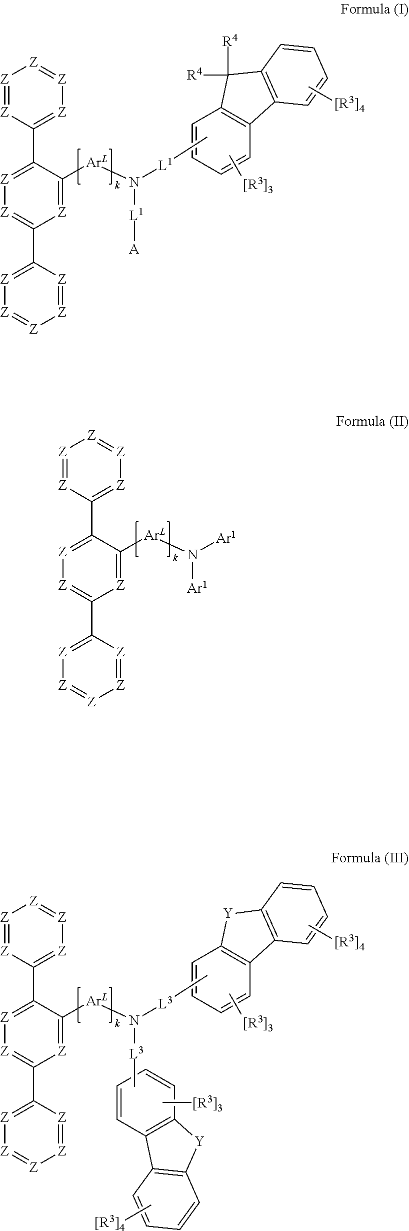

where:
A is a group selected from the following formulae:

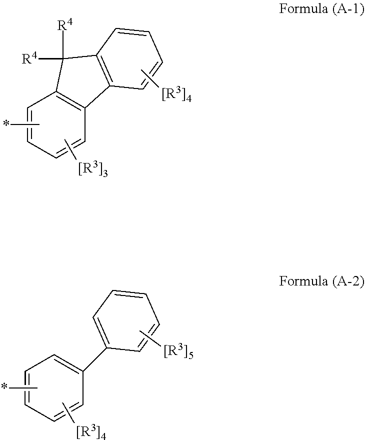

which is bonded to L¹ via the bond marked *;
Z is the same or different at each instance and is selected from CR¹ and N;
Ar^{L} is the same or different at each instance and is selected from aromatic ring systems which have 6 to 40 aromatic ring atoms and are substituted by R² radicals, and heteroaromatic ring systems which have 5 to 40 aromatic ring atoms and are substituted by R² radicals;
k is 0, 1, 2 or 3, where, when k=0, the Ar^{L} group is absent and the two groups that bind to Ar^{L} in formula (I), (II) and (III) are bonded directly to one another, where, when k=2, two Ar^{L} groups are bonded successively in a chain, and where, when k=3, three Ar^{L} groups are bonded successively in a chain;
in the case that k=0, Ar¹ is the same or different at each instance and is selected from aromatic ring systems which have 6 to 40 aromatic ring atoms and are substituted by R³ radicals, and heteroaromatic ring systems which have 5 to 40 aromatic ring atoms and are substituted by R³ radicals, where at least one Ar¹ group is selected from the following groups in which the bond to the nitrogen atom in formula (II) is labelled *:

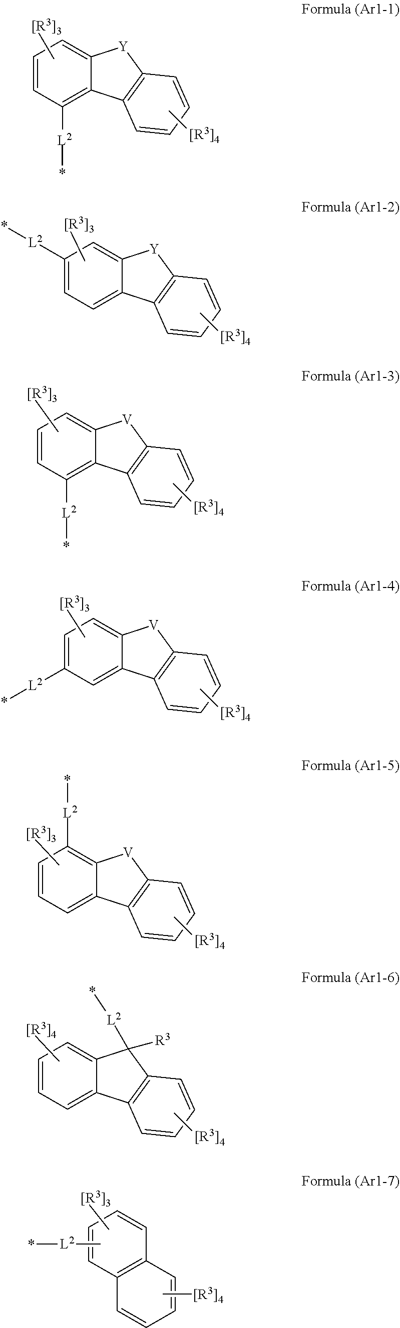

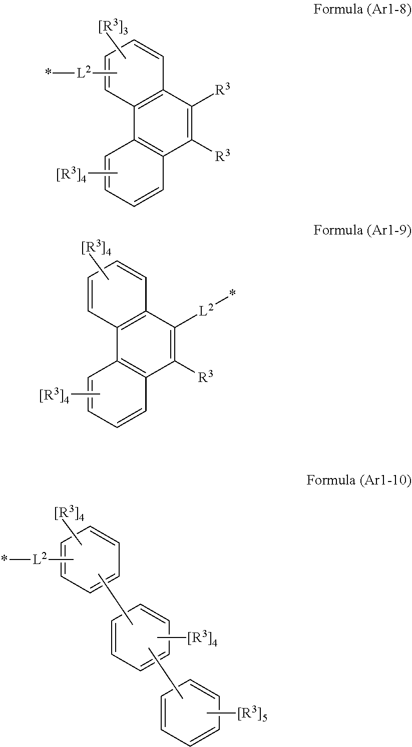

when k=1, 2 or 3, Ar¹ is the same or different at each instance and is selected from aromatic ring systems which have 6 to 40 aromatic ring atoms and are substituted by R³ radicals, and heteroaromatic ring systems which have 5 to 40 aromatic ring atoms and are substituted by R³ radicals;
L¹ is the same or different at each instance and is a single bond, an aromatic ring system which has 6 to 40 aromatic ring atoms and is substituted by R⁵ radicals, or a heteroaromatic ring system which has 5 to 40 aromatic ring atoms and is substituted by R⁵ radicals;
L² is the same or different at each instance and is a single bond, an aromatic ring system which has 6 to 40 aromatic ring atoms and is substituted by R⁵ radicals, or a heteroaromatic ring system which has 5 to 40 aromatic ring atoms and is substituted by R⁵ radicals;
L³ is the same or different at each instance and is an aromatic ring system which has 6 to 40 aromatic ring atoms and is substituted by R⁵ radicals, or a heteroaromatic ring system which has 5 to 40 aromatic ring atoms and is substituted by R⁵ radicals;
Y is the same or different at each instance and is selected from O and S,
V is the same or different at each instance and is selected from Si(R³)₂, C(R³)₂ and a group

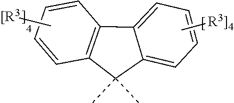

where the dotted bonds are the bonds to the radical of the formula Ar¹-3, Ar¹-4 or Ar¹-5;
R¹ is the same or different at each instance and is selected from H, D, F, Cl, Br, I, C(═O)R⁶, CN, Si(R⁶)₃, N(R⁶)₂, P(═O)(R⁶)₂, OR⁶, S(═O)R⁶, S(═O)₂R⁶, straight-chain alkyl or alkoxy groups having 1 to 20 carbon atoms, branched or cyclic alkyl or alkoxy groups having 3 to 20 carbon atoms, alkenyl or alkynyl groups having 2 to 20 carbon atoms, aromatic ring systems having 6 to 40 aromatic ring atoms excluding fluorenyl, and heteroaromatic ring systems having 5 to 40 aromatic ring atoms; where two or more R¹ radicals may be joined to one another and may form a ring; where the alkyl, alkoxy, alkenyl and alkynyl groups mentioned and the aromatic ring systems and heteroaromatic ring systems mentioned are each substituted by R⁶ radicals; and where one or more CH₂ groups in the alkyl, alkoxy, alkenyl and alkynyl groups mentioned may be replaced by —R⁶C═CR⁶—, —C≡C—, Si(R⁶)₂, C═O, C═NR⁶, —C(═O)O—, —C(═O)NR⁶—, NR⁶, P(═O)(R⁶), —O—, —S—, SO or SO₂;
R² is the same or different at each instance and is selected from H, D, F, Cl, Br, I, C(═O)R⁶, CN, Si(R⁶)₃, N(R⁶)₂, P(═O)(R⁶)₂, OR⁶, S(═O)R⁶, S(═O)₂R⁶, straight-chain alkyl or alkoxy groups having 1 to 20 carbon atoms, branched or cyclic alkyl or alkoxy groups having 3 to 20 carbon atoms, alkenyl or alkynyl groups having 2 to 20 carbon atoms, aromatic ring systems having 6 to 40 aromatic ring atoms, and heteroaromatic ring systems having 5 to 40 aromatic ring atoms; where two or more R² radicals may be joined to one another and may form a ring; where the alkyl, alkoxy, alkenyl and alkynyl groups mentioned and the aromatic ring systems and heteroaromatic ring systems mentioned are each substituted by R⁶ radicals; and where one or more CH₂ groups in the alkyl, alkoxy, alkenyl and alkynyl groups mentioned may be replaced by —R⁶C═CR⁶—, —C≡C—, Si(R⁶)₂, C═O, C═NR⁶, —C(═O)O—, —C(═O)NR⁶—, NR⁶, P(═O)(R⁶), —O—, —S—, SO or SO₂;
R³ is the same or different at each instance and is selected from H, D, F, Cl, Br, I, C(═O)R⁶, CN, Si(R⁶)₃, N(R⁶)₂, P(═O)(R⁶)₂, OR⁶, S(═O)R⁶, S(═O)₂R⁶, straight-chain alkyl or alkoxy groups having 1 to 20 carbon atoms, branched or cyclic alkyl or alkoxy groups having 3 to 20 carbon atoms, alkenyl or alkynyl groups having 2 to 20 carbon atoms, aromatic ring systems having 6 to 40 aromatic ring atoms, and heteroaromatic ring systems having 5 to 40 aromatic ring atoms; where two or more R³ radicals may be joined to one another and may form a ring; where the alkyl, alkoxy, alkenyl and alkynyl groups mentioned and the aromatic ring systems and heteroaromatic ring systems mentioned are each substituted by R⁶ radicals; and where one or more CH₂ groups in the alkyl, alkoxy, alkenyl and alkynyl groups mentioned may be replaced by —R⁶C═CR⁶—, —C≡C—, Si(R⁶)₂, C═O, C═NR⁶, —C(═O)O—, —C(═O)NR⁶—, NR⁶, P(═O)(R⁶), —O—, —S—, SO or SO₂;
R⁴ is the same or different at each instance and is selected from straight-chain alkyl or alkoxy groups having 1 to 20 carbon atoms, branched or cyclic alkyl or alkoxy groups having 3 to 20 carbon atoms, alkenyl or alkynyl groups having 2 to 20 carbon atoms, aromatic ring systems having 6 to 40 aromatic ring atoms, and heteroaromatic ring systems having 5 to 40 aromatic ring atoms; where any two R⁴ radicals may be joined to one another and may form a ring; where the alkyl, alkoxy, alkenyl and alkynyl groups mentioned and the aromatic ring systems and heteroaromatic ring systems mentioned are each substituted by R⁶ radicals; and where one or more CH₂ groups in the alkyl, alkoxy, alkenyl and alkynyl groups mentioned may be replaced by —R⁶C═CR⁶—, —C≡C—, Si(R⁶)₂, C═O, C═NR⁶, —C(═O)O—, —C(═O)NR⁶—, NR⁶, P(═O)(R⁶), —O—, —S—, SO or SO₂; with the proviso that no two R⁴ radicals bonded to the same carbon atom may both be aromatic ring systems;
R⁵ is the same or different at each instance and is selected from H, D, F, Cl, Br, I, C(═O)R⁶, CN, Si(R⁶)₃, N(R⁶)₂, P(═O)(R⁶)₂, OR⁶, S(═O)R⁶, S(═O)₂R⁶, straight-chain alkyl or alkoxy groups having 1 to 20 carbon atoms, branched or cyclic alkyl or alkoxy groups having 3 to 20 carbon atoms, alkenyl or alkynyl groups having 2 to 20 carbon atoms, aromatic ring systems having 6 to 40 aromatic ring atoms, and heteroaromatic ring systems having 5 to 40 aromatic ring atoms; where two or more R⁵ radicals may be joined to one another and may form a ring; where the alkyl, alkoxy, alkenyl and alkynyl groups mentioned and the aromatic ring systems and heteroaromatic ring systems mentioned are each substituted by R⁶ radicals; and where one or more CH₂ groups in the alkyl, alkoxy, alkenyl and alkynyl groups mentioned may be replaced by —R⁶C═CR⁶—, —C≡C—, Si(R⁶)₂, C═O, C═NR⁶, —C(═O)O—, —C(═O)NR⁶—, NR⁶, P(═O)(R⁶), —O—, —S—, SO or SO₂;
R⁶ is the same or different at each instance and is selected from H, D, F, Cl, Br, I, C(═O)R⁷, CN, Si(R⁷)₃, N(R⁷)₂, P(═O)(R⁷)₂, OR⁷, S(═O)R⁷, S(═O)₂R⁷, straight-chain alkyl or alkoxy groups having 1 to 20 carbon atoms, branched or cyclic alkyl or alkoxy groups having 3 to 20 carbon atoms, alkenyl or alkynyl groups having 2 to 20 carbon atoms, aromatic ring systems having 6 to 40 aromatic ring atoms, and heteroaromatic ring systems having 5 to 40 aromatic ring atoms; where two or more R⁶ radicals may be joined to one another and may form a ring; where the alkyl, alkoxy, alkenyl and alkynyl groups mentioned and the aromatic ring systems and heteroaromatic ring systems mentioned are each substituted by R⁷ radicals; and where one or more CH₂ groups in the alkyl, alkoxy, alkenyl and alkynyl groups mentioned may be replaced by —R⁷C═CR⁷—, —C≡C—, Si(R⁷)₂, C═O, C═NR⁷, —C(═O)O—, —C(═O)NR⁷—, NR⁷, P(═O)(R⁷), —O—, —S—, SO or SO₂;
R⁷ is the same or different at each instance and is selected from H, D, F, Cl, Br, I, CN, alkyl or alkoxy groups having 1 to 20 carbon atoms, alkenyl or alkynyl groups having 2 to 20 carbon atoms, aromatic ring systems having 6 to 40 aromatic ring atoms and heteroaromatic ring systems having 5 to 40 aromatic ring atoms; where two or more R⁷ radicals may be joined to one another and may form a ring; and where the alkyl, alkoxy, alkenyl and alkynyl groups, aromatic ring systems and heteroaromatic ring systems mentioned may be substituted by one or more radicals selected from F and CN.

The representation

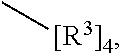

shown in a ring, is understood to mean that one R³ radical is bonded to each of the four free positions on the ring, where the R³ radicals may be the same or different at each instance.

The definitions which follow are applicable to the chemical groups that are used in the present application. They are applicable unless any more specific definitions are given.

An aryl group in the context of this invention is understood to mean either a single aromatic cycle, i.e. benzene, or a fused aromatic polycycle, for example naphthalene, phenanthrene or anthracene. A fused aromatic polycycle in the context of the present application consists of two or more single aromatic cycles fused to one another. Fusion between cycles is understood here to mean that the cycles share at least one edge with one another. An aryl group in the context of this invention contains 6 to 40 aromatic ring atoms. In addition, an aryl group does not contain any heteroatom as aromatic ring atom, but only carbon atoms.

A heteroaryl group in the context of this invention is understood to mean either a single heteroaromatic cycle, for example pyridine, pyrimidine or thiophene, or a fused heteroaromatic polycycle, for example quinoline or carbazole. A fused heteroaromatic polycycle in the context of the present application consists of two or more single aromatic or heteroaromatic cycles that are fused to one another, where at least one of the aromatic and heteroaromatic cycles is a heteroaromatic cycle. Fusion between cycles is understood here to mean that the cycles share at least one edge with one another. A heteroaryl group in the context of this invention contains 5 to 40 aromatic ring atoms of which at least one is a heteroatom. The heteroatoms of the heteroaryl group are preferably selected from N, O and S.

An aryl or heteroaryl group, each of which may be substituted by the abovementioned radicals, is especially understood to mean groups derived from benzene, naphthalene, anthracene, phenanthrene, pyrene, dihydropyrene, chrysene, perylene, triphenylene, fluoranthene, benzanthracene, benzophenanthrene, tetracene, pentacene, benzopyrene, furan, benzofuran, isobenzofuran, dibenzofuran, thiophene, benzothiophene, isobenzothiophene, dibenzothiophene, pyrrole, indole, isoindole, carbazole, pyridine, quinoline, isoquinoline, acridine, phenanthridine, benzo-5,6-quinoline, benzo-6,7-quinoline, benzo-7,8-quinoline, phenothiazine, phenoxazine, pyrazole, indazole, imidazole, benzimidazole, benzimidazolo[1,2-a]benzimidazole, naphthimidazole, phenanthrimidazole, pyridimidazole, pyrazinimidazole, quinoxalinimidazole, oxazole, benzoxazole, naphthoxazole, anthroxazole, phenanthroxazole, isoxazole, 1,2-thiazole, 1,3-thiazole, benzothiazole, pyridazine, benzopyridazine, pyrimidine, benzopyrimidine, quinoxaline, pyrazine, phenazine, naphthyridine, azacarbazole, benzocarboline, phenanthroline, 1,2,3-triazole, 1,2,4-triazole, benzotriazole, 1,2,3-oxadiazole, 1,2,4-oxadiazole, 1,2,5-oxadiazole, 1,3,4-oxadiazole, 1,2,3-thiadiazole, 1,2,4-thiadiazole, 1,2,5-thiadiazole, 1,3,4-thiadiazole, 1,3,5-triazine, 1,2,4-triazine, 1,2,3-triazine, tetrazole, 1,2,4,5-tetrazine, 1,2,3,4-tetrazine, 1,2,3,5-tetrazine, purine, pteridine, indolizine and benzothiadiazole.

An aromatic ring system in the context of this invention is a system which does not necessarily contain solely aryl groups, but which may additionally contain one or more nonaromatic rings fused to at least one aryl group. These nonaromatic rings contain exclusively carbon atoms as ring atoms. Examples of groups covered by this definition are tetrahydronaphthalene, fluorene and spirobifluorene. In addition, the term “aromatic ring system” includes systems that consist of two or more aromatic ring systems joined to one another via single bonds, for example biphenyl, terphenyl, 7-phenyl-2-fluorenyl, quaterphenyl and 3,5-diphenyl-1-phenyl. An aromatic ring system in the context of this invention contains 6 to 40 carbon atoms and no heteroatoms in the ring system. The definition of “aromatic ring system” does not include heteroaryl groups.

A heteroaromatic ring system conforms to the abovementioned definition of an aromatic ring system, except that it must contain at least one heteroatom as ring atom. As is the case for the aromatic ring system, the heteroaromatic ring system need not contain exclusively aryl groups and heteroaryl groups, but may additionally contain one or more nonaromatic rings fused to at least one aryl or heteroaryl group. The nonaromatic rings may contain exclusively carbon atoms as ring atoms, or they may additionally contain one or more heteroatoms, where the heteroatoms are preferably selected from N, O and S. One example of such a heteroaromatic ring system is benzopyranyl. In addition, the term “heteroaromatic ring system” is understood to mean systems that consist of two or more aromatic or heteroaromatic ring systems that are bonded to one another via single bonds, for example 4,6-diphenyl-2-triazinyl. A heteroaromatic ring system in the context of this invention contains 5 to 40 ring atoms selected from carbon and heteroatoms, where at least one of the ring atoms is a heteroatom. The heteroatoms of the heteroaromatic ring system are preferably selected from N, O and S.

The terms “heteroaromatic ring system” and “aromatic ring system” as defined in the present application thus differ from one another in that an aromatic ring system cannot have a heteroatom as ring atom, whereas a heteroaromatic ring system must have at least one heteroatom as ring atom. This heteroatom may be present as a ring atom of a nonaromatic heterocyclic ring or as a ring atom of an aromatic heterocyclic ring.

In accordance with the above definitions, any aryl group is covered by the term “aromatic ring system”, and any heteroaryl group is covered by the term “heteroaromatic ring system”.

An aromatic ring system having 6 to 40 aromatic ring atoms or a heteroaromatic ring system having 5 to 40 aromatic ring atoms is especially understood to mean groups derived from the groups mentioned above under aryl groups and heteroaryl groups, and from biphenyl, terphenyl, quaterphenyl, fluorene, spirobifluorene, dihydrophenanthrene, dihydropyrene, tetrahydropyrene, indenofluorene, truxene, isotruxene, spirotruxene, spiroisotruxene, indenocarbazole, or from combinations of these groups.

In the context of the present invention, a straight-chain alkyl group having 1 to 20 carbon atoms and a branched or cyclic alkyl group having 3 to 20 carbon atoms and an alkenyl or alkynyl group having 2 to 40 carbon atoms in which individual hydrogen atoms or CH₂ groups may also be substituted by the groups mentioned above in the definition of the radicals are preferably understood to mean the methyl, ethyl, n-propyl, i-propyl, n-butyl, i-butyl, s-butyl, t-butyl, 2-methylbutyl, n-pentyl, s-pentyl, cyclopentyl, neopentyl, n-hexyl, cyclohexyl, neohexyl, n-heptyl, cycloheptyl, n-octyl, cyclooctyl, 2-ethylhexyl, trifluoromethyl, pentafluoroethyl, 2,2,2-trifluoroethyl, ethenyl, propenyl, butenyl, pentenyl, cyclopentenyl, hexenyl, cyclohexenyl, heptenyl, cycloheptenyl, octenyl, cyclooctenyl, ethynyl, propynyl, butynyl, pentynyl, hexynyl or octynyl radicals.

An alkoxy or thioalkyl group having 1 to 20 carbon atoms in which individual hydrogen atoms or CH₂ groups may also be substituted by the groups mentioned above in the definition of the radicals is preferably understood to mean methoxy, trifluoromethoxy, ethoxy, n-propoxy, i-propoxy, n-butoxy, i-butoxy, s-butoxy, t-butoxy, n-pentoxy, s-pentoxy, 2-methylbutoxy, n-hexoxy, cyclohexyloxy, n-heptoxy, cycloheptyloxy, n-octyloxy, cyclooctyloxy, 2-ethylhexyloxy, pentafluoroethoxy, 2,2,2-trifluoroethoxy, methylthio, ethylthio, n-propylthio, i-propylthio, n-butylthio, i-butylthio, s-butylthio, t-butylthio, n-pentylthio, s-pentylthio, n-hexylthio, cyclohexylthio, n-heptylthio, cycloheptylthio, n-octylthio, cyclooctylthio, 2-ethylhexylthio, trifluoromethylthio, pentafluoroethylthio, 2,2,2-trifluoroethylthio, ethenylthio, propenylthio, butenylthio, pentenylthio, cyclopentenylthio, hexenylthio, cyclohexenylthio, heptenylthio, cycloheptenylthio, octenylthio, cyclooctenylthio, ethynylthio, propynylthio, butynylthio, pentynylthio, hexynylthio, heptynylthio or octynylthio.

The wording that two or more radicals together may form a ring, in the context of the present application, shall be understood to mean, inter alia, that the two radicals are joined to one another by a chemical bond. In addition, however, the abovementioned wording shall also be understood to mean that, if one of the two radicals is hydrogen, the second radical binds to the position to which the hydrogen atom was bonded, forming a ring.

In a preferred embodiment, Z is CR¹. In an alternative preferred embodiment, Z is the same or different at each instance and is selected from CR¹ and N, where not more than one Z group per ring is N.

Ar^{L} is the same or different at each instance and is selected from aromatic ring systems which have 6 to 25 aromatic ring atoms and are substituted by R² radicals, and heteroaromatic ring systems which have 5 to 25 aromatic ring atoms and are substituted by R² radicals; and is more preferably the same or different at each instance and is selected from phenyl, biphenyl, naphthyl and fluorenyl, each substituted by R² radicals; and is most preferably selected from phenyl substituted by R² radicals.

Preferably, Ar^{L} is the same or different at each instance and is selected from groups of the following formulae:

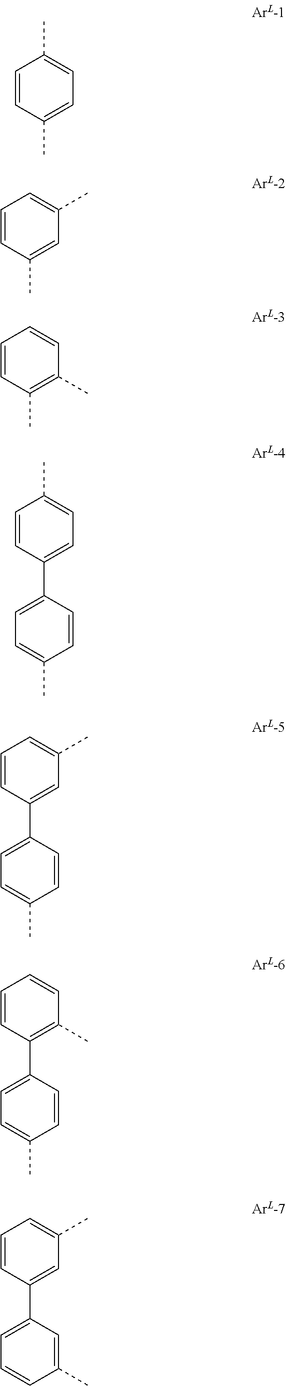

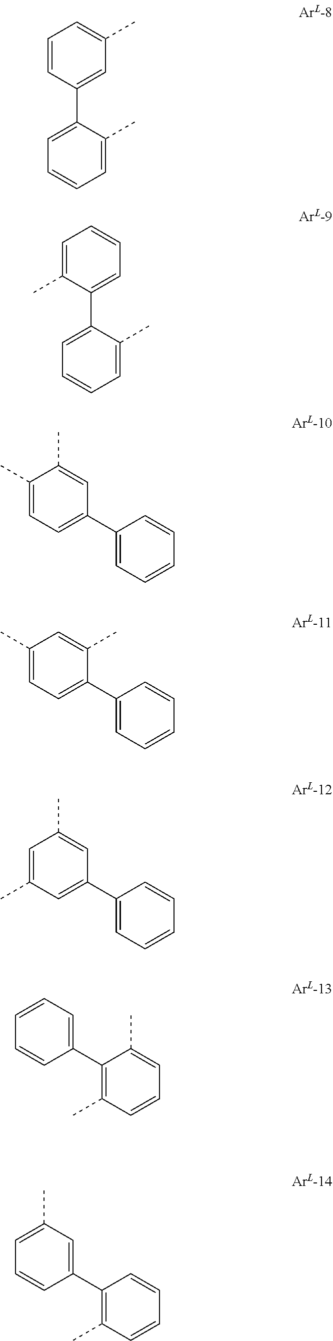

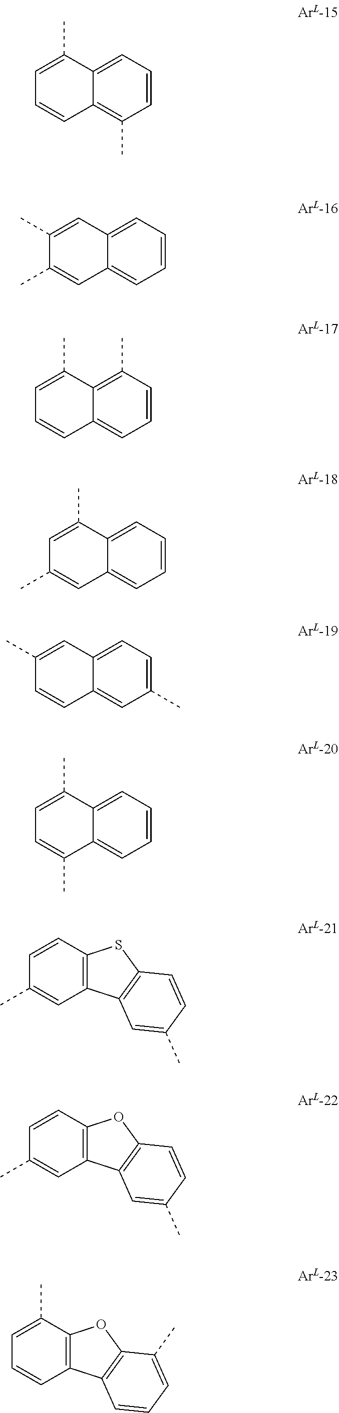

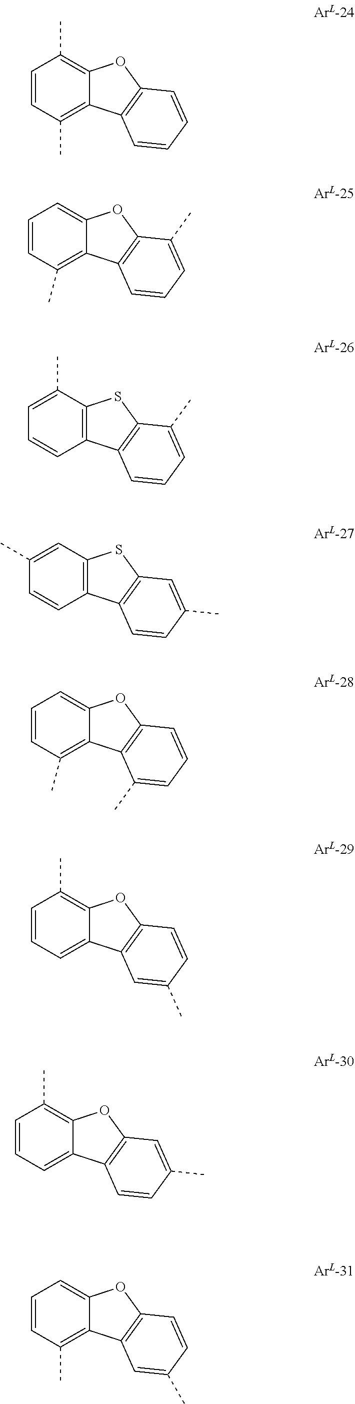

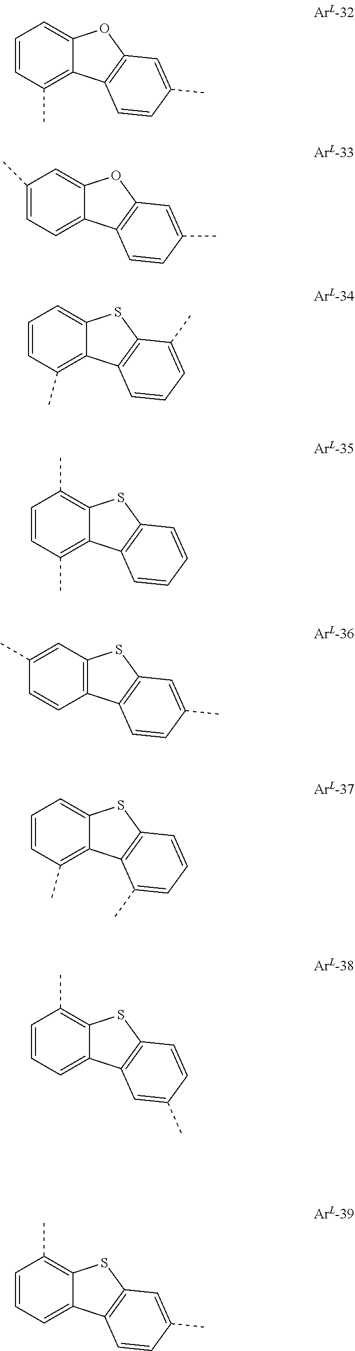

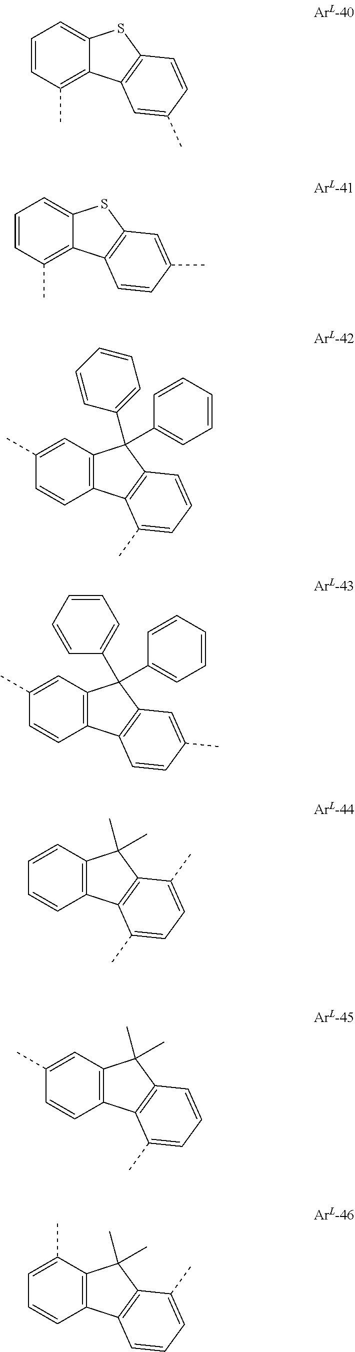

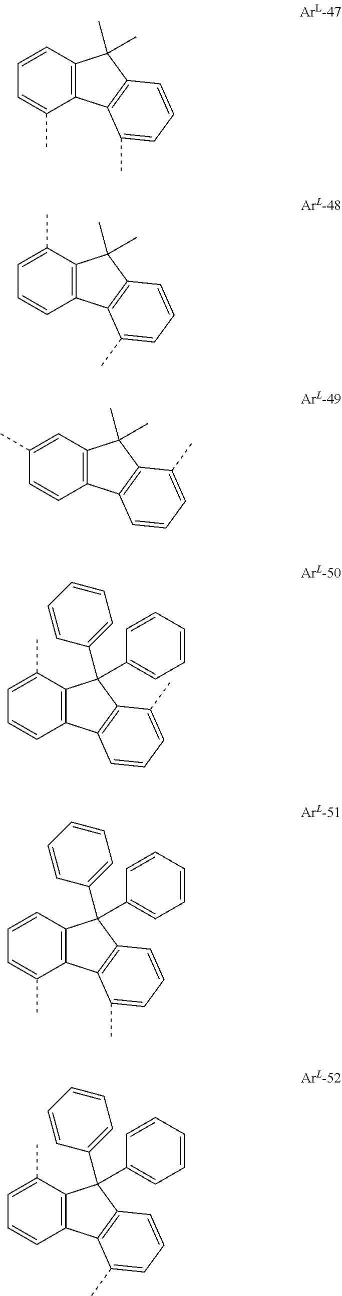

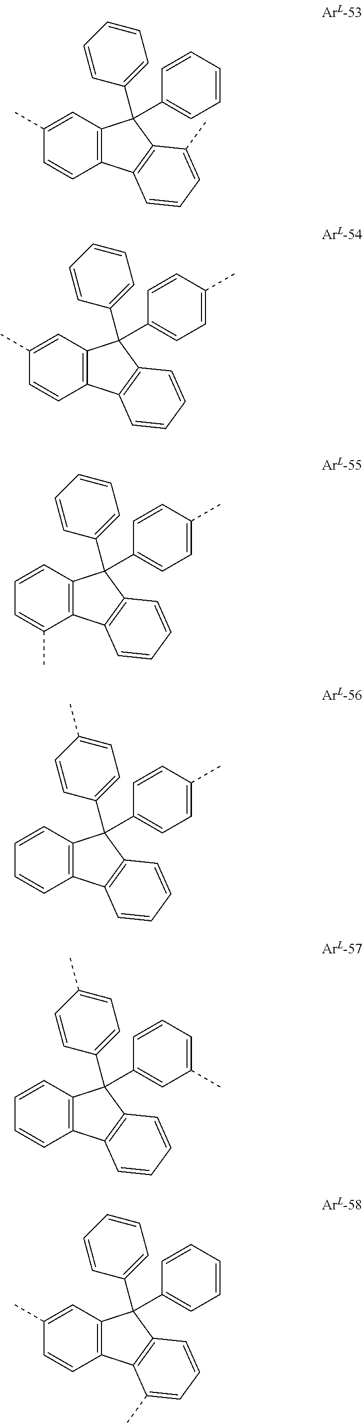

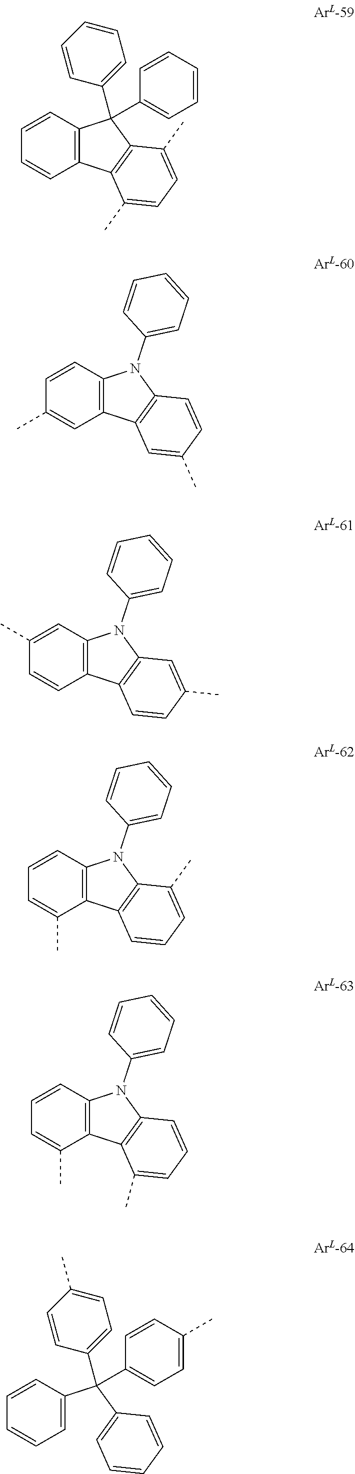

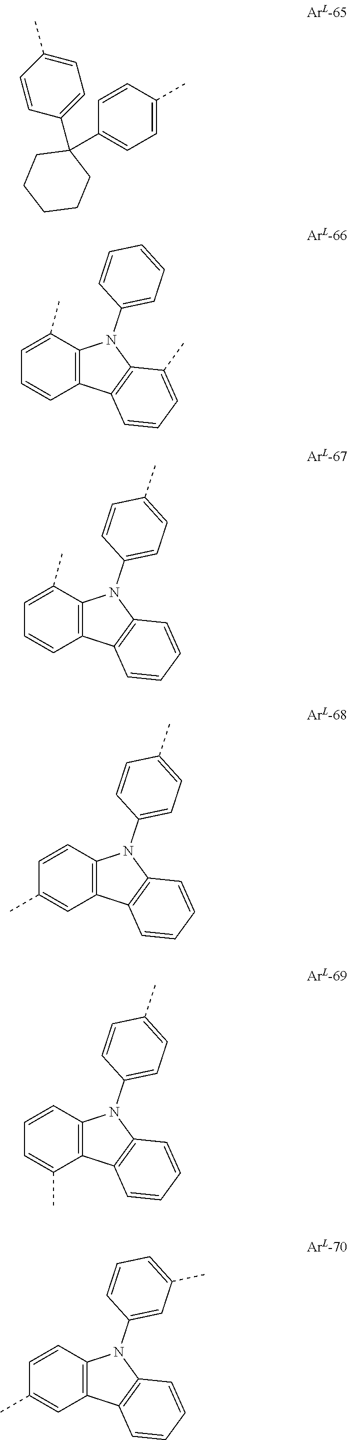

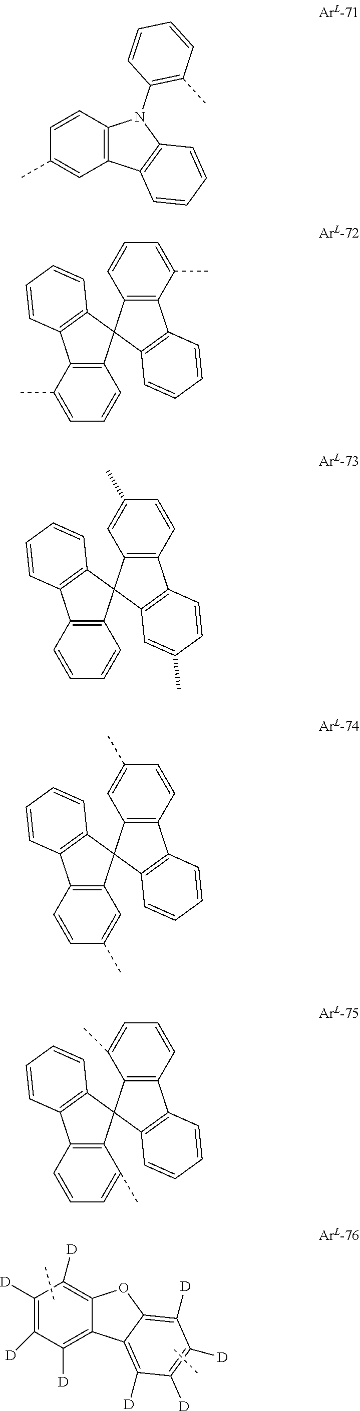

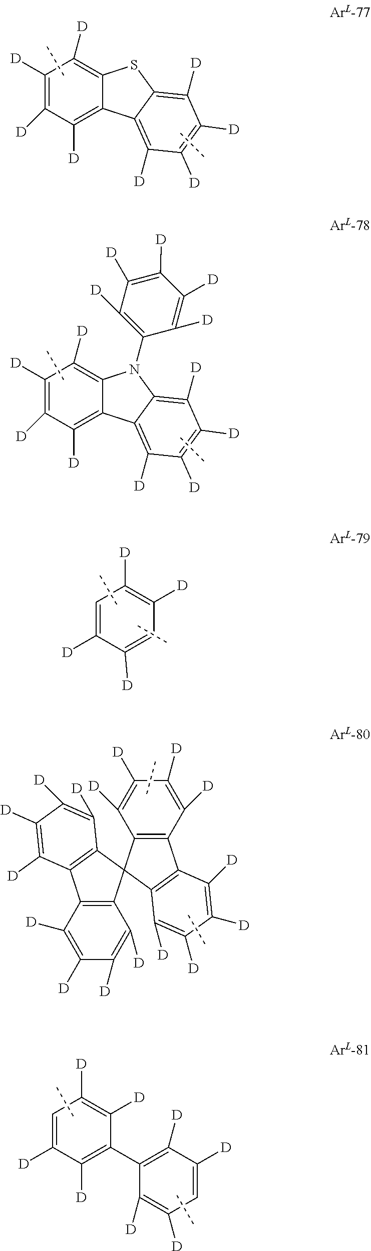

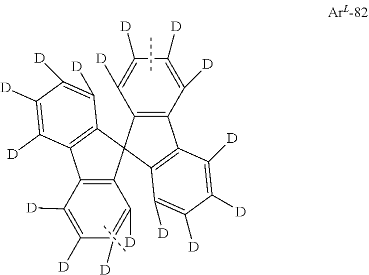

where the dotted lines represent the bonds to the rest of the formula, and where particular preference is given to the formulae Ar^{L}-1, Ar^{L}-2 and Ar^{L}-3.

L¹ is preferably the same or different at each instance and is selected from single bond, aromatic ring system which has 6 to 25 aromatic ring atoms and is substituted by R⁵ radicals, and heteroaromatic ring system which has 5 to 25 aromatic ring atoms and is substituted by R⁵ radicals. In a preferred embodiment, L¹ is the same or different at each instance and is selected from single bond, phenylene substituted by R⁵ radicals, naphthylene substituted by R⁵ radicals, fluorenylene substituted by R⁵ radicals, and biphenylene substituted by R⁵ radicals. In a particularly preferred embodiment, L¹ at each instance is a single bond.

L² is preferably the same or different at each instance and is selected from single bond, aromatic ring system which has 6 to 25 aromatic ring atoms and is substituted by R⁵ radicals, and heteroaromatic ring system which has 5 to 25 aromatic ring atoms and is substituted by R⁵ radicals. In a preferred embodiment, L² is the same or different at each instance and is selected from single bond, phenylene substituted by R⁵ radicals, naphthylene substituted by R⁵ radicals, fluorenylene substituted by R⁵ radicals, and biphenylene substituted by R⁵ radicals. In a particularly preferred embodiment, L² at each instance is a single bond.

L³ is preferably the same or different at each instance and is selected from aromatic ring system which has 6 to 25 aromatic ring atoms and is substituted by R⁵ radicals, and heteroaromatic ring system which has 5 to 25 aromatic ring atoms and is substituted by R⁵ radicals. In a preferred embodiment, L³ is the same or different at each instance and is selected from phenylene substituted by R⁵ radicals, naphthylene substituted by R⁵ radicals, fluorenylene substituted by R⁵ radicals, and biphenylene substituted by R⁵ radicals. In a particularly preferred embodiment, L³ at each instance is phenylene substituted by R⁵ radicals.

If k=1, 2 or 3, Ar¹ is the same or different at each instance and is selected from aromatic ring systems which have 6 to 25 aromatic ring atoms and are substituted by R³ radicals, and heteroaromatic ring systems which have 5 to 25 aromatic ring atoms and are substituted by R³ radicals.

If k=1, 2 or 3 and if k=0, the Ar¹ group that does not conform to one of the formulae (Ar1-1) to (Ar1-10) is as follows: Preferred Ar¹ groups are the same or different at each instance and are selected from monovalent groups derived from benzene, biphenyl, terphenyl, quaterphenyl, naphthalene, fluorene, especially 9,9′-dimethylfluorene and 9,9′-diphenylfluorene, benzofluorene, spirobifluorene, indenofluorene, indenocarbazole, dibenzofuran, dibenzothiophene, benzocarbazole, carbazole, benzofuran, benzothiophene, indole, quinoline, pyridine, pyrimidine, pyrazine, pyridazine and triazine, where each of the monovalent groups is substituted by R³ radicals. In these cases, the Ar¹ groups are preferably the same or different at each instance and are selected from combinations of 2 to 4 groups derived from benzene, biphenyl, terphenyl, quaterphenyl, naphthalene, fluorene, especially 9,9′-dimethylfluorene and 9,9′-diphenylfluorene, benzofluorene, spirobifluorene, indenofluorene, indenocarbazole, dibenzofuran, dibenzothiophene, benzocarbazole, carbazole, benzofuran, benzothiophene, indole, quinoline, pyridine, pyrimidine, pyrazine, pyridazine and triazine, where each of the monovalent groups is substituted by R³ radicals.

If k=1, 2 or 3 and if k=0, the Ar¹ group that does not conform to one of the formulae (Ar¹-1) to (Ar¹-10) is as follows: Particularly preferred Ar¹ groups are the same or different at each instance and are selected from benzene, biphenyl, terphenyl, quaterphenyl, naphthyl, fluorenyl, especially 9,9′-dimethylfluorenyl and 9,9′-diphenylfluorenyl, benzofluorenyl, spirobifluorenyl, indenofluorenyl, indenocarbazolyl, dibenzofuranyl, dibenzothiophenyl, carbazolyl, benzofuranyl, benzothiophenyl, benzofused dibenzofuranyl, benzofused dibenzothiophenyl, and phenyl substituted by a group selected from naphthyl, fluorenyl, spirobifluorenyl, dibenzofuranyl, dibenzothiophenyl, carbazolyl, pyridyl, pyrimidyl and triazinyl, where the abovementioned embodiments are each substituted by R³ radicals.

Ar¹ is preferably the same or different at each instance and is selected from groups of the following formulae:

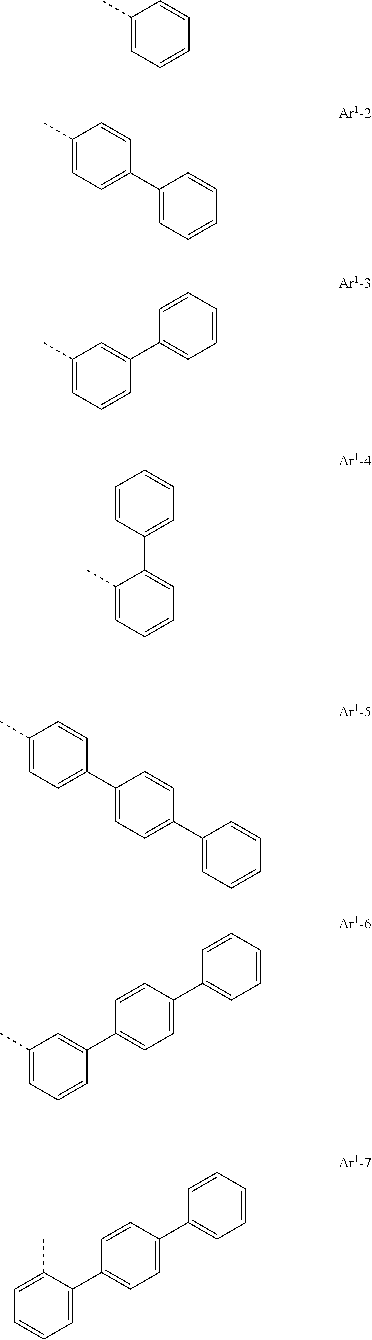

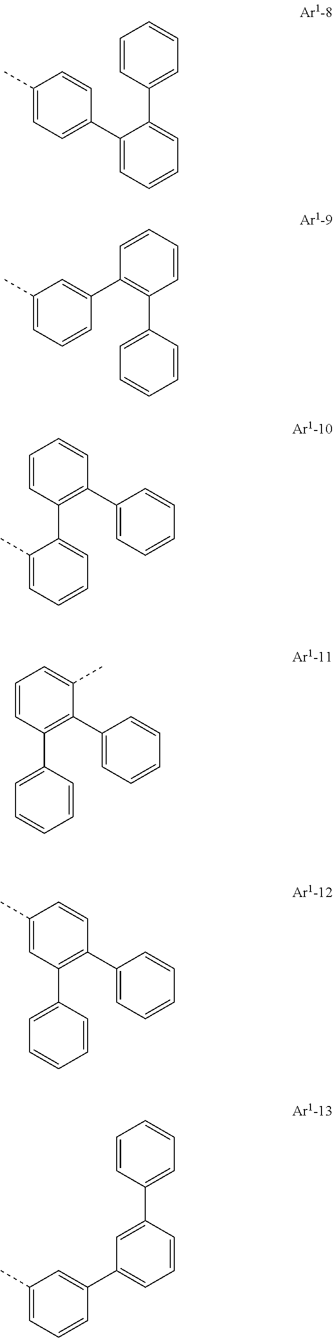

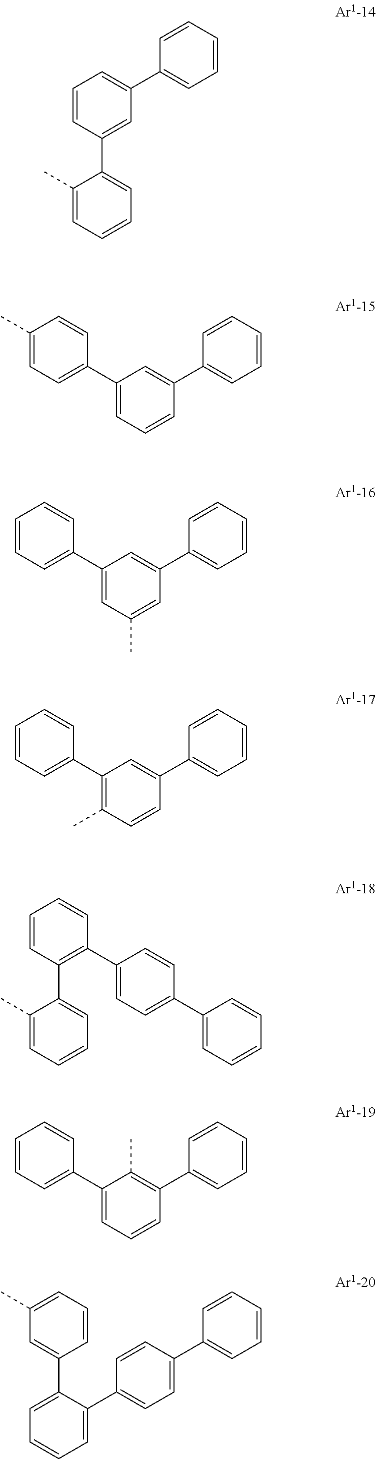

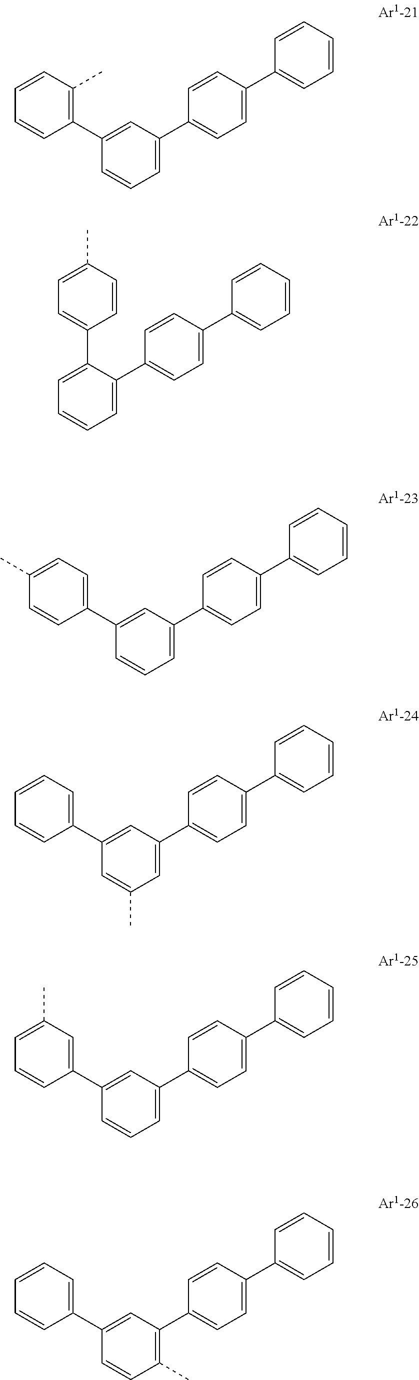

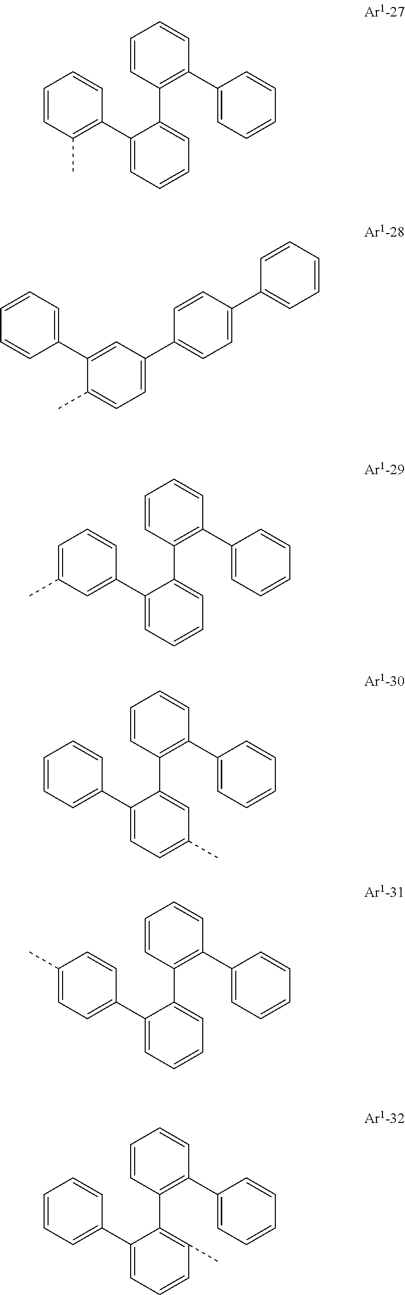

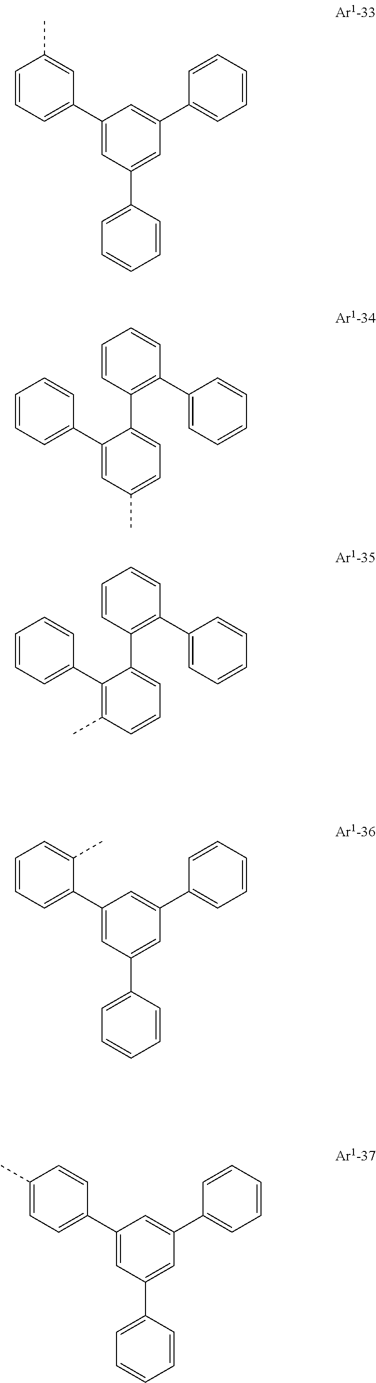

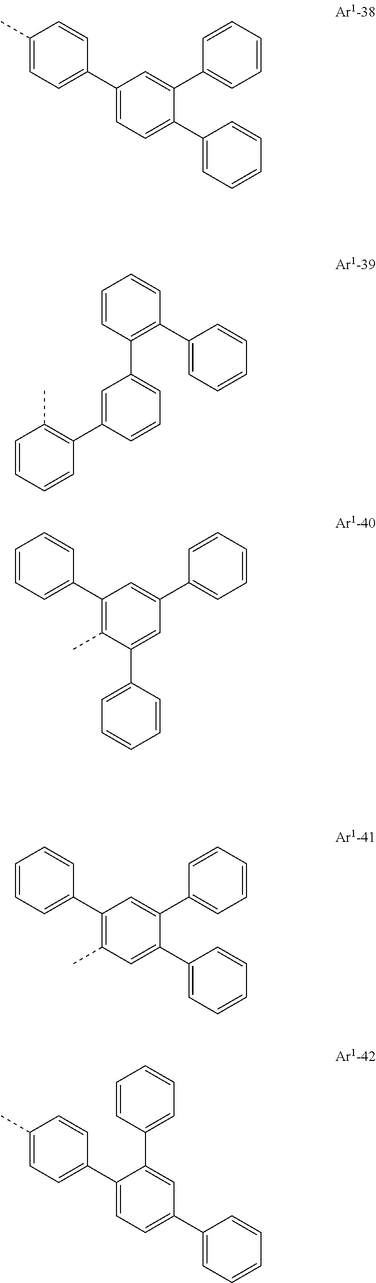

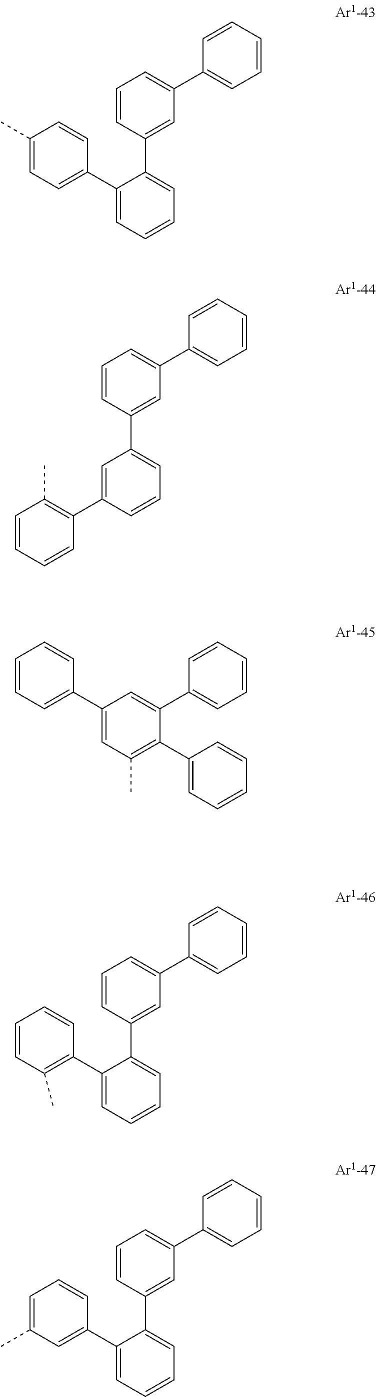

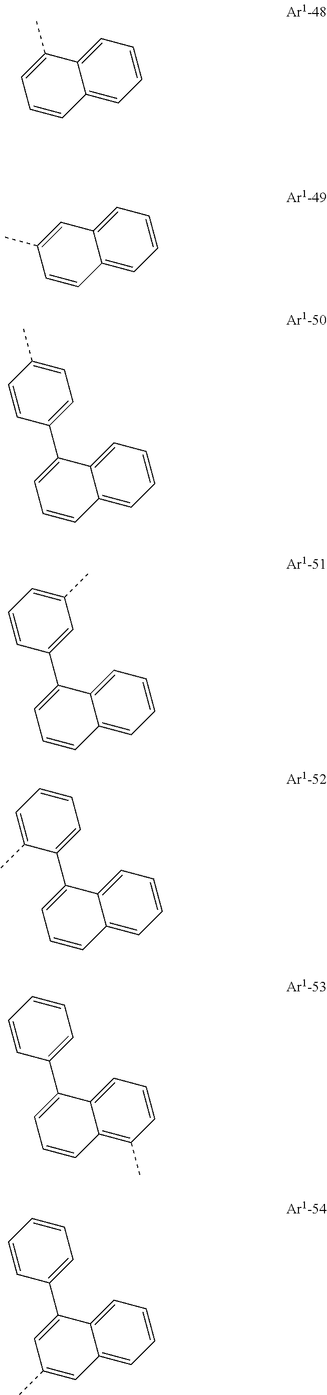

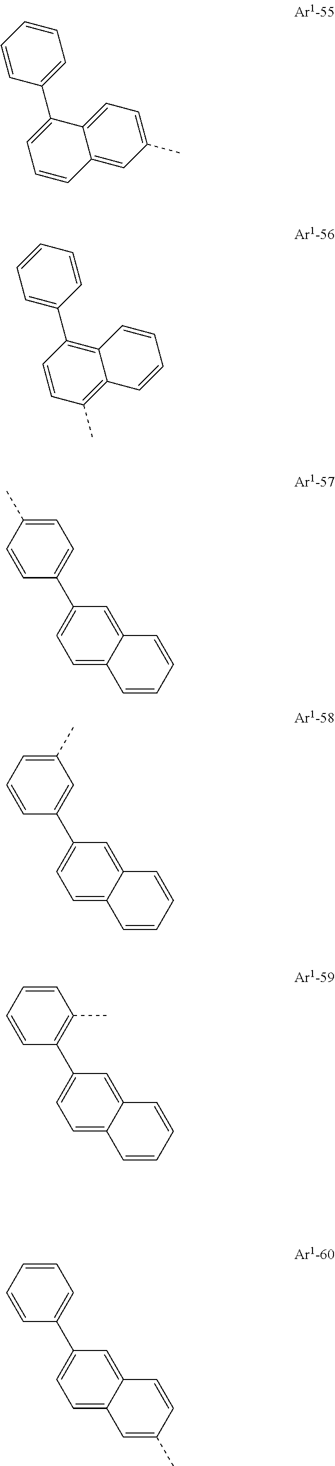

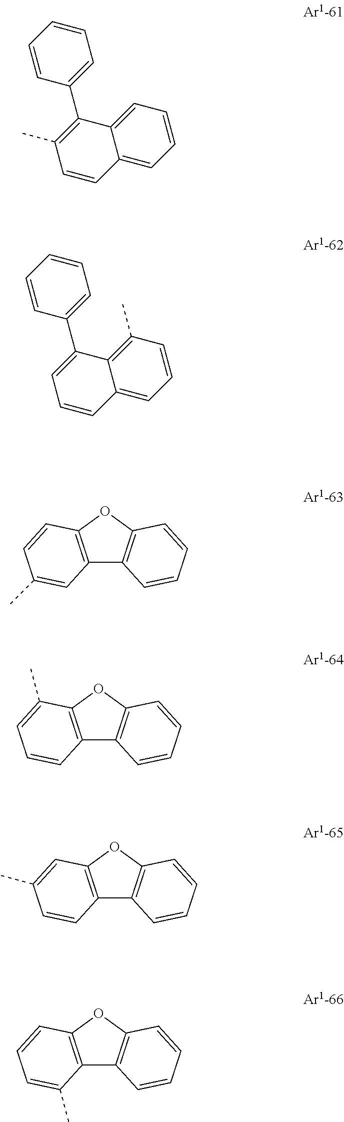

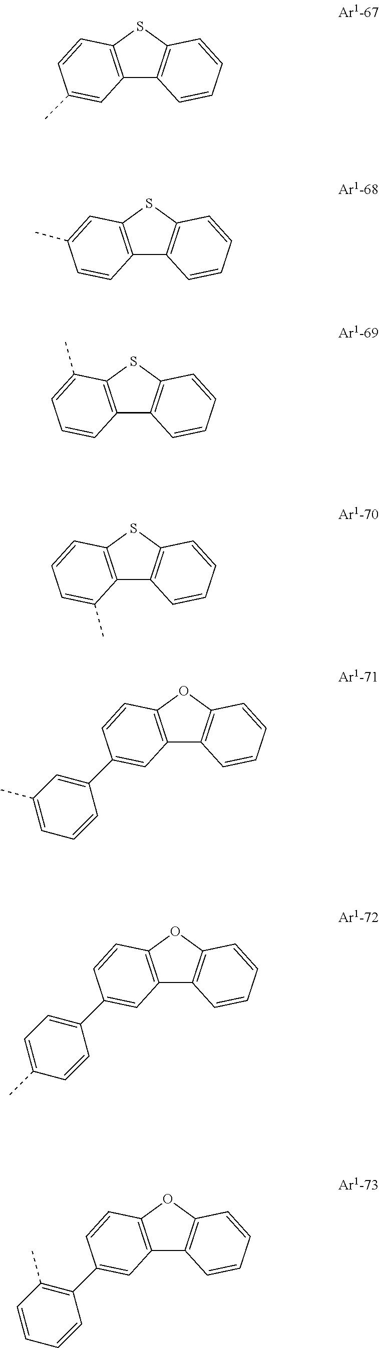

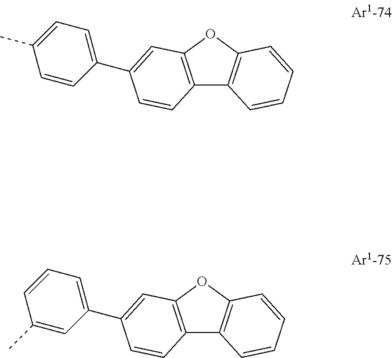

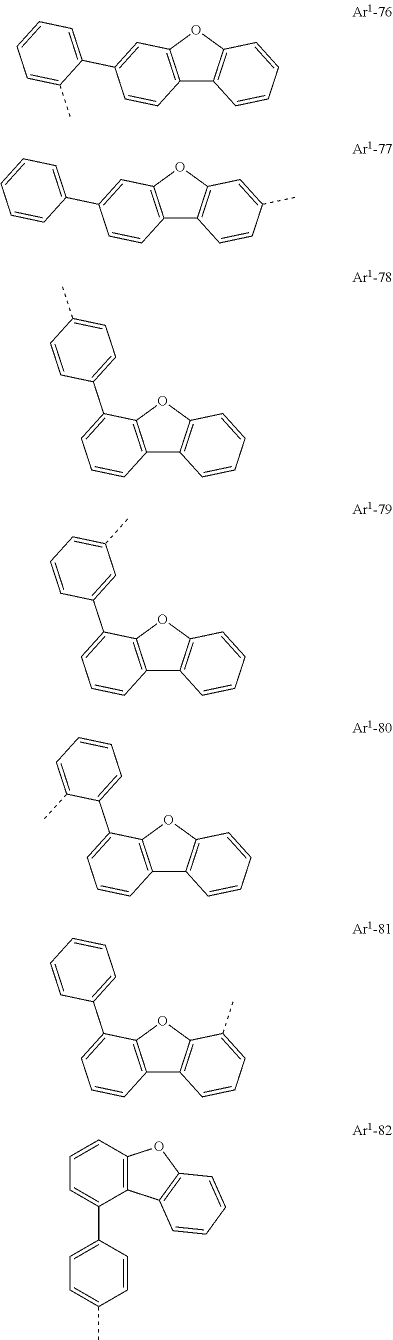

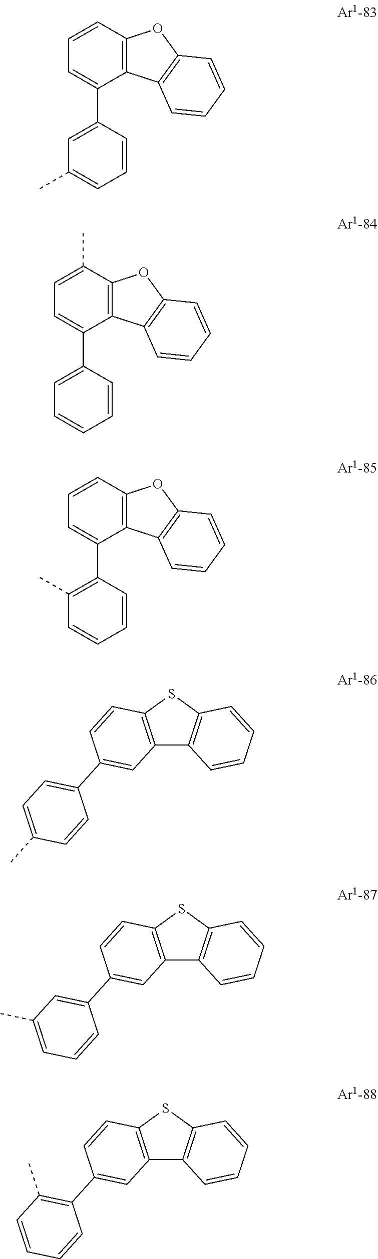

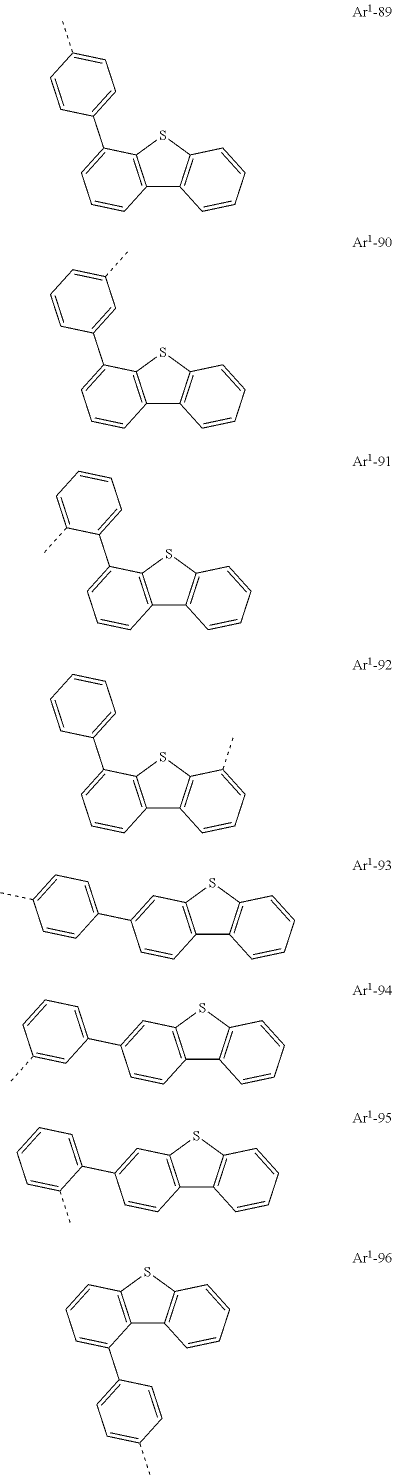

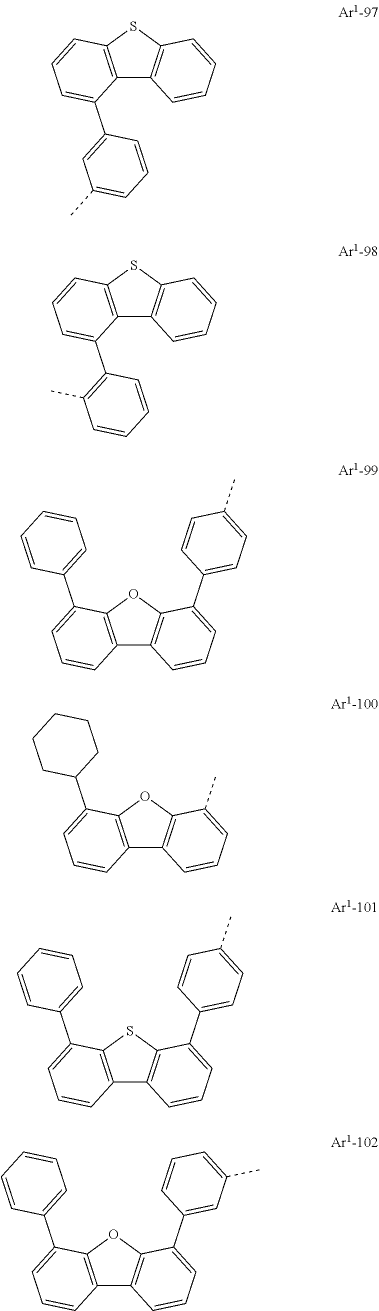

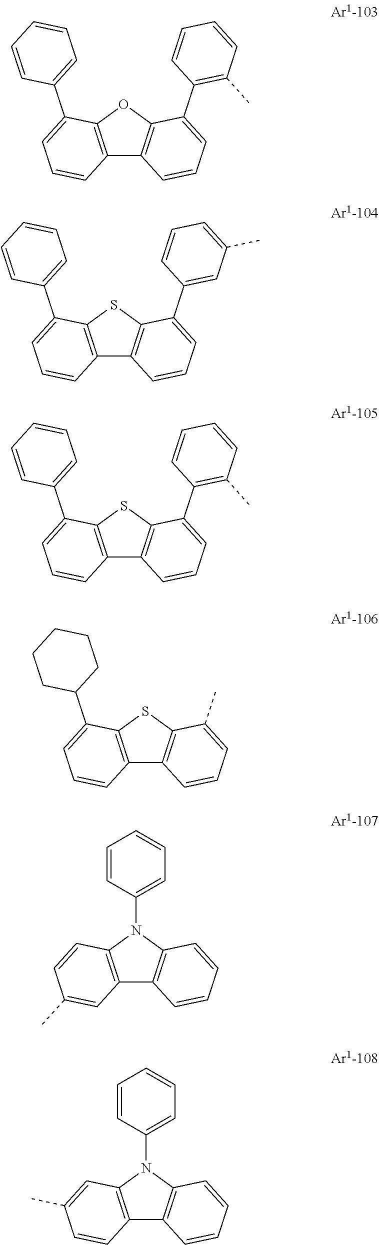

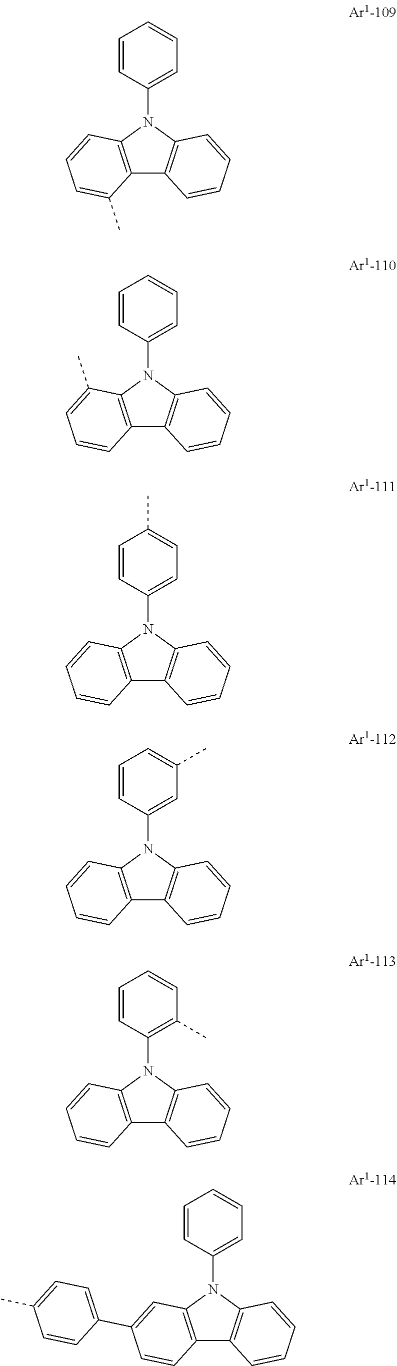

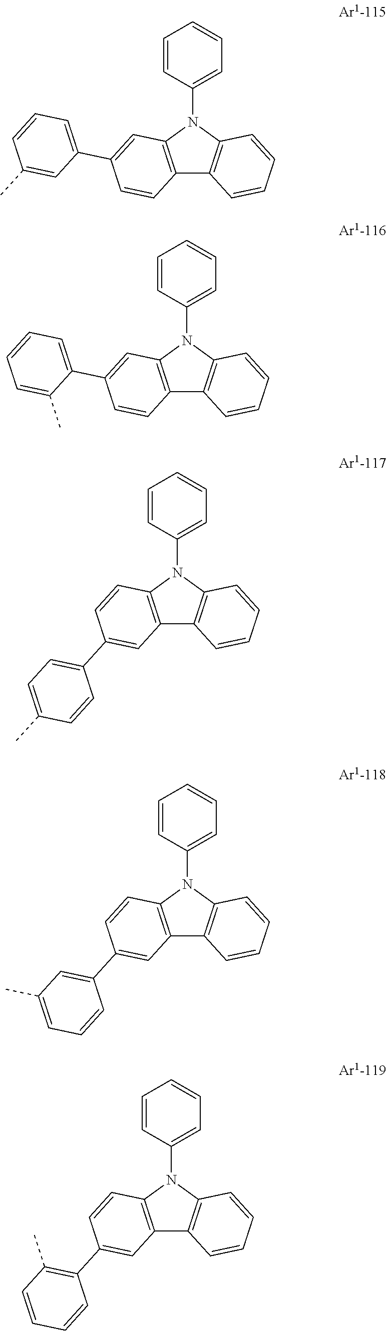

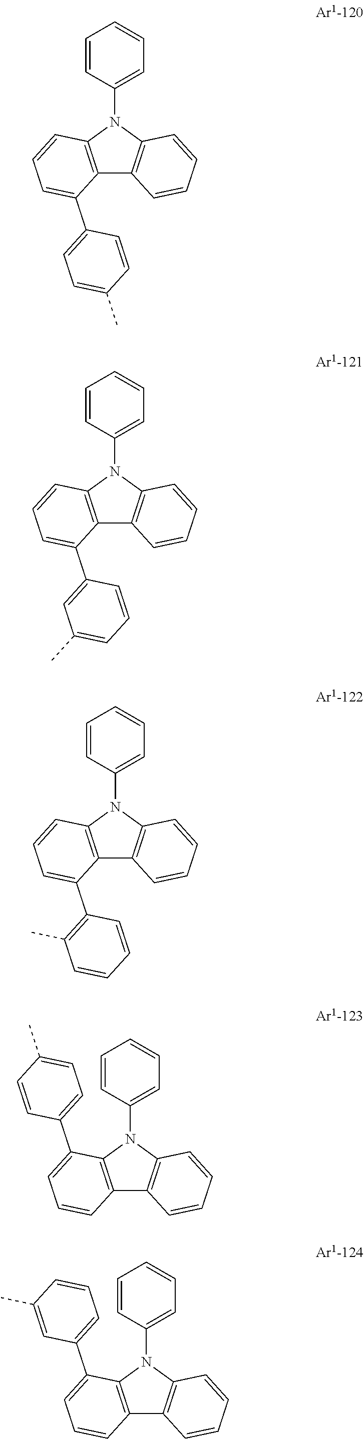

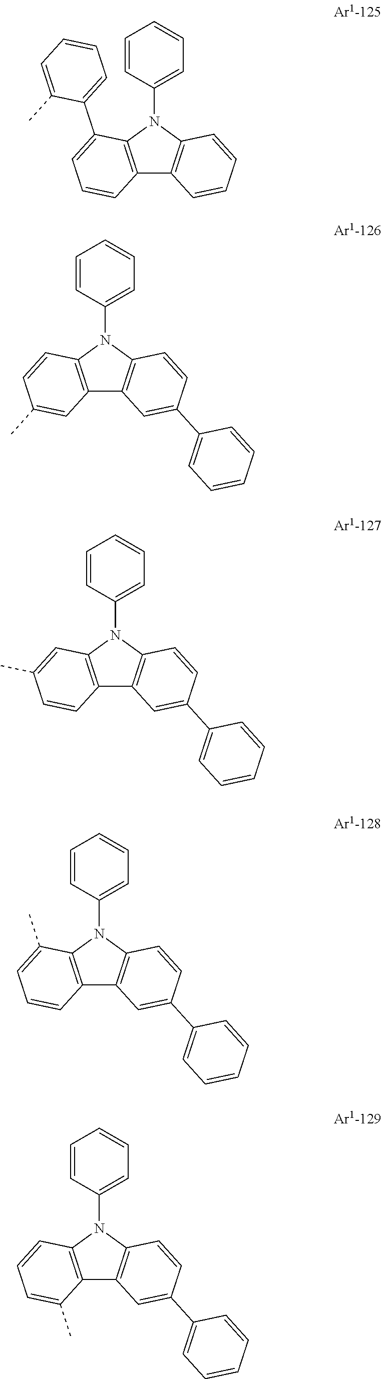

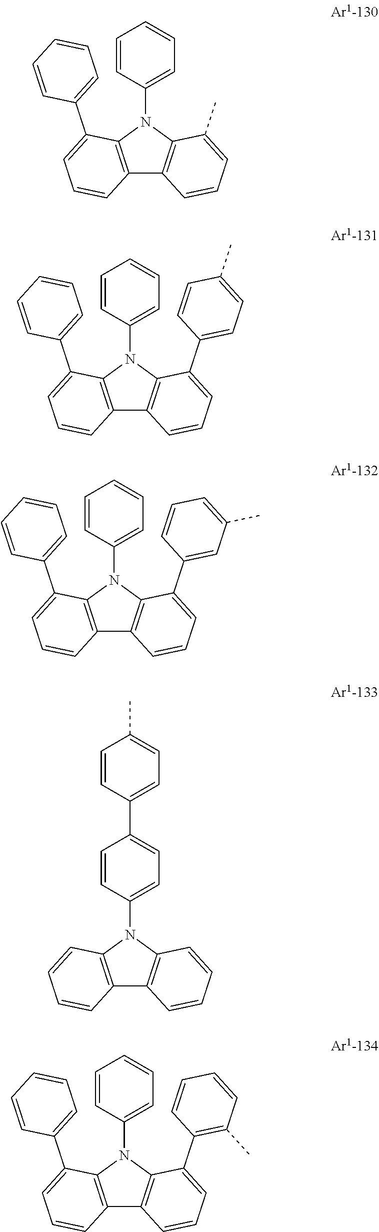

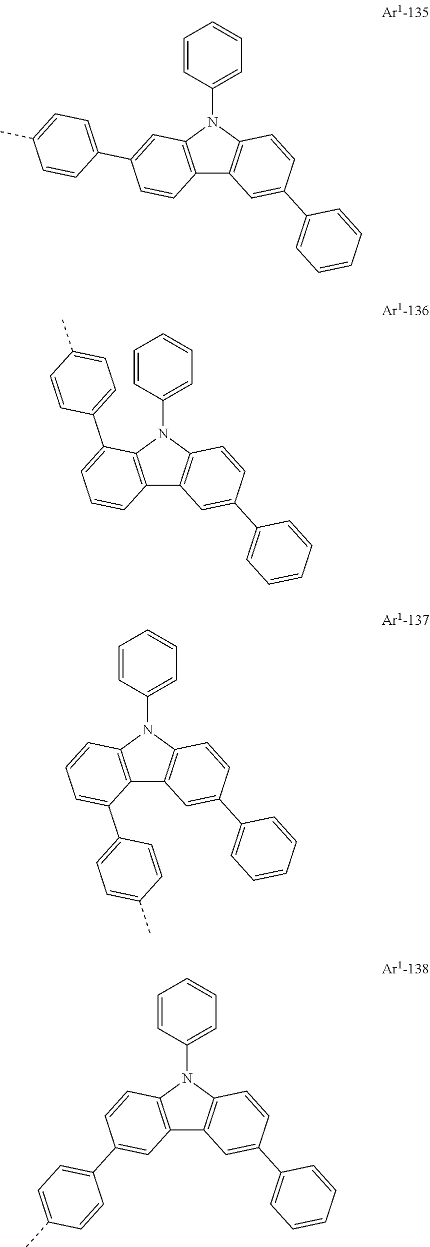

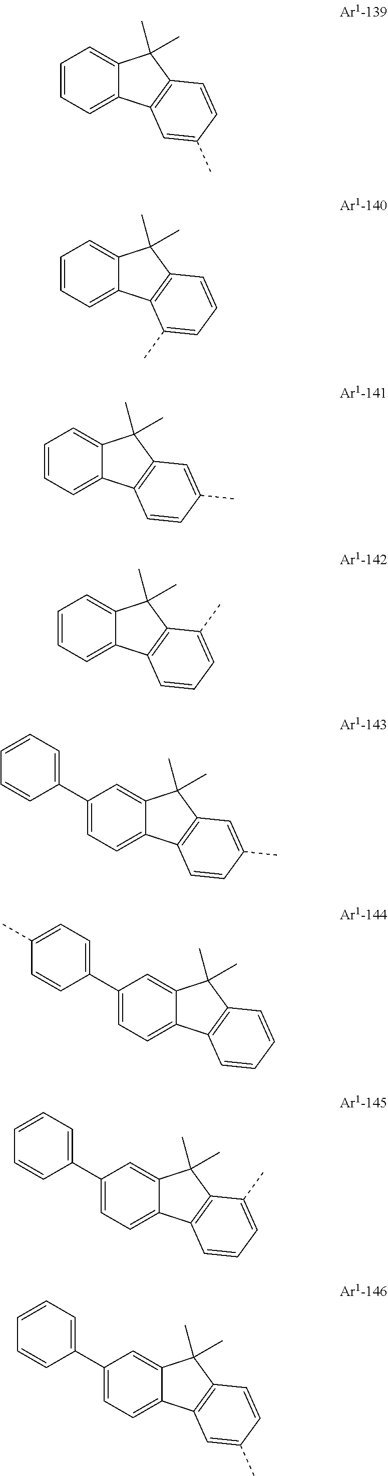

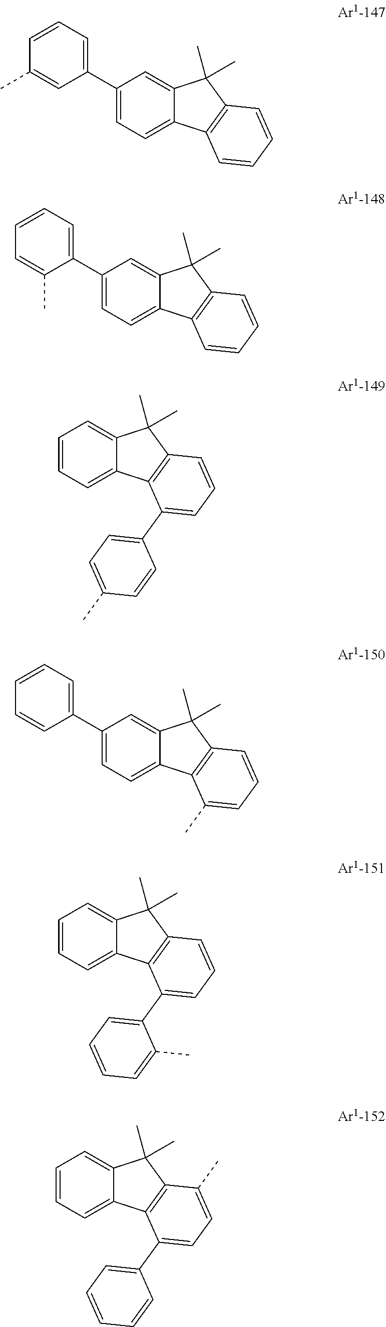

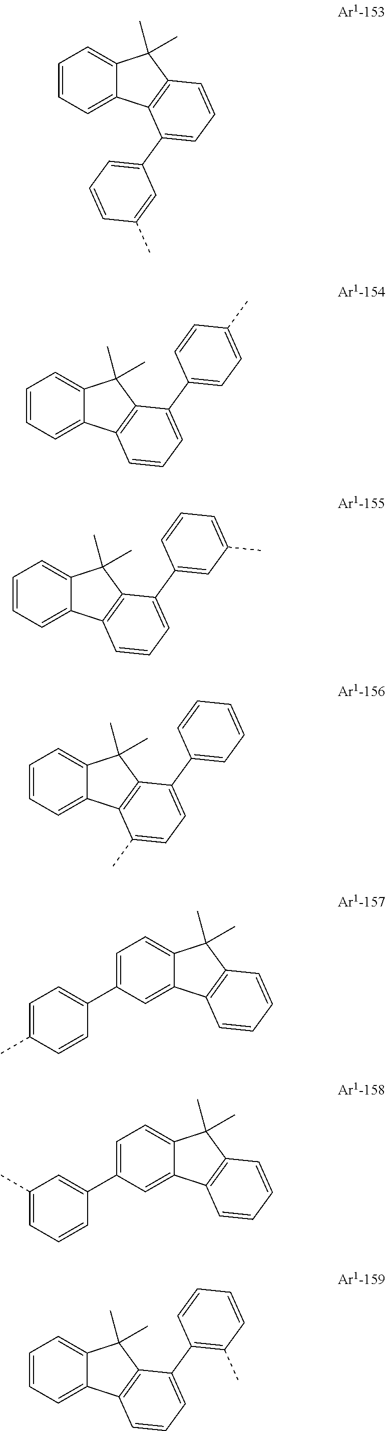

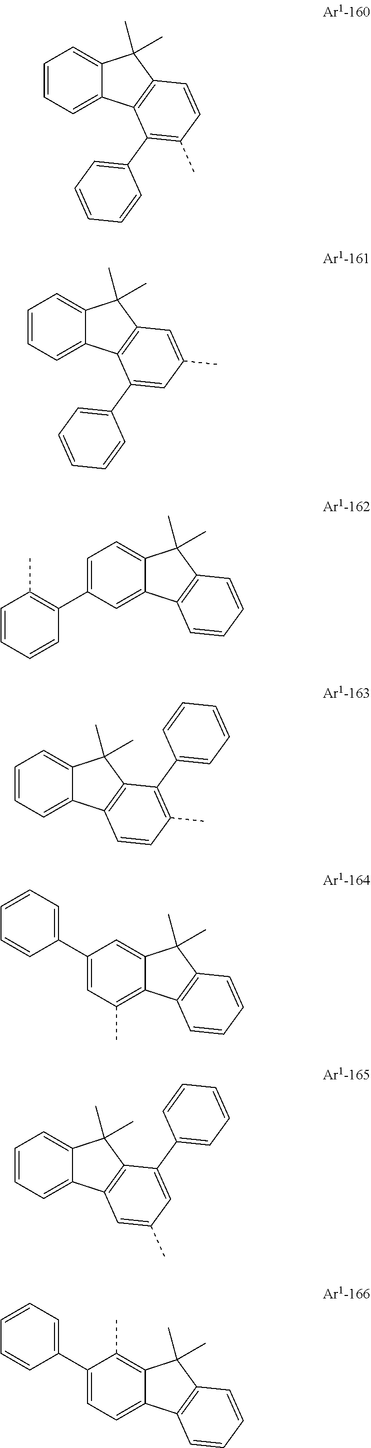

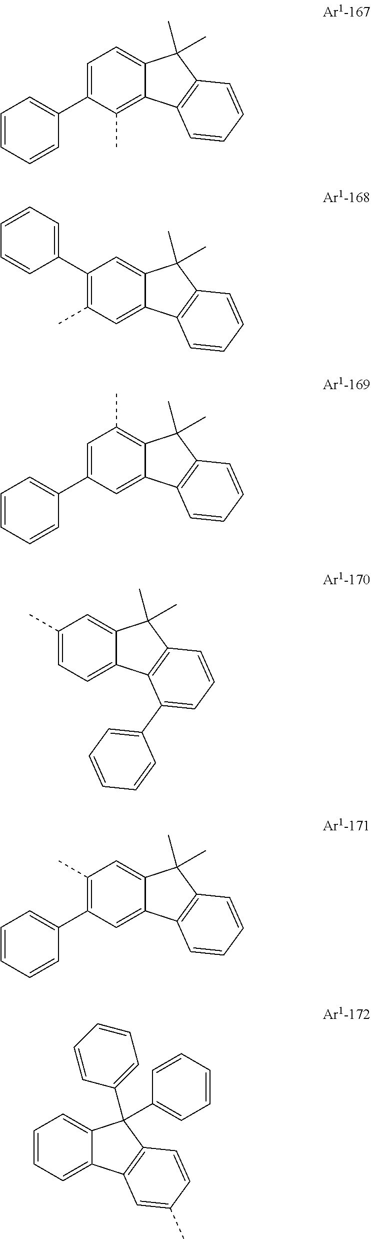

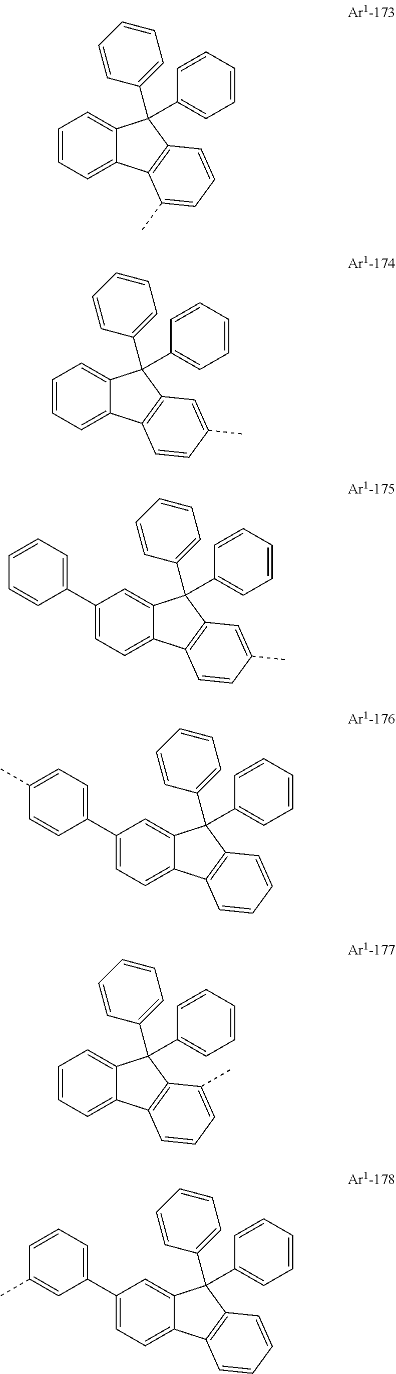

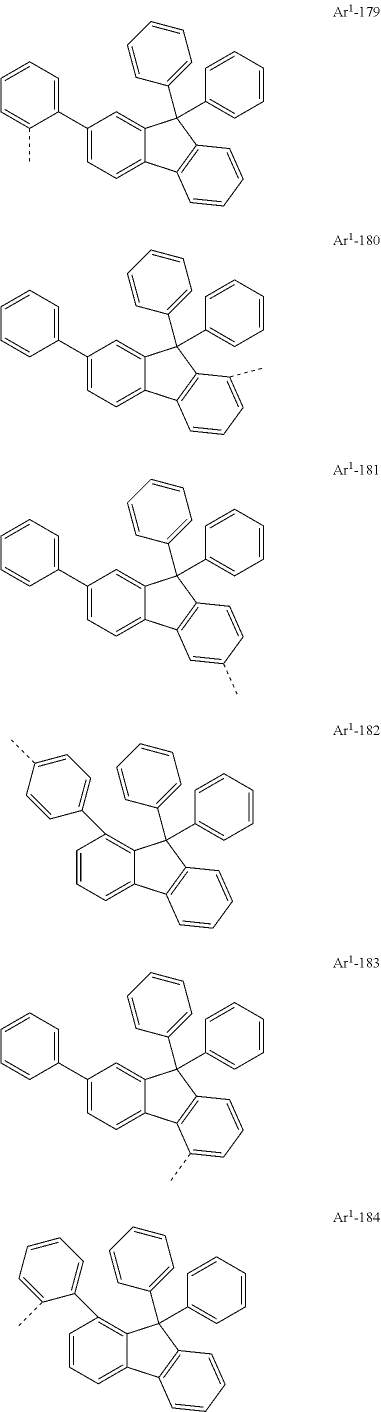

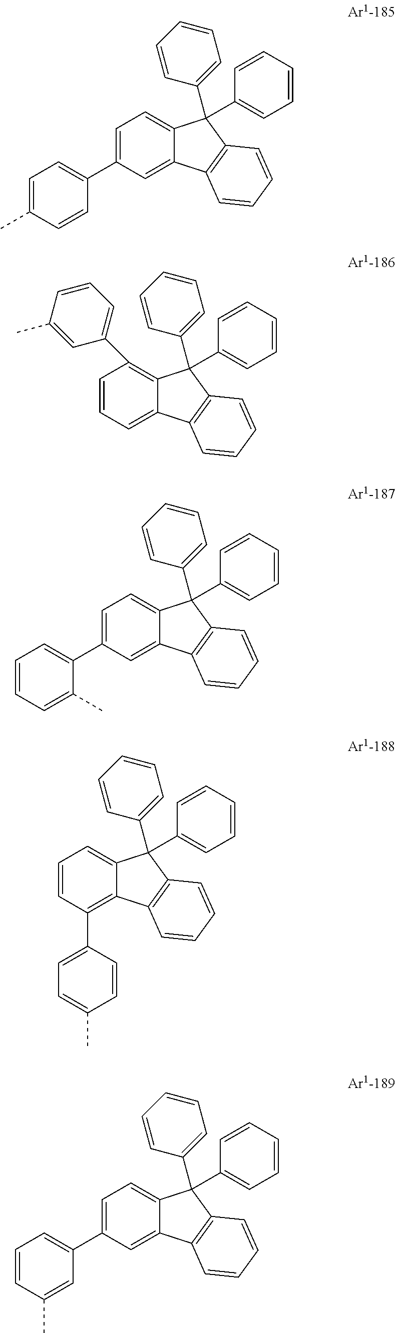

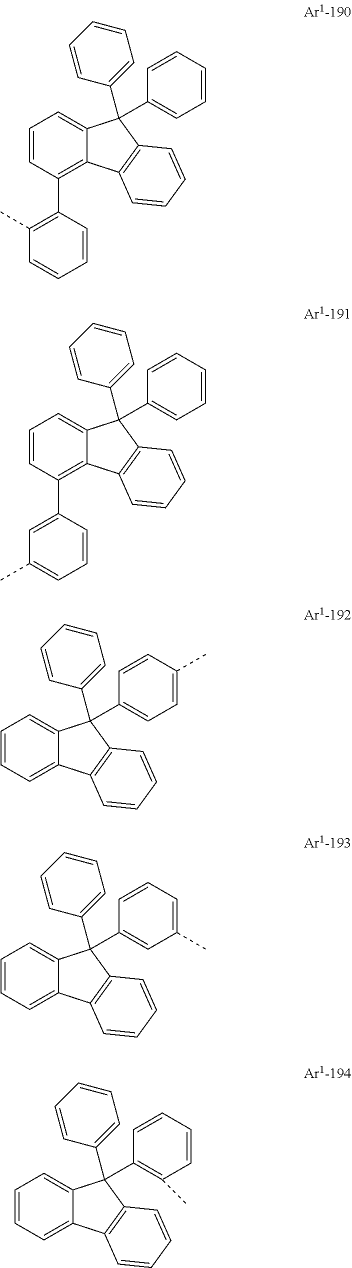

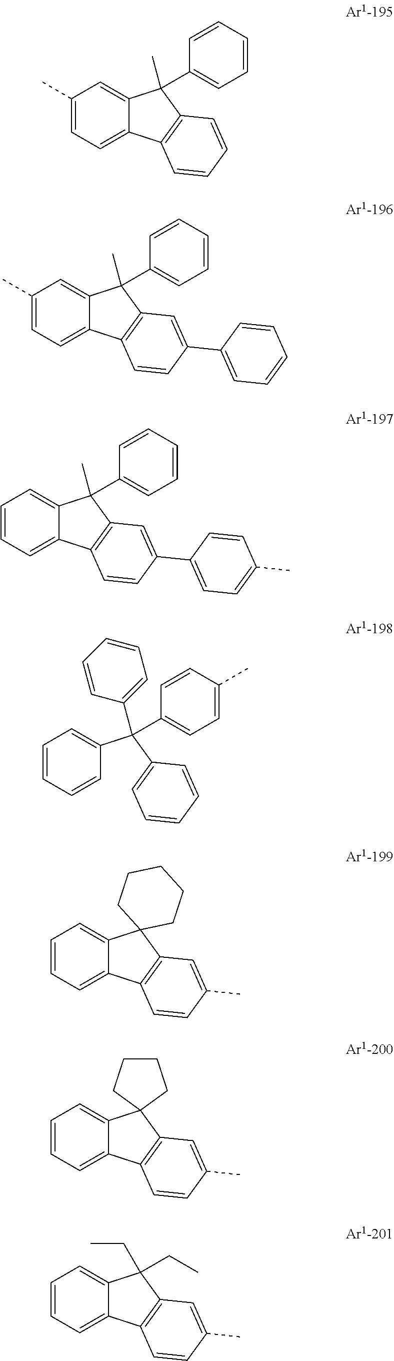

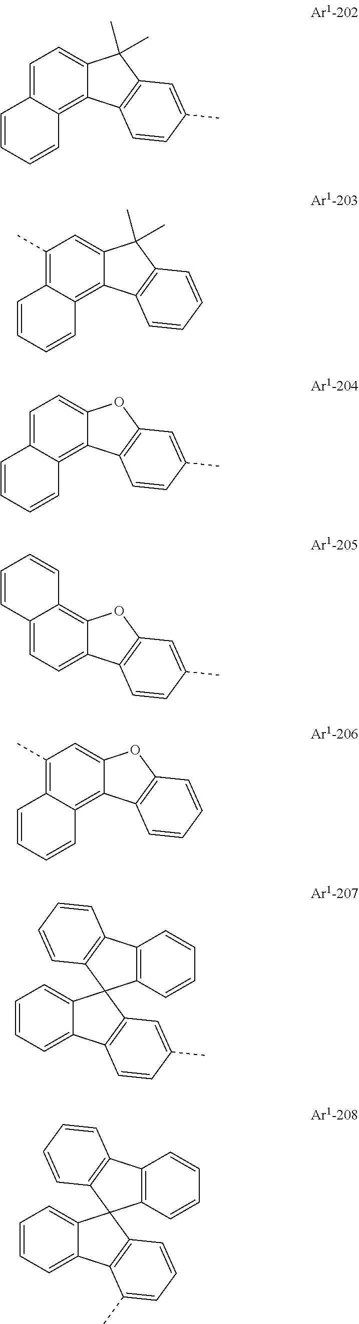

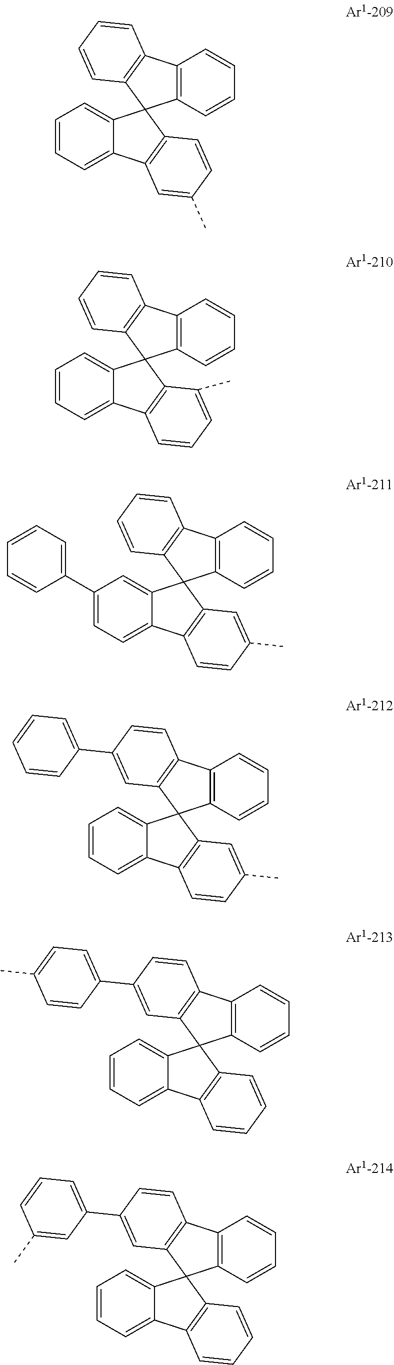

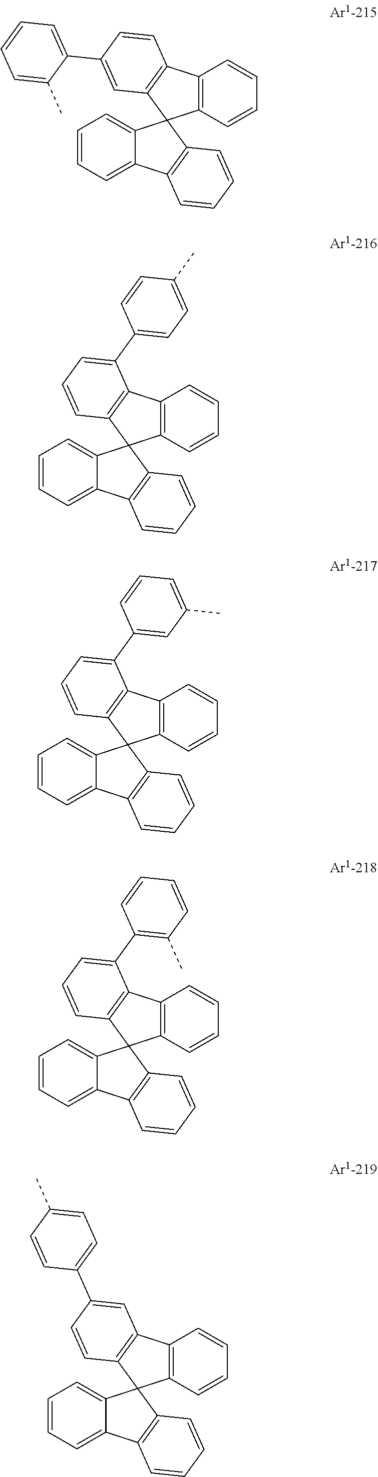

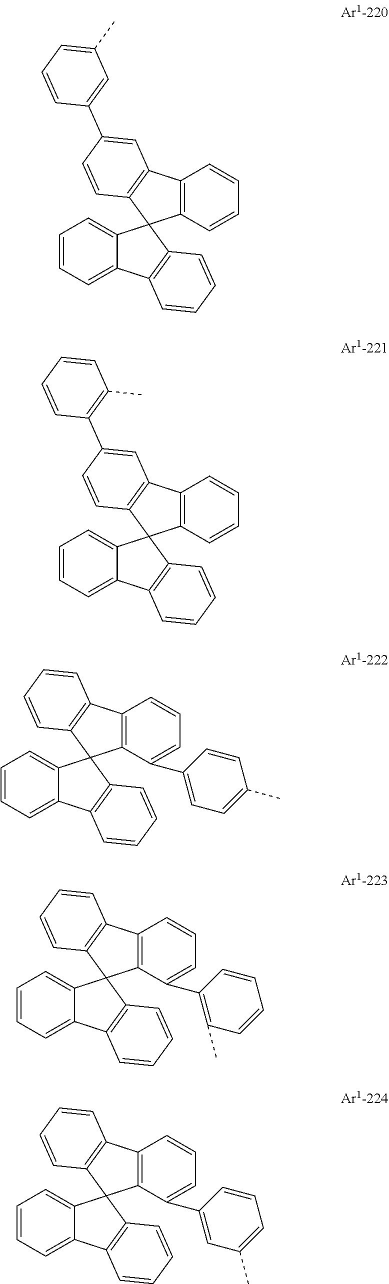

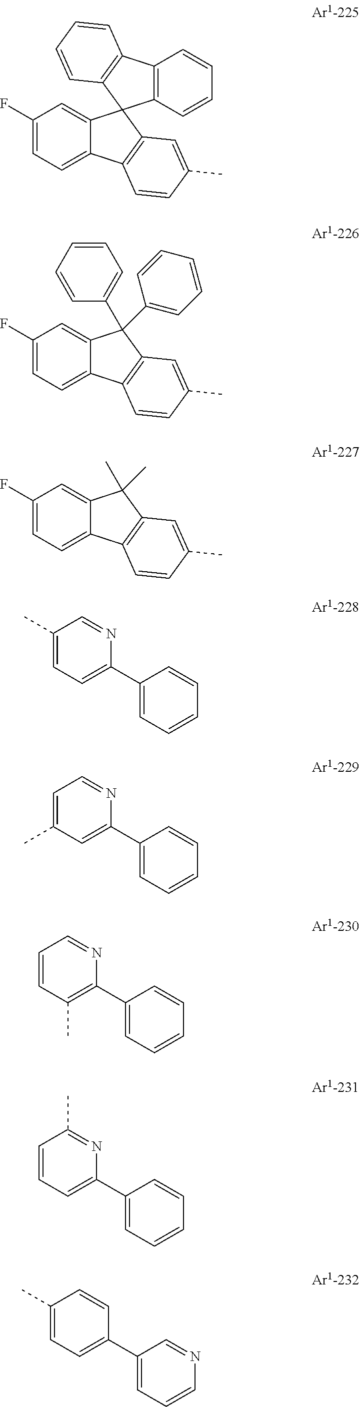

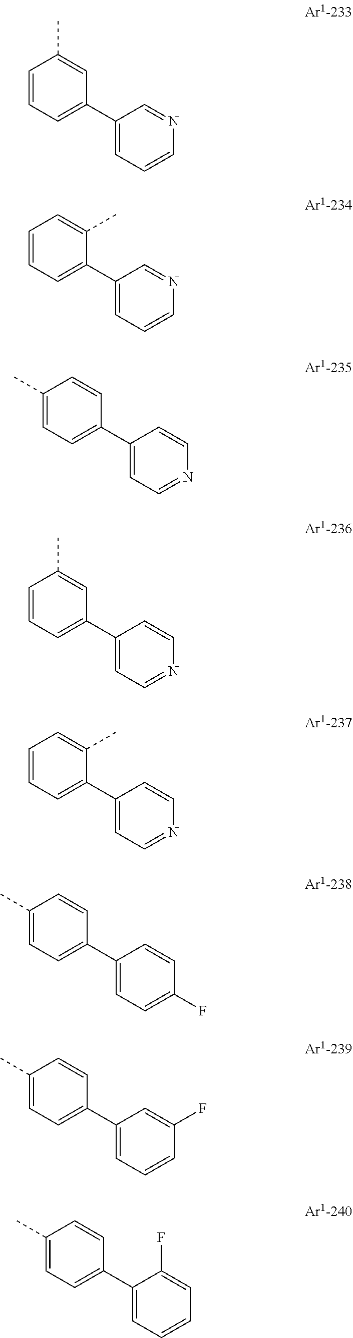

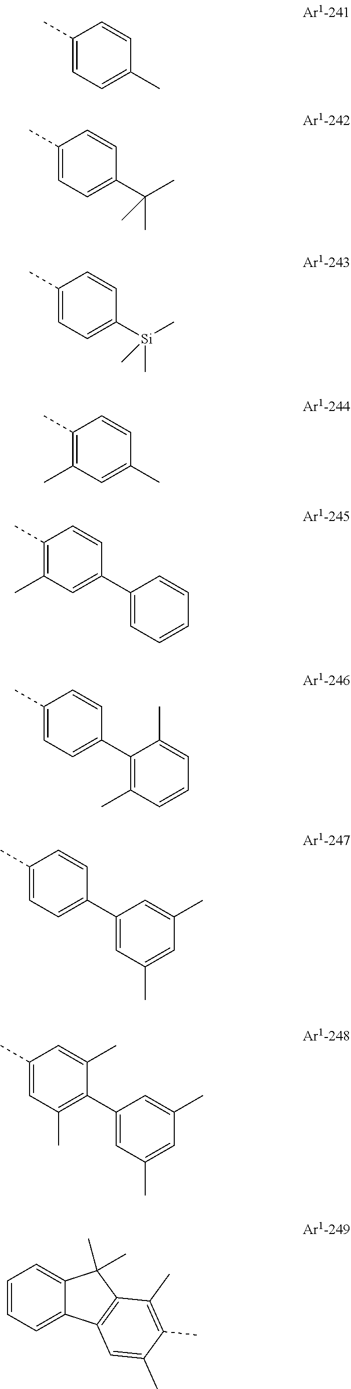

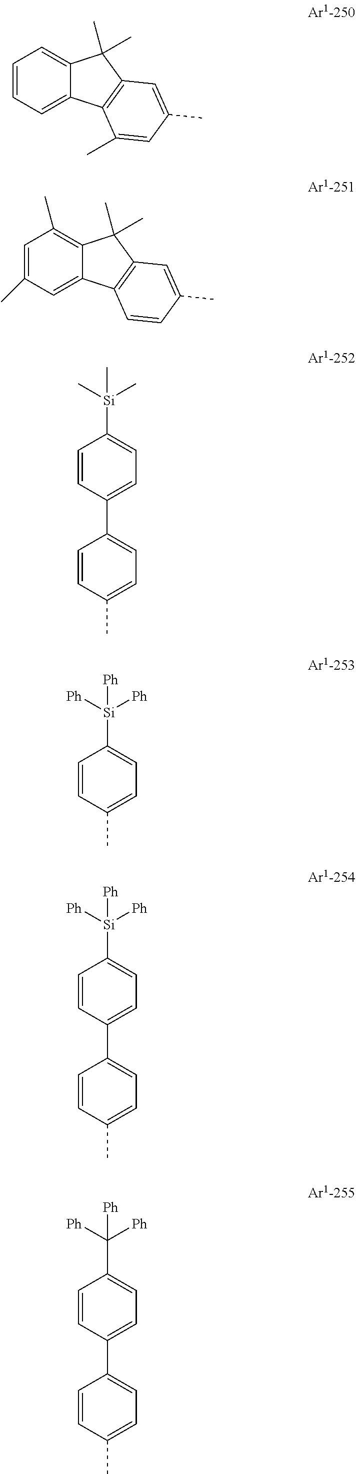

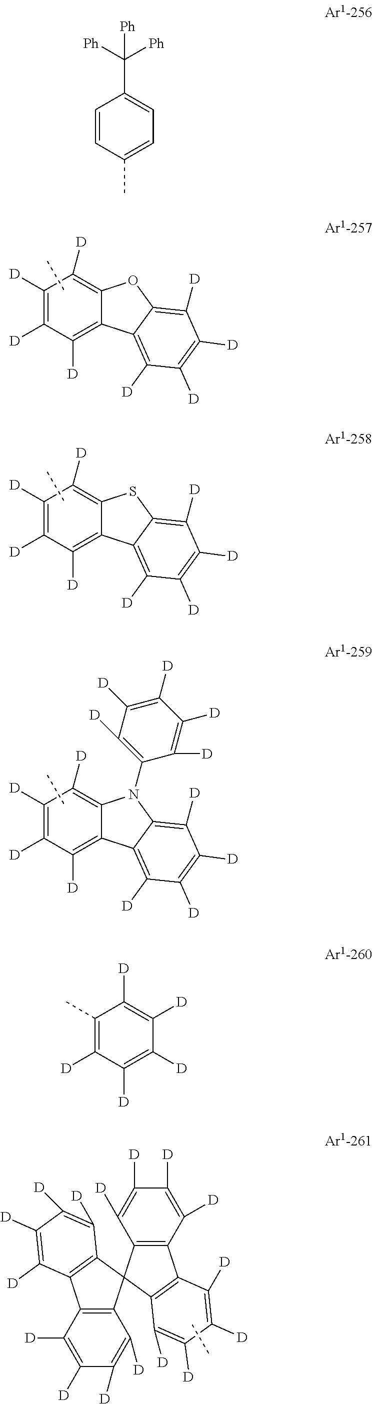

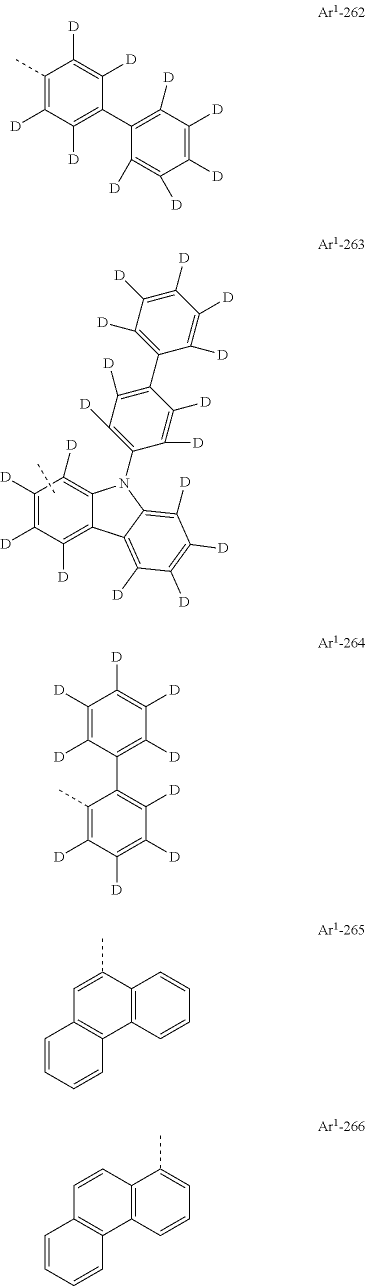

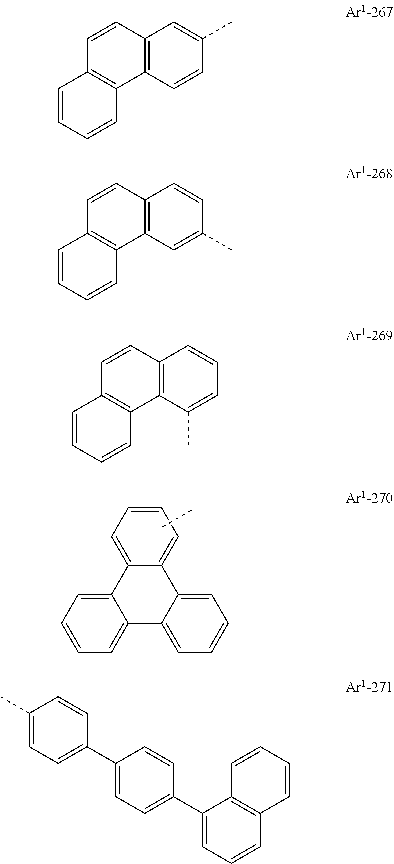

where the dotted line represents the bond to the nitrogen atom and where the groups at the positions shown as unsubstituted may be substituted by R³ radicals, and preferably have only H in the positions shown as unsubstituted. Among the abovementioned groups, preference is given to groups Ar¹-1 to Ar¹-106 and Ar¹-139 to Ar¹-271, and particular preference to groups Ar¹-2 to Ar¹-106 and Ar¹-139 to Ar¹-271. It is very particularly preferable when one or both of the Ar¹ groups, preferably both of the Ar¹ groups, are selected from Ar¹-2, Ar¹-5, Ar¹-48, Ar¹-50, Ar¹-63, Ar¹-64, Ar¹-65, Ar¹-66, Ar¹-74, Ar¹-78, Ar¹-140, Ar¹-141, Ar¹-144, Ar¹-149, Ar¹-193, Ar¹-195, Ar¹-265, Ar¹-266 Ar¹-268 and Ar¹-271 groups.

In the case that index k=0, both Ar¹ groups are preferably selected from groups of the formulae (Ar1-1) to (Ar1-10) as defined above, in which the bond to the nitrogen atom in formula (II) is labelled *.

Among the formulae (Ar1-1) to (Ar1-10), preference is given to the formulae (Ar1-1), (Ar1-6), (Ar1-7), (Ar1-8) and (Ar1-9). In an alternative preferred embodiment, if index k=0, at least one Ar¹ group is of the formula (Ar1-1). In an alternative preferred embodiment, if index k=0, at least one Ar¹ group is of a formula selected from formulae (Ar1-6), (Ar1-7), (Ar1-8) and (Ar1-9).

It is preferable when, if k=0, one or both of the Ar¹, preferably both of the Ar¹ groups, are selected from Ar¹-2, Ar¹-5, Ar¹-48, Ar¹-50, Ar¹-63, Ar¹-64, Ar¹-66, Ar¹-78, Ar¹-140, Ar¹-141, Ar¹-149, Ar¹-193, Ar¹-265, Ar¹-266 Ar¹-268 and Ar¹-271 groups.

If index k=0, the Ar¹ group not selected from groups of the formulae (Ar1-1) to (Ar1-10) is preferably selected from aromatic ring systems which have 6 to 25 aromatic ring atoms and are substituted by R³ radicals, and heteroaromatic ring systems which have 5 to 25 aromatic ring atoms and are substituted by R³ radicals. Especially preferred for this purpose are the Ar¹ groups mentioned above as preferred for when k=1, 2 or 3, excluding groups covered by one of the formulae (Ar1-1) to (Ar1-10).

In a preferred embodiment, the Ar¹ groups do not contain a carbazole group as a substituent R³, R⁶ or R⁷.

In a preferred embodiment, A is a group of the formula (A-1) bonded to L¹ via the bond marked * in the formula.

In a preferred embodiment, the formula (A-1) conforms to the following formula:

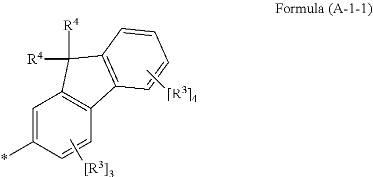

which is bonded to L¹ via the bond marked *.

In a preferred embodiment, the formula (A-2) conforms to the following formula:

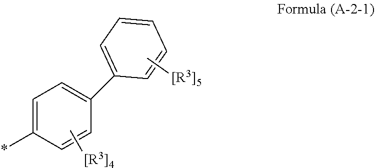

which is bonded to L¹ via the bond marked *.

In a preferred embodiment, V is the same or different at each instance and is selected from C(R³)₂ and a group

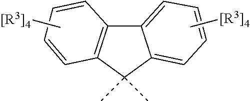

where the dotted bonds are the bonds to the radical of the formula Ar¹-3, Ar¹-4 or Ar¹-5. In a particularly preferred embodiment, V at each instance is C(R³)₂.

In a preferred embodiment, Y at each instance is O.

R¹ is preferably the same or different at each instance and is selected from H, D, F, CN, Si(R⁶)₃, N(R⁶)₂, straight-chain alkyl or alkoxy groups having 1 to 20 carbon atoms, branched or cyclic alkyl or alkoxy groups having 3 to 20 carbon atoms, aromatic ring systems having 6 to 40 aromatic ring atoms excluding fluorenyl, and heteroaromatic ring systems having 5 to 40 aromatic ring atoms; where the alkyl and alkoxy groups mentioned, the aromatic ring systems mentioned and the heteroaromatic ring systems mentioned are each substituted by R⁶ radicals; and where one or more CH₂ groups in the alkyl or alkoxy groups mentioned may be replaced by —C≡C—, —R⁶C═CR⁶—, Si(R⁶)₂, C═O, C═NR⁶, —NR⁶—, —O—, —S—, —C(═O)O— or —C(═O)NR⁶—. More preferably, R¹ is the same or different at each instance and is selected from H, D, F, CN, Si(R⁶)₃, straight-chain alkyl groups having 1 to 20 carbon atoms, branched or cyclic alkyl groups having 3 to 20 carbon atoms, aryl groups having 6 to 25, preferably 6 to 14, aromatic ring atoms, and heteroaryl groups having 5 to 40 aromatic ring atoms, where said alkyl groups, said aryl groups and said heteroaryl groups are each substituted by R⁶ radicals.

Preferably, in the compounds of one of the formulae (I) to (III), zero, one, two or three R¹ groups per formula are not H or D. These groups that are not H or D are preferably selected from F, CN, Si(R⁶)₃, straight-chain alkyl groups having 1 to 20 carbon atoms, branched or cyclic alkyl groups having 3 to 20 carbon atoms, aryl groups having 6 to 25, preferably 6 to 14, aromatic ring atoms, and heteroaryl groups having 5 to 40 aromatic ring atoms, where said alkyl groups, said aryl groups and said heteroaryl groups are each substituted by R⁶ radicals.

The compounds of one of the formulae (I) to (III) preferably have at least one R¹ group selected from aromatic ring systems which have 6 to 40 aromatic ring atoms, excluding fluorenyl, and are substituted by R⁶ radicals; the compounds of one of the formulae (I) to (III) more preferably have at least one R¹ group selected from aryl groups having 6 to 25, preferably 6 to 14, aromatic ring atoms substituted by R⁶ radicals.

In a particularly preferred embodiment, the compounds of one of the formulae (I) to (III) have at least one R¹ group that is a phenyl groups substituted by R⁶ radicals.

In an alternative preferred embodiment, all R¹ radicals in formulae (I) to (III) are H or D, more preferably H.

R² is preferably the same or different at each instance and is selected from H, D, F, CN, Si(R⁶)₃, N(R⁶)₂, straight-chain alkyl or alkoxy groups having 1 to 20 carbon atoms, branched or cyclic alkyl or alkoxy groups having 3 to 20 carbon atoms, aromatic ring systems having 6 to 40 aromatic ring atoms and heteroaromatic ring systems having 5 to 40 aromatic ring atoms; where the alkyl and alkoxy groups mentioned, the aromatic ring systems mentioned and the heteroaromatic ring systems mentioned are each substituted by R⁶ radicals; and where one or more CH₂ groups in the alkyl or alkoxy groups mentioned may be replaced by —C≡C—, —R⁶C═CR⁶—, Si(R⁶)₂, C═O, C═NR⁶, —NR⁶—, —O—, —S—, —C(═O)O— or —C(═O)NR⁶—.

R³ is preferably the same or different at each instance and is selected from H, D, F, CN, Si(R⁶)₃, N(R⁶)₂, straight-chain alkyl or alkoxy groups having 1 to 20 carbon atoms, branched or cyclic alkyl or alkoxy groups having 3 to 20 carbon atoms, aromatic ring systems having 6 to 40 aromatic ring atoms and heteroaromatic ring systems having 5 to 40 aromatic ring atoms; where the alkyl and alkoxy groups mentioned, the aromatic ring systems mentioned and the heteroaromatic ring systems mentioned are each substituted by R⁶ radicals; and where one or more CH₂ groups in the alkyl or alkoxy groups mentioned may be replaced by —C≡C—, —R⁶C═CR⁶—, Si(R⁶)₂, C═O, C═NR⁶, —NR⁶—, —O—, —S—, —C(═O)O— or —C(═O)NR⁶—.

It is preferable that R⁴ is the same or different at each instance and is selected from straight-chain alkyl or alkoxy groups having 1 to 20 carbon atoms, branched or cyclic alkyl or alkoxy groups having 3 to 20 carbon atoms, and alkenyl or alkynyl groups having 2 to 20 carbon atoms; where any two R⁴ radicals may be joined to one another and may form a ring; where the alkyl, alkoxy, alkenyl and alkynyl groups mentioned are each substituted by R⁶ radicals; and where one or more CH₂ groups in the alkyl, alkoxy, alkenyl and alkynyl groups mentioned may be replaced by —R⁶C═CR⁶—, —C≡C—, Si(R⁶)₂, C═O, C═NR⁶, —C(═O)O—, —C(═O)NR⁶—, NR⁶, P(═O)(R⁶), —O—, —S—, SO or SO₂.

More preferably, R⁴ is the same or different at each instance and is selected from straight-chain alkyl groups which have 1 to 20 carbon atoms and are each substituted by R⁶ radicals, and branched or cyclic alkyl groups which have 3 to 20 carbon atoms and are each substituted by R⁶ radicals; where any two R⁴ radicals may be joined to one another and may form a ring. Even more preferably, the alkyl groups mentioned are unsubstituted, meaning that R⁶ in these cases is H or D, preferably H. Most preferably, R⁴ is the same or different, preferably the same, and is selected from methyl, ethyl, n-propyl, isopropyl and tert-butyl, or two R⁴ radicals bonded to the same carbon atom are joined to form a cyclohexyl or cyclopentyl group.

In a preferred embodiment, two R⁴ radicals bonded to the same carbon atom are selected to be the same. In this case, it is preferable that these two identical R⁴ are selected from straight-chain alkyl or alkoxy groups having 1 to 20 carbon atoms, branched or cyclic alkyl or alkoxy groups having 3 to 20 carbon atoms, and alkenyl or alkynyl groups having 2 to 20 carbon atoms; where any two R⁴ radicals may be joined to one another and may form a ring; where the alkyl, alkoxy, alkenyl and alkynyl groups mentioned are each substituted by R⁶ radicals; and where one or more CH₂ groups in the alkyl, alkoxy, alkenyl and alkynyl groups mentioned may be replaced by —R⁶C═CR⁶—, —C≡C—, Si(R⁶)₂, C═O, C═NR⁶, —C(═O)O—, —C(═O)NR⁶—, NR⁶, P(═O)(R⁶), —O—, —S—, SO or SO₂. In this case, it is particularly preferable that the two identical R⁴ are selected from straight-chain alkyl groups which have 1 to 20 carbon atoms and are each substituted by R⁶ radicals, and branched or cyclic alkyl groups which have 3 to 20 carbon atoms and are each substituted by R⁶ radicals; where any two R⁴ radicals may be joined to one another and may form a ring. In that case, the alkyl groups mentioned are preferably unsubstituted, meaning that R⁶ in these cases is H or D, preferably H. Even more preferably in that case, R⁴ is the same or different, preferably the same, and is selected from methyl, ethyl, n-propyl, isopropyl and tert-butyl, or two R⁴ radicals bonded to the same carbon atom are joined to form a cyclohexyl or cyclopentyl group.

In an alternative preferred embodiment, two R⁴ radicals bonded to the same carbon atom may be selected differently. In this case, the abovementioned preferred embodiments for R⁴ are applicable.

R⁵ is preferably the same or different at each instance and is selected from H, D, F, CN, Si(R⁶)₃, N(R⁶)₂, straight-chain alkyl or alkoxy groups having 1 to 20 carbon atoms, branched or cyclic alkyl or alkoxy groups having 3 to 20 carbon atoms, aromatic ring systems having 6 to 40 aromatic ring atoms and heteroaromatic ring systems having 5 to 40 aromatic ring atoms; where the alkyl and alkoxy groups mentioned, the aromatic ring systems mentioned and the heteroaromatic ring systems mentioned are each substituted by R⁶ radicals; and where one or more CH₂ groups in the alkyl or alkoxy groups mentioned may be replaced by —C≡C—, —R⁶C═CR⁶—, Si(R⁶)₂, C═O, C═NR⁶, —NR⁶—, —O—, —S—, —C(═O)O— or —C(═O)NR⁶—.

R⁶ is preferably the same or different at each instance and is selected from H, D, F, CN, Si(R⁷)₃, N(R⁷)₂, straight-chain alkyl or alkoxy groups having 1 to 20 carbon atoms, branched or cyclic alkyl or alkoxy groups having 3 to 20 carbon atoms, aromatic ring systems having 6 to 40 aromatic ring atoms and heteroaromatic ring systems having 5 to 40 aromatic ring atoms; where the alkyl and alkoxy groups mentioned, the aromatic ring systems mentioned and the heteroaromatic ring systems mentioned are each substituted by R⁷ radicals; and where one or more CH₂ groups in the alkyl or alkoxy groups mentioned may be replaced by —C≡C—, —R⁷C═CR⁷—, Si(R⁷)₂, C═O, C═NR⁷, —NR⁷—, —O—, —S—, —C(═O)O— or —C(═O)NR⁷—.

In a preferred embodiment, index k is selected from 0 and 1; in a particularly preferred embodiment, index k is 0.

Formula (I) preferably conforms to one of the following formulae:

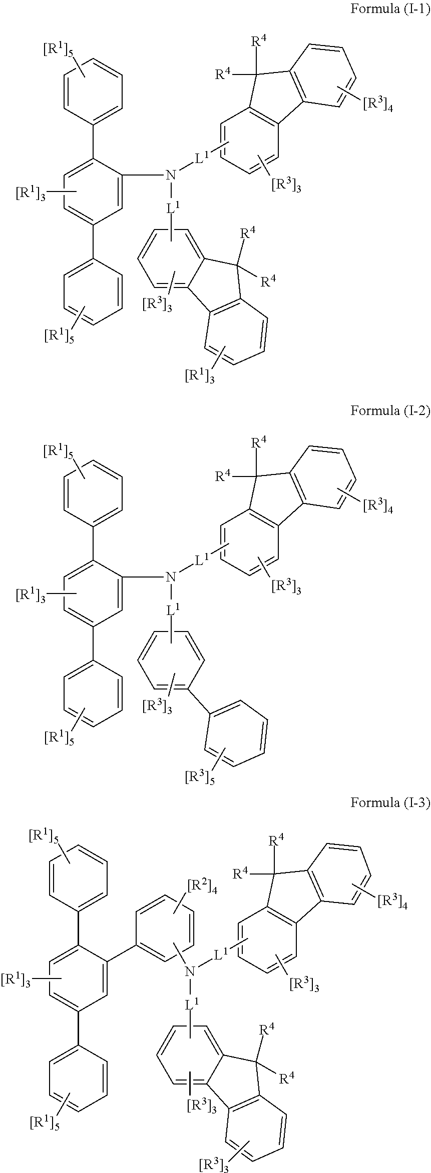

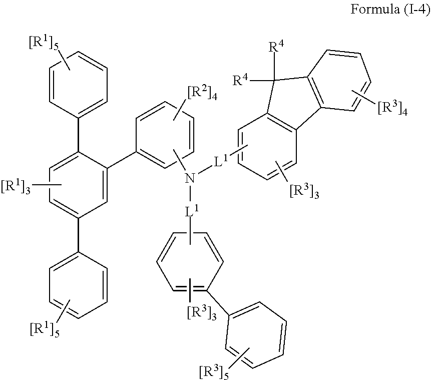

where the groups that occur are as defined above, and L¹ at each instance is preferably a single bond.

Preferred embodiments of the abovementioned formulae conform to the following formulae:

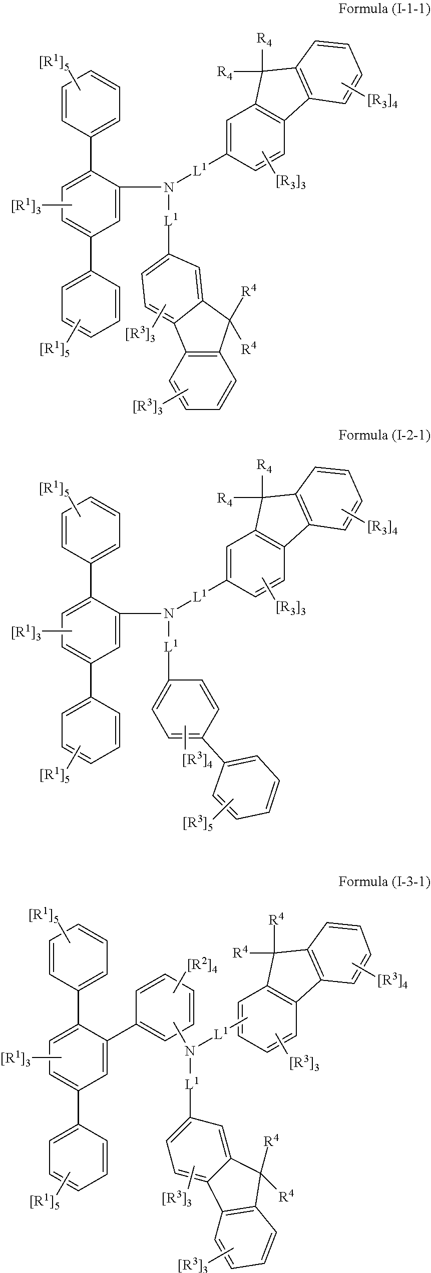

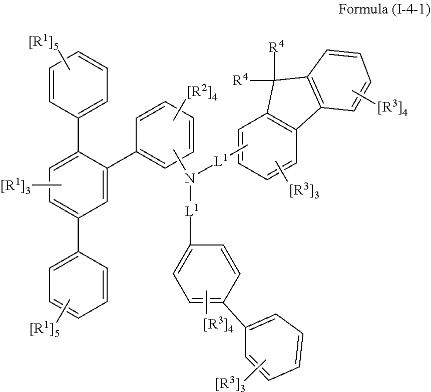

where the groups that occur are as defined above, and L¹ at each instance is preferably a single bond.

Formula (II) preferably conforms to one of the following formulae:

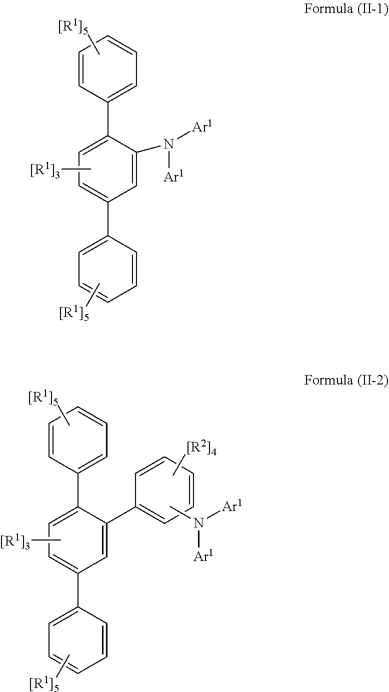

wherein the groups that occur are as defined above.

Formula (II-2) preferably conforms to one of the following formulae:

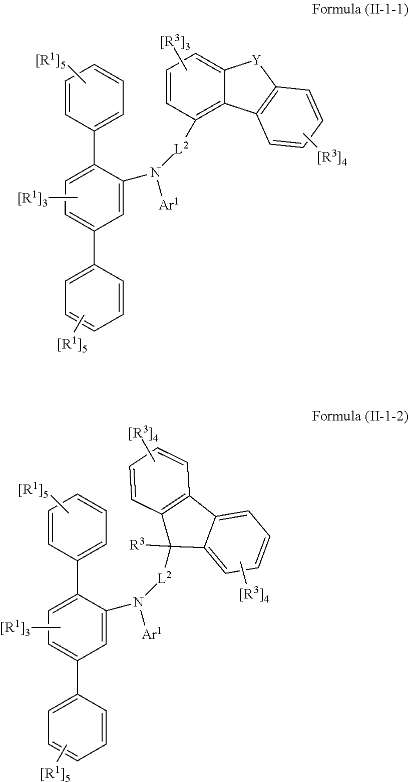

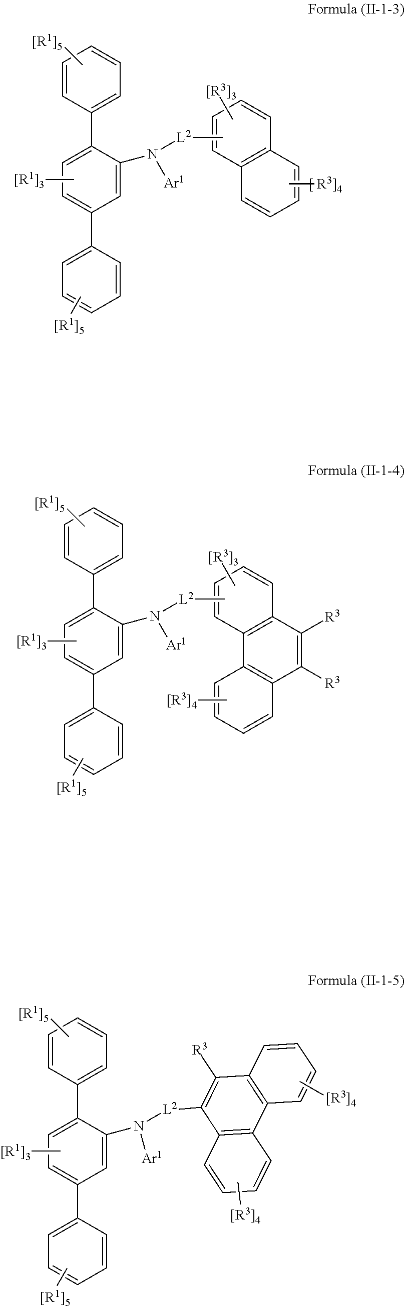

where the groups that occur are as defined above, and where Ar¹ is selected from aromatic ring systems which have 6 to 40 aromatic ring atoms and are substituted by R³ radicals, and heteroaromatic ring systems which have 5 to 40 aromatic ring atoms and are substituted by R³ radicals.

Preferred embodiments of the formula (III) are the following formulae:

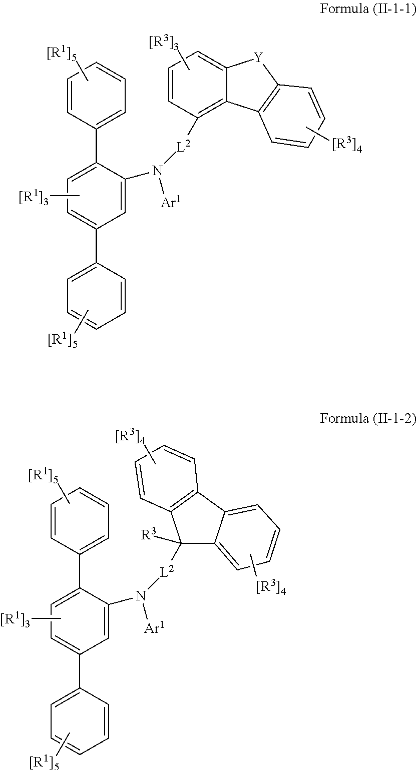

wherein the groups that occur are as defined above.

Preferred embodiments of the formula (III) are the following formulae

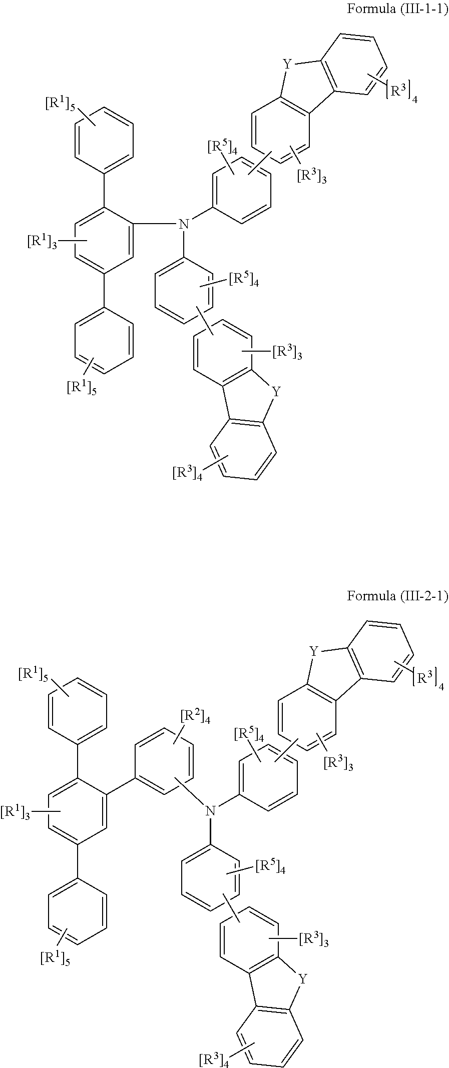

wherein the groups that occur are as defined above.

Preferred embodiments of the formula (III) are the following formulae:

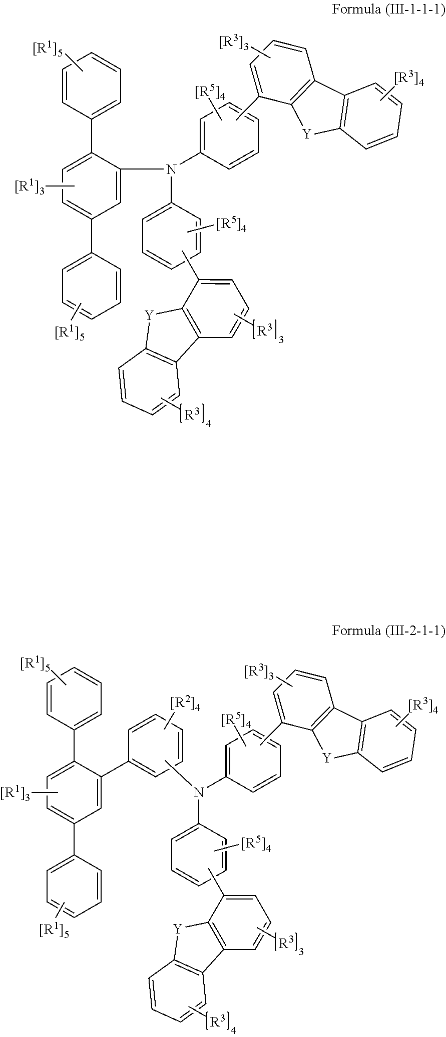

wherein the groups that occur are as defined above.

In a preferred embodiment, the unit

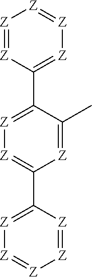

in formulae (I), (II) and (III) has one of the following structures:

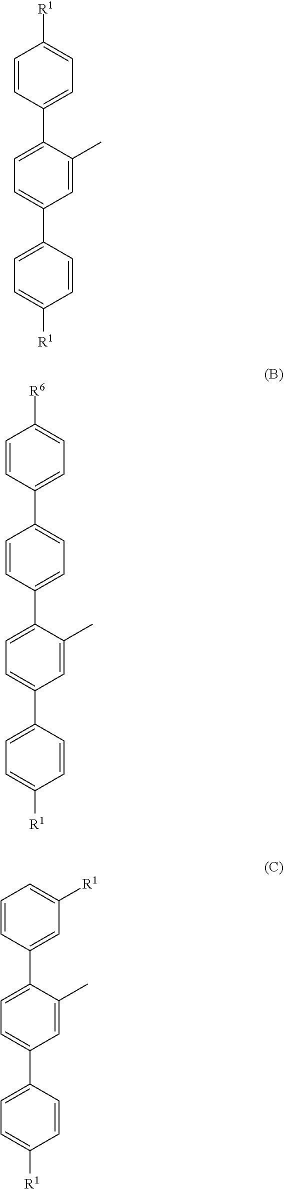

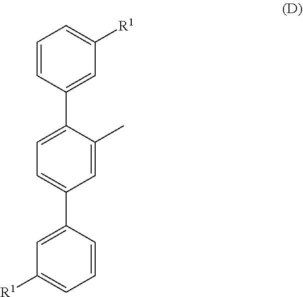

where R¹ and R⁶ are as defined above, and preferably are defined as above in the preferred embodiments stated. More preferably, R¹ and R⁶ here are H. Among the embodiments (A) to (D), particular preference is given to embodiments (A) and (B), especially when R¹ and R⁶ are H.

Preferred compounds according to the present application are shown below:

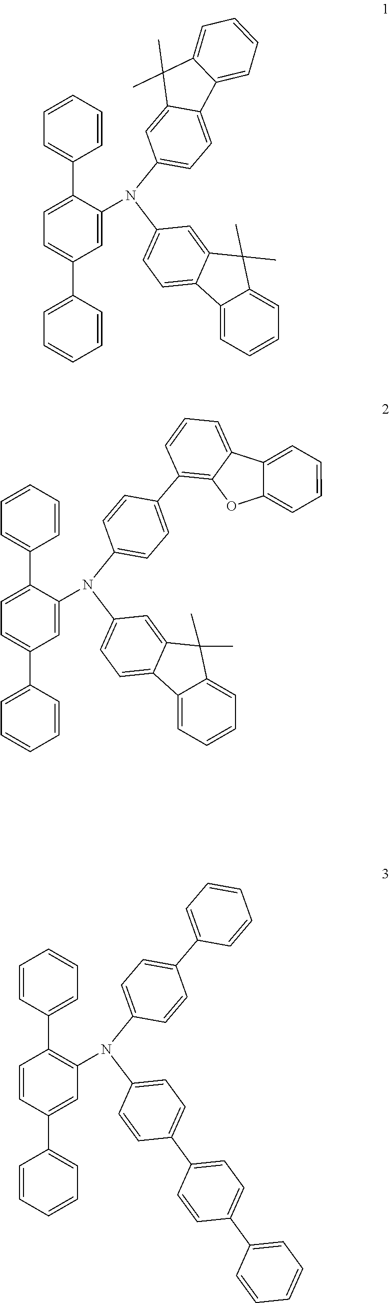 [SMILES: CC1(C)c2ccccc2-c2ccc(N(c3ccc4c(c3)C(C)(C)c3ccccc3-4)c3cc(-c4ccccc4)ccc3-c3ccccc3)cc21 ; CC1(C)c2ccccc2-c2ccc(N(c3ccc(-c4cccc5c4oc4ccccc45)cc3)c3cc(-c4ccccc4)ccc3-c3ccccc3)cc21 ; c1ccc(-c2ccc(-c3ccc(N(c4ccc(-c5ccccc5)cc4)c4cc(-c5ccccc5)ccc4-c4ccccc4)cc3)cc2)cc1]

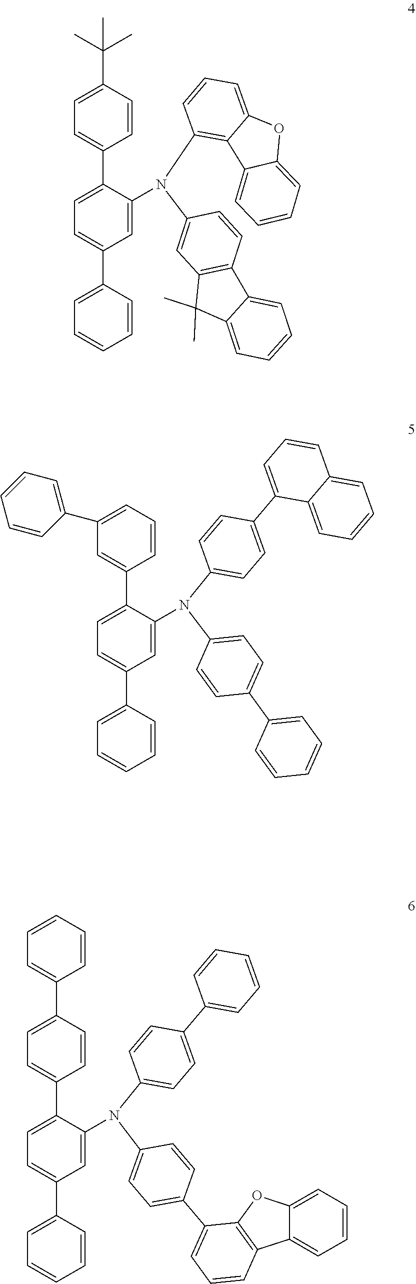 [SMILES: CC(C)(C)c1ccc(-c2ccc(-c3ccccc3)cc2N(c2ccc3c(c2)C(C)(C)c2ccccc2-3)c2cccc3oc4ccccc4c23)cc1 ; c1ccc(-c2ccc(N(c3ccc(-c4cccc5ccccc45)cc3)c3cc(-c4ccccc4)ccc3-c3cccc(-c4ccccc4)c3)cc2)cc1 ; c1ccc(-c2ccc(-c3ccc(-c4ccccc4)cc3N(c3ccc(-c4ccccc4)cc3)c3ccc(-c4cccc5c4oc4ccccc45)cc3)cc2)cc1]

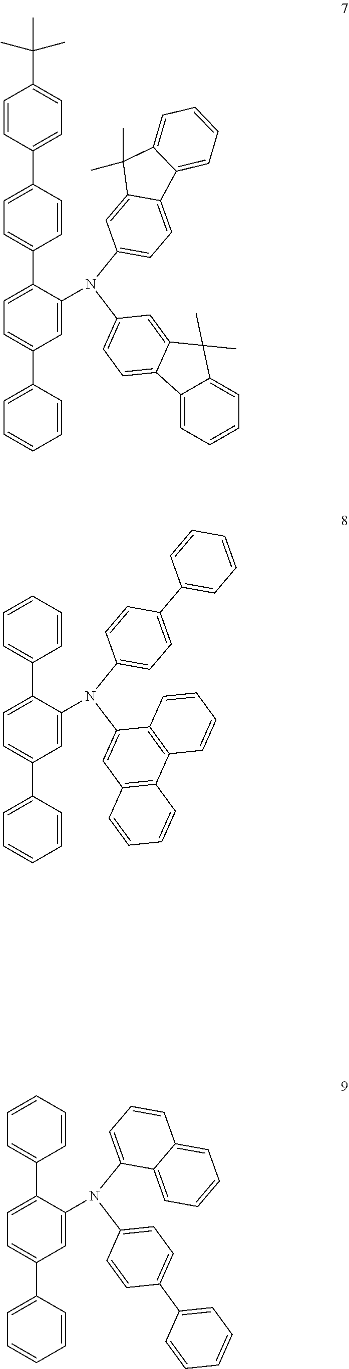 [SMILES: CC(C)(C)c1ccc(-c2ccc(-c3ccc(-c4ccccc4)cc3N(c3ccc4c(c3)C(C)(C)c3ccccc3-4)c3ccc4c(c3)C(C)(C)c3ccccc3-4)cc2)cc1 ; c1ccc(-c2ccc(N(c3cc(-c4ccccc4)ccc3-c3ccccc3)c3cc4ccccc4c4ccccc34)cc2)cc1 ; c1ccc(-c2ccc(N(c3cc(-c4ccccc4)ccc3-c3ccccc3)c3cccc4ccccc34)cc2)cc1]

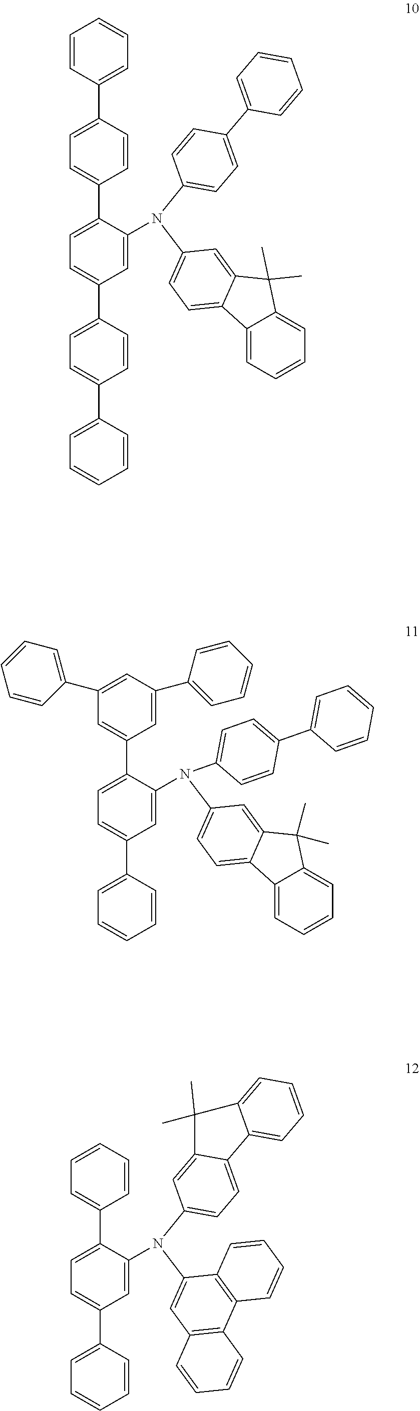 [SMILES: CC1(C)c2ccccc2-c2ccc(N(c3ccc(-c4ccccc4)cc3)c3cc(-c4ccc(-c5ccccc5)cc4)ccc3-c3ccc(-c4ccccc4)cc3)cc21 ; CC1(C)c2ccccc2-c2ccc(N(c3ccc(-c4ccccc4)cc3)c3cc(-c4ccccc4)ccc3-c3cc(-c4ccccc4)cc(-c4ccccc4)c3)cc21 ; CC1(C)c2ccccc2-c2ccc(N(c3cc(-c4ccccc4)ccc3-c3ccccc3)c3cc4ccccc4c4ccccc34)cc21]

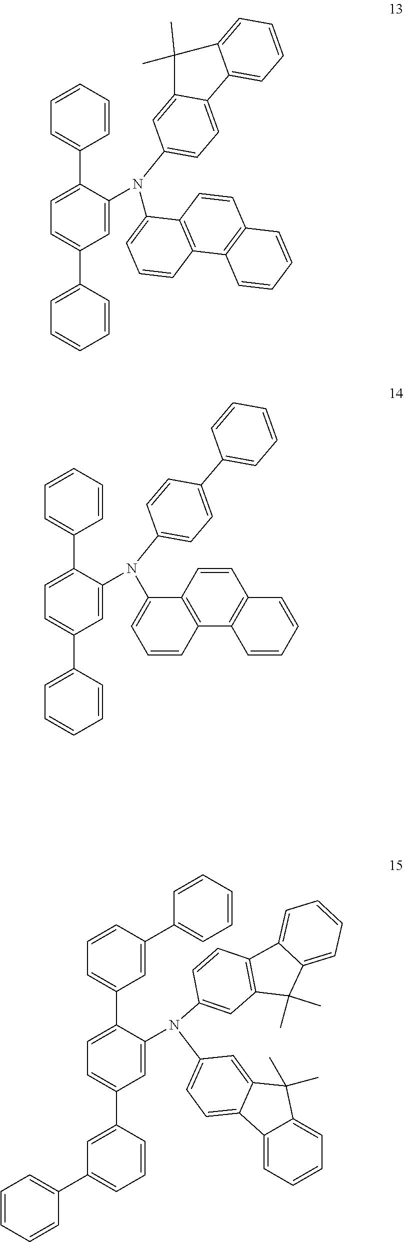 [SMILES: CC1(C)c2ccccc2-c2ccc(N(c3cc(-c4ccccc4)ccc3-c3ccccc3)c3cccc4c3ccc3ccccc34)cc21 ; c1ccc(-c2ccc(N(c3cc(-c4ccccc4)ccc3-c3ccccc3)c3cccc4c3ccc3ccccc34)cc2)cc1 ; CC1(C)c2ccccc2-c2ccc(N(c3ccc4c(c3)C(C)(C)c3ccccc3-4)c3cc(-c4cccc(-c5ccccc5)c4)ccc3-c3cccc(-c4ccccc4)c3)cc21]

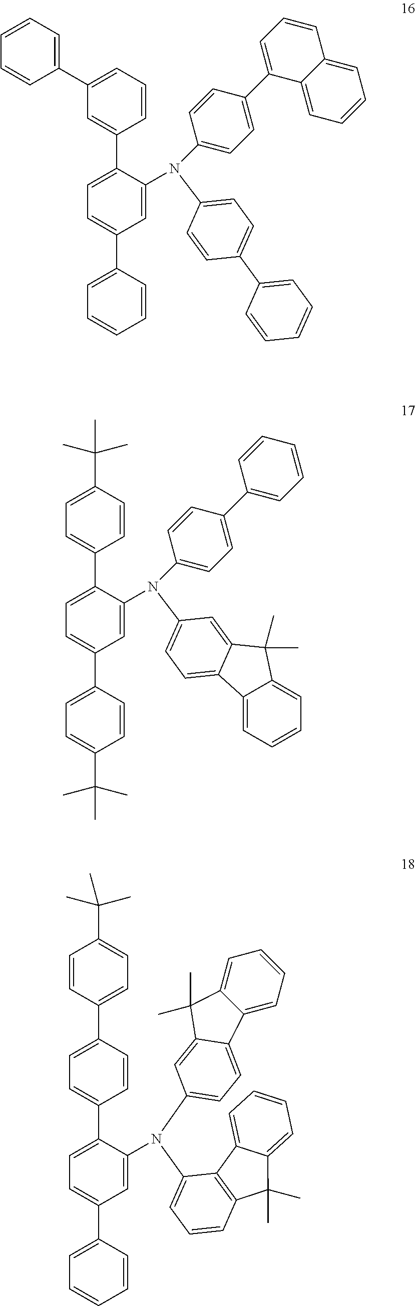 [SMILES: c1ccc(-c2ccc(N(c3ccc(-c4cccc5ccccc45)cc3)c3cc(-c4ccccc4)ccc3-c3cccc(-c4ccccc4)c3)cc2)cc1 ; CC(C)(C)c1ccc(-c2ccc(-c3ccc(C(C)(C)C)cc3)c(N(c3ccc(-c4ccccc4)cc3)c3ccc4c(c3)C(C)(C)c3ccccc3-4)c2)cc1 ; CC(C)(C)c1ccc(-c2ccc(-c3ccc(-c4ccccc4)cc3N(c3ccc4c(c3)C(C)(C)c3ccccc3-4)c3cccc4c3-c3ccccc3C4(C)C)cc2)cc1]

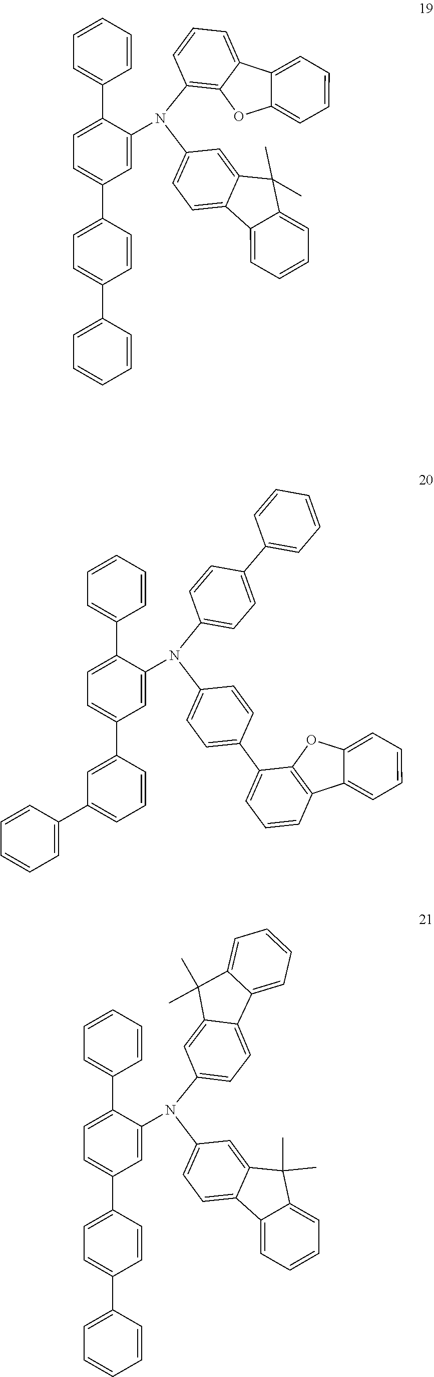 [SMILES: CC1(C)c2ccccc2-c2ccc(N(c3cc(-c4ccc(-c5ccccc5)cc4)ccc3-c3ccccc3)c3cccc4c3oc3ccccc34)cc21 ; c1ccc(-c2ccc(N(c3ccc(-c4cccc5c4oc4ccccc45)cc3)c3cc(-c4cccc(-c5ccccc5)c4)ccc3-c3ccccc3)cc2)cc1 ; CC1(C)c2ccccc2-c2ccc(N(c3ccc4c(c3)C(C)(C)c3ccccc3-4)c3cc(-c4ccc(-c5ccccc5)cc4)ccc3-c3ccccc3)cc21]

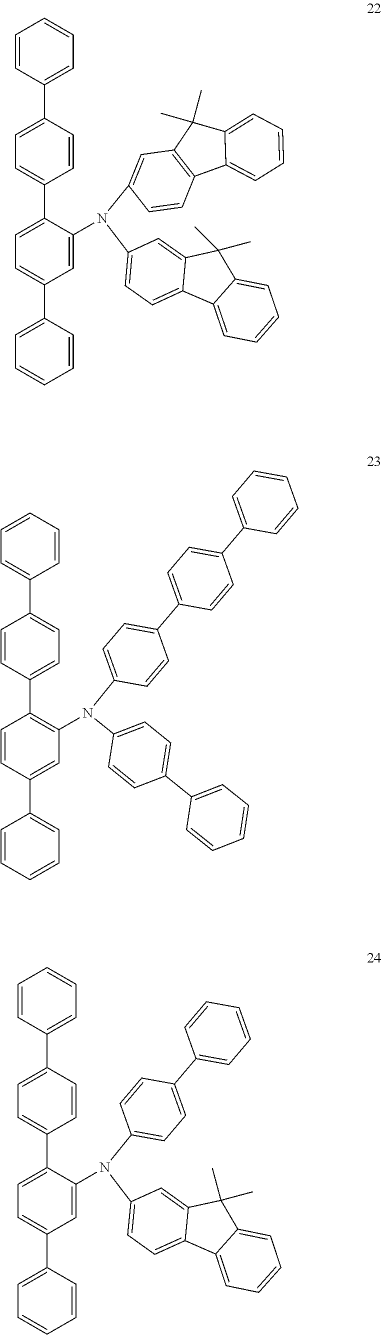 [SMILES: CC1(C)c2ccccc2-c2ccc(N(c3ccc4c(c3)C(C)(C)c3ccccc3-4)c3cc(-c4ccccc4)ccc3-c3ccc(-c4ccccc4)cc3)cc21 ; c1ccc(-c2ccc(-c3ccc(N(c4ccc(-c5ccccc5)cc4)c4cc(-c5ccccc5)ccc4-c4ccc(-c5ccccc5)cc4)cc3)cc2)cc1 ; CC1(C)c2ccccc2-c2ccc(N(c3ccc(-c4ccccc4)cc3)c3cc(-c4ccccc4)ccc3-c3ccc(-c4ccccc4)cc3)cc21]

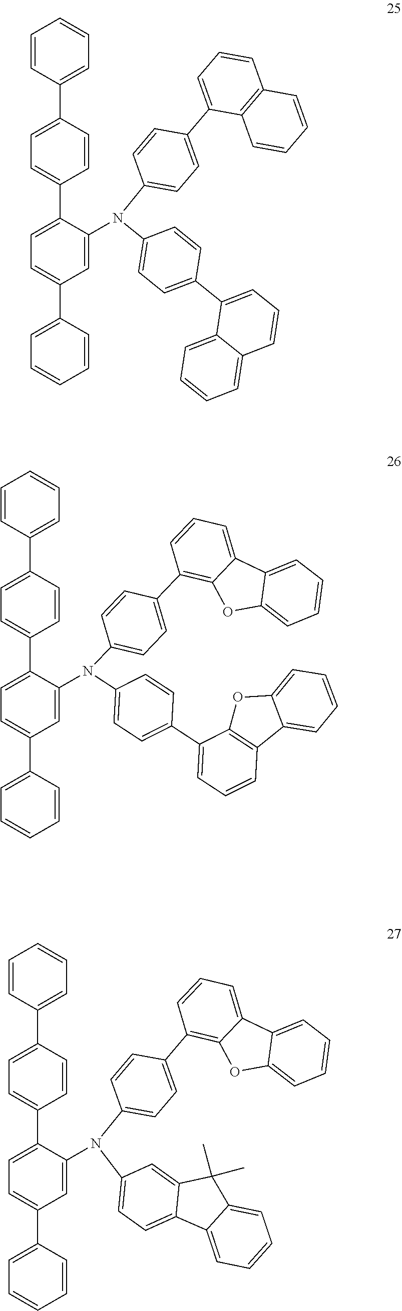 [SMILES: c1ccc(-c2ccc(-c3ccc(-c4ccccc4)cc3N(c3ccc(-c4cccc5ccccc45)cc3)c3ccc(-c4cccc5ccccc45)cc3)cc2)cc1 ; c1ccc(-c2ccc(-c3ccc(-c4ccccc4)cc3N(c3ccc(-c4cccc5c4oc4ccccc45)cc3)c3ccc(-c4cccc5c4oc4ccccc45)cc3)cc2)cc1 ; CC1(C)c2ccccc2-c2ccc(N(c3ccc(-c4cccc5c4oc4ccccc45)cc3)c3cc(-c4ccccc4)ccc3-c3ccc(-c4ccccc4)cc3)cc21]

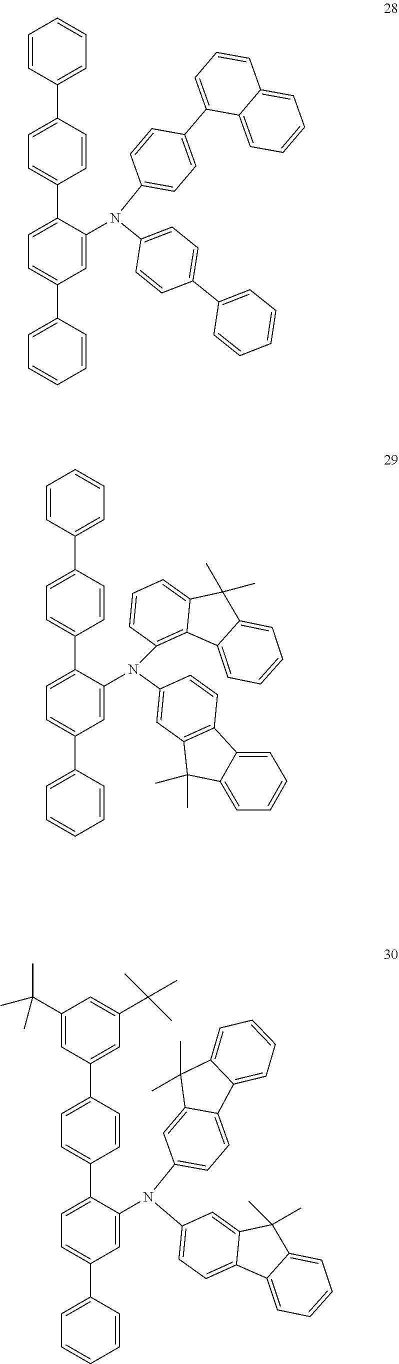 [SMILES: c1ccc(-c2ccc(-c3ccc(-c4ccccc4)cc3N(c3ccc(-c4ccccc4)cc3)c3ccc(-c4cccc5ccccc45)cc3)cc2)cc1 ; CC1(C)c2ccccc2-c2ccc(N(c3cc(-c4ccccc4)ccc3-c3ccc(-c4ccccc4)cc3)c3cccc4c3-c3ccccc3C4(C)C)cc21 ; CC(C)(C)c1cc(-c2ccc(-c3ccc(-c4ccccc4)cc3N(c3ccc4c(c3)C(C)(C)c3ccccc3-4)c3ccc4c(c3)C(C)(C)c3ccccc3-4)cc2)cc(C(C)(C)C)c1]

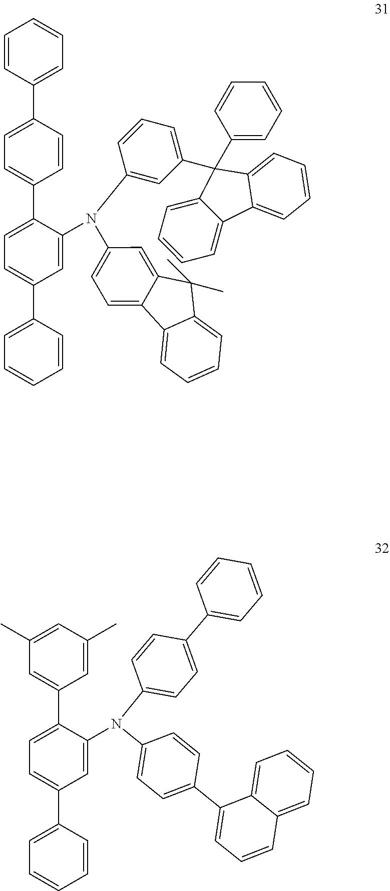 [SMILES: CC1(C)c2ccccc2-c2ccc(N(c3cccc(C4(c5ccccc5)c5ccccc5-c5ccccc54)c3)c3cc(-c4ccccc4)ccc3-c3ccc(-c4ccccc4)cc3)cc21 ; Cc1cc(C)cc(-c2ccc(-c3ccccc3)cc2N(c2ccc(-c3ccccc3)cc2)c2ccc(-c3cccc4ccccc34)cc2)c1]

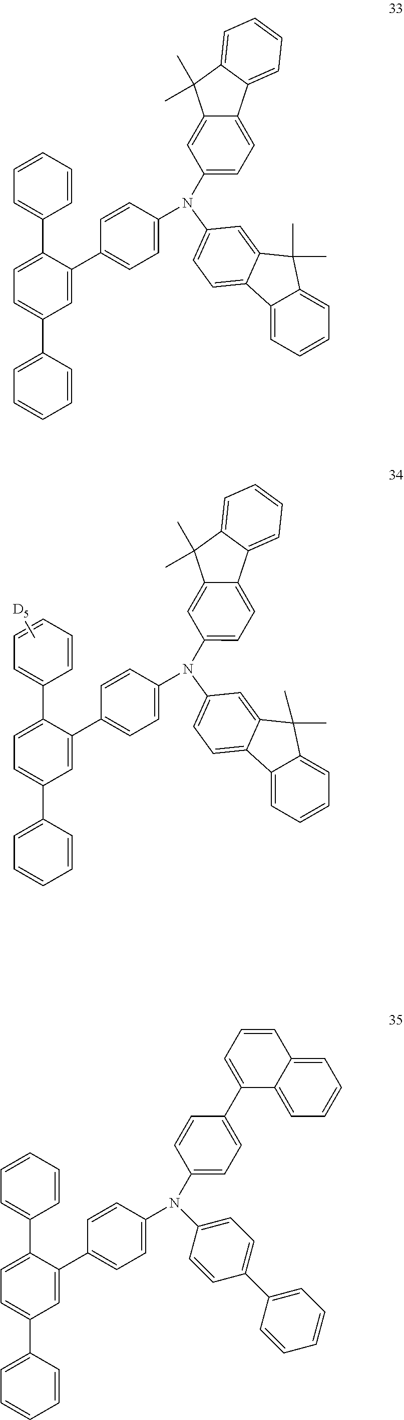

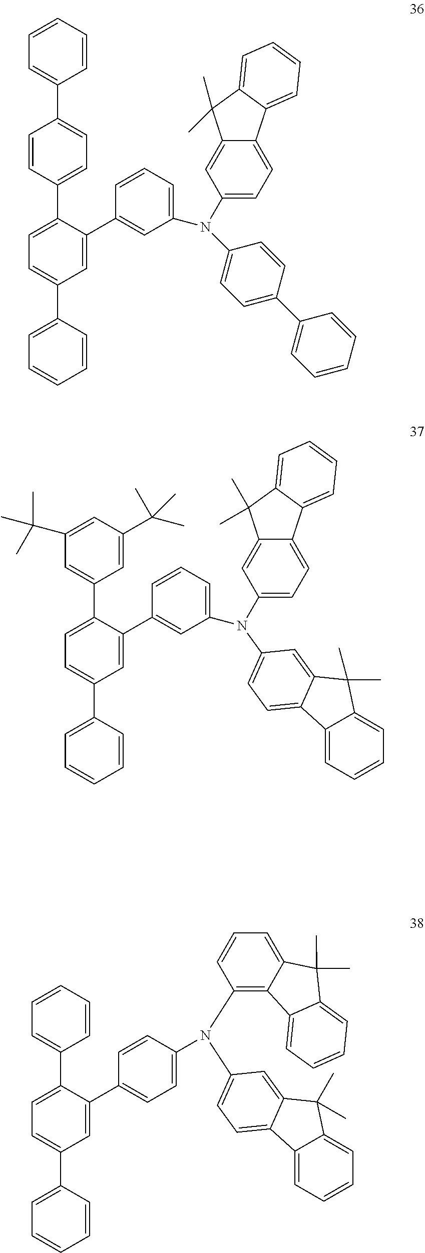 [SMILES: CC1(C)c2ccccc2-c2ccc(N(c3ccc(-c4ccccc4)cc3)c3cccc(-c4cc(-c5ccccc5)ccc4-c4ccc(-c5ccccc5)cc4)c3)cc21 ; CC(C)(C)c1cc(-c2ccc(-c3ccccc3)cc2-c2cccc(N(c3ccc4c(c3)C(C)(C)c3ccccc3-4)c3ccc4c(c3)C(C)(C)c3ccccc3-4)c2)cc(C(C)(C)C)c1 ; CC1(C)c2ccccc2-c2ccc(N(c3ccc(-c4cc(-c5ccccc5)ccc4-c4ccccc4)cc3)c3cccc4c3-c3ccccc3C4(C)C)cc21]

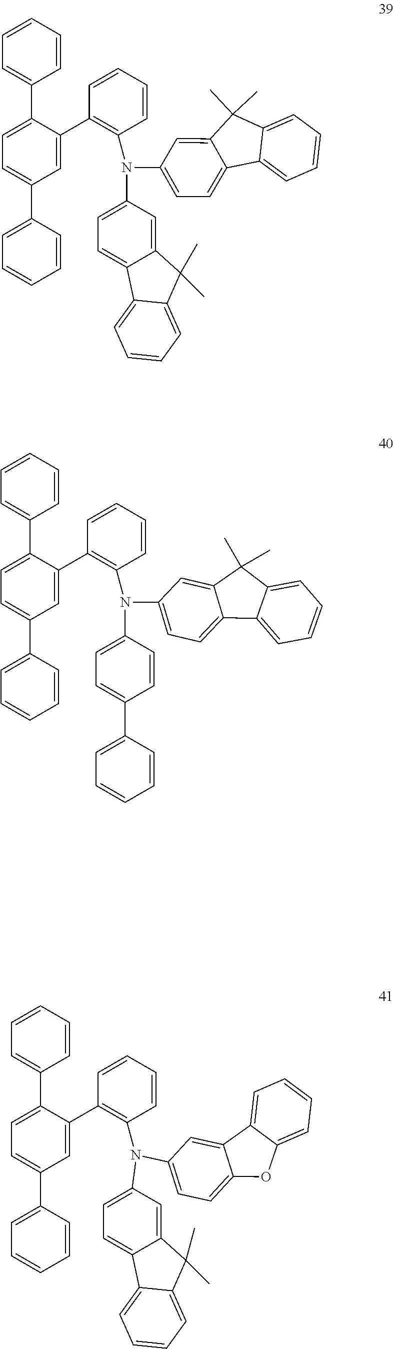 [SMILES: CC1(C)c2ccccc2-c2ccc(N(c3ccc4c(c3)C(C)(C)c3ccccc3-4)c3ccccc3-c3cc(-c4ccccc4)ccc3-c3ccccc3)cc21 ; CC1(C)c2ccccc2-c2ccc(N(c3ccc(-c4ccccc4)cc3)c3ccccc3-c3cc(-c4ccccc4)ccc3-c3ccccc3)cc21 ; CC1(C)c2ccccc2-c2ccc(N(c3ccc4oc5ccccc5c4c3)c3ccccc3-c3cc(-c4ccccc4)ccc3-c3ccccc3)cc21]

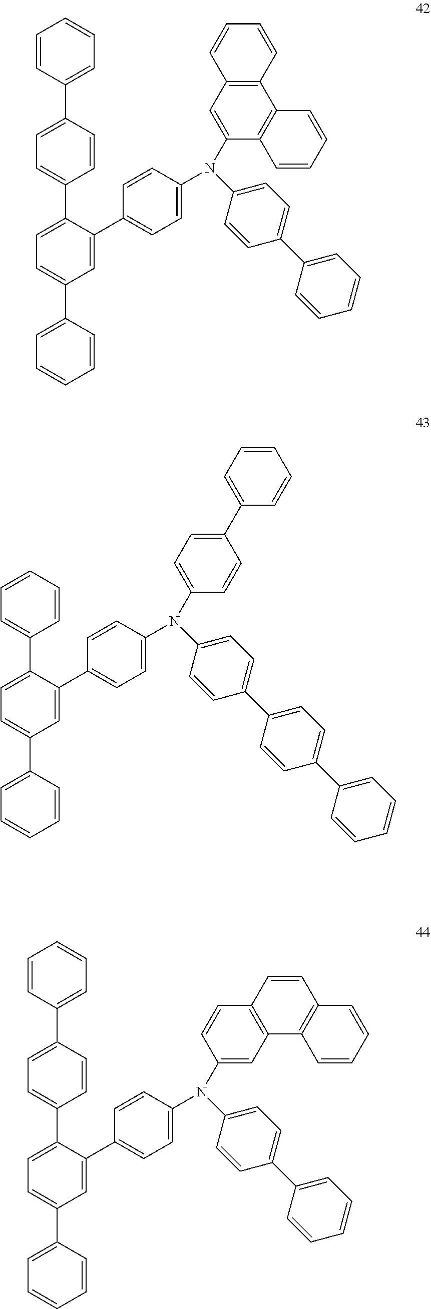 [SMILES: c1ccc(-c2ccc(-c3ccc(-c4ccccc4)cc3-c3ccc(N(c4ccc(-c5ccccc5)cc4)c4cc5ccccc5c5ccccc45)cc3)cc2)cc1 ; c1ccc(-c2ccc(-c3ccc(N(c4ccc(-c5ccccc5)cc4)c4ccc(-c5cc(-c6ccccc6)ccc5-c5ccccc5)cc4)cc3)cc2)cc1 ; c1ccc(-c2ccc(-c3ccc(-c4ccccc4)cc3-c3ccc(N(c4ccc(-c5ccccc5)cc4)c4ccc5ccc6ccccc6c5c4)cc3)cc2)cc1]

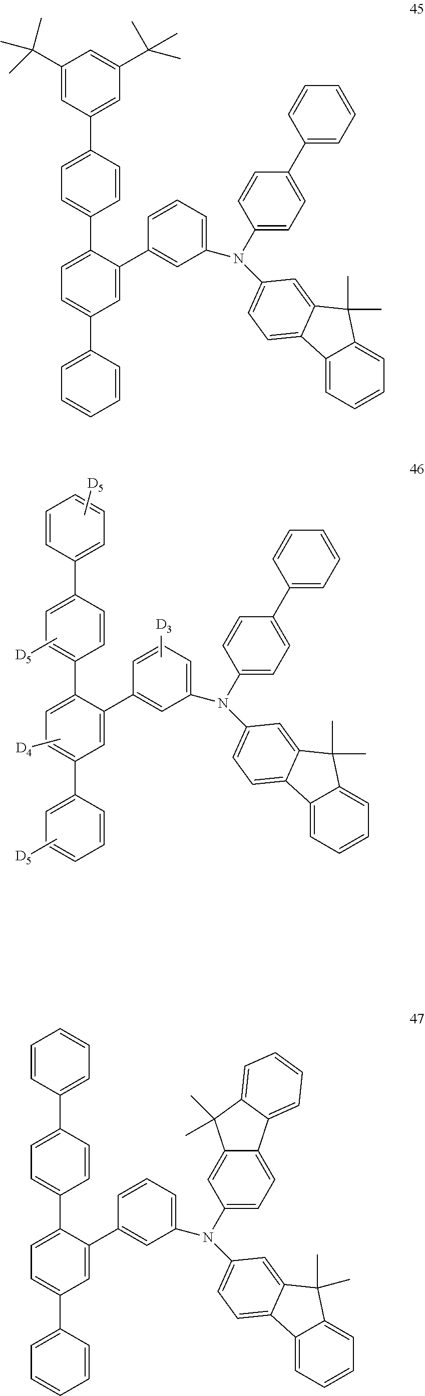

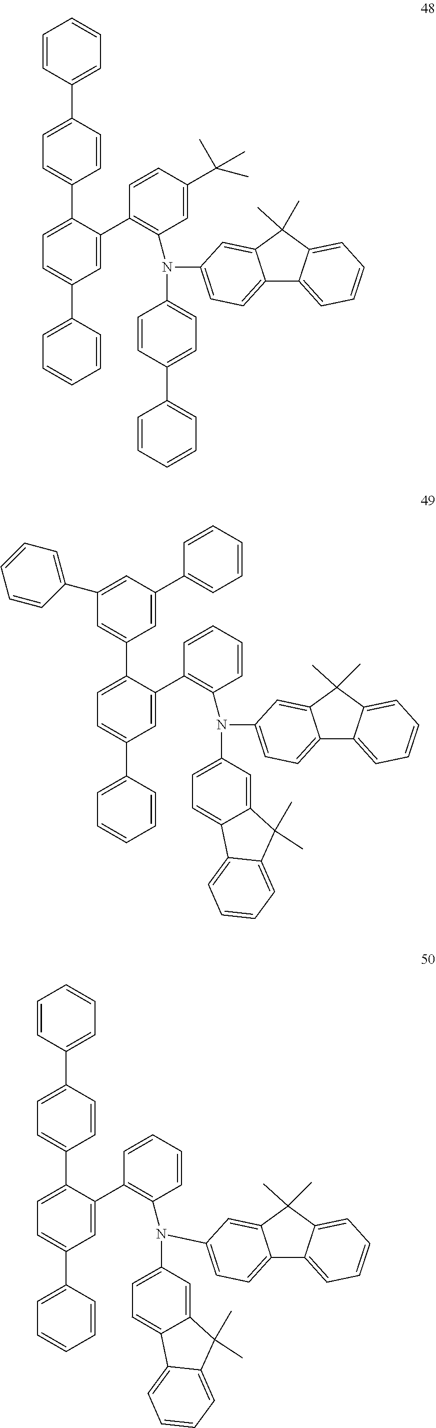 [SMILES: CC(C)(C)c1ccc(-c2cc(-c3ccccc3)ccc2-c2ccc(-c3ccccc3)cc2)c(N(c2ccc(-c3ccccc3)cc2)c2ccc3c(c2)C(C)(C)c2ccccc2-3)c1 ; CC1(C)c2ccccc2-c2ccc(N(c3ccc4c(c3)C(C)(C)c3ccccc3-4)c3ccccc3-c3cc(-c4ccccc4)ccc3-c3cc(-c4ccccc4)cc(-c4ccccc4)c3)cc21 ; CC1(C)c2ccccc2-c2ccc(N(c3ccc4c(c3)C(C)(C)c3ccccc3-4)c3ccccc3-c3cc(-c4ccccc4)ccc3-c3ccc(-c4ccccc4)cc3)cc21]

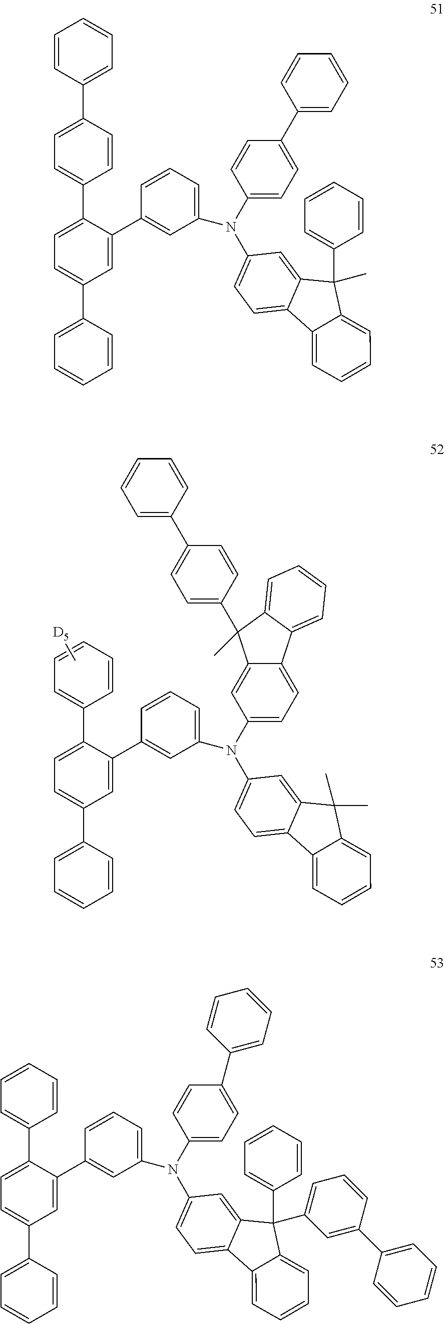

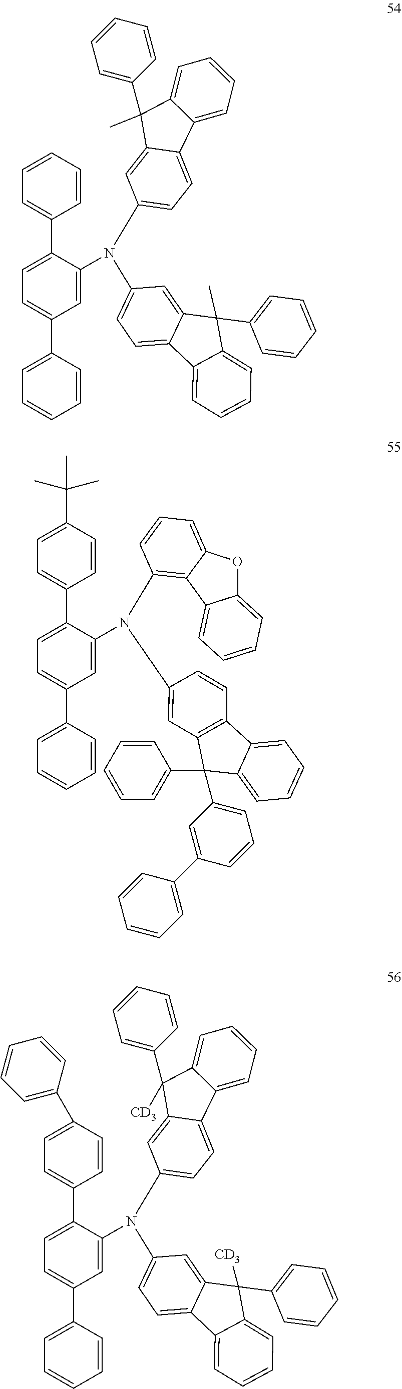

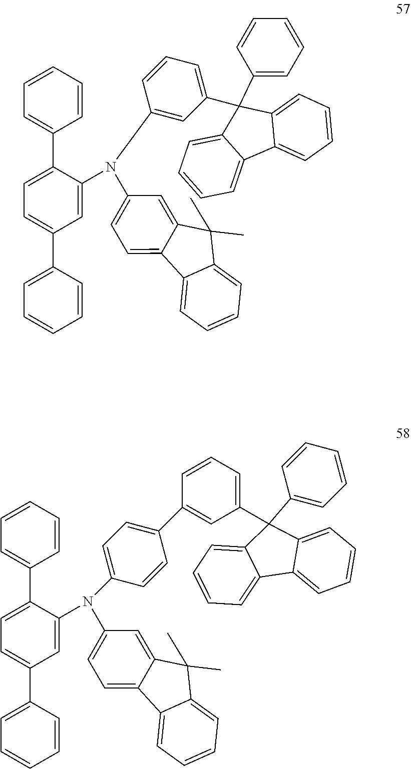 [SMILES: CC1(C)c2ccccc2-c2ccc(N(c3cccc(C4(c5ccccc5)c5ccccc5-c5ccccc54)c3)c3cc(-c4ccccc4)ccc3-c3ccccc3)cc21 ; CC1(C)c2ccccc2-c2ccc(N(c3ccc(-c4cccc(C5(c6ccccc6)c6ccccc6-c6ccccc65)c4)cc3)c3cc(-c4ccccc4)ccc3-c3ccccc3)cc21]

The compounds according to the present application may be prepared by means of the synthesis methods described hereinafter.

By the method shown in Scheme 1, proceeding from a biphenyl derivative substituted by two reactive groups, in a Suzuki coupling, it is possible to prepare a terphenyl derivative substituted by a reactive group in a position ortho to a phenyl-phenyl bond.

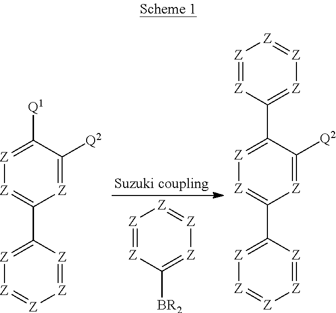

In a subsequent step, as shown in Scheme 2, a Hartwig-Buchwald coupling can be effected, by means of which an amino group is introduced into the molecule. This affords a compound according to the present application in which the index k=0.

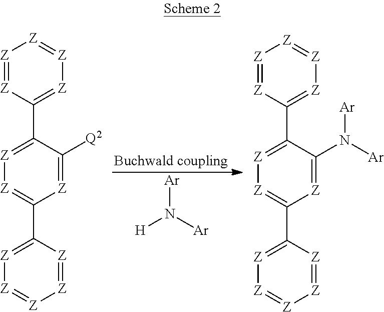

Alternatively, in a subsequent step, as shown in Scheme 3, a Suzuki coupling can be effected, by means of which an aromatic ring system is introduced into the molecule. This affords a compound according to the present application in which the index k>0.

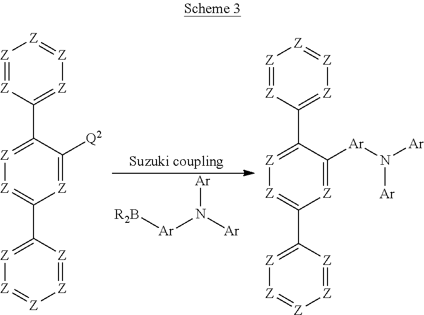

The definitions of the variable groups in the schemes shown above are as follows:
Z=N or CR
R=H or organic radical
Q¹, Q²=reactive group
Ar=optionally substituted aromatic or heteroaromatic

The present application thus provides a process for preparing a compound according to the present application, characterized in that a terphenyl derivative a) substituted by a reactive group is reacted in a coupling reaction with a secondary amine, or b) is reacted in a coupling reaction with an aromatic or heteroaromatic species bearing a boron-containing group.

The reactive group here is preferably selected from Cl, Br and I, more preferably from Br and I. The coupling reaction in the reaction under a) is preferably a Hartwig-Buchwald coupling reaction. The coupling reaction under b) is preferably a Suzuki coupling reaction.

The terphenyl derivative substituted by a reactive group is preferably prepared proceeding from a biphenyl derivative substituted by two reactive groups which is prepared by means of a Suzuki coupling reaction.

The above-described compounds of the invention, especially compounds substituted by reactive leaving groups, such as bromine, iodine, chlorine, boronic acid or boronic ester, may find use as monomers for production of corresponding oligomers, dendrimers or polymers. Suitable reactive leaving groups are, for example, bromine, iodine, chlorine, boronic acids, boronic esters, amines, alkenyl or alkynyl groups having a terminal C═C double bond or C—C triple bond, oxiranes, oxetanes, groups which enter into a cycloaddition, for example a 1,3-dipolar cycloaddition, for example dienes or azides, carboxylic acid derivatives, alcohols and silanes.

The invention therefore further provides oligomers, polymers or dendrimers containing one or more compounds of formula (I), (II) or (III), wherein the bond(s) to the polymer, oligomer or dendrimer may be localized at any desired positions substituted by R¹, R², R³, R⁴ or R⁵ in formula I), (II) or (III). According to the linkage of the compound of formula (I), (II) or (III), the compound is part of a side chain of the oligomer or polymer or part of the main chain. An oligomer in the context of this invention is understood to mean a compound formed from at least three monomer units. A polymer in the context of the invention is understood to mean a compound formed from at least ten monomer units. The polymers, oligomers or dendrimers of the invention may be conjugated, partly conjugated or nonconjugated. The oligomers or polymers of the invention may be linear, branched or dendritic. In the structures having linear linkage, the units of formula (I), (II) or (III) may be joined directly to one another, or they may be joined to one another via a bivalent group, for example via a substituted or unsubstituted alkylene group, via a heteroatom or via a bivalent aromatic or heteroaromatic group. In branched and dendritic structures, it is possible, for example, for three or more units of formula (I), (II) or (III) to be joined via a trivalent or higher-valency group, for example via a trivalent or higher-valency aromatic or heteroaromatic group, to give a branched or dendritic oligomer or polymer.

For the repeat units of formula (I), (II) or (III) in oligomers, dendrimers and polymers, the same preferences apply as described above for compounds of formula (I), (II) or (III).

For preparation of the oligomers or polymers, the monomers of the invention are homopolymerized or copolymerized with further monomers. Suitable and preferred comonomers are selected from fluorenes, spirobifluorenes, paraphenylenes, carbazoles, thiophenes, dihydrophenanthrenes, cis- and trans-indenofluorenes, ketones, phenanthrenes or else two or more of these units. The polymers, oligomers and dendrimers typically contain still further units, for example emitting (fluorescent or phosphorescent) units, for example vinyltriarylamines or phosphorescent metal complexes, and/or charge transport units, especially those based on triarylamines.

The polymers, oligomers and dendrimers of the invention have advantageous properties, especially high lifetimes, high efficiencies and good colour coordinates.

The polymers and oligomers of the invention are generally prepared by polymerization of one or more monomer types, of which at least one monomer leads to repeat units of the formula (I), (II) or (III) in the polymer.

Suitable polymerization reactions are known to those skilled in the art and are described in the literature. Particularly suitable and preferred polymerization reactions which lead to C—C and C—N couplings are as follows:
(A) SUZUKI polymerization;
(B) YAMAMOTO polymerization;
(C) STILLE polymerization; and
(D) HARTWIG-BUCHWALD polymerization.

How the polymerization can be conducted by these methods and how the polymers can then be separated from the reaction medium and purified is known to those skilled in the art and is described in detail in the literature.

For the processing of the compounds of the invention from a liquid phase, for example by spin-coating or by printing methods, formulations of the compounds of the invention are required. These formulations may, for example, be solutions, dispersions or emulsions. For this purpose, it may be preferable to use mixtures of two or more solvents. Suitable and preferred solvents are, for example, toluene, anisole, o-, m- or p-xylene, methyl benzoate, mesitylene, tetralin, veratrole, THF, methyl-THF, THP, chlorobenzene, dioxane, phenoxytoluene, especially 3-phenoxytoluene, (−)-fenchone, 1,2,3,5-tetramethylbenzene, 1,2,4,5-tetramethylbenzene, 1-methylnaphthalene, 2-methylbenzothiazole, 2-phenoxyethanol, 2-pyrrolidinone, 3-methylanisole, 4-methylanisole, 3,4-dimethylanisole, 3,5-dimethylanisole, acetophenone, alpha-terpineol, benzothiazole, butyl benzoate, cumene, cyclohexanol, cyclohexanone, cyclohexylbenzene, decalin, dodecylbenzene, ethyl benzoate, indane, methyl benzoate, NMP, p-cymene, phenetole, 1,4-diisopropylbenzene, dibenzyl ether, diethylene glycol butyl methyl ether, triethylene glycol butyl methyl ether, diethylene glycol dibutyl ether, triethylene glycol dimethyl ether, diethylene glycol monobutyl ether, tripropylene glycol dimethyl ether, tetraethylene glycol dimethyl ether, 2-isopropylnaphthalene, pentylbenzene, hexylbenzene, heptylbenzene, octylbenzene, 1,1-bis(3,4-dimethylphenyl)ethane, or mixtures of these solvents.

The invention therefore further provides a formulation, especially a solution, dispersion or emulsion, comprising at least one compound of formula (I), (II) or (III) or at least one polymer, oligomer or dendrimer containing at least one unit of formula (I), (II) or (III) and at least one solvent, preferably an organic solvent. The way in which such solutions can be prepared is known to those skilled in the art.

The compound of formula (I), (II) or (III) is suitable for use in an electronic device, especially an organic electroluminescent device (OLED). Depending on the substitution, the compound of the formula (I), (II) or (III) can be used in different functions and layers. Preference is given to use as a hole-transporting material in a hole-transporting layer and/or as matrix material in an emitting layer, more preferably in combination with a phosphorescent emitter.

The invention therefore further provides for the use of a compound of formula (I), (II) or (III) in an electronic device. This electronic device is preferably selected from the group consisting of organic integrated circuits (OICs), organic field-effect transistors (OFETs), organic thin-film transistors (OTFTs), organic light-emitting transistors (OLETs), organic solar cells (OSCs), organic optical detectors, organic photoreceptors, organic field-quench devices (OFQDs), organic light-emitting electrochemical cells (OLECs), organic laser diodes (O-lasers) and more preferably organic electroluminescent devices (OLEDs).

The invention further provides an electronic device comprising at least one compound of formula (I), (II) or (III). This electronic device is preferably selected from the abovementioned devices.

Particular preference is given to an organic electroluminescent device comprising anode, cathode and at least one emitting layer, characterized in that at least one organic layer comprising at least one compound of formula (I), (II) or (III) is present in the device. Preference is given to an organic electroluminescent device comprising anode, cathode and at least one emitting layer, characterized in that at least one organic layer in the device, selected from hole-transporting and emitting layers, comprises at least one compound of formula (I), (II) or (III).

A hole-transporting layer is understood here to mean all layers disposed between anode and emitting layer, preferably hole injection layer, hole transport layer and electron blocker layer. A hole injection layer is understood here to mean a layer that directly adjoins the anode. A hole transport layer is understood here to mean a layer which is between the anode and emitting layer but does not directly adjoin the anode, and preferably does not directly adjoin the emitting layer either. An electron blocker layer is understood here to mean a layer which is between the anode and emitting layer and directly adjoins the emitting layer. An electron blocker layer preferably has a high-energy LUMO and hence prevents electrons from exiting from the emitting layer.

Apart from the cathode, anode and emitting layer, the electronic device may comprise further layers. These are selected, for example, from in each case one or more hole injection layers, hole transport layers, hole blocker layers, electron transport layers, electron injection layers, electron blocker layers, exciton blocker layers, interlayers, charge generation layers and/or organic or inorganic p/n junctions. However, it should be pointed out that not every one of these layers need necessarily be present and the choice of layers always depends on the compounds used and especially also on whether the device is a fluorescent or phosphorescent electroluminescent device.

The sequence of layers in the electronic device is preferably as follows:
-anode-
-hole injection layer-
-hole transport layer-
-optionally further hole transport layers-
-emitting layer-
-optionally hole blocker layer-
-electron transport layer-
-electron injection layer-
-cathode-.

At the same time, it should be pointed out again that not all the layers mentioned need be present and/or that further layers may additionally be present.

The organic electroluminescent device of the invention may contain two or more emitting layers. More preferably, these emission layers have several emission maxima between 380 nm and 750 nm overall, such that the overall result is white emission, in other words, various emitting compounds which may fluoresce or phosphoresce and which emit blue, green, yellow, orange or red light are used in the emitting layers. Especially preferred are three-layer systems, i.e. systems having three emitting layers, wherein one of the three layers in each case shows blue emission, one of the three layers in each case shows green emission, and one of the three layers in each case shows orange or red emission. The compounds of the invention here are preferably present in a hole-transporting layer or in the emitting layer. It should be noted that, for the production of white light, rather than a plurality of colour-emitting emitter compounds, an emitter compound used individually which emits over a broad wavelength range may also be suitable.

It is preferable that the compound of the formula (I), (II) or (III) is used as hole transport material. The emitting layer here may be a fluorescent emitting layer, or it may be a phosphorescent emitting layer. The emitting layer is preferably a blue-fluorescing layer or a green-phosphorescing layer.

When the device containing the compound of the formula (I), (II) or (III) contains a phosphorescent emitting layer, it is preferable that this layer contains two or more, preferably exactly two, different matrix materials (mixed matrix system). Preferred embodiments of mixed matrix systems are described in detail further down.

If the compound of formula (I), (II) or (III) is used as hole transport material in a hole transport layer, a hole injection layer or an electron blocker layer, the compound can be used as pure material, i.e. in a proportion of 100%, in the hole transport layer, or it can be used in combination with one or more further compounds.

In a preferred embodiment, a hole-transporting layer comprising the compound of the formula (I), (II) or (III) additionally comprises one or more further hole-transporting compounds. These further hole-transporting compounds are preferably selected from triarylamine compounds, more preferably from monotriarylamine compounds. They are most preferably selected from the preferred embodiments of hole transport materials that are specified further down. In the preferred embodiment described, the compound of the formula (I), (II) or (III) and the one or more further hole-transporting compounds are preferably each present in a proportion of at least 10%, more preferably each in a proportion of at least 20%.

In a preferred embodiment, a hole-transporting layer comprising the compound of the formula (I), (II) or (III) additionally contains one or more p-dopants. p-Dopants used according to the present invention are preferably those organic electron acceptor compounds capable of oxidizing one or more of the other compounds in the mixture.

Particularly preferred as p-dopants are quinodimethane compounds, azaindenofluorenediones, azaphenalenes, azatriphenylenes, 12, metal halides, preferably transition metal halides, metal oxides, preferably metal oxides comprising at least one transition metal or a metal from main group 3, and transition metal complexes, preferably complexes of Cu, Co, Ni, Pd and Pt with ligands containing at least one oxygen atom as binding site. Preference is further given to transition metal oxides as dopants, preferably oxides of rhenium, molybdenum and tungsten, more preferably Re₂O₇, MoO₃, WO₃ and ReO₃. Still further preference is given to complexes of bismuth in the (III) oxidation state, more particularly bismuth(III) complexes with electron-deficient ligands, more particularly carboxylate ligands.

The p-dopants are preferably in substantially homogeneous distribution in the p-doped layers. This can be achieved, for example, by co-evaporation of the p-dopant and the hole transport material matrix. The p-dopant is preferably present in a proportion of 1% to 10% in the p-doped layer.

Preferred p-Dopants are Especially the Following Compounds:

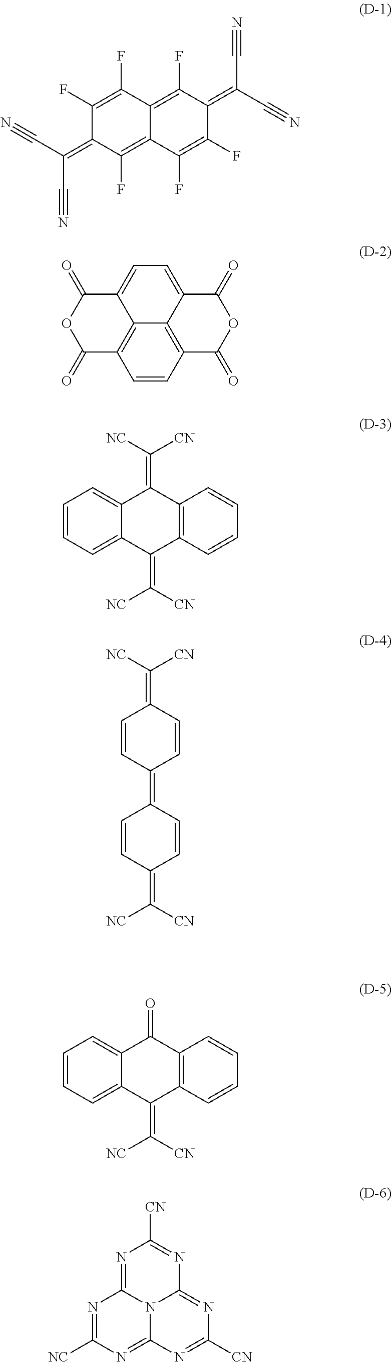

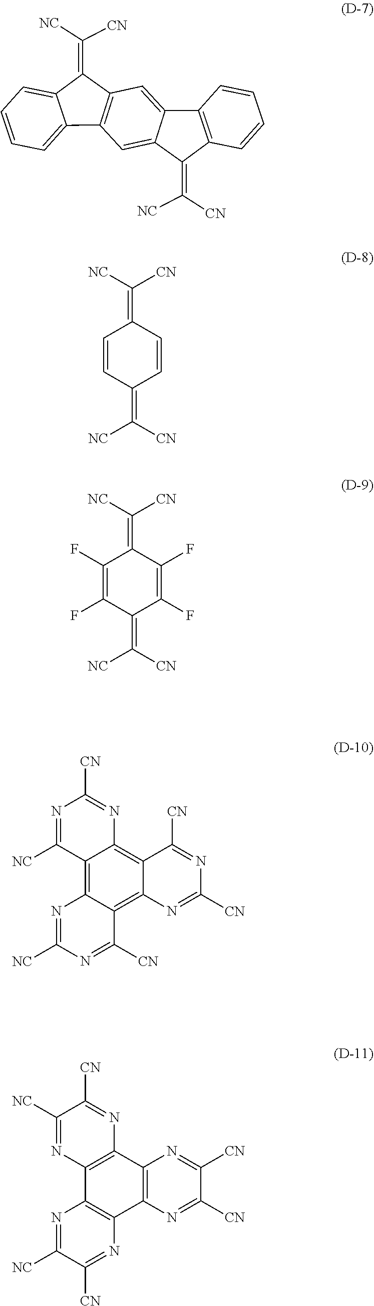 [SMILES: N#CC(C#N)=C1c2ccccc2-c2cc3c(cc21)-c1ccccc1C3=C(C#N)C#N ; N#CC(C#N)=c1ccc(=C(C#N)C#N)cc1 ; N#CC(C#N)=c1c(F)c(F)c(=C(C#N)C#N)c(F)c1F ; N#Cc1nc(C#N)c2c(n1)c1c(C#N)nc(C#N)nc1c1c(C#N)nc(C#N)nc21 ; N#Cc1nc2c3nc(C#N)c(C#N)nc3c3nc(C#N)c(C#N)nc3c2nc1C#N]

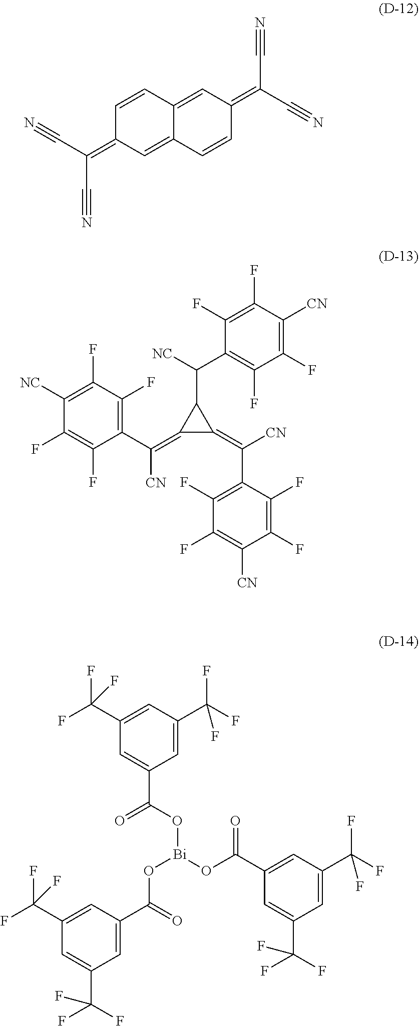

In a preferred embodiment, a hole injection layer that conforms to one of the following embodiments is present in the device: a) it contains a triarylamine and a p-dopant; or b) it contains a single electron-deficient material (electron acceptor). In a preferred embodiment of embodiment a), the triarylamine is a monotriarylamine, especially one of the preferred triarylamine derivatives mentioned further down. In a preferred embodiment of embodiment b), the electron-deficient material is a hexaazatriphenylene derivative as described in US 2007/0092755.

The compound of the formula (I), (II) or (III) may be present in a hole injection layer, in a hole transport layer and/or in an electron blocker layer of the device. When the compound is present in a hole injection layer or in a hole transport layer, it has preferably been p-doped, meaning that it is in mixed form with a p-dopant, as described above, in the layer.

The compound of the formula (I), (II) or (III) is preferably present in an electron blocker layer. In this case, it is preferably not p-doped. Further preferably, in this case, it is preferably in the form of a single compound in the layer without addition of a further compound.

In an alternative preferred embodiment, the compound of the formula (I), (II) or (III) is used in an emitting layer as matrix material in combination with one or more emitting compounds, preferably phosphorescent emitting compounds. The phosphorescent emitting compounds here are preferably selected from red-phosphorescing and green-phosphorescing compounds.

The proportion of the matrix material in the emitting layer in this case is between 50.0% and 99.9% by volume, preferably between 80.0% and 99.5% by volume, and more preferably between 85.0% and 97.0% by volume.

Correspondingly, the proportion of the emitting compound is between 0.1% and 50.0% by volume, preferably between 0.5% and 20.0% by volume, and more preferably between 3.0% and 15.0% by volume.

An emitting layer of an organic electroluminescent device may also contain systems comprising a plurality of matrix materials (mixed matrix systems) and/or a plurality of emitting compounds. In this case too, the emitting compounds are generally those compounds having the smaller proportion in the system and the matrix materials are those compounds having the greater proportion in the system. In individual cases, however, the proportion of a single matrix material in the system may be less than the proportion of a single emitting compound.

It is preferable that the compounds of formula (I), (II) or (III) are used as a component of mixed matrix systems, preferably for phosphorescent emitters. The mixed matrix systems preferably comprise two or three different matrix materials, more preferably two different matrix materials. Preferably, in this case, one of the two materials is a material having hole-transporting properties and the other material is a material having electron-transporting properties. It is further preferable when one of the materials is selected from compounds having a large energy differential between HOMO and LUMO (wide-bandgap materials). The compound of the formula (I), (II) or (III) in a mixed matrix system is preferably the matrix material having hole-transporting properties. Correspondingly, when the compound of the formula (I), (II) or (III) is used as matrix material for a phosphorescent emitter in the emitting layer of an OLED, a second matrix compound having electron-transporting properties is present in the emitting layer. The two different matrix materials may be present here in a ratio of 1:50 to 1:1, preferably 1:20 to 1:1, more preferably 1:10 to 1:1 and most preferably 1:4 to 1:1.

The desired electron-transporting and hole-transporting properties of the mixed matrix components may, however, also be combined mainly or entirely in a single mixed matrix component, in which case the further mixed matrix component(s) fulfil(s) other functions.

Preference is given to using the following material classes in the abovementioned layers of the device:

### Phosphorescent Emitters:

The term “phosphorescent emitters” typically encompasses compounds where the emission of light is effected through a spin-forbidden transition, for example a transition from an excited triplet state or a state having a higher spin quantum number, for example a quintet state.

Suitable phosphorescent emitters are especially compounds which, when suitably excited, emit light, preferably in the visible region, and also contain at least one atom of atomic number greater than 20, preferably greater than 38, and less than 84, more preferably greater than 56 and less than 80. Preference is given to using, as phosphorescent emitters, compounds containing copper, molybdenum, tungsten, rhenium, ruthenium, osmium, rhodium, iridium, palladium, platinum, silver, gold or europium, especially compounds containing iridium, platinum or copper.

In the context of the present invention, all luminescent iridium, platinum or copper complexes are considered to be phosphorescent compounds.

In general, all phosphorescent complexes as used for phosphorescent OLEDs according to the prior art and as known to those skilled in the art in the field of organic electroluminescent devices are suitable for use in the devices of the invention. Further examples of suitable phosphorescent emitters are shown in the following table:

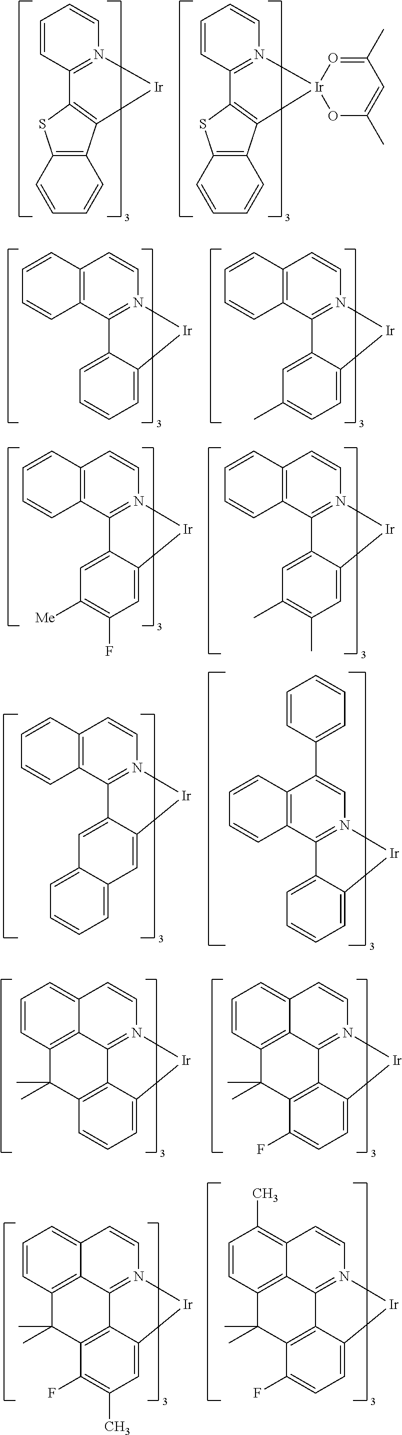

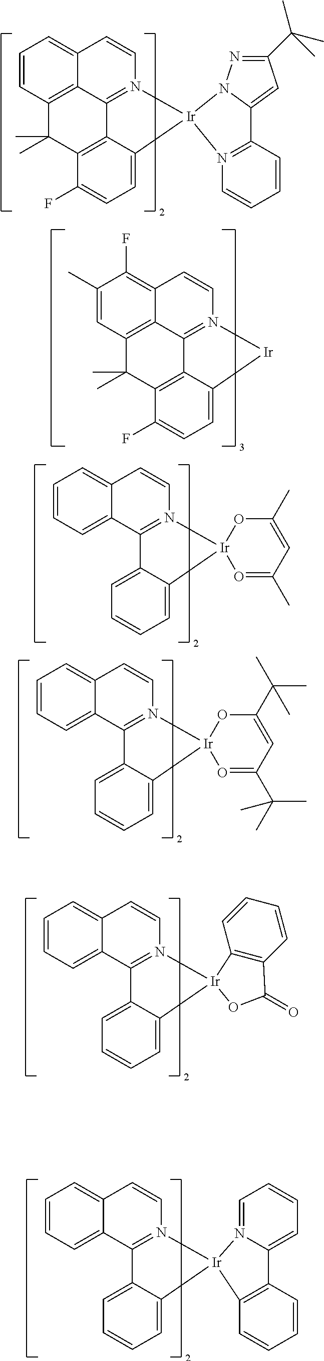

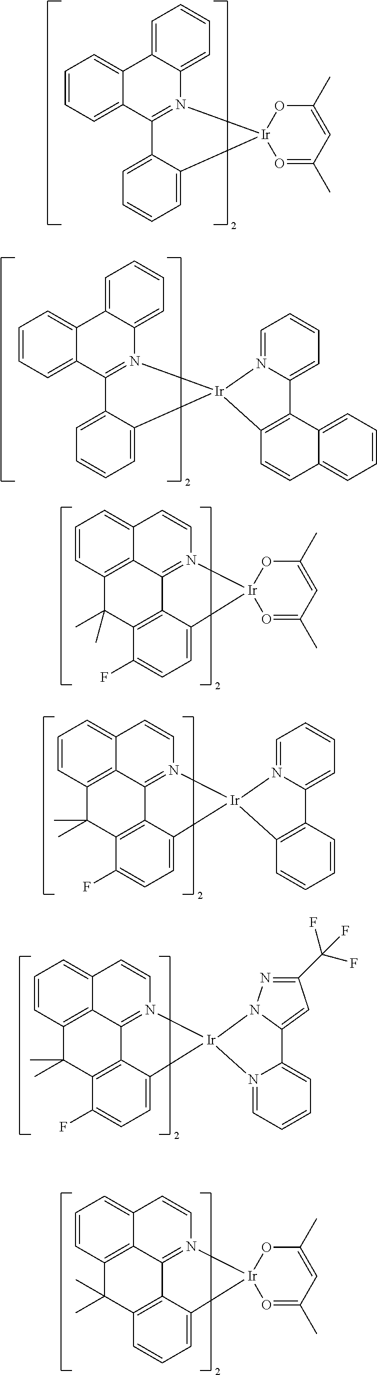

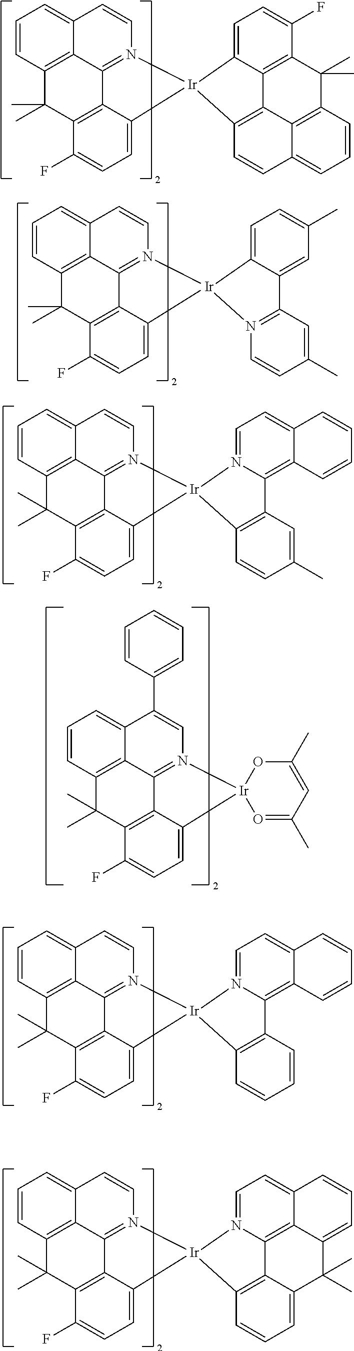

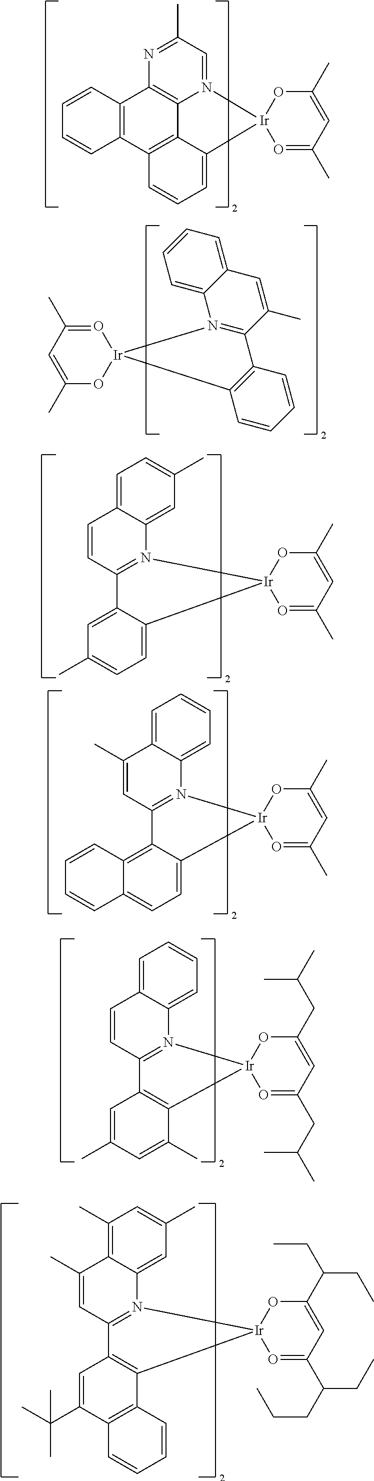

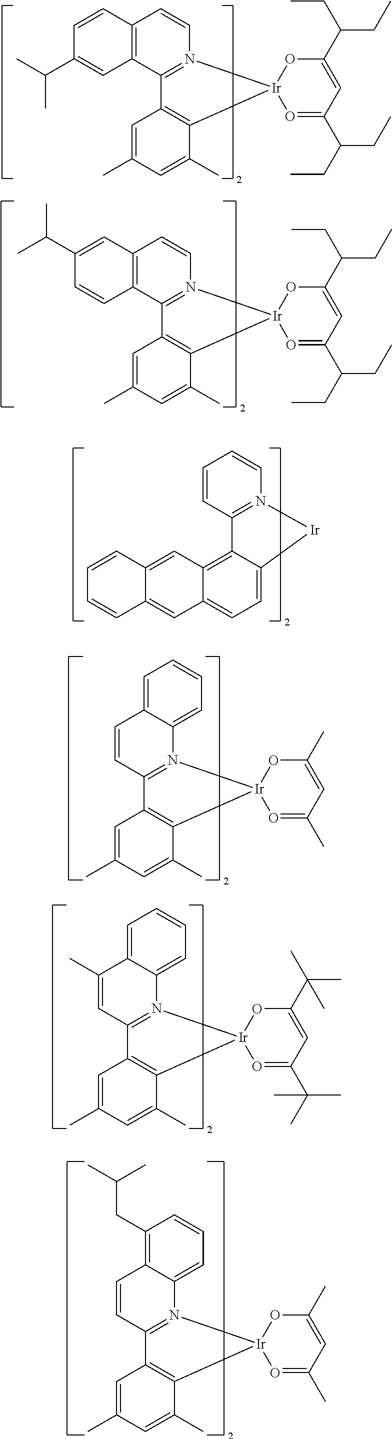

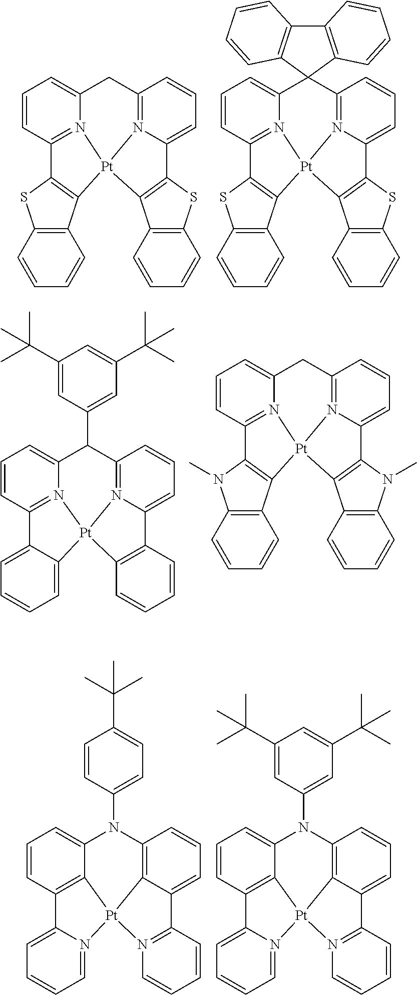

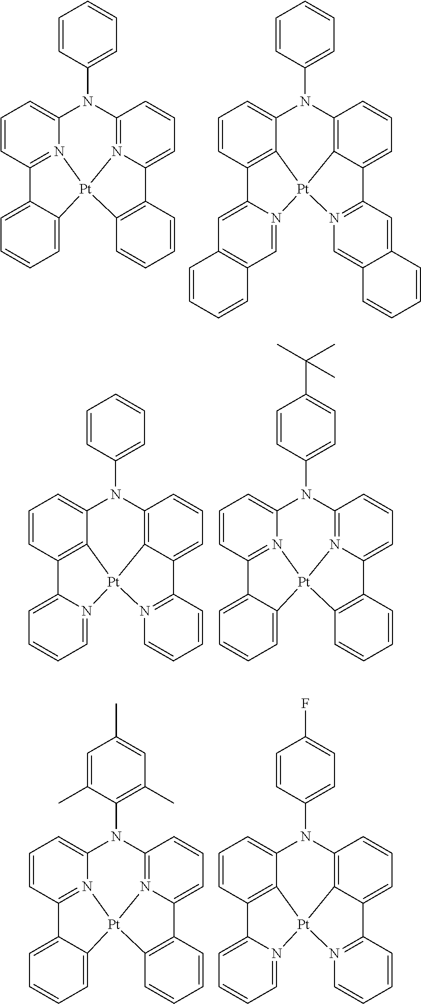

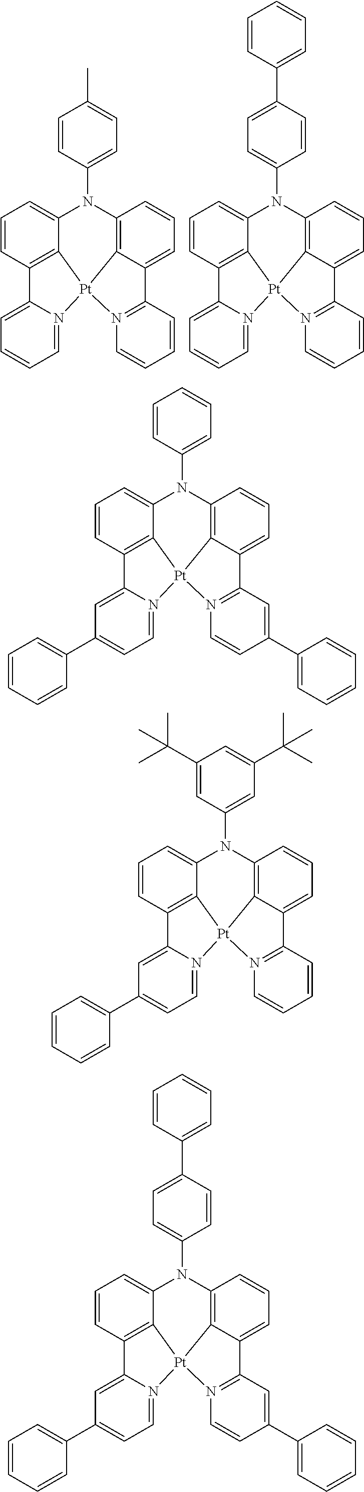

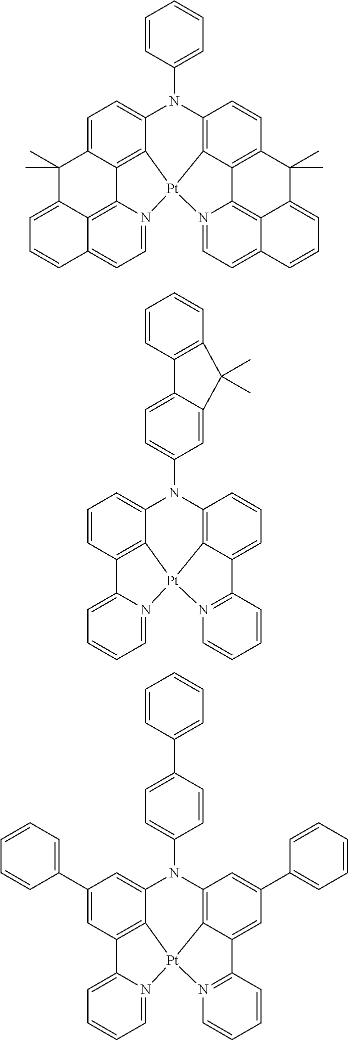

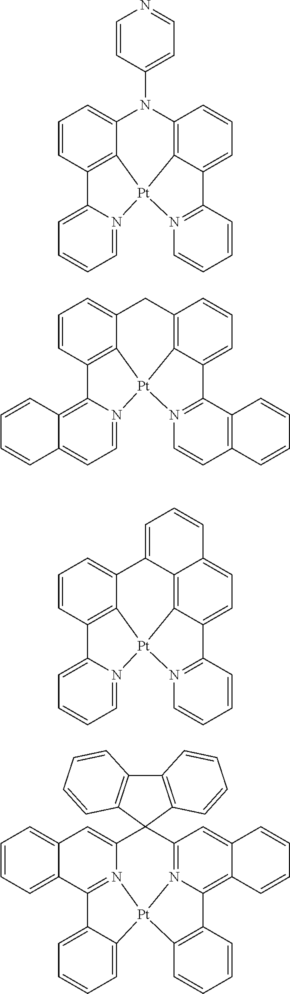

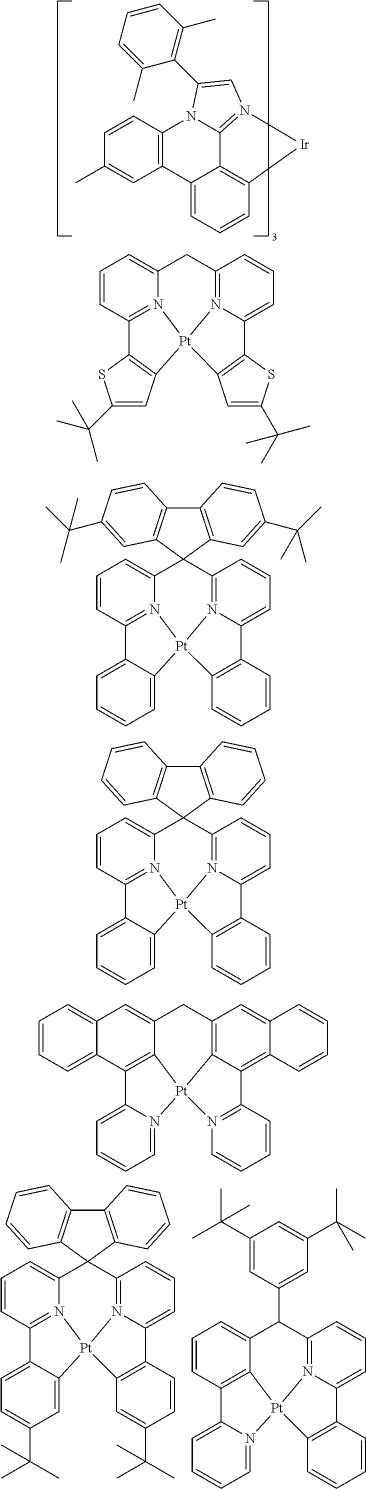

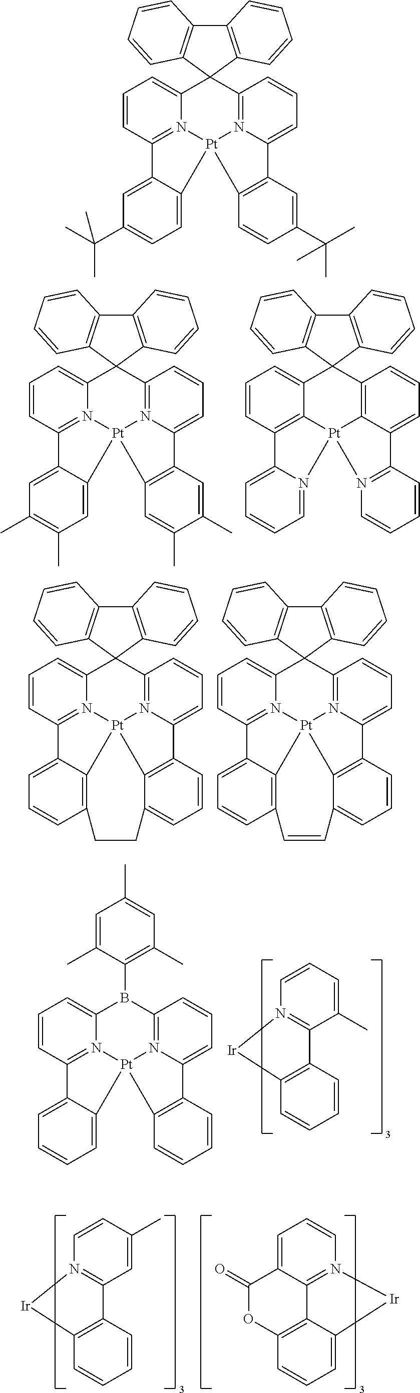

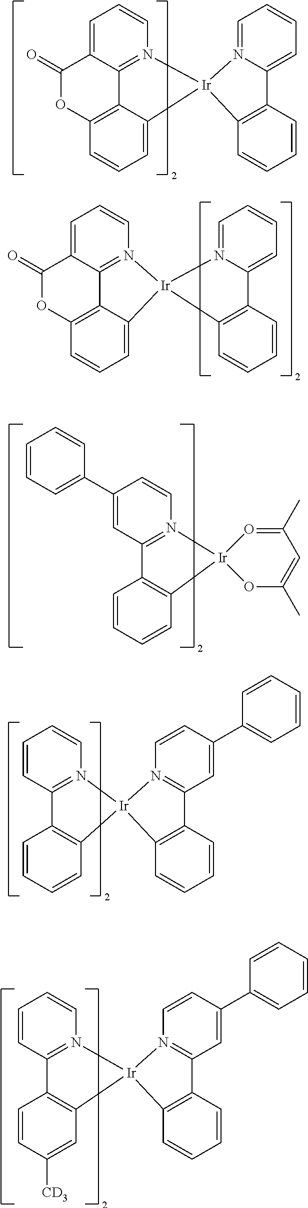

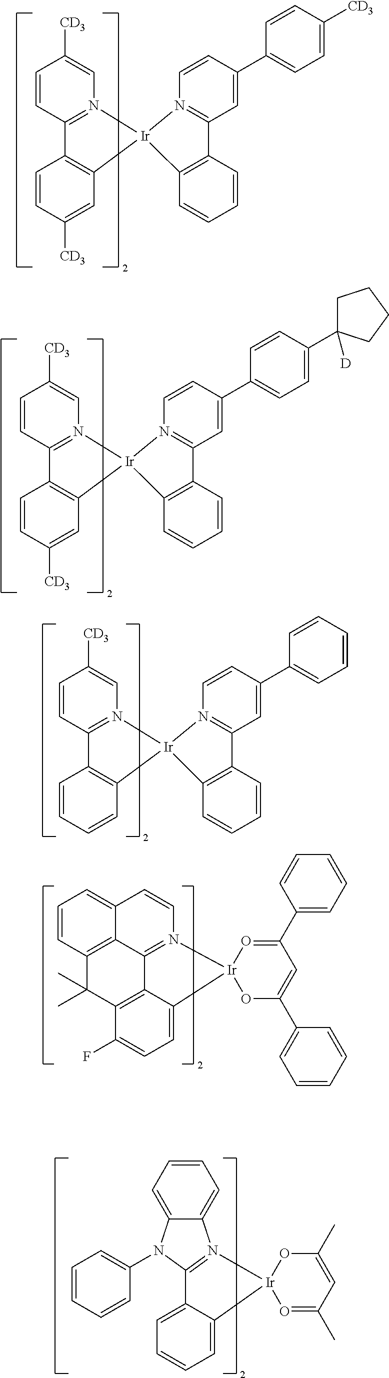

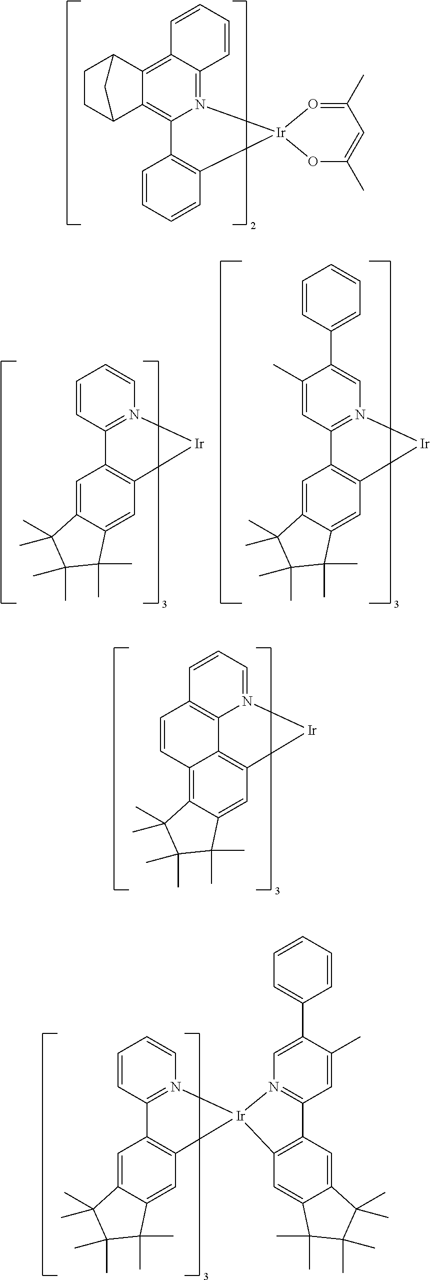

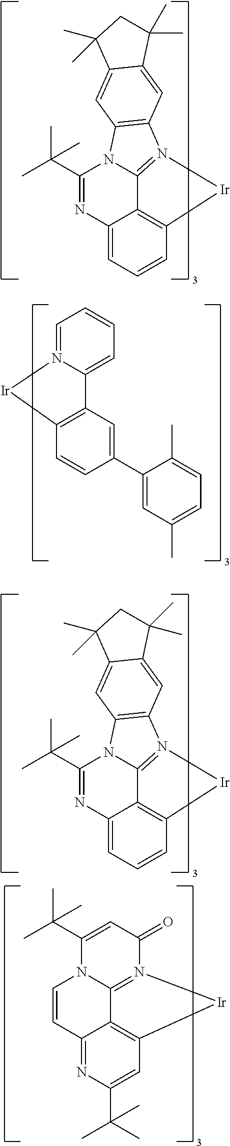

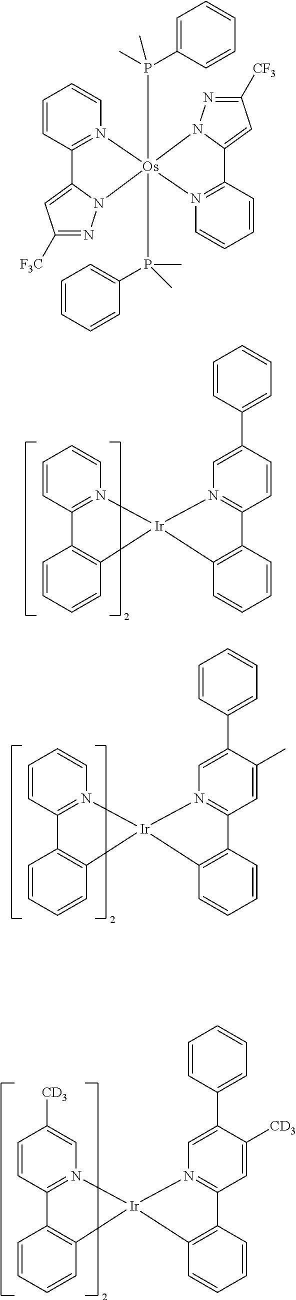

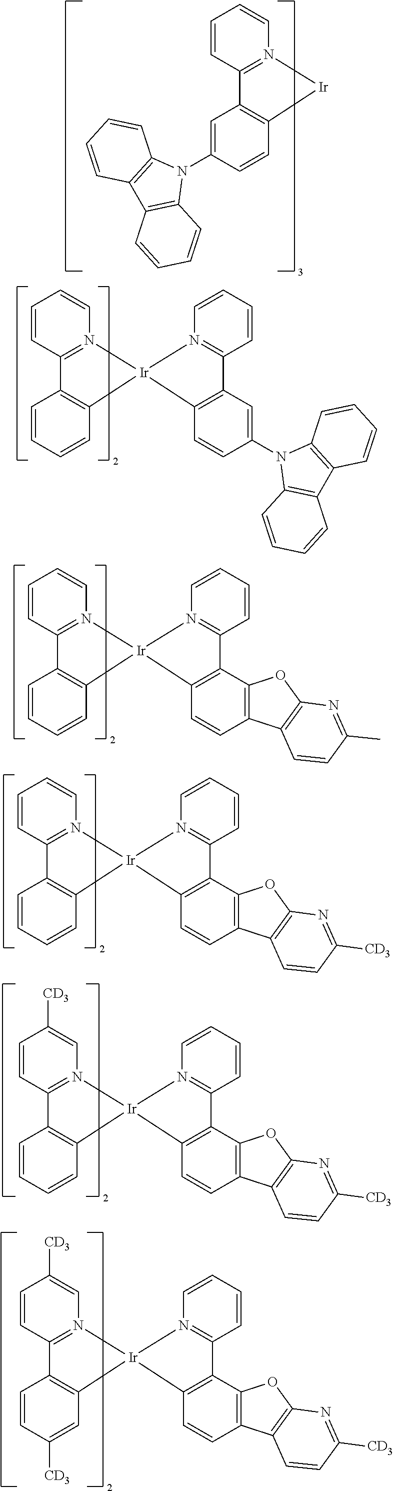

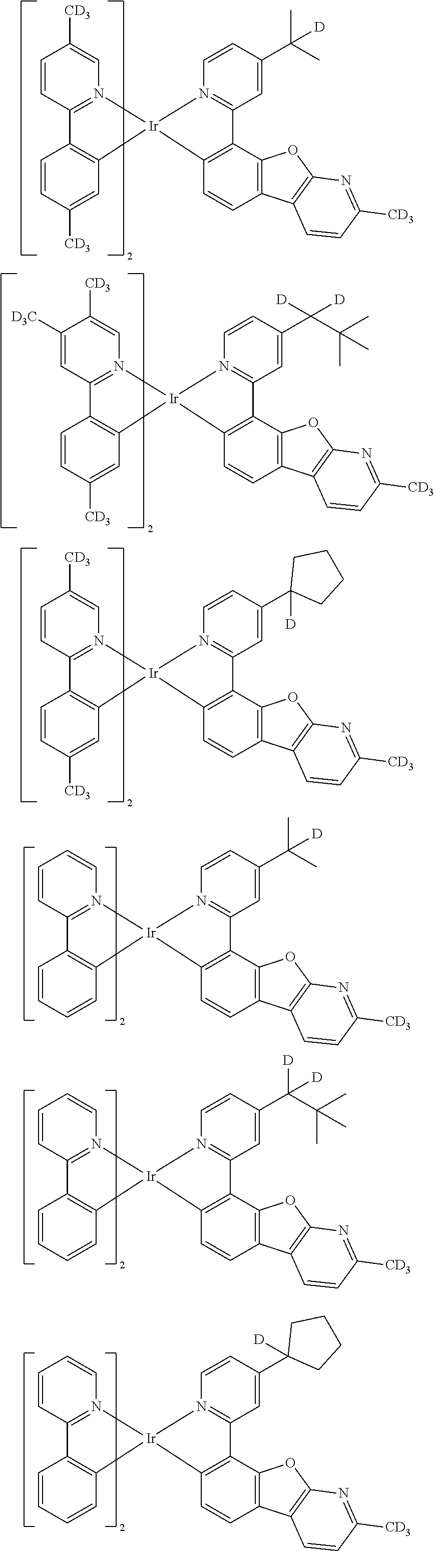

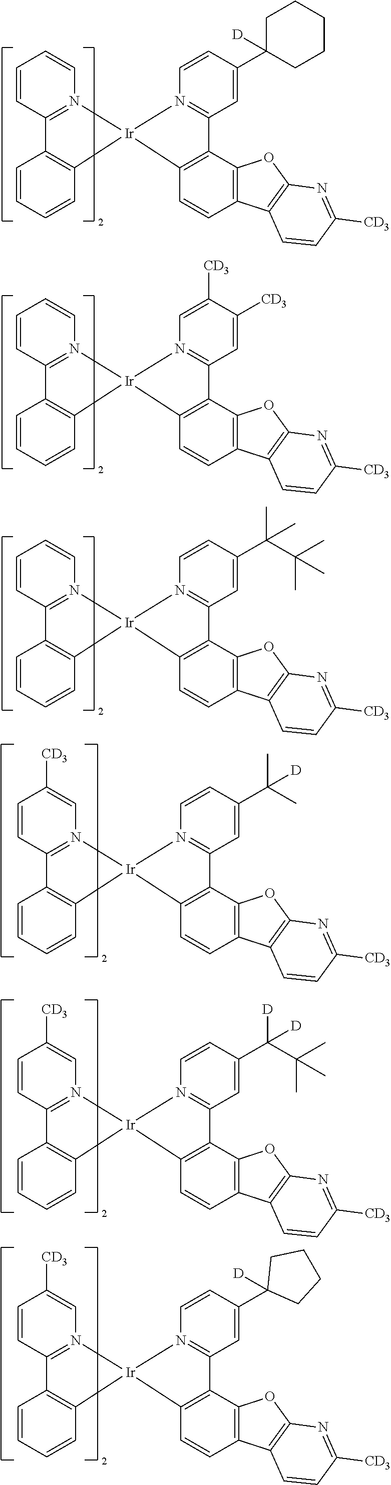

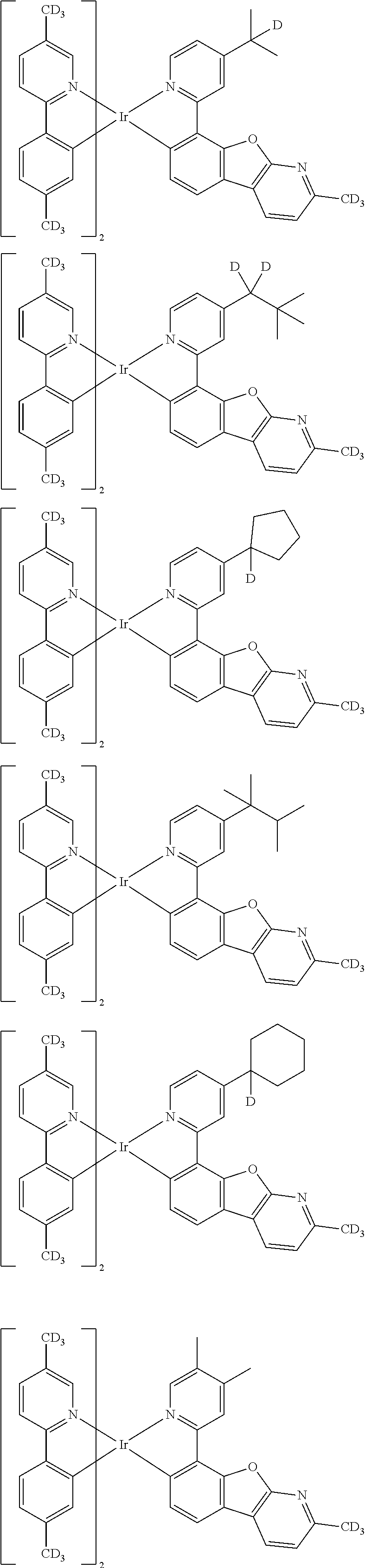

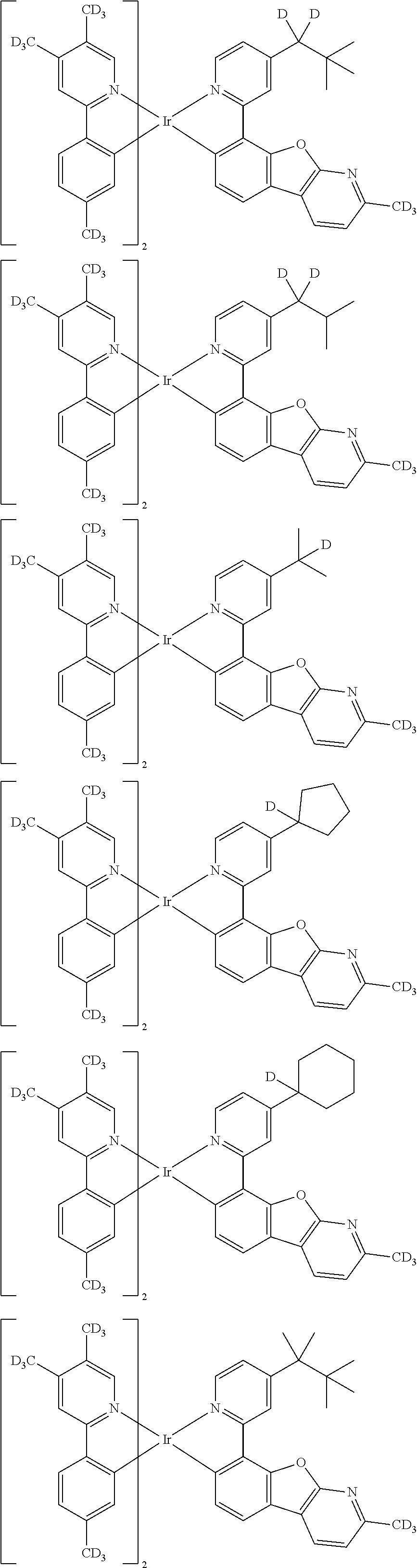

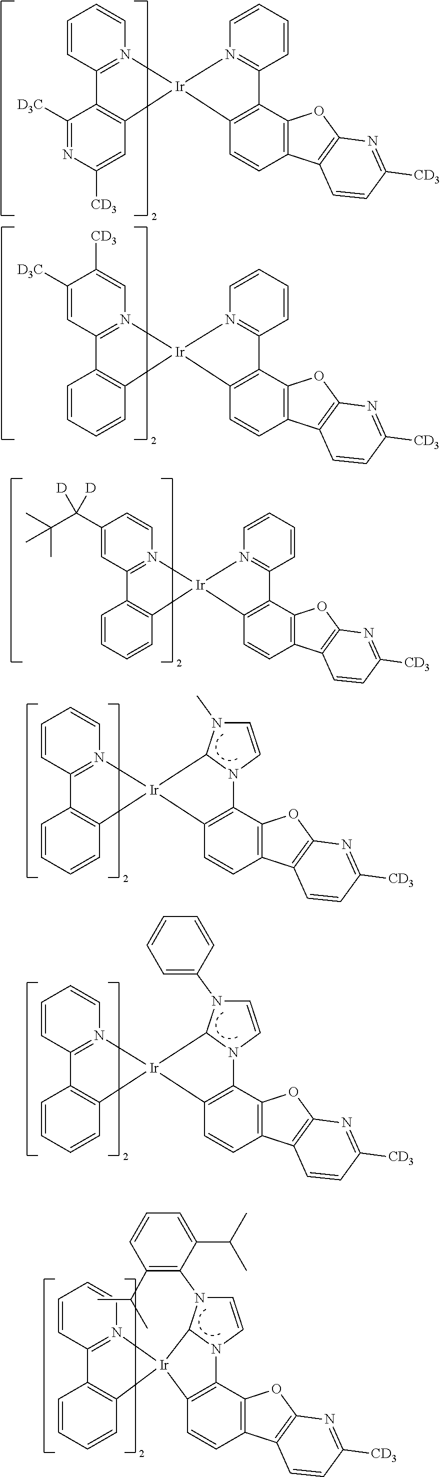

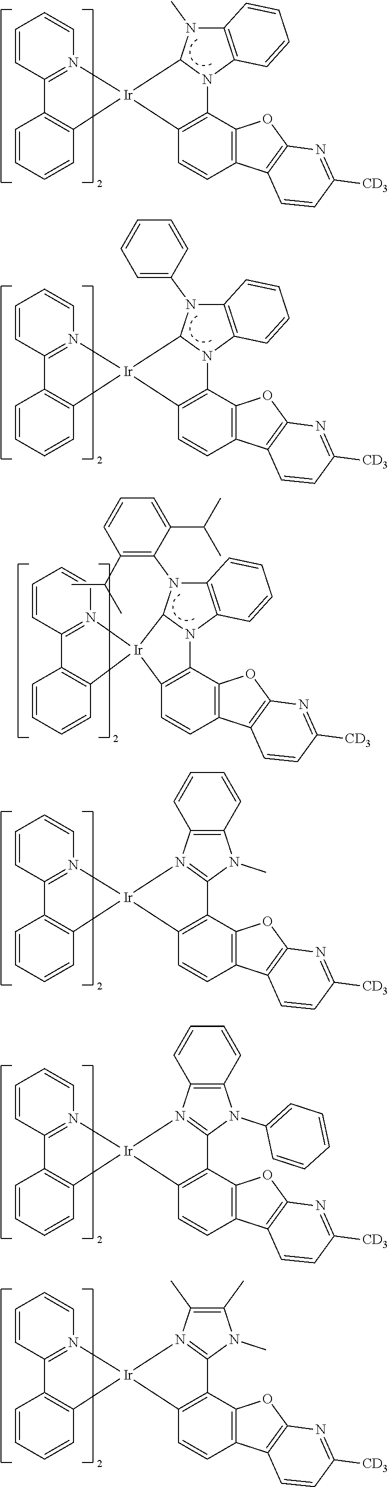

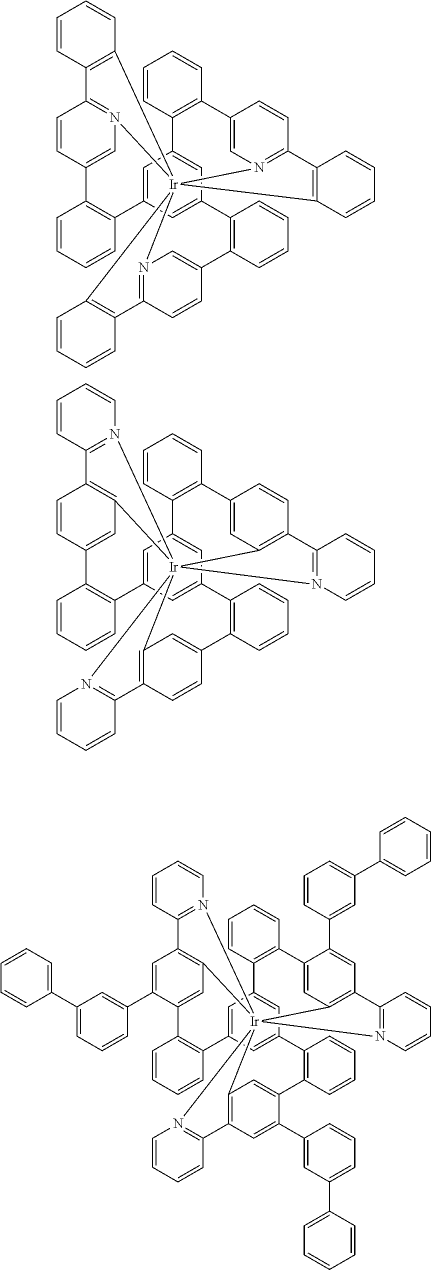

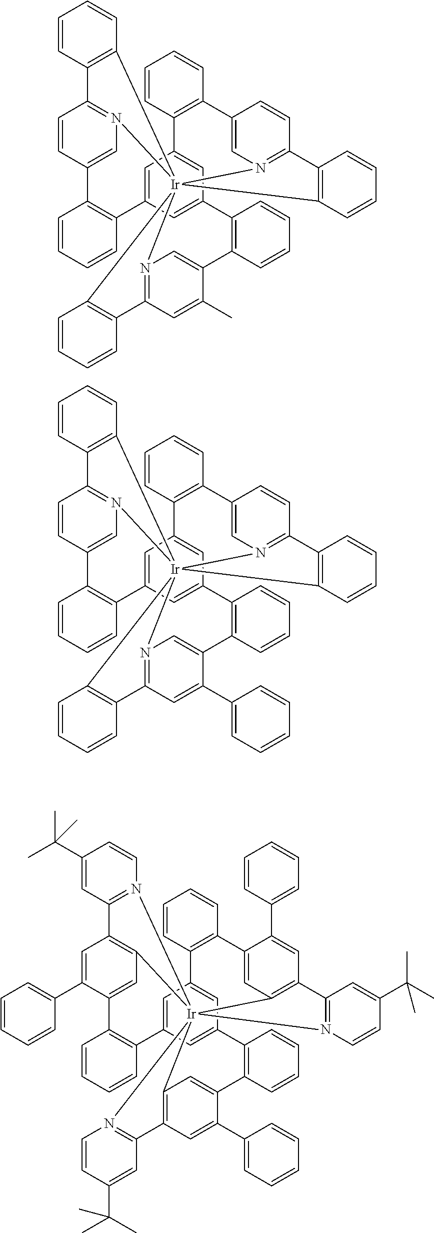

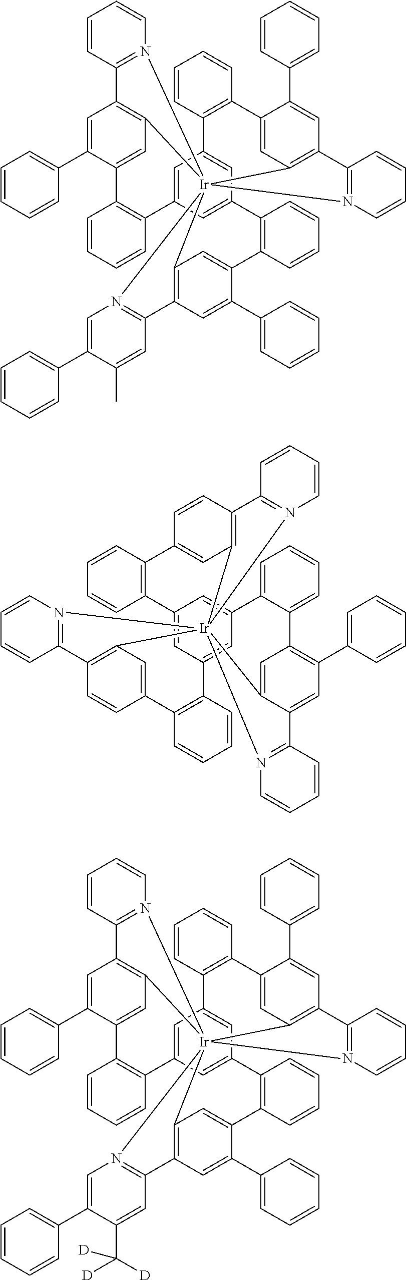

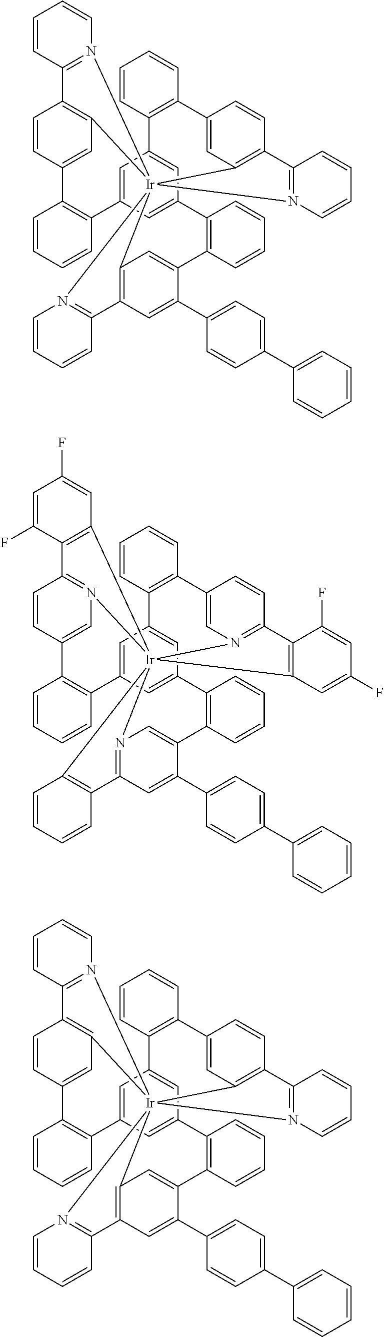

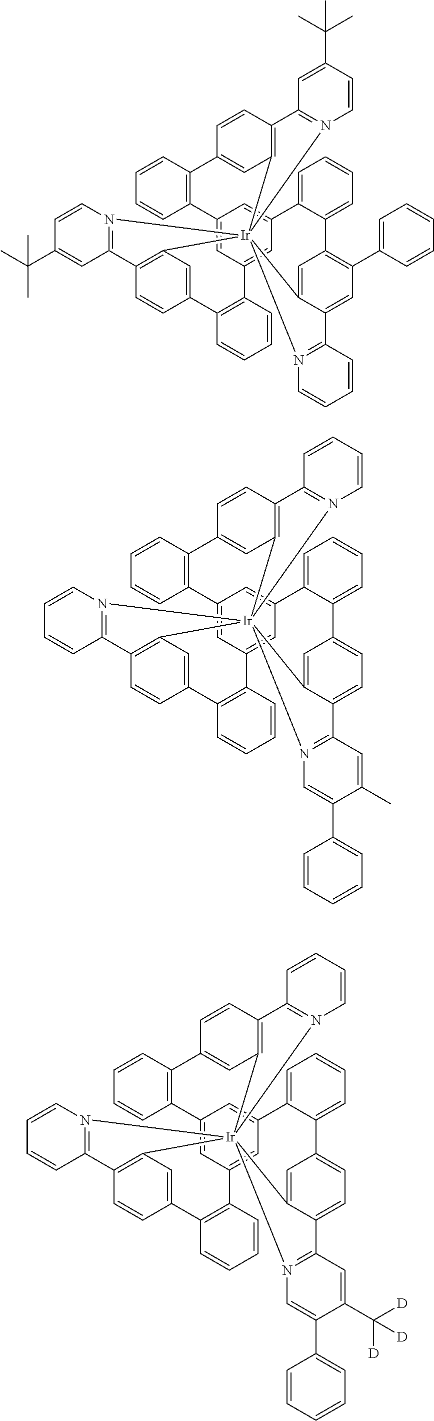

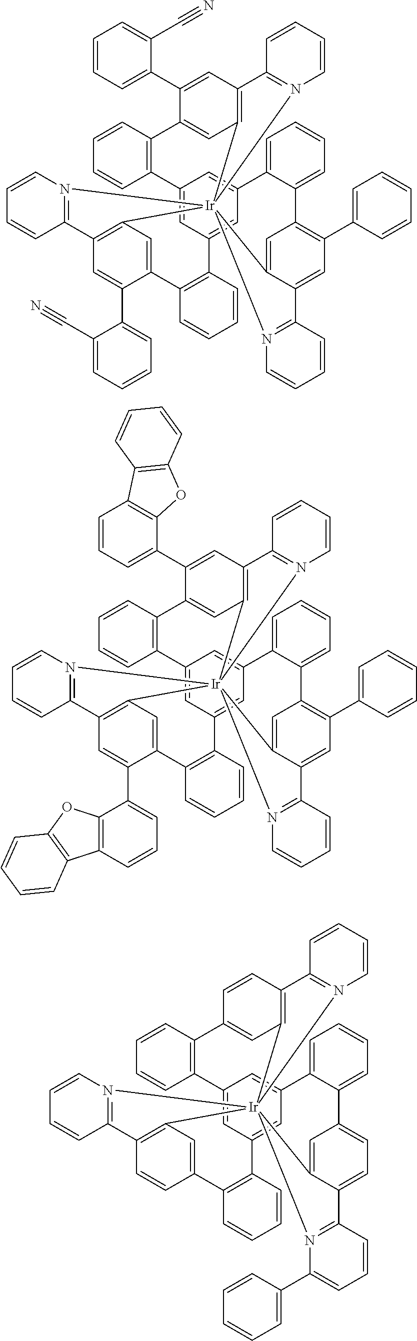

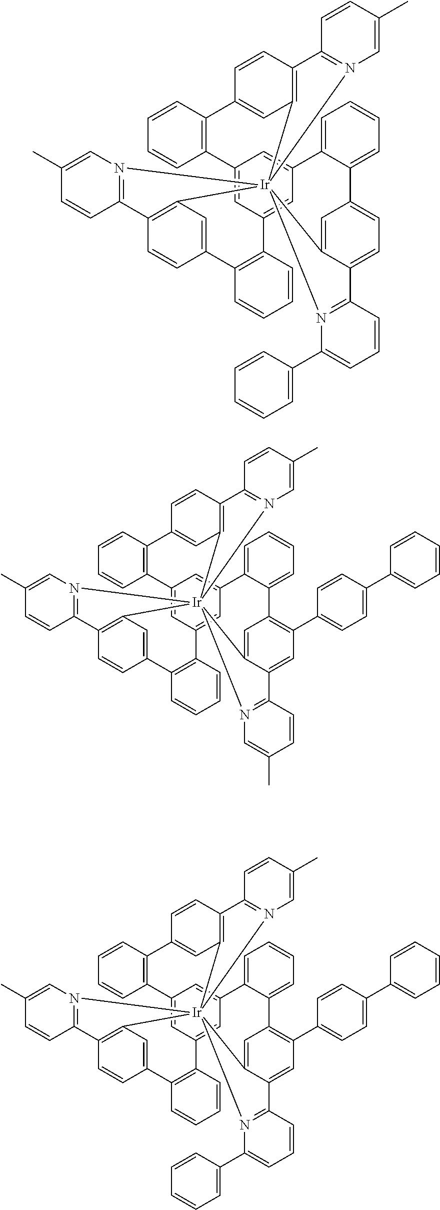

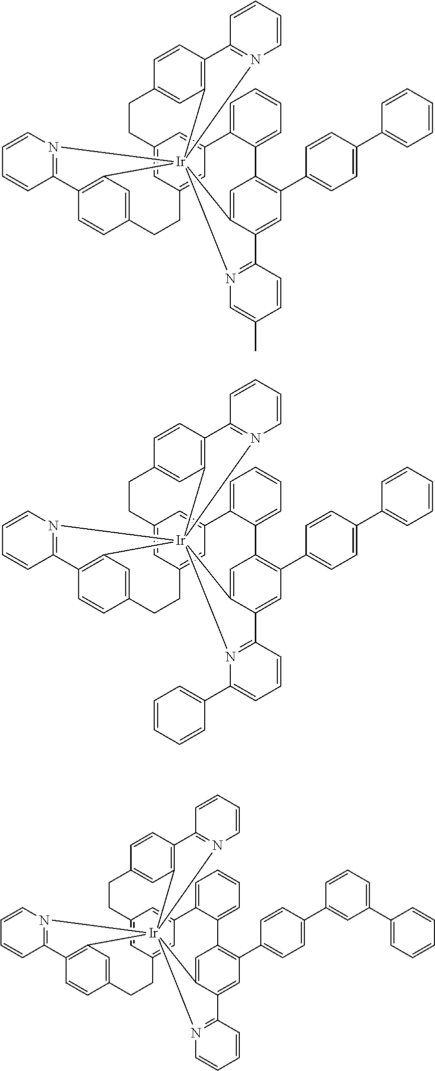

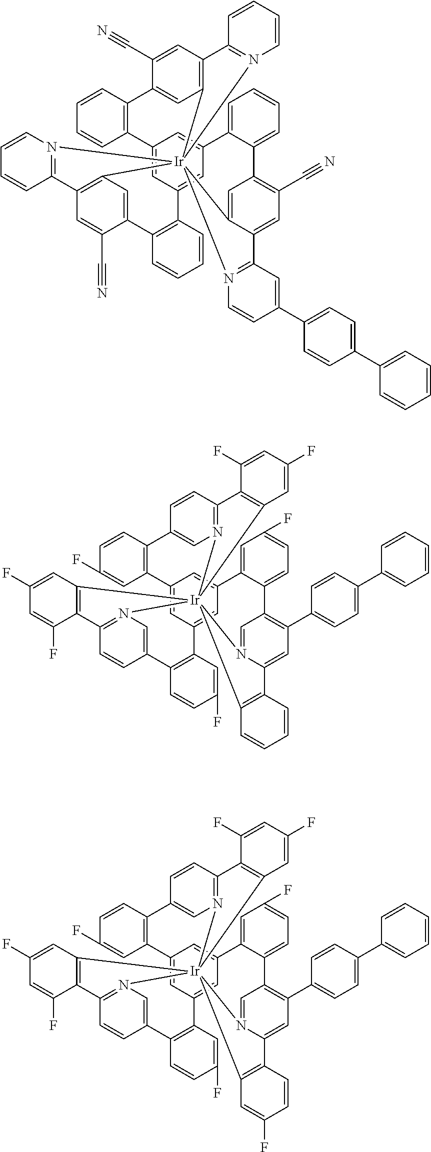

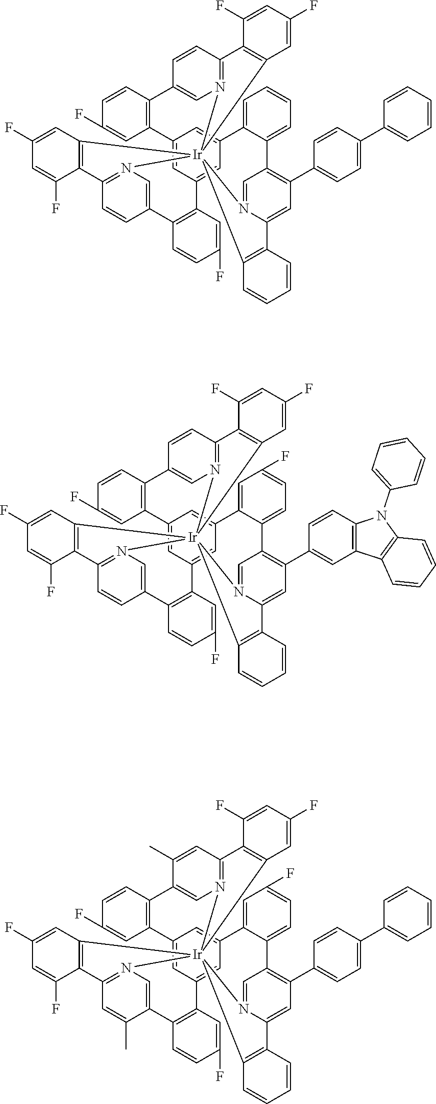

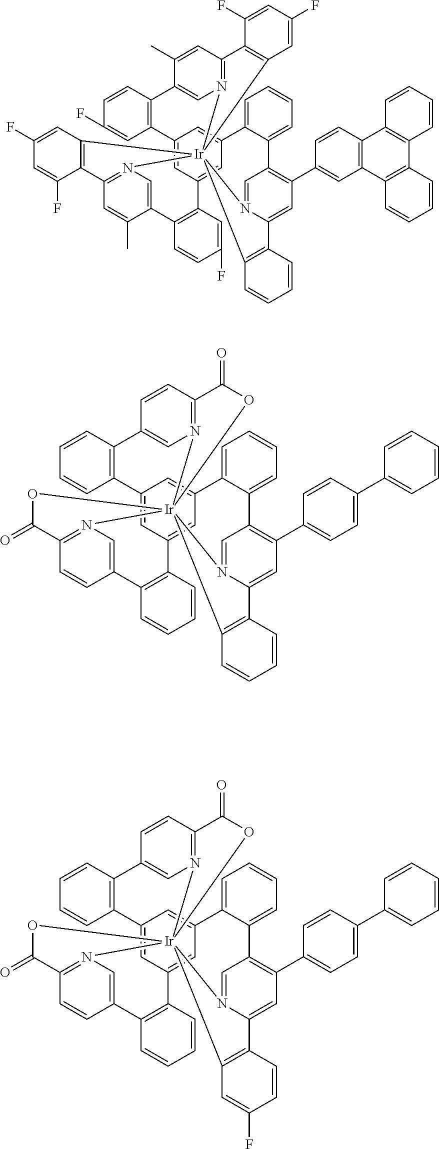

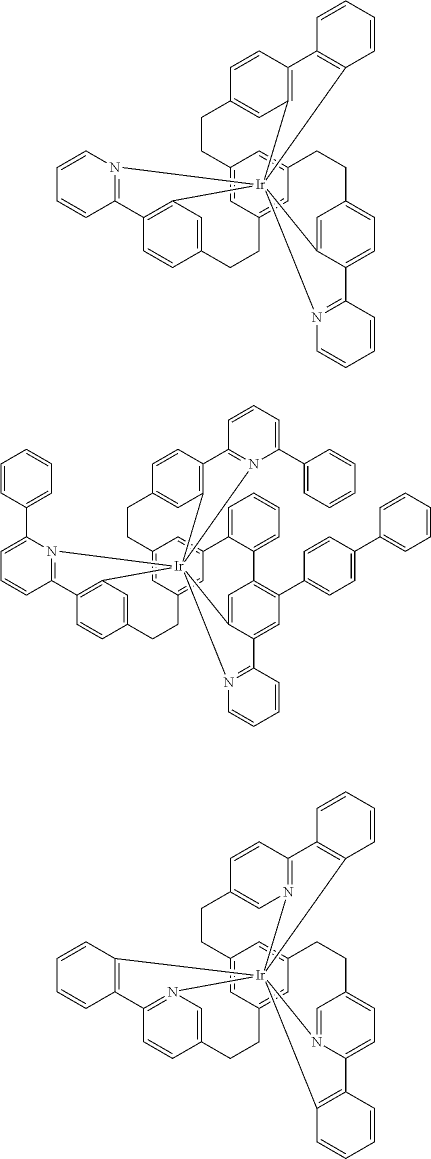

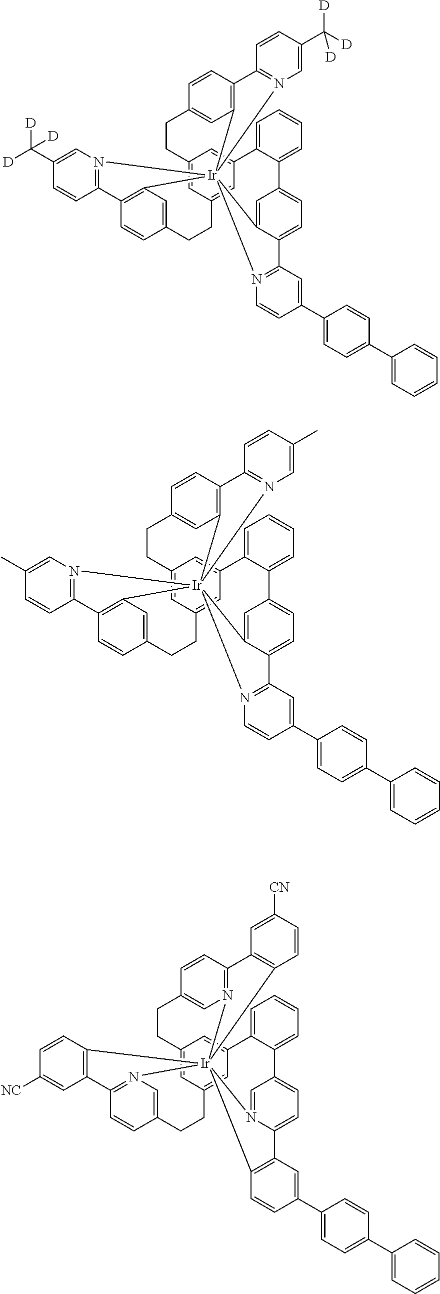

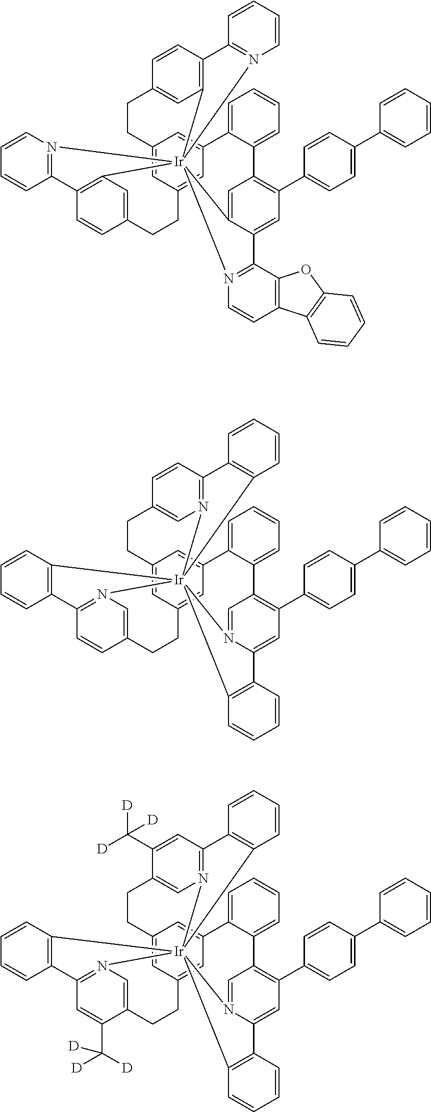

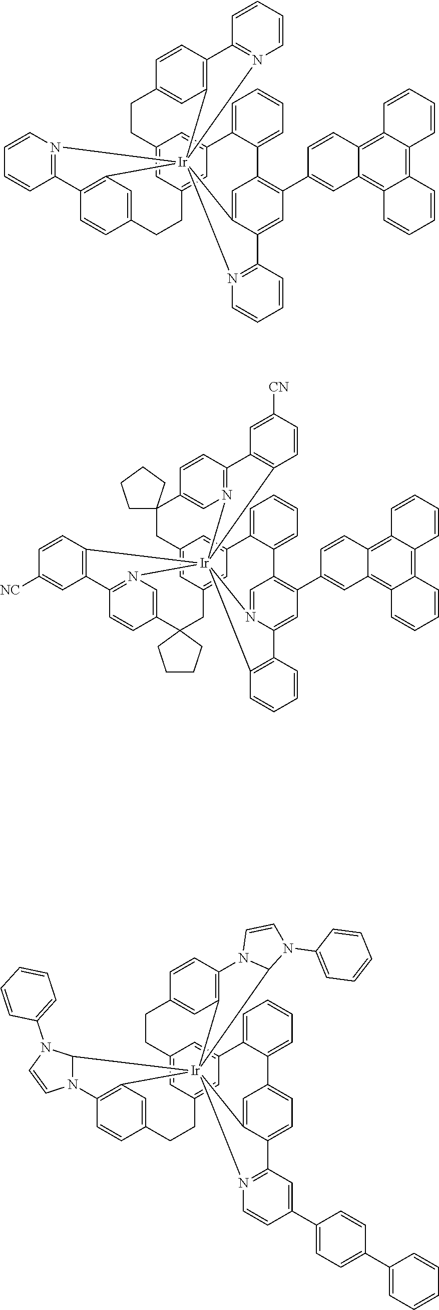

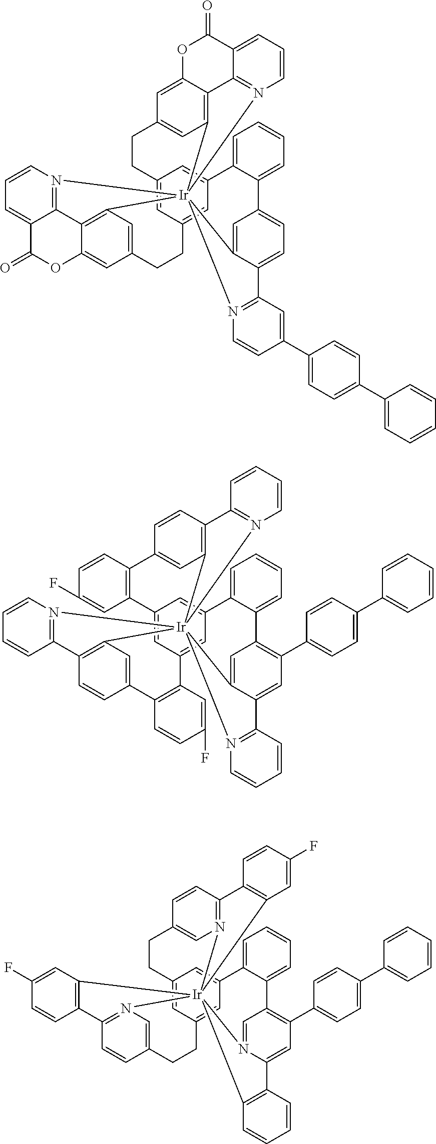

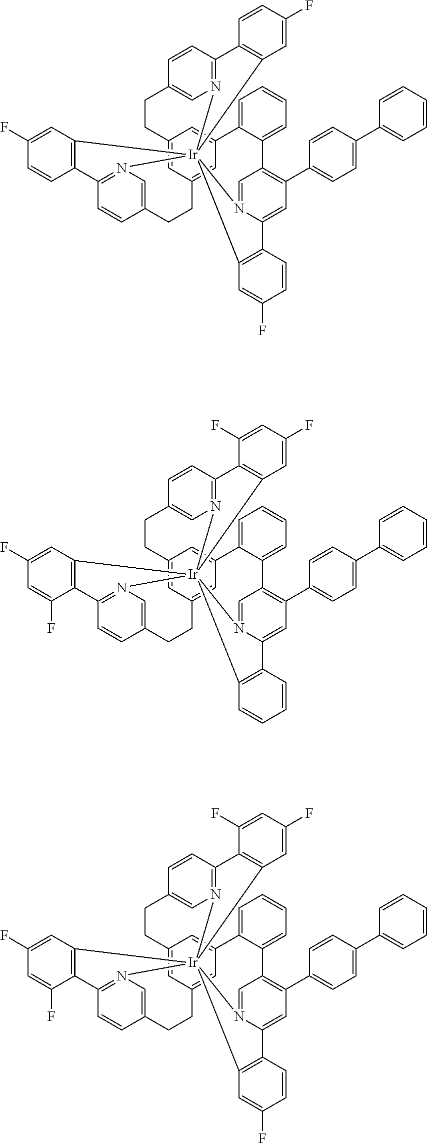

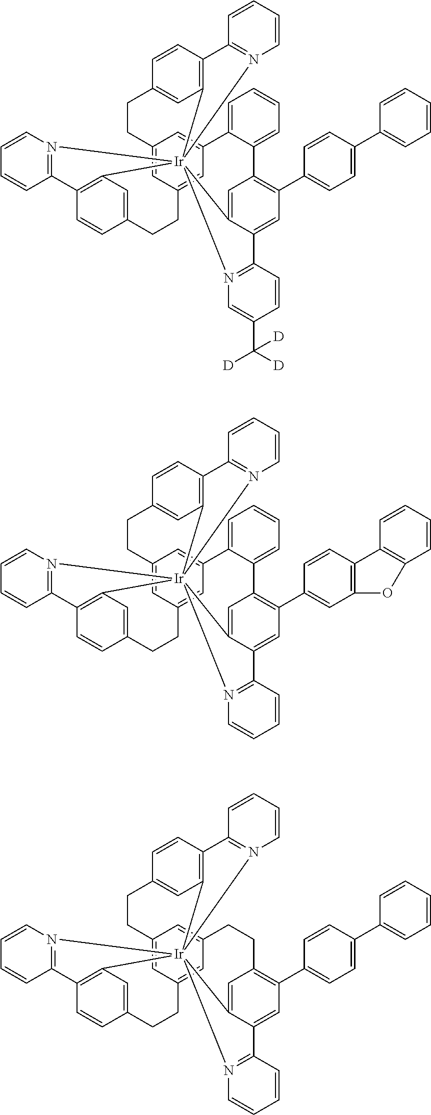

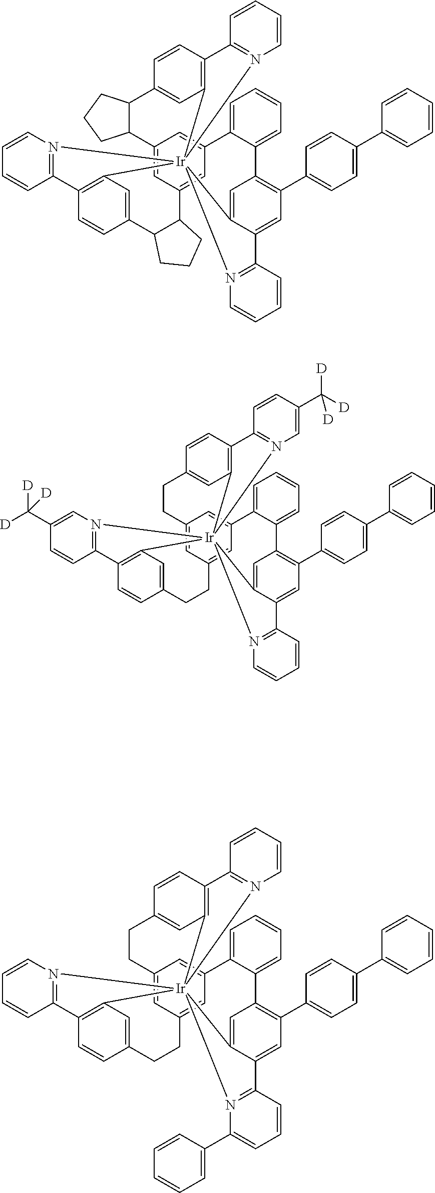

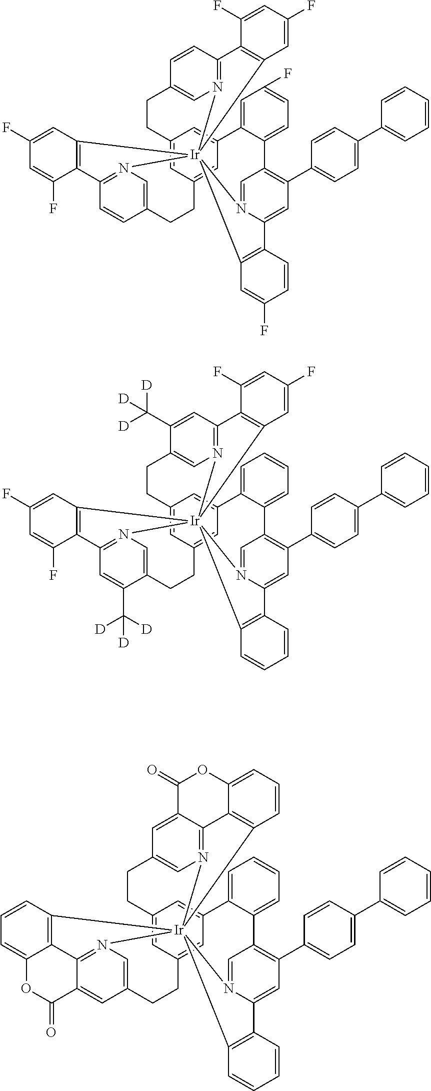

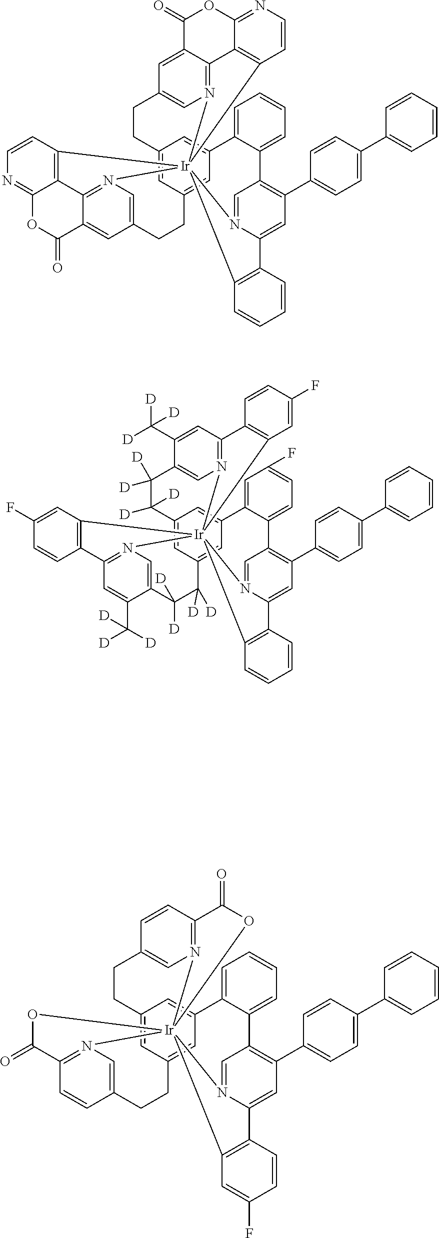

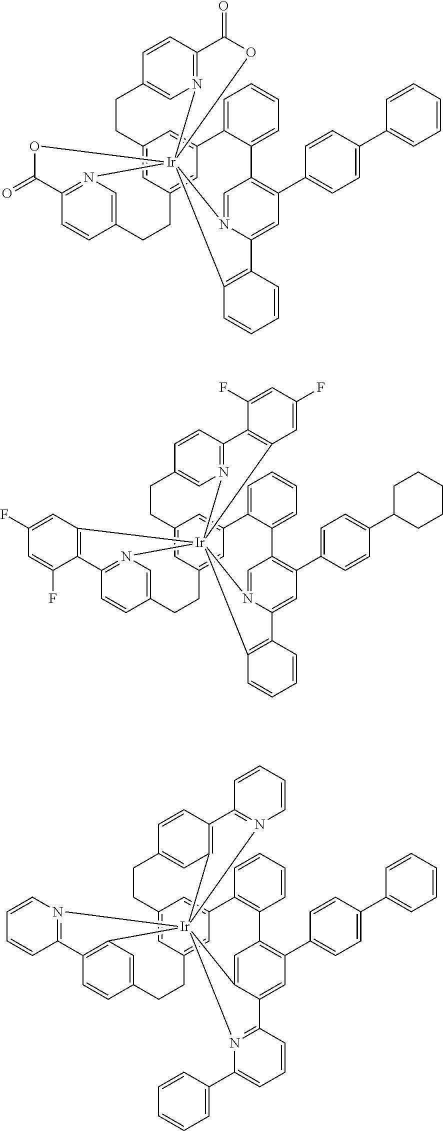

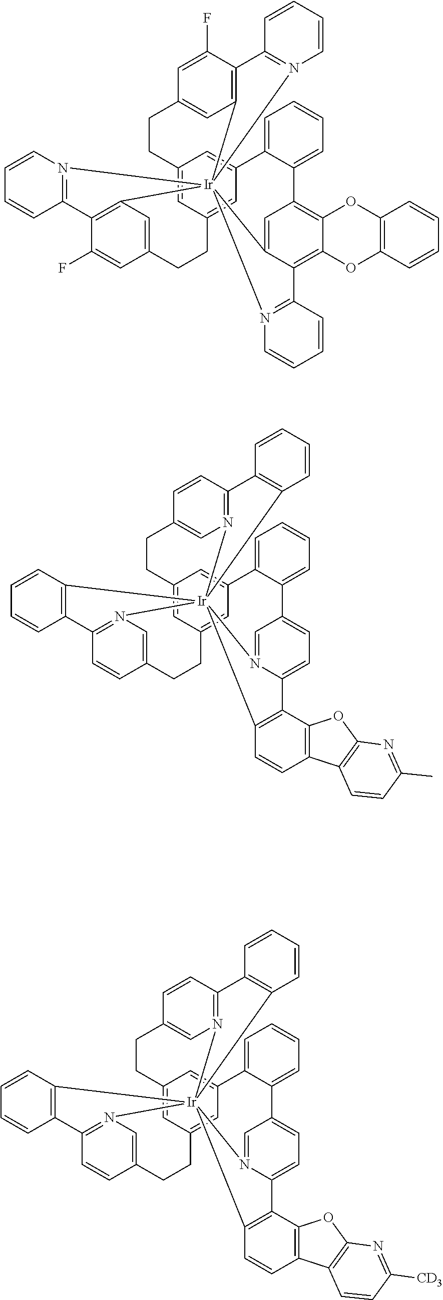

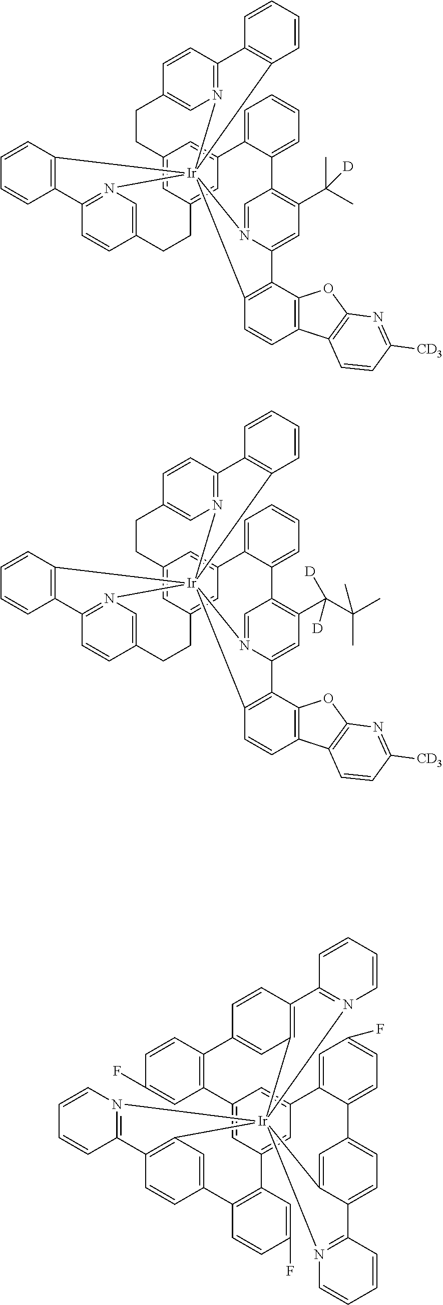

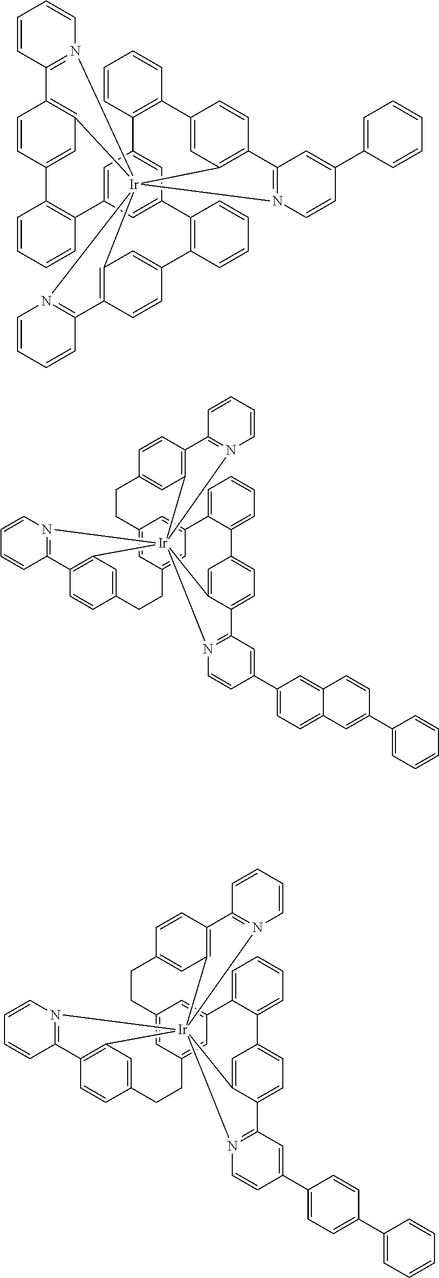

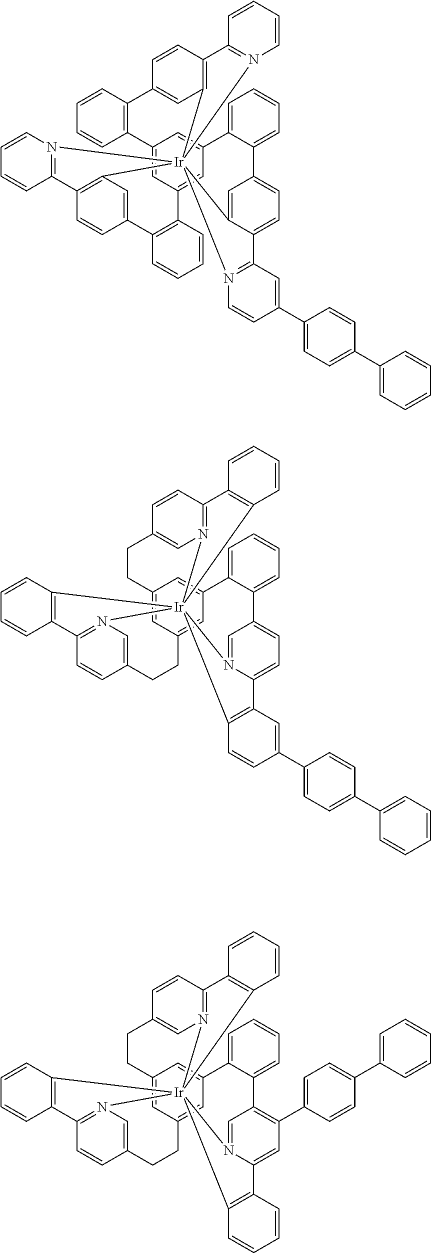

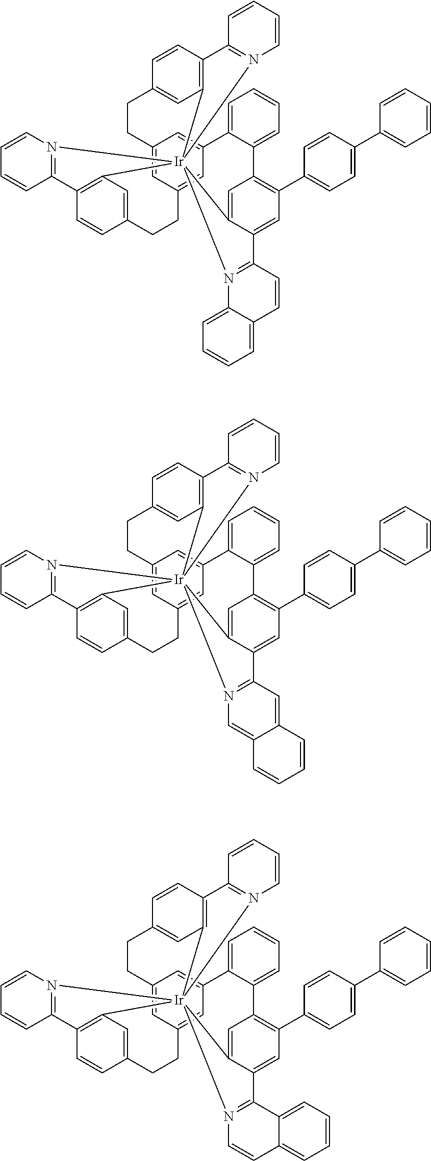

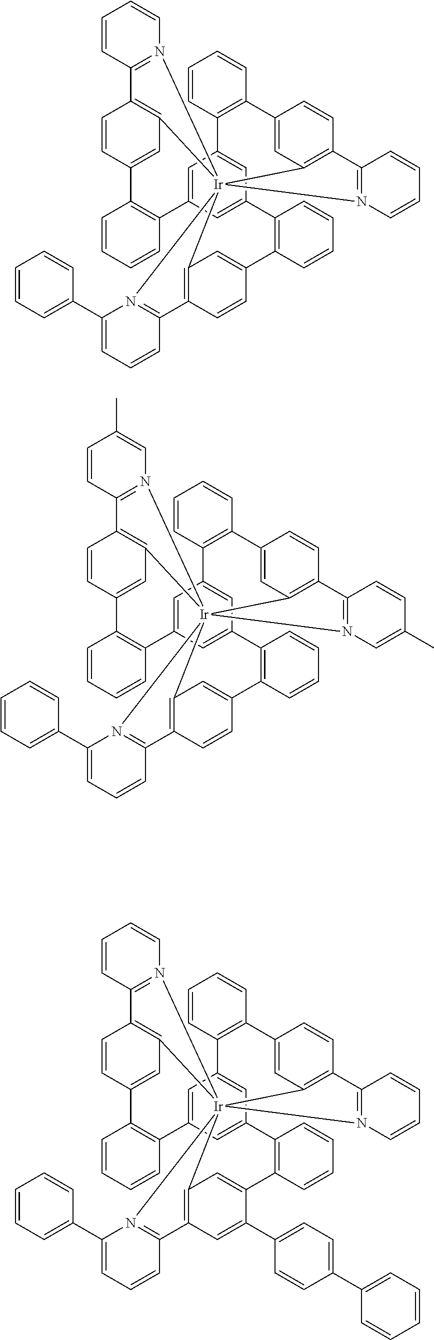

### Fluorescent Emitters:

Preferred fluorescent emitting compounds are selected from the class of the arylamines. An arylamine or an aromatic amine in the context of this invention is understood to mean a compound containing three substituted or unsubstituted aromatic or heteroaromatic ring systems bonded directly to the nitrogen. Preferably, at least one of these aromatic or heteroaromatic ring systems is a fused ring system, more preferably having at least 14 aromatic ring atoms. Preferred examples of these are aromatic anthraceneamines, aromatic anthracenediamines, aromatic pyreneamines, aromatic pyrenediamines, aromatic chryseneamines or aromatic chrysenediamines. An aromatic anthraceneamine is understood to mean a compound in which a diarylamino group is bonded directly to an anthracene group, preferably in the 9 position. An aromatic anthracenediamine is understood to mean a compound in which two diarylamino groups are bonded directly to an anthracene group, preferably in the 9,10 position. Aromatic pyreneamines, pyrenediamines, chryseneamines and chrysenediamines are defined analogously, where the diarylamino groups are bonded to the pyrene preferably in the 1 position or 1,6 position. Further preferred emitting compounds are indenofluoreneamines or -diamines, benzoindenofluoreneamines or -diamines, and dibenzoindenofluoreneamines or -diamines, and indenofluorene derivatives having fused aryl groups. Likewise preferred are pyrenearylamines. Likewise preferred are benzoindenofluoreneamines, benzofluoreneamines, extended benzoindenofluorenes, phenoxazines, and fluorene derivatives joined to furan units or to thiophene units.

### Matrix Materials for Fluorescent Emitters:

Preferred matrix materials for fluorescent emitters are selected from the classes of the oligoarylenes (e.g. 2,2′,7,7′-tetraphenylspirobifluorene), especially the oligoarylenes containing fused aromatic groups, the oligoarylenevinylenes, the polypodal metal complexes, the hole-conducting compounds, the electron-conducting compounds, especially ketones, phosphine oxides and sulfoxides; the atropisomers, the boronic acid derivatives or the benzanthracenes. Particularly preferred matrix materials are selected from the classes of the oligoarylenes comprising naphthalene, anthracene, benzanthracene and/or pyrene or atropisomers of these compounds, the oligoarylenevinylenes, the ketones, the phosphine oxides and the sulfoxides. Very particularly preferred matrix materials are selected from the classes of the oligoarylenes comprising anthracene, benzanthracene, benzophenanthrene and/or pyrene or atropisomers of these compounds. An oligoarylene in the context of this invention shall be understood to mean a compound in which at least three aryl or arylene groups are bonded to one another.

### Matrix Materials for Phosphorescent Emitters:

Preferred matrix materials for phosphorescent emitters are, as well as the compounds of the formula (I), (II) or (III), aromatic ketones, aromatic phosphine oxides or aromatic sulfoxides or sulfones, triarylamines, carbazole derivatives, e.g. CBP (N,N-biscarbazolylbiphenyl) or carbazole derivatives, indolocarbazole derivatives, indenocarbazole derivatives, azacarbazole derivatives, bipolar matrix materials, silanes, azaboroles or boronic esters, triazine derivatives, zinc complexes, diazasilole or tetraazasilole derivatives, diazaphosphole derivatives, bridged carbazole derivatives, triphenylene derivatives, or lactams.

### Electron-Transporting Materials:

Suitable electron-transporting materials are, for example, the compounds disclosed in Y. Shirota et al., Chem. Rev. 2007, 107(4), 953-1010, or other materials used in these layers according to the prior art.

Materials used for the electron transport layer may be any materials that are used as electron transport materials in the electron transport layer according to the prior art. Especially suitable are aluminium complexes, for example Alq₃, zirconium complexes, for example Zrq₄, lithium complexes, for example Liq, benzimidazole derivatives, triazine derivatives, pyrimidine derivatives, pyridine derivatives, pyrazine derivatives, quinoxaline derivatives, quinoline derivatives, oxadiazole derivatives, aromatic ketones, lactams, boranes, diazaphosphole derivatives and phosphine oxide derivatives.

Preferred electron transport and electron injection materials are shown in the following table:

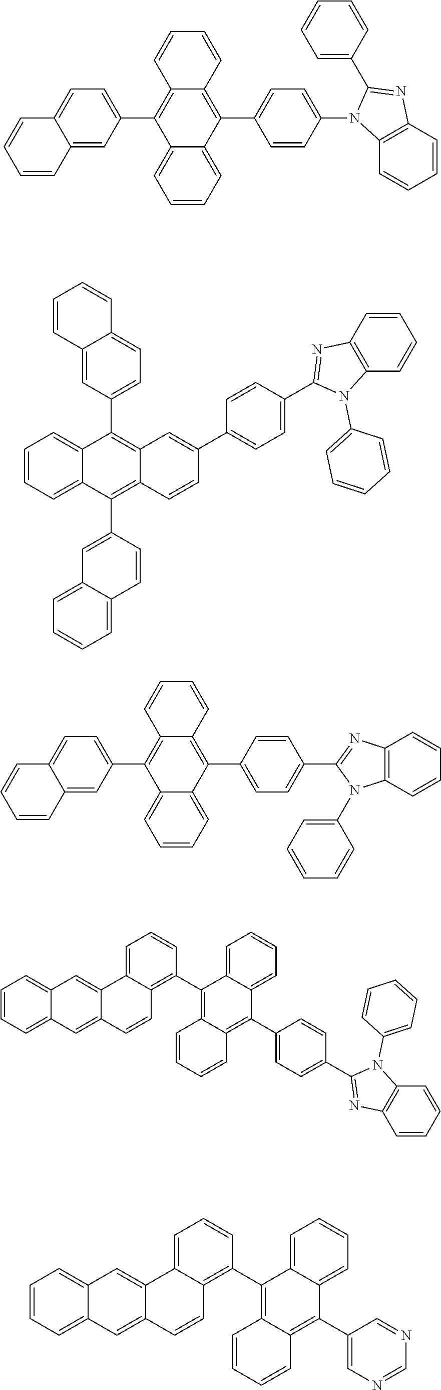 [SMILES: c1ccc(-c2nc3ccccc3n2-c2ccc(-c3c4ccccc4c(-c4ccc5ccccc5c4)c4ccccc34)cc2)cc1 ; c1ccc(-n2c(-c3ccc(-c4ccc5c(-c6ccc7ccccc7c6)c6ccccc6c(-c6ccc7ccccc7c6)c5c4)cc3)nc3ccccc32)cc1 ; c1ccc(-n2c(-c3ccc(-c4c5ccccc5c(-c5ccc6ccccc6c5)c5ccccc45)cc3)nc3ccccc32)cc1 ; c1ccc(-n2c(-c3ccc(-c4c5ccccc5c(-c5cccc6c5ccc5cc7ccccc7cc56)c5ccccc45)cc3)nc3ccccc32)cc1 ; c1ccc2cc3c(ccc4c(-c5c6ccccc6c(-c6cncnc6)c6ccccc56)cccc43)cc2c1]

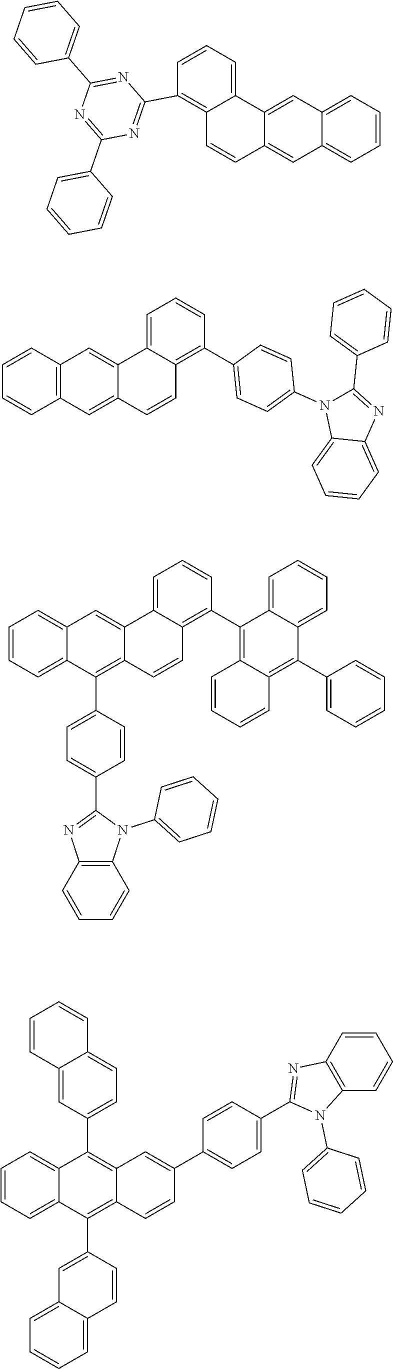 [SMILES: c1ccc(-c2nc(-c3ccccc3)nc(-c3cccc4c3ccc3cc5ccccc5cc34)n2)cc1 ; c1ccc(-c2nc3ccccc3n2-c2ccc(-c3cccc4c3ccc3cc5ccccc5cc34)cc2)cc1 ; c1ccc(-c2c3ccccc3c(-c3cccc4c3ccc3c(-c5ccc(-c6nc7ccccc7n6-c6ccccc6)cc5)c5ccccc5cc34)c3ccccc23)cc1 ; c1ccc(-n2c(-c3ccc(-c4ccc5c(-c6ccc7ccccc7c6)c6ccccc6c(-c6ccc7ccccc7c6)c5c4)cc3)nc3ccccc32)cc1]

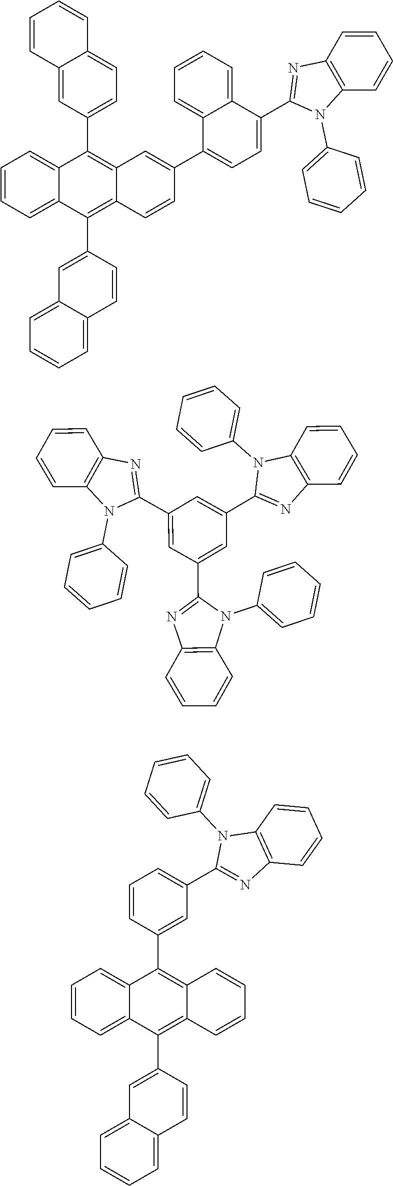 [SMILES: c1ccc(-n2c(-c3ccc(-c4ccc5c(-c6ccc7ccccc7c6)c6ccccc6c(-c6ccc7ccccc7c6)c5c4)c4ccccc34)nc3ccccc32)cc1 ; c1ccc(-n2c(-c3cc(-c4nc5ccccc5n4-c4ccccc4)cc(-c4nc5ccccc5n4-c4ccccc4)c3)nc3ccccc32)cc1 ; c1ccc(-n2c(-c3cccc(-c4c5ccccc5c(-c5ccc6ccccc6c5)c5ccccc45)c3)nc3ccccc32)cc1]

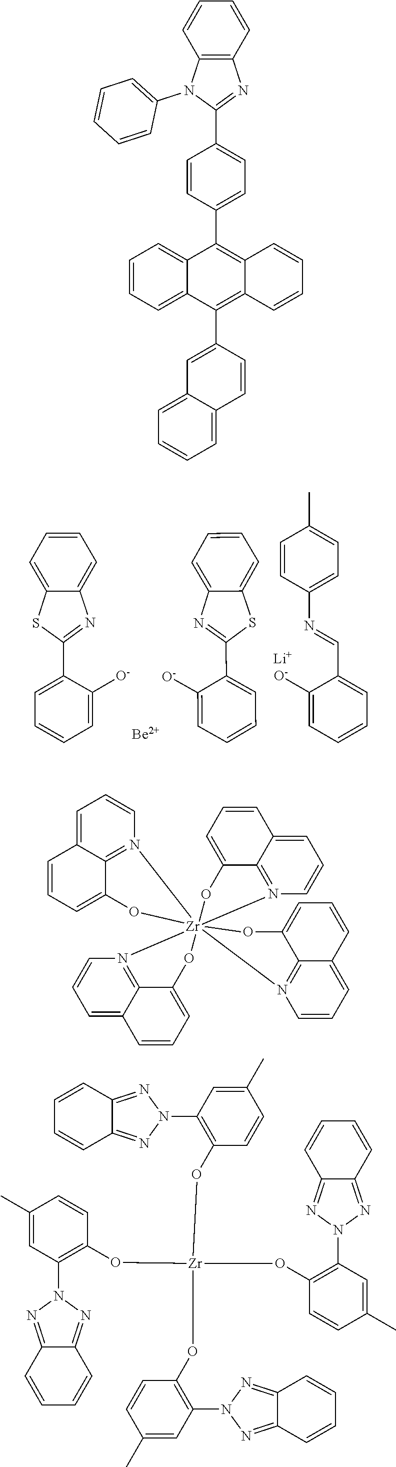

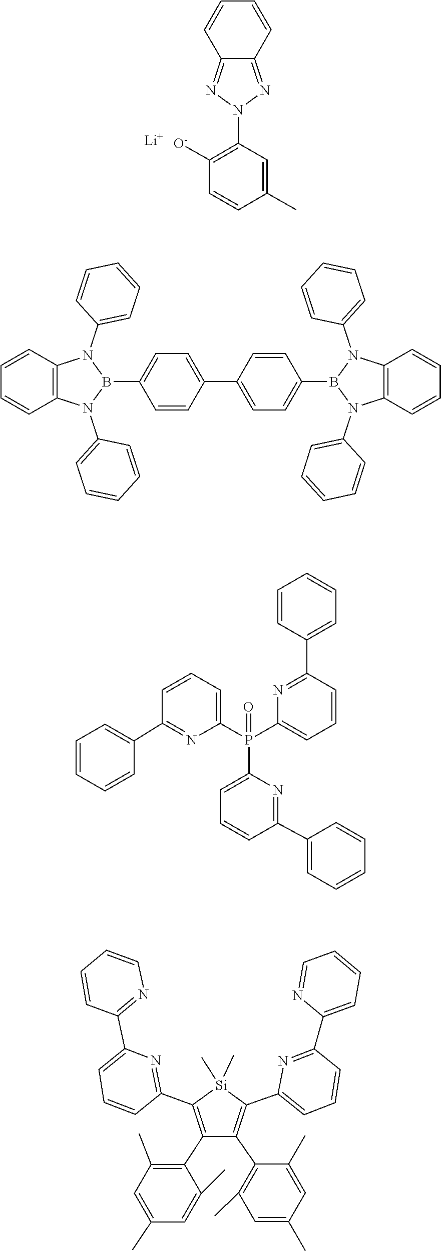 [SMILES: [Li+] ; Cc1ccc([O-])c(-n2nc3ccccc3n2)c1 ; c1ccc(N2B(c3ccc(-c4ccc(B5N(c6ccccc6)c6ccccc6N5c5ccccc5)cc4)cc3)N(c3ccccc3)c3ccccc32)cc1 ; O=P(c1cccc(-c2ccccc2)n1)(c1cccc(-c2ccccc2)n1)c1cccc(-c2ccccc2)n1 ; Cc1cc(C)c(C2=C(c3cccc(-c4ccccn4)n3)[Si](C)(C)C(c3cccc(-c4ccccn4)n3)=C2c2c(C)cc(C)cc2C)c(C)c1]

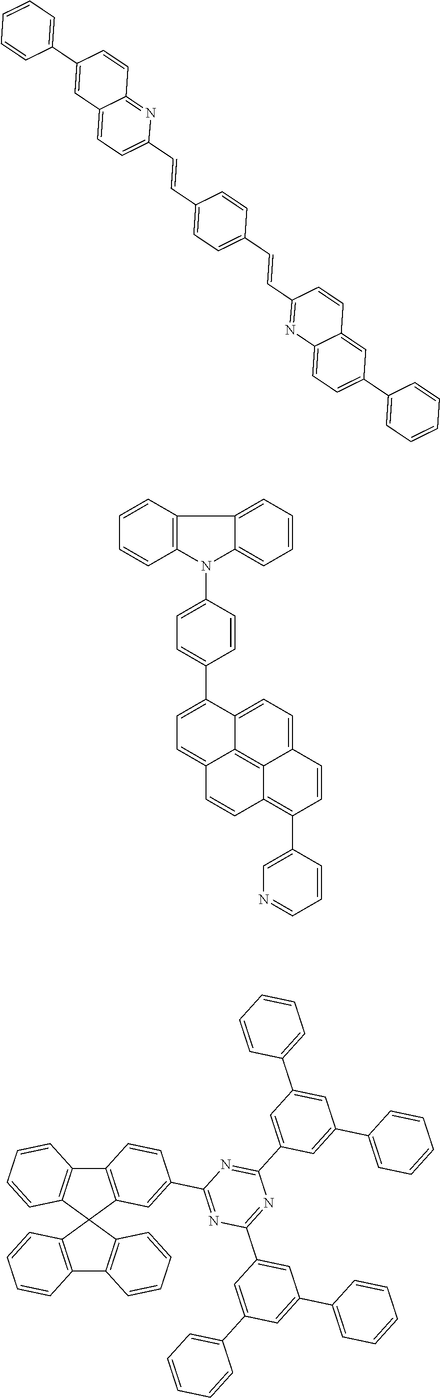 [SMILES: C(=C\c1ccc2cc(-c3ccccc3)ccc2n1)/c1ccc(/C=C/c2ccc3cc(-c4ccccc4)ccc3n2)cc1 ; c1cncc(-c2ccc3ccc4c(-c5ccc(-n6c7ccccc7c7ccccc76)cc5)ccc5ccc2c3c54)c1 ; c1ccc(-c2cc(-c3ccccc3)cc(-c3nc(-c4cc(-c5ccccc5)cc(-c5ccccc5)c4)nc(-c4ccc5c(c4)C4(c6ccccc6-c6ccccc64)c4ccccc4-5)n3)c2)cc1]

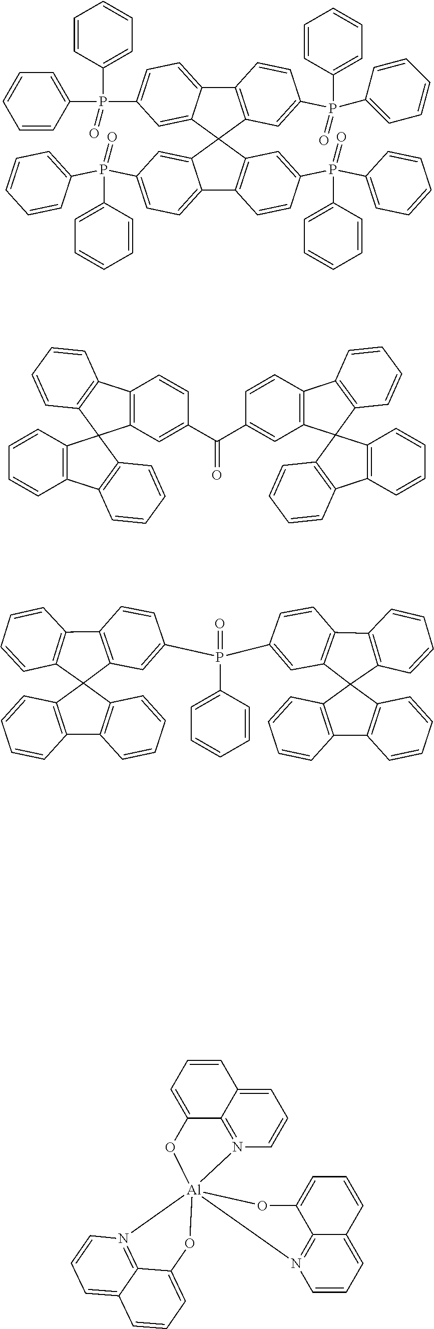

### Hole-Transporting Materials:

Further compounds which, in addition to the compounds of the formula (I), (II) or (III), are preferably used in hole-transporting layers of the OLEDs of the invention are indenofluoreneamine derivatives, amine derivatives, hexaazatriphenylene derivatives, amine derivatives with fused aromatic systems, monobenzoindenofluoreneamines, dibenzoindenofluoreneamines, spirobifluoreneamines, fluoreneamines, spirodibenzopyranamines, dihydroacridine derivatives, spirodibenzofurans and spirodibenzothiophenes, phenanthrenediarylamines, spirotribenzotropolones, spirobifluorenes having meta-phenyldiamine groups, spirobisacridines, xanthenediarylamines, and 9,10-dihydroanthracene spiro compounds having diarylamino groups. Preferred hole-transporting compounds are shown in the following table:

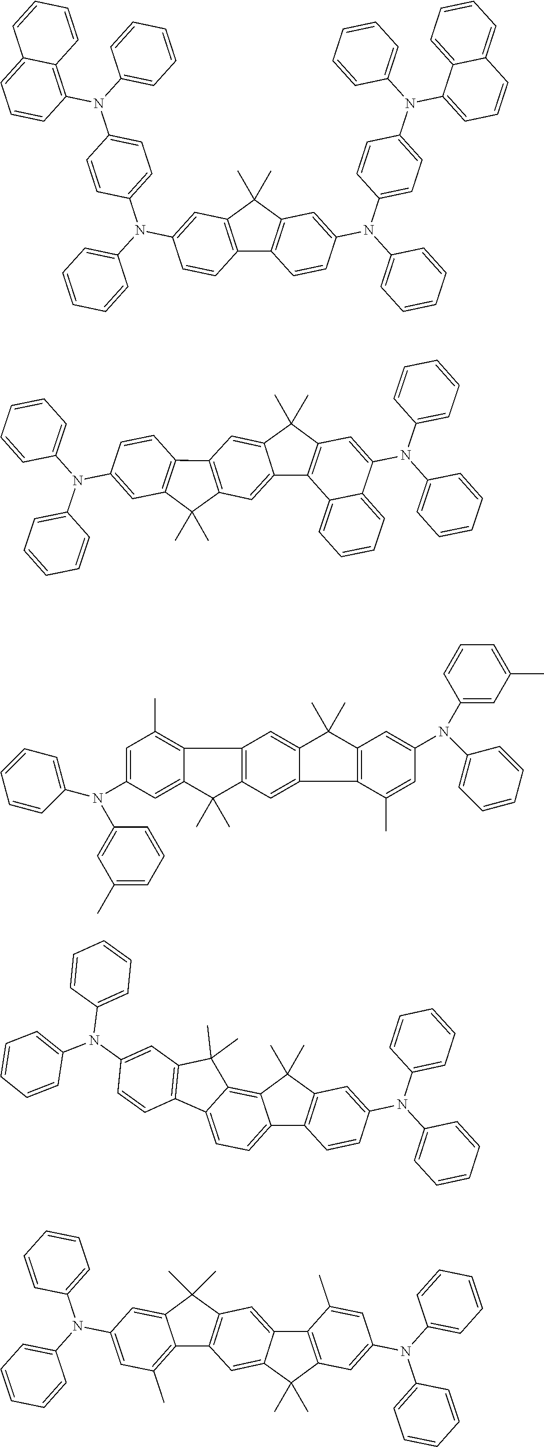

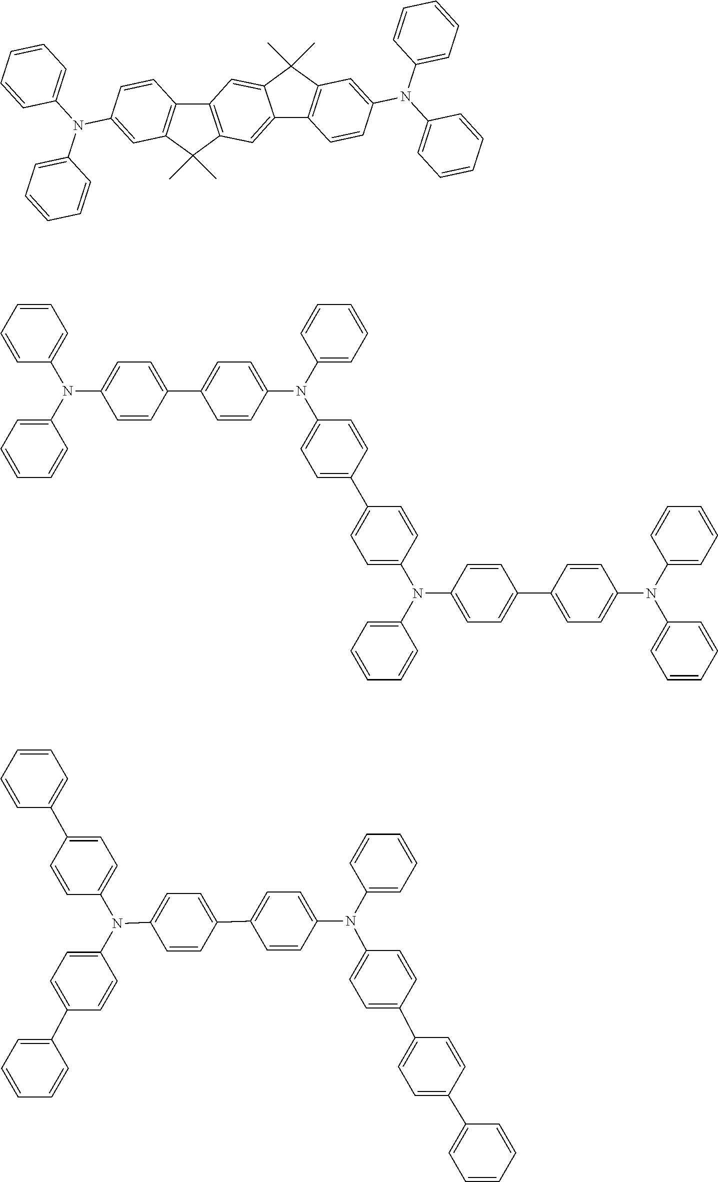 [SMILES: CC1(C)c2cc(N(c3ccccc3)c3ccccc3)ccc2-c2cc3c(cc21)-c1ccc(N(c2ccccc2)c2ccccc2)cc1C3(C)C ; c1ccc(N(c2ccccc2)c2ccc(-c3ccc(N(c4ccccc4)c4ccc(-c5ccc(N(c6ccccc6)c6ccc(-c7ccc(N(c8ccccc8)c8ccccc8)cc7)cc6)cc5)cc4)cc3)cc2)cc1 ; c1ccc(-c2ccc(-c3ccc(N(c4ccccc4)c4ccc(-c5ccc(N(c6ccc(-c7ccccc7)cc6)c6ccc(-c7ccccc7)cc6)cc5)cc4)cc3)cc2)cc1]

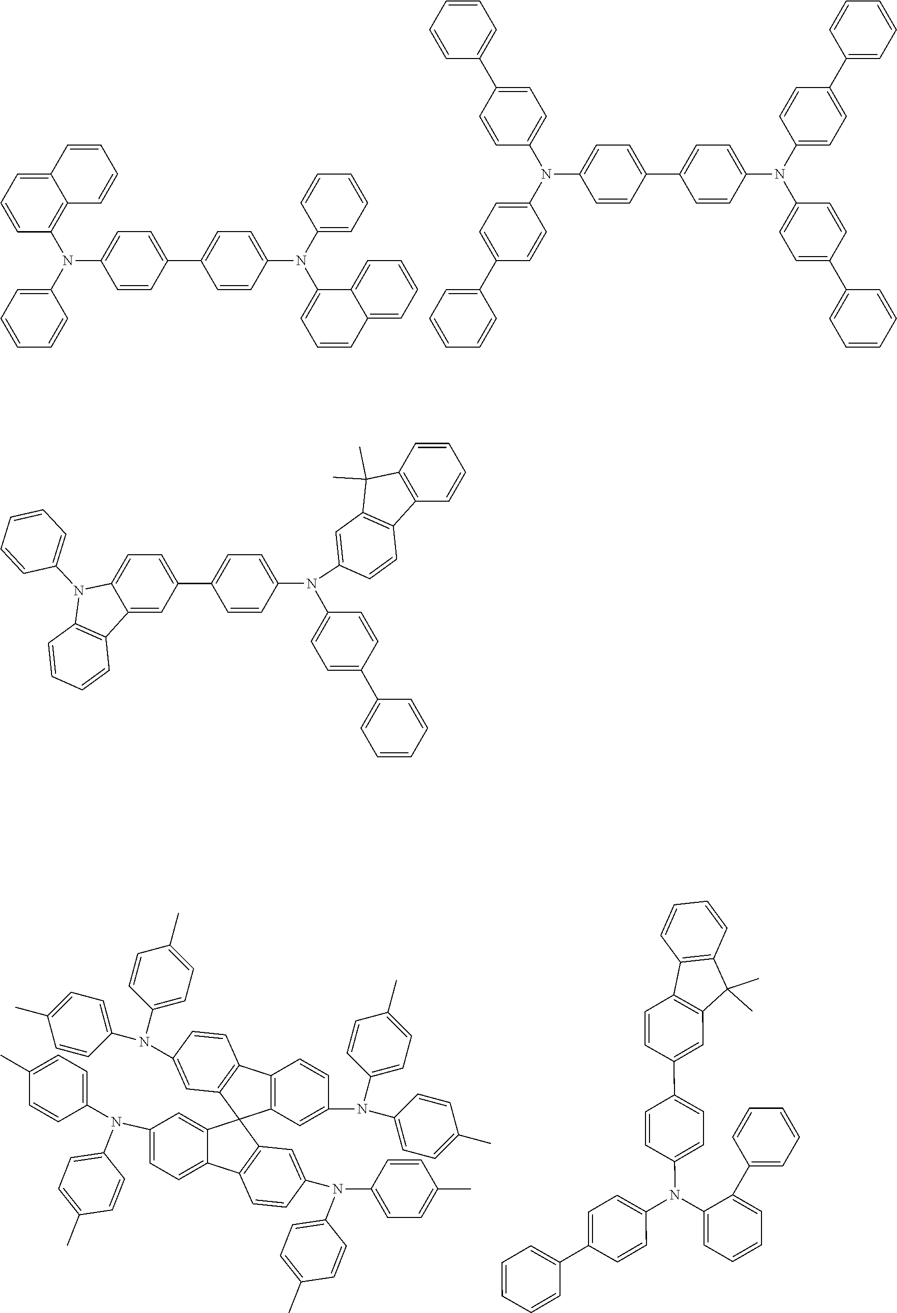

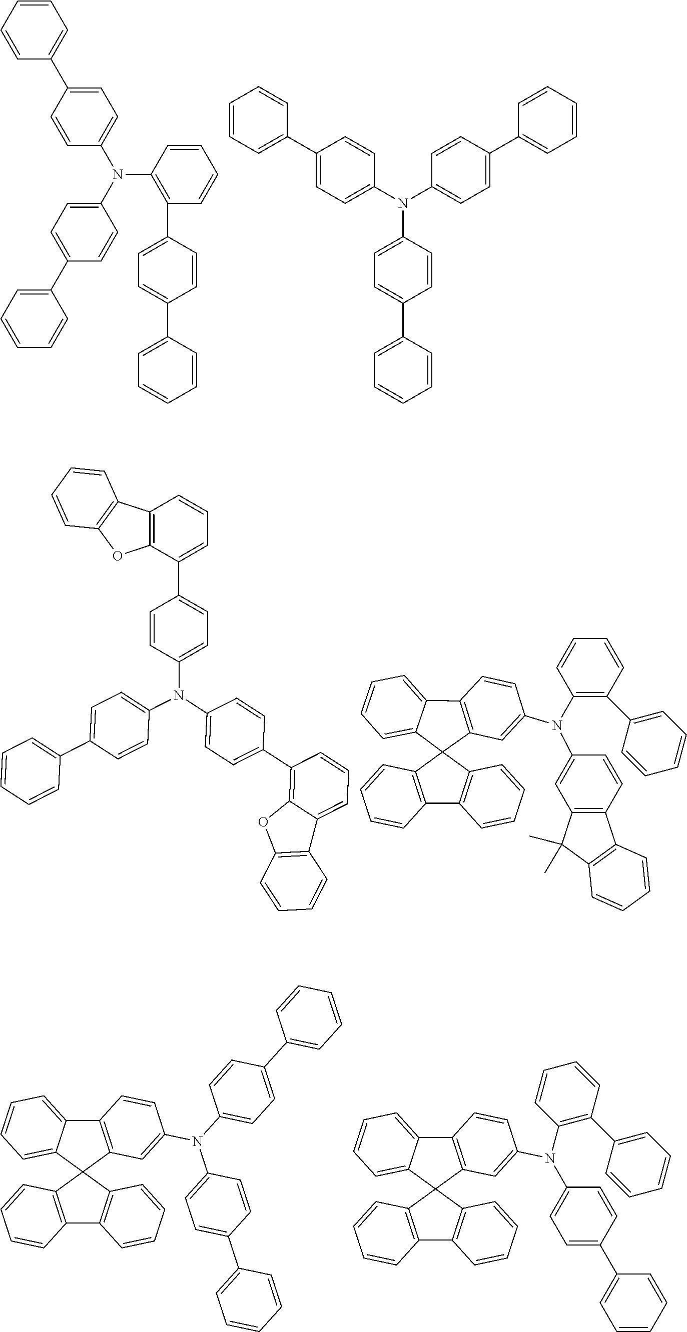

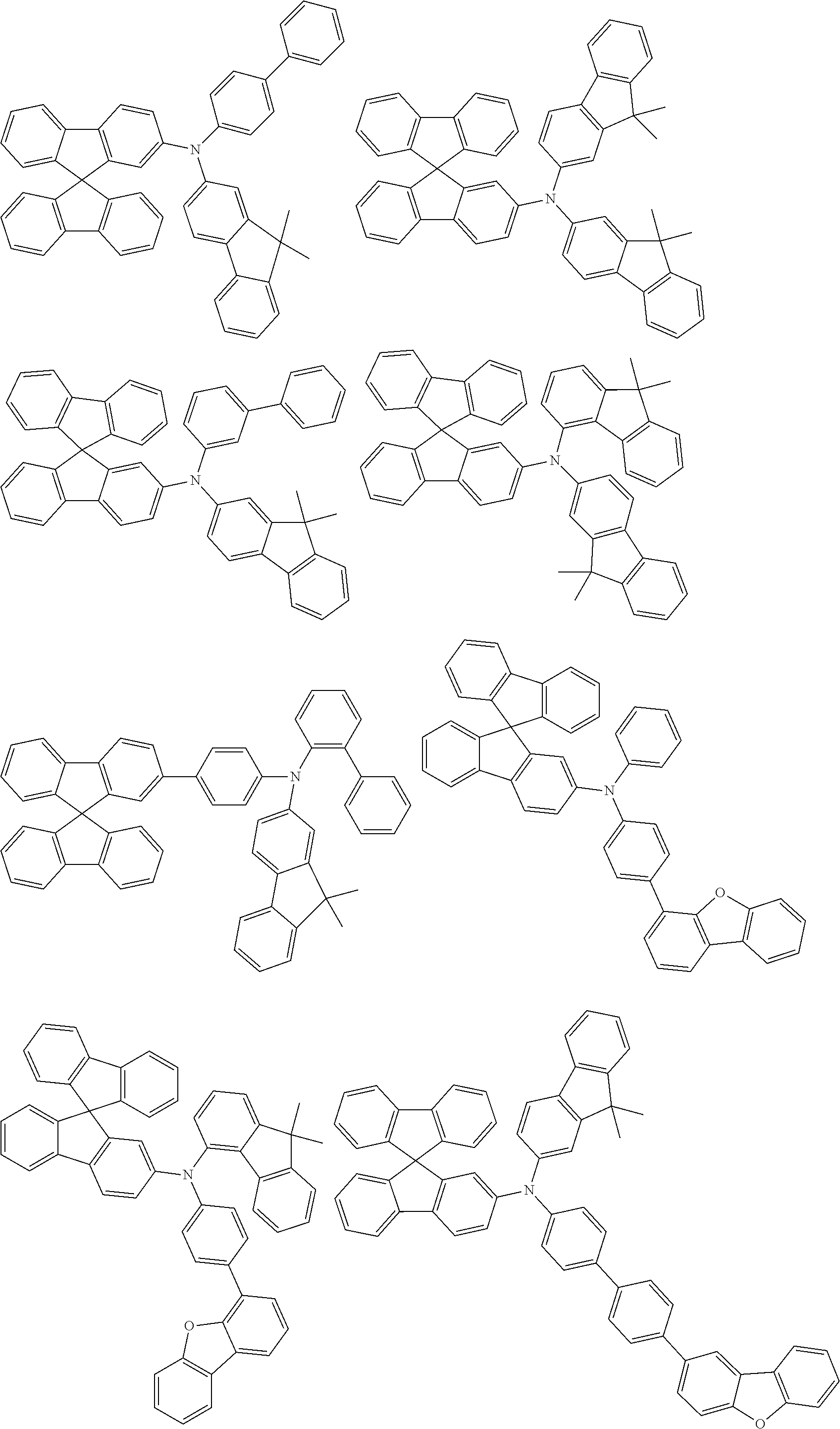

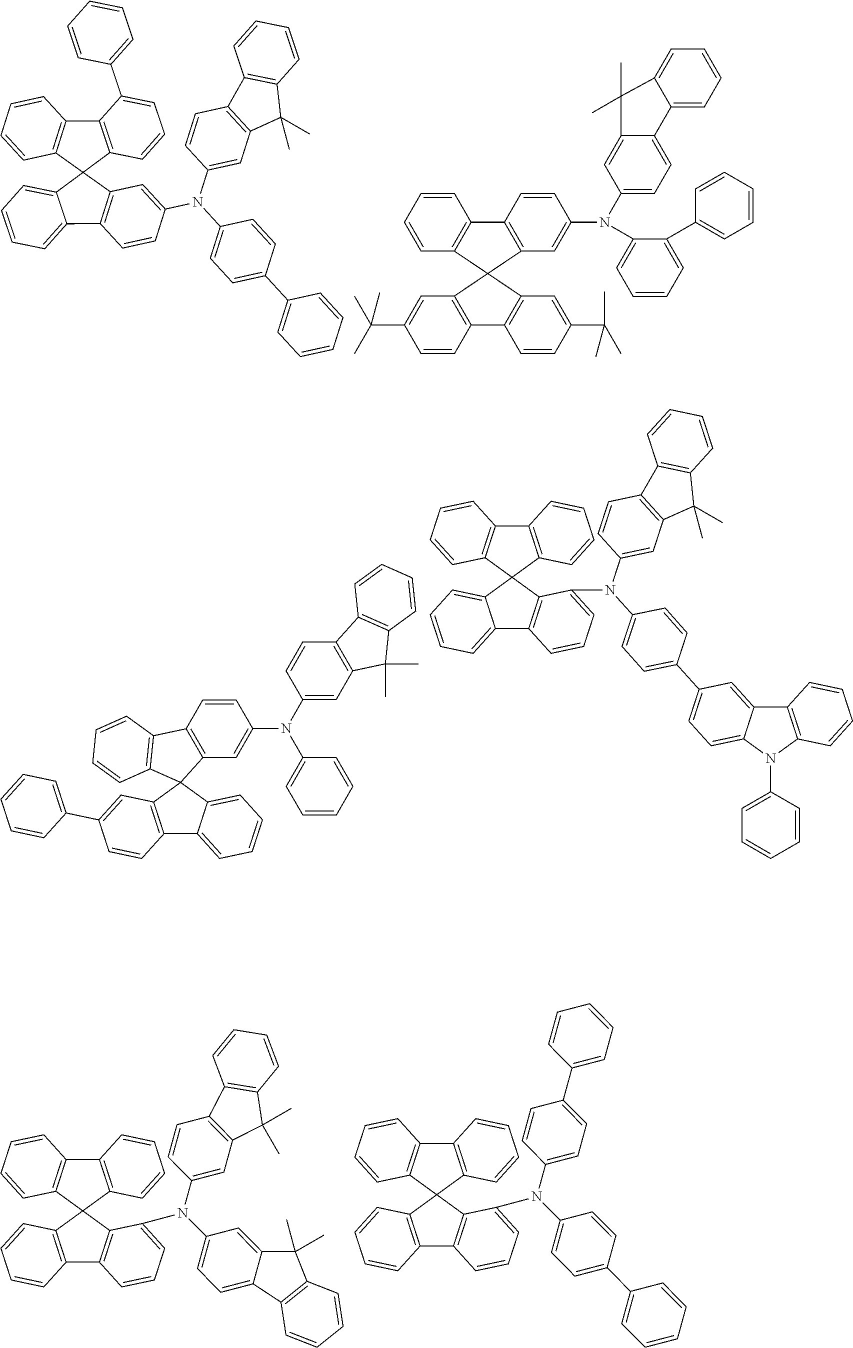

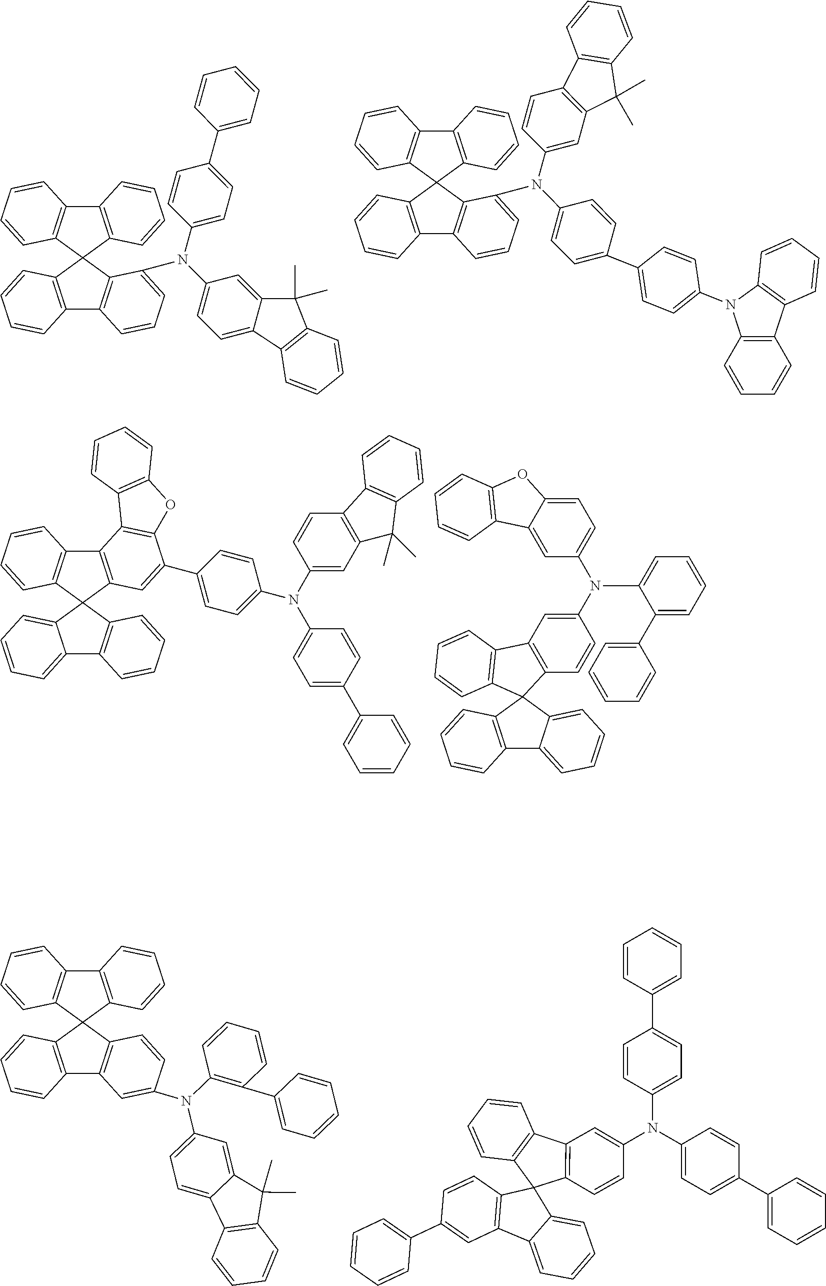

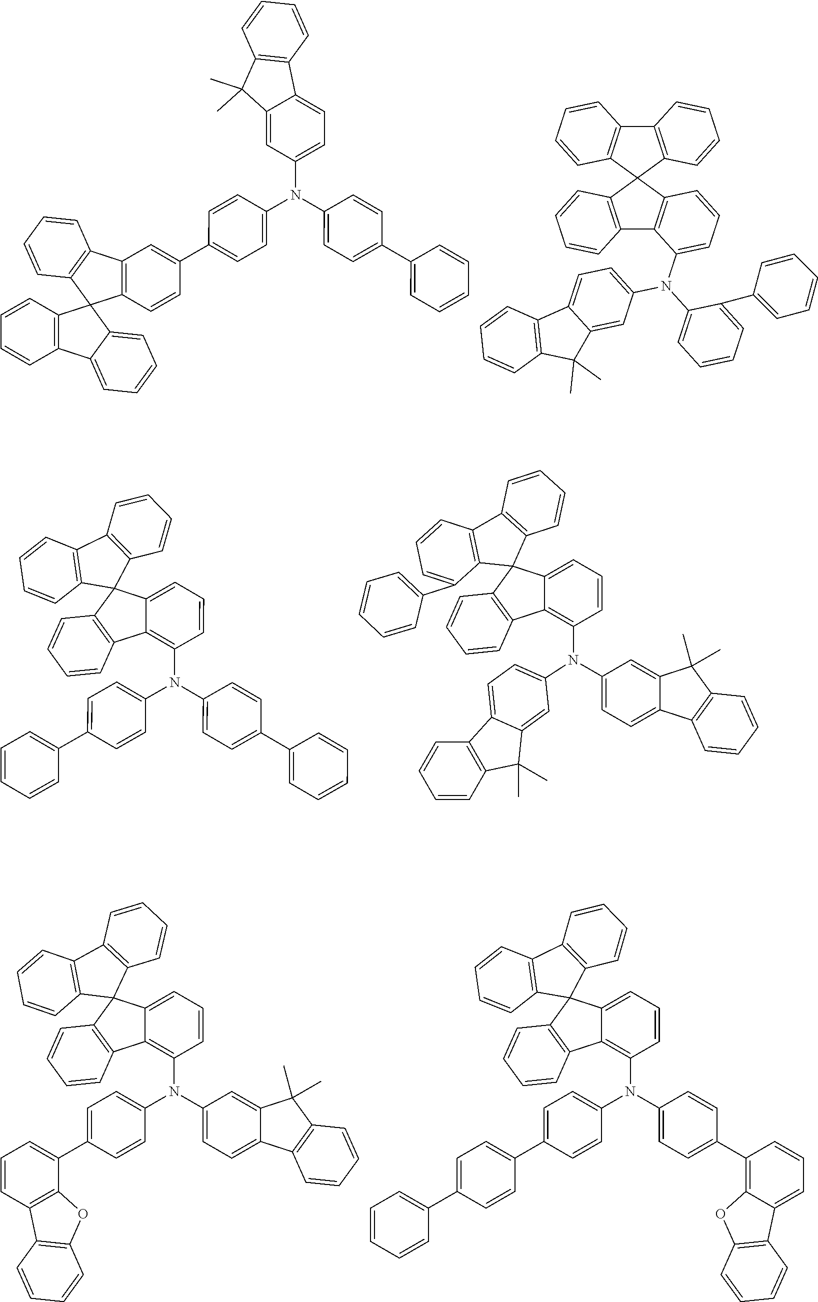

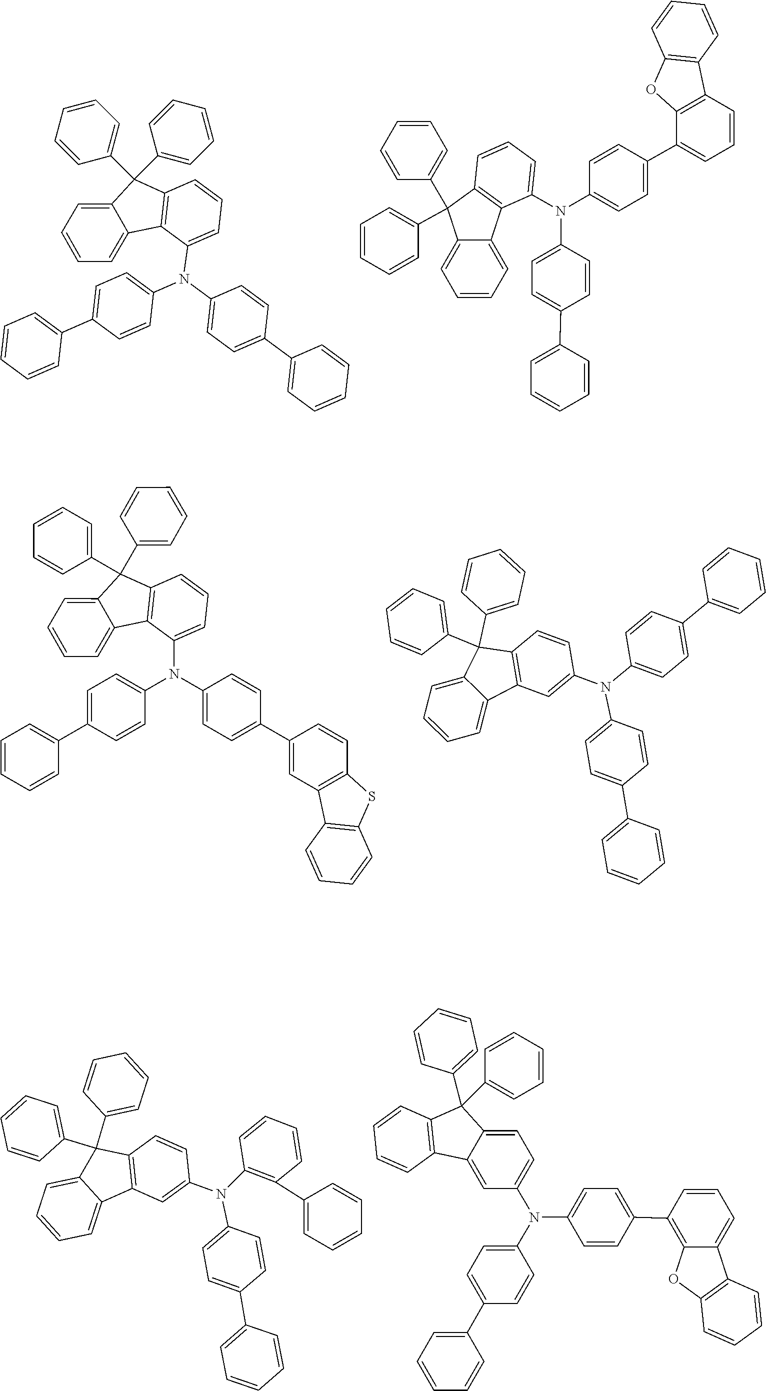

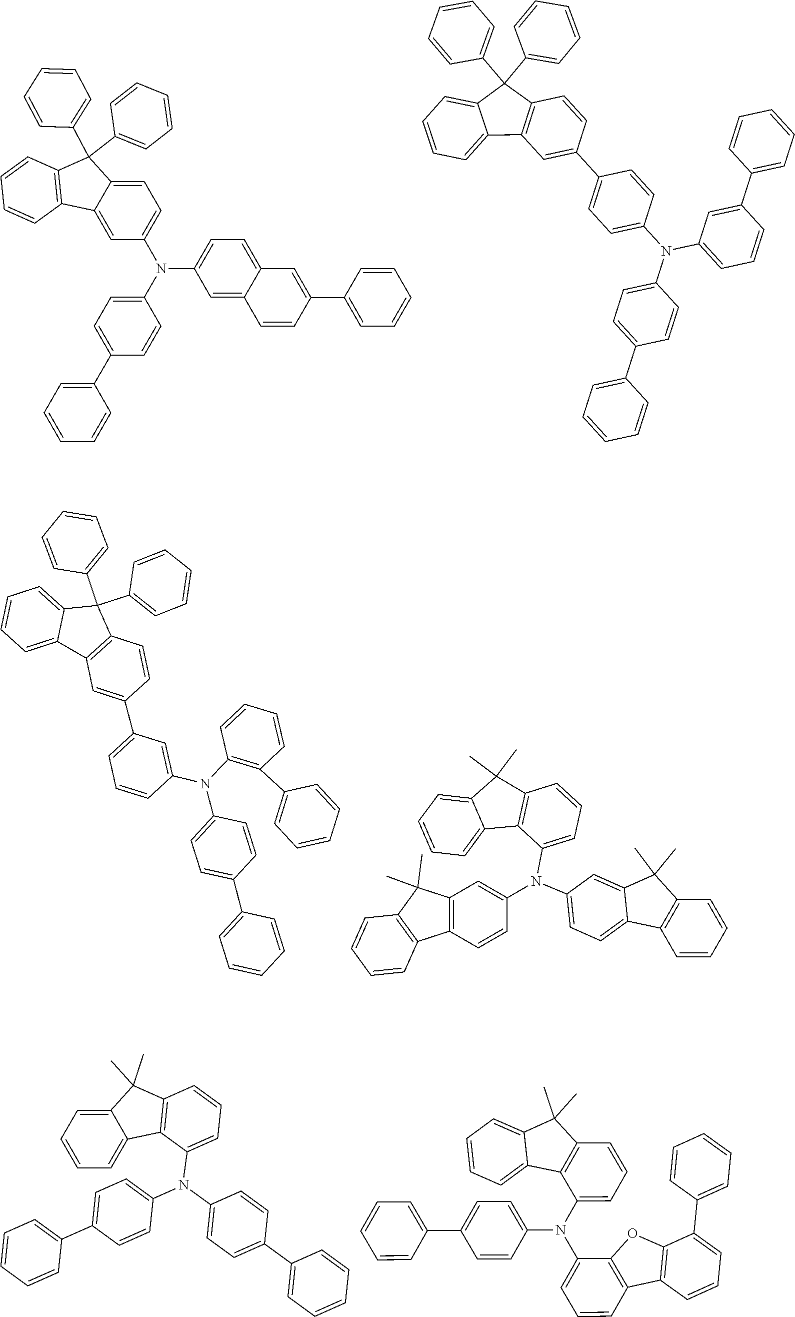

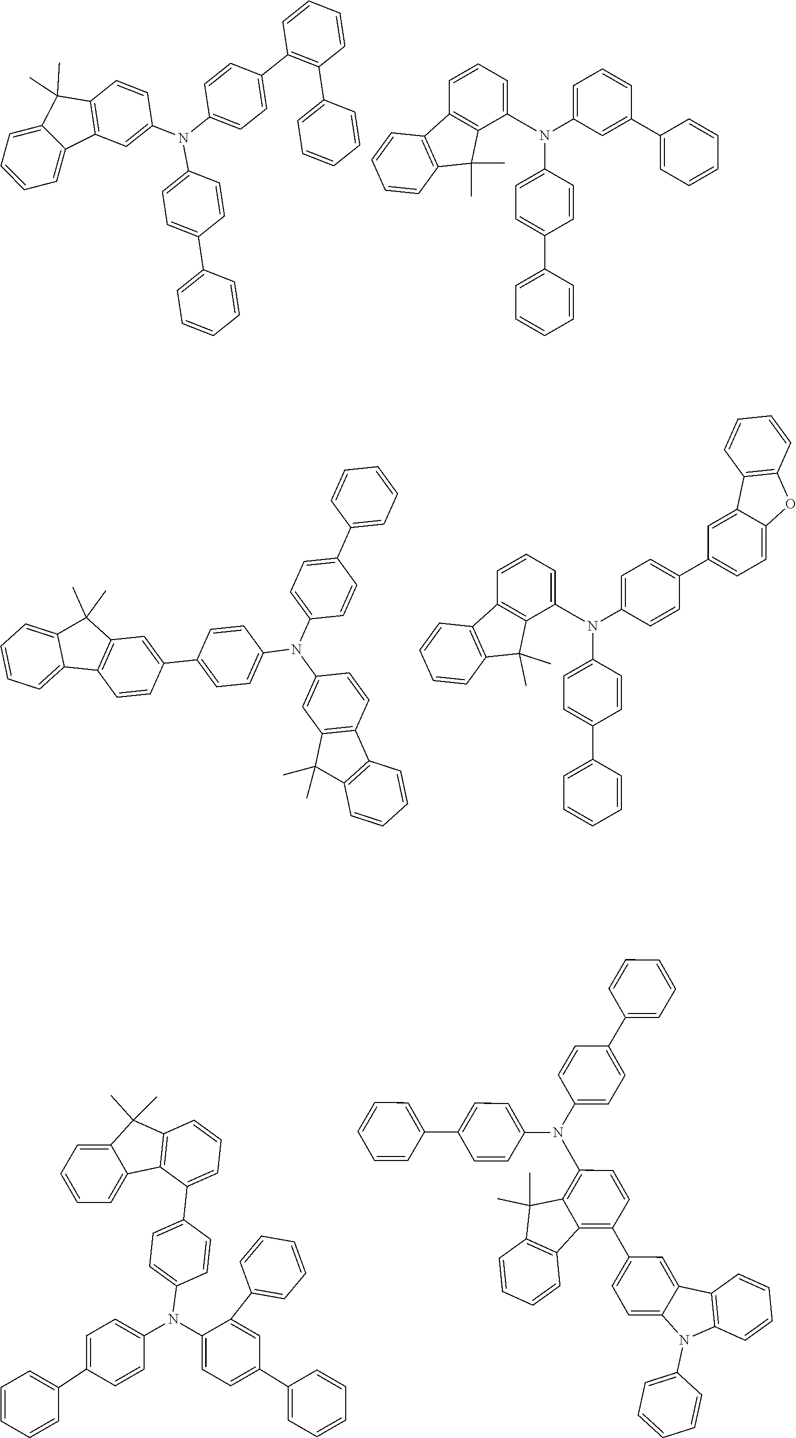

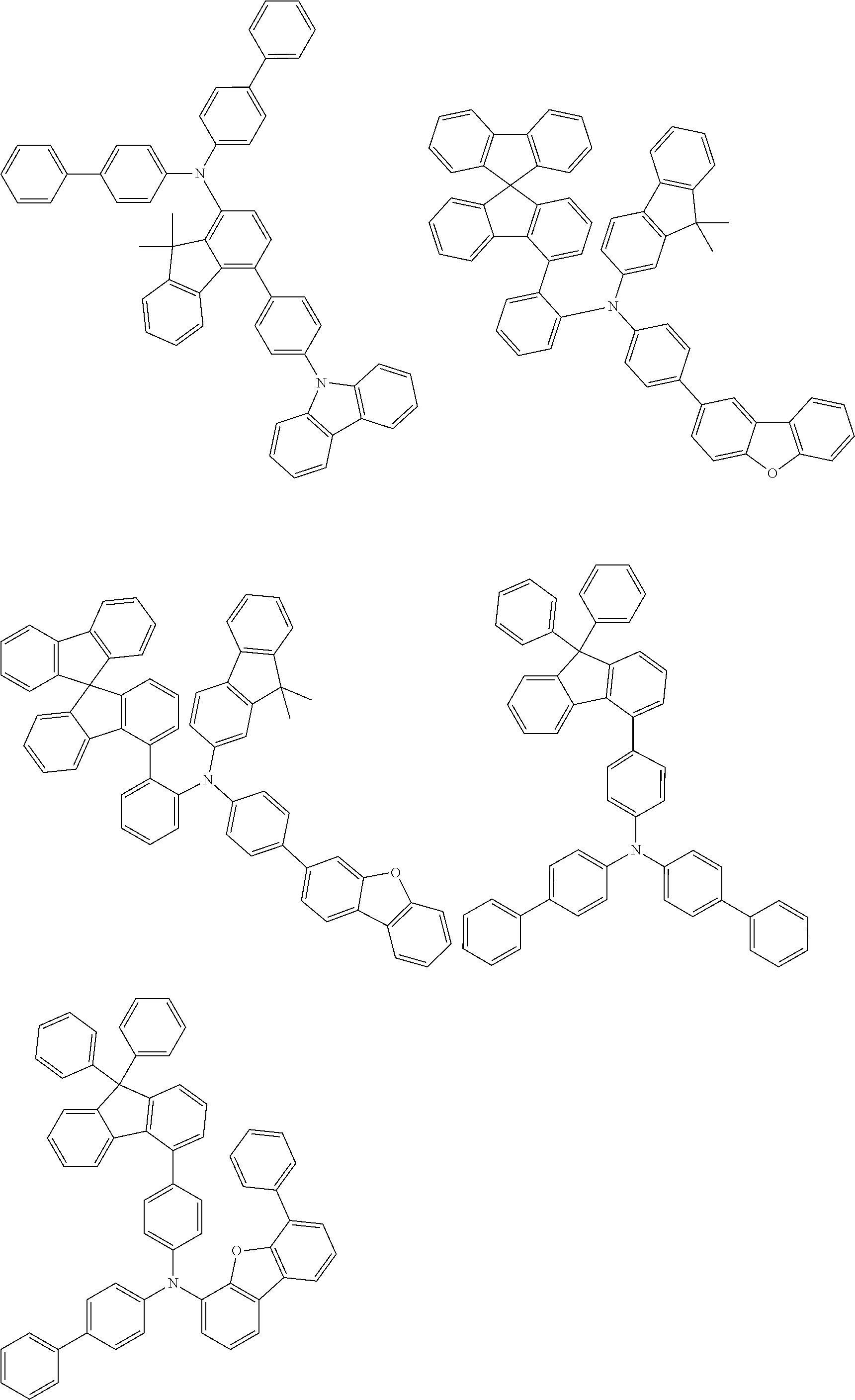

Compounds especially suitable for use in layers having a hole-transporting function in any OLEDs, not only the OLEDs according to the definitions of the present application, further include the following:

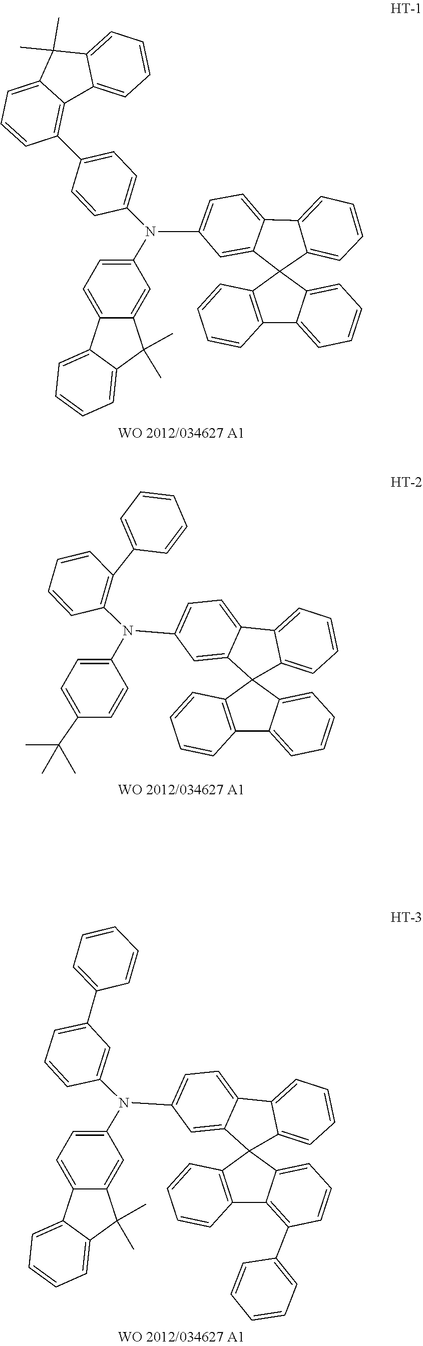 [SMILES: CC1(C)c2ccccc2-c2ccc(N(c3ccc(-c4cccc5c4-c4ccccc4C5(C)C)cc3)c3ccc4c(c3)C3(c5ccccc5-c5ccccc53)c3ccccc3-4)cc21 ; CC(C)(C)c1ccc(N(c2ccc3c(c2)C2(c4ccccc4-c4ccccc42)c2ccccc2-3)c2ccccc2-c2ccccc2)cc1 ; CC1(C)c2ccccc2-c2ccc(N(c3cccc(-c4ccccc4)c3)c3ccc4c(c3)C3(c5ccccc5-4)c4ccccc4-c4c(-c5ccccc5)cccc43)cc21]

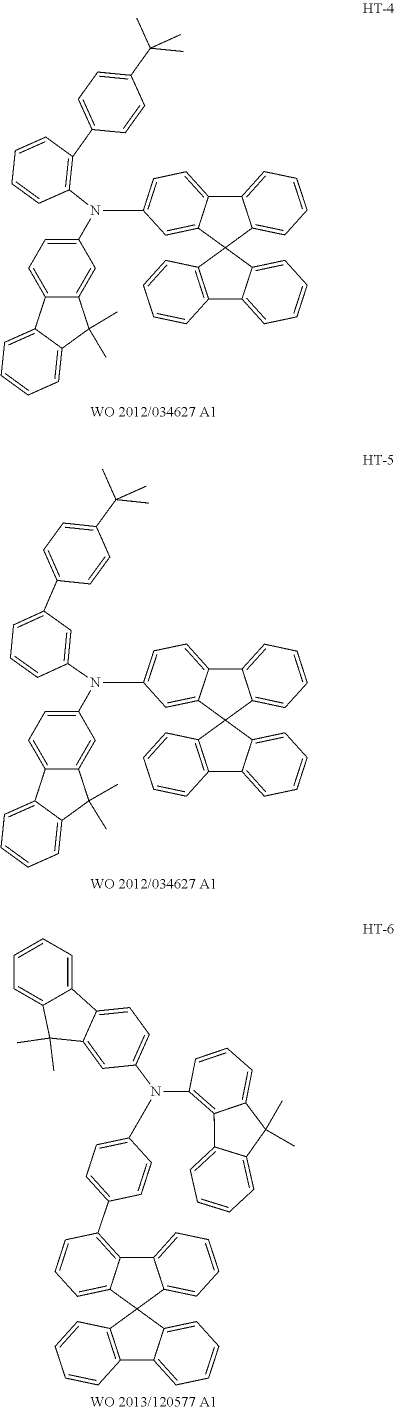 [SMILES: CC(C)(C)c1ccc(-c2ccccc2N(c2ccc3c(c2)C(C)(C)c2ccccc2-3)c2ccc3c(c2)C2(c4ccccc4-c4ccccc42)c2ccccc2-3)cc1 ; CC(C)(C)c1ccc(-c2cccc(N(c3ccc4c(c3)C(C)(C)c3ccccc3-4)c3ccc4c(c3)C3(c5ccccc5-c5ccccc53)c3ccccc3-4)c2)cc1 ; CC1(C)c2ccccc2-c2ccc(N(c3ccc(-c4cccc5c4-c4ccccc4C54c5ccccc5-c5ccccc54)cc3)c3cccc4c3-c3ccccc3C4(C)C)cc21]

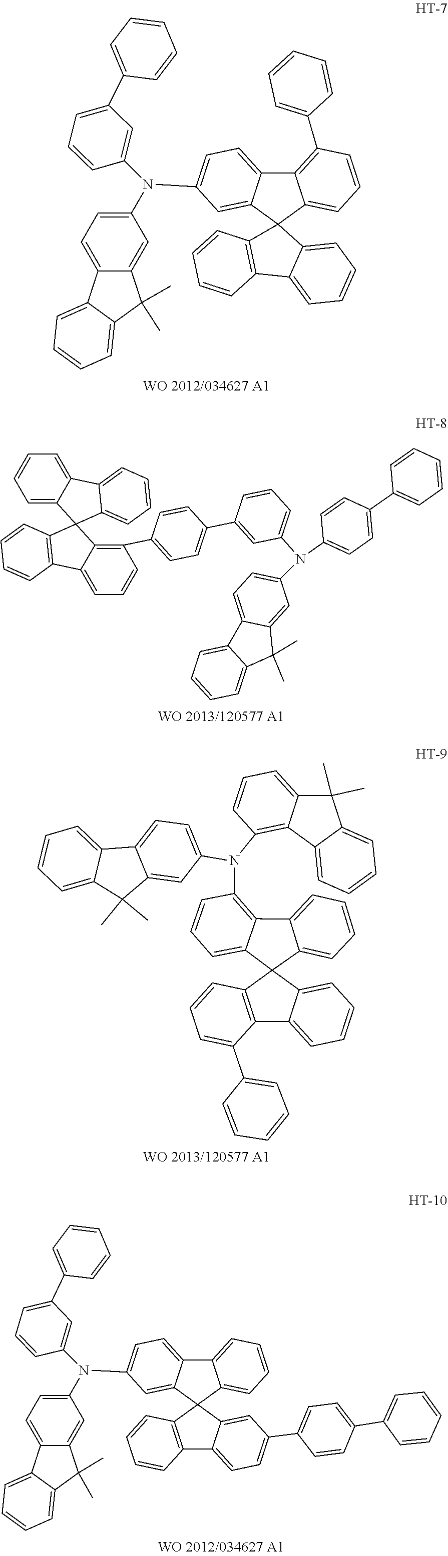 [SMILES: CC1(C)c2ccccc2-c2ccc(N(c3cccc(-c4ccccc4)c3)c3ccc4c(c3)C3(c5ccccc5-c5ccccc53)c3cccc(-c5ccccc5)c3-4)cc21 ; CC1(C)c2ccccc2-c2ccc(N(c3ccc(-c4ccccc4)cc3)c3cccc(-c4ccc(-c5cccc6c5C5(c7ccccc7-c7ccccc75)c5ccccc5-6)cc4)c3)cc21 ; CC1(C)c2ccccc2-c2ccc(N(c3cccc4c3-c3ccccc3C4(C)C)c3cccc4c3-c3ccccc3C43c4ccccc4-c4c(-c5ccccc5)cccc43)cc21 ; CC1(C)c2ccccc2-c2ccc(N(c3cccc(-c4ccccc4)c3)c3ccc4c(c3)C3(c5ccccc5-c5ccc(-c6ccc(-c7ccccc7)cc6)cc53)c3ccccc3-4)cc21]

The compounds HT-1 to HT-10 are generally suitable for use in hole-transporting layers. Their use is not limited to particular OLEDs, such as for example the OLEDs described in the present application.

The compounds HT-1 to HT-10 may be prepared by the methods disclosed in the published specifications cited in the table above. The further teaching relating to the use and preparation of the compounds disclosed in the published specifications cited in the above table is hereby explicitly incorporated by reference and is preferably to be combined with the teaching given above relating to the use of the abovementioned compounds as hole-transporting materials. The compounds HT-1 to HT-10 show exceptional properties when used in OLEDs, in particular exceptional lifetime and efficiency.

Preferred cathodes of the electronic device are metals having a low work function, metal alloys or multilayer structures composed of various metals, for example alkaline earth metals, alkali metals, main group metals or lanthanoids (e.g. Ca, Ba, Mg, Al, In, Mg, Yb, Sm, etc.). Additionally suitable are alloys composed of an alkali metal or alkaline earth metal and silver, for example an alloy composed of magnesium and silver. In the case of multilayer structures, in addition to the metals mentioned, it is also possible to use further metals having a relatively high work function, for example Ag or Al, in which case combinations of the metals such as Ca/Ag, Mg/Ag or Ba/Ag, for example, are generally used. It may also be preferable to introduce a thin interlayer of a material having a high dielectric constant between a metallic cathode and the organic semiconductor. Examples of useful materials for this purpose are alkali metal or alkaline earth metal fluorides, but also the corresponding oxides or carbonates (e.g. LiF, Li₂O, BaF₂, MgO, NaF, CsF, Cs₂CO₃, etc.). It is also possible to use lithium quinolinate (LiQ) for this purpose. The layer thickness of this layer is preferably between 0.5 and 5 nm.

Preferred anodes are materials having a high work function. Preferably, the anode has a work function of greater than 4.5 eV versus vacuum. Firstly, metals having a high redox potential are suitable for this purpose, for example Ag, Pt or Au. Secondly, metal/metal oxide electrodes (e.g. Al/Ni/NiOₓ, Al/PtOₓ) may also be preferred. For some applications, at least one of the electrodes has to be transparent or partly transparent in order to enable either the irradiation of the organic material (organic solar cell) or the emission of light (OLED, O-LASER). Preferred anode materials here are conductive mixed metal oxides. Particular preference is given to indium tin oxide (ITO) or indium zinc oxide (IZO). Preference is further given to conductive doped organic materials, especially conductive doped polymers. In addition, the anode may also consist of two or more layers, for example of an inner layer of ITO and an outer layer of a metal oxide, preferably tungsten oxide, molybdenum oxide or vanadium oxide.

In a preferred embodiment, the electronic device is characterized in that one or more layers are coated by a sublimation process. In this case, the materials are applied by vapour deposition in vacuum sublimation systems at an initial pressure of less than 10⁻⁵ mbar, preferably less than 10⁻⁶ mbar. In this case, however, it is also possible that the initial pressure is even lower, for example less than 10⁻⁷ mbar.

Preference is likewise given to an electronic device, characterized in that one or more layers are coated by the OVPD (organic vapour phase deposition) method or with the aid of a carrier gas sublimation. In this case, the materials are applied at a pressure between 10⁻⁵ mbar and 1 bar. A special case of this method is the OVJP (organic vapour jet printing) method, in which the materials are applied directly by a nozzle and thus structured (for example M. S. Arnold et al., Appl. Phys. Lett. 2008, 92, 053301).

Preference is additionally given to an electronic device, characterized in that one or more layers are produced from solution, for example by spin-coating, or by any printing method, for example screen printing, flexographic printing, nozzle printing or offset printing, but more preferably LITI (light-induced thermal imaging, thermal transfer printing) or inkjet printing. For this purpose, soluble compounds of formula (I), (II) or (III) are needed. High solubility can be achieved by suitable substitution of the compounds.

It is further preferable that an electronic device of the invention is produced by applying one or more layers from solution and one or more layers by a sublimation method.

After application of the layers, according to the use, the device is structured, contact-connected and finally sealed, in order to rule out damaging effects of water and air.

According to the invention, the electronic devices comprising one or more compounds of formula (I), (II) or (III) can be used in displays, as light sources in lighting applications and as light sources in medical and/or cosmetic applications.

### EXAMPLES

### A) Synthesis Examples

### 1) Synthesis of 4-{[1,1′-biphenyl]-4-yl}-3-bromo-1,1′-biphenyl 1a

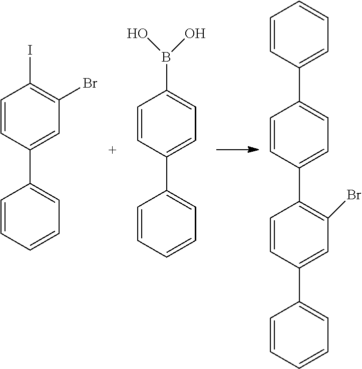 [SMILES: Brc1cc(-c2ccccc2)ccc1I ; OB(O)c1ccc(-c2ccccc2)cc1 ; Brc1cc(-c2ccccc2)ccc1-c1ccc(-c2ccccc2)cc1]

16.5 g (83.5 mmol) of biphenylboronic acid, 30 g (83.5 mmol) of 3-bromo-4-iodo-1,1′-biphenyl and 1.2 g (2 mmol) of bis(triphenylphosphine)Pd(II) chloride and 23 g (167 mmol) of potassium carbonate are suspended in 520 ml of acetonitrile and 220 ml of methanol. The reaction mixture is heated to boiling under a protective atmosphere overnight. The mixture is then filtered with suction and the filtride is washed with MeOH, water and MeOH again. The residue is purified by crystallization with MeOH. Yield: 29 g (85% of theory), purity by GC-MS >94%.

The following compounds are prepared in an analogous manner:

| Ex. | Halogen derivative | Boronic acid | Product |
|---|---|---|---|
| 1b | 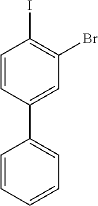 [SMILES: Brc1cc(-c2ccccc2)ccc1I] | 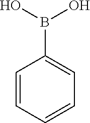 | 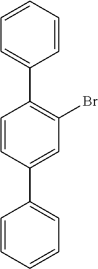 [SMILES: Brc1cc(-c2ccccc2)ccc1-c1ccccc1] |
| 1c | 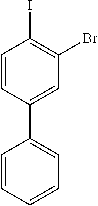 [SMILES: Brc1cc(-c2ccccc2)ccc1I] | 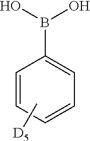 | 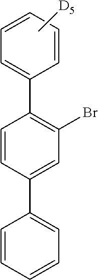 |
| 1d | 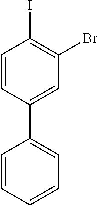 [SMILES: Brc1cc(-c2ccccc2)ccc1I] | 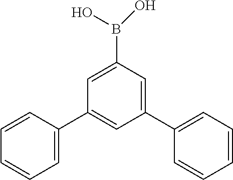 [SMILES: OB(O)c1cc(-c2ccccc2)cc(-c2ccccc2)c1] | 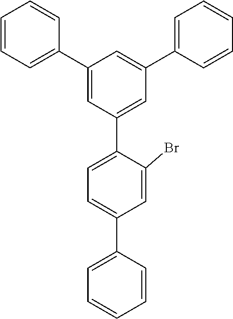 [SMILES: Brc1cc(-c2ccccc2)ccc1-c1cc(-c2ccccc2)cc(-c2ccccc2)c1] |
| 1e | 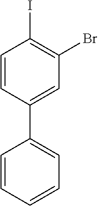 [SMILES: Brc1cc(-c2ccccc2)ccc1I] | 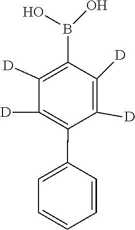 [SMILES: [2H]c1c([2H])c(-c2ccccc2)c([2H])c([2H])c1B(O)O] | 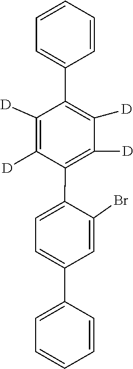 [SMILES: [2H]c1c([2H])c(-c2ccc(-c3ccccc3)cc2Br)c([2H])c([2H])c1-c1ccccc1] |
| 1f | 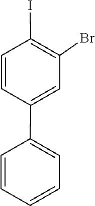 [SMILES: Brc1cc(-c2ccccc2)ccc1I] | 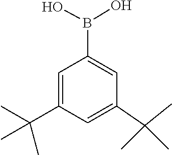 [SMILES: CC(C)(C)c1cc(B(O)O)cc(C(C)(C)C)c1] | 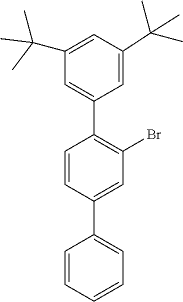 [SMILES: CC(C)(C)c1cc(-c2ccc(-c3ccccc3)cc2Br)cc(C(C)(C)C)c1] |

### 2) Synthesis of N-(4-{[1,1′-biphenyl]-4-yl}-[1,1′-biphenyl]-3-yl)-N-(9,9-dimethyl-9H-fluoren-2-yl)-9,9-dimethyl-9H-fluoren-2-amine 2a

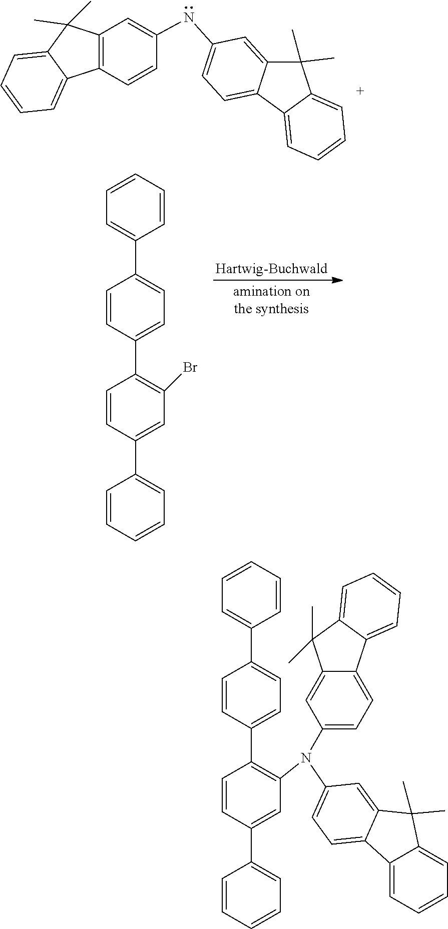 [SMILES: CC1(C)c2ccccc2-c2ccc(Nc3ccc4c(c3)C(C)(C)c3ccccc3-4)cc21 ; Brc1cc(-c2ccccc2)ccc1-c1ccc(-c2ccccc2)cc1 ; CC1(C)c2ccccc2-c2ccc(N(c3ccc4c(c3)C(C)(C)c3ccccc3-4)c3cc(-c4ccccc4)ccc3-c3ccc(-c4ccccc4)cc3)cc21]

N-(9,9-dimethyl-9H-fluoren-2-yl)-9,9-dimethyl-9H-fluoren-2-amine (30 g, 75 mmol), 4-{[1,1′-biphenyl]-4-yl}-3-bromo-1,1′-biphenyl (29 g, 75 mmol) and sodium tert-butoxide (14.7 g, 150 mmol) are dissolved in 350 ml of toluene. The solution is degassed and saturated with N₂. Then tri-tert-butylphosphine (7.5 ml; 7.5 mmol, 1 M in xylene) and 3.4 g (3.8 mmol) of Pd₂(dba)₃ are added thereto. The reaction mixture is heated to boiling under a protective atmosphere overnight. The mixture is cooled and partitioned between toluene and water and the organic phase is washed three times with water and dried over Na₂SO₄ and concentrated by rotary evaporation. After the crude product has been filtered through silica gel with toluene, the remaining residue is recrystallized from toluene and finally sublimed under high vacuum; purity is 99.9%. The yield is 23.9 g (45% of theory).

The following compounds are prepared in an analogous manner:

| Ex. | Halide | Amine | Product |
|---|---|---|---|
| 2b | 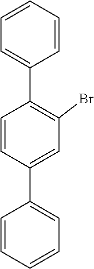 [SMILES: Brc1cc(-c2ccccc2)ccc1-c1ccccc1] | 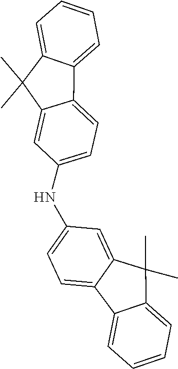 | 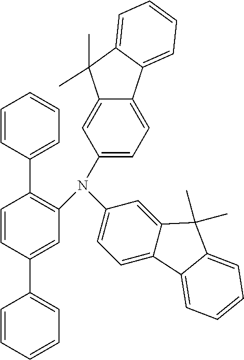 [SMILES: CC1(C)c2ccccc2-c2ccc(N(c3ccc4c(c3)C(C)(C)c3ccccc3-4)c3cc(-c4ccccc4)ccc3-c3ccccc3)cc21] |
| 2c | 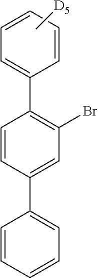 | 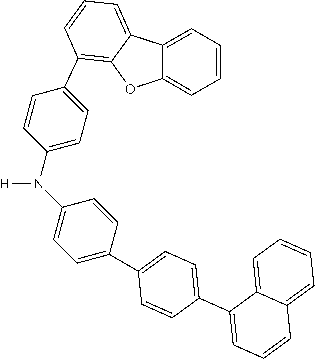 [SMILES: c1ccc2c(-c3ccc(-c4ccc(Nc5ccc(-c6cccc7c6oc6ccccc67)cc5)cc4)cc3)cccc2c1] | 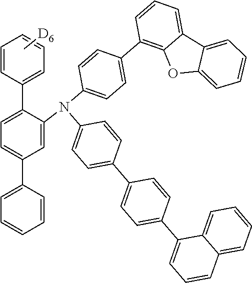 |
| 2d | 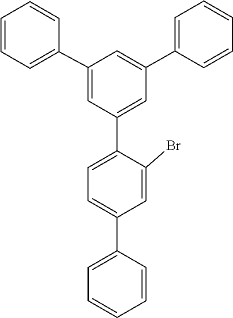 [SMILES: Brc1cc(-c2ccccc2)ccc1-c1cc(-c2ccccc2)cc(-c2ccccc2)c1] | 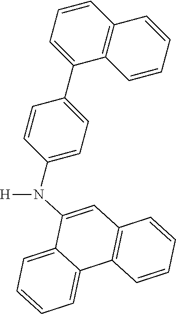 [SMILES: c1ccc2c(-c3ccc(Nc4cc5ccccc5c5ccccc45)cc3)cccc2c1] | 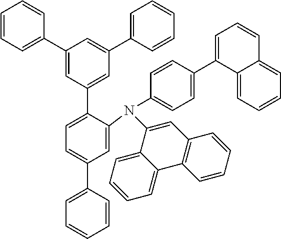 [SMILES: c1ccc(-c2cc(-c3ccccc3)cc(-c3ccc(-c4ccccc4)cc3N(c3ccc(-c4cccc5ccccc45)cc3)c3cc4ccccc4c4ccccc34)c2)cc1] |
| 2e | 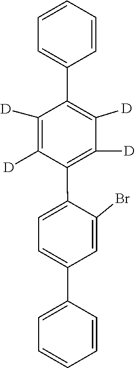 [SMILES: [2H]c1c([2H])c(-c2ccc(-c3ccccc3)cc2Br)c([2H])c([2H])c1-c1ccccc1] | 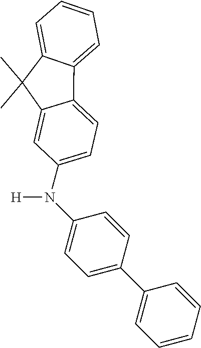 [SMILES: CC1(C)c2ccccc2-c2ccc(Nc3ccc(-c4ccccc4)cc3)cc21] | 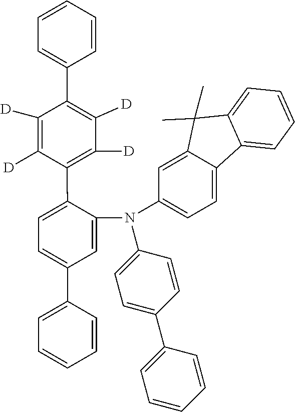 [SMILES: [2H]c1c([2H])c(-c2ccc(-c3ccccc3)cc2N(c2ccc(-c3ccccc3)cc2)c2ccc3c(c2)C(C)(C)c2ccccc2-3)c([2H])c([2H])c1-c1ccccc1] |
| 2f | 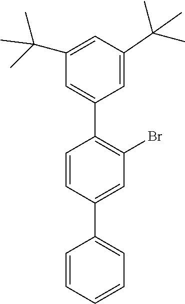 [SMILES: CC(C)(C)c1cc(-c2ccc(-c3ccccc3)cc2Br)cc(C(C)(C)C)c1] | 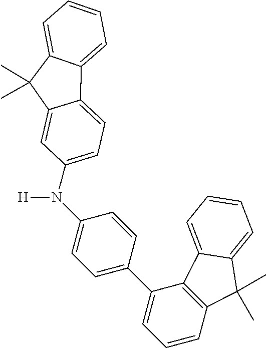 | 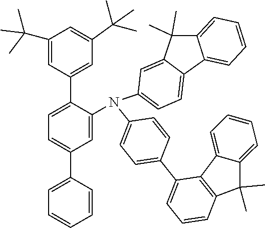 [SMILES: CC(C)(C)c1cc(-c2ccc(-c3ccccc3)cc2N(c2ccc(-c3cccc4c3-c3ccccc3C4(C)C)cc2)c2ccc3c(c2)C(C)(C)c2ccccc2-3)cc(C(C)(C)C)c1] |
| 2g | 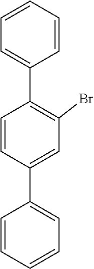 [SMILES: Brc1cc(-c2ccccc2)ccc1-c1ccccc1] | 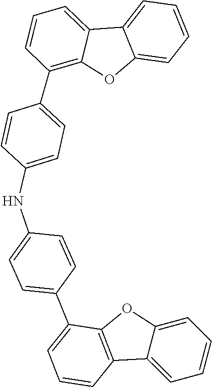 [SMILES: c1ccc2c(c1)oc1c(-c3ccc(Nc4ccc(-c5cccc6c5oc5ccccc56)cc4)cc3)cccc12] | 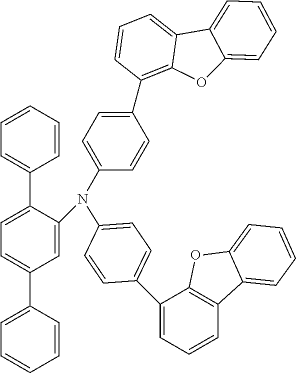 [SMILES: c1ccc(-c2ccc(-c3ccccc3)c(N(c3ccc(-c4cccc5c4oc4ccccc45)cc3)c3ccc(-c4cccc5c4oc4ccccc45)cc3)c2)cc1] |
| 2h | 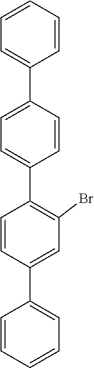 [SMILES: Brc1cc(-c2ccccc2)ccc1-c1ccc(-c2ccccc2)cc1] | 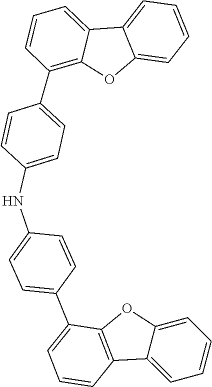 [SMILES: c1ccc2c(c1)oc1c(-c3ccc(Nc4ccc(-c5cccc6c5oc5ccccc56)cc4)cc3)cccc12] | 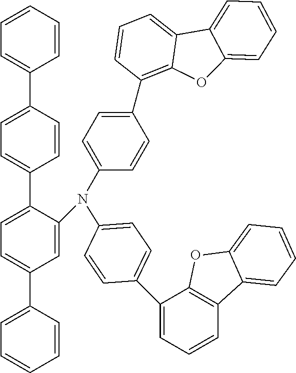 [SMILES: c1ccc(-c2ccc(-c3ccc(-c4ccccc4)cc3N(c3ccc(-c4cccc5c4oc4ccccc45)cc3)c3ccc(-c4cccc5c4oc4ccccc45)cc3)cc2)cc1] |
| 2i | 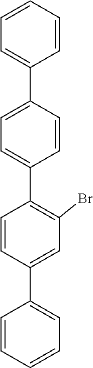 [SMILES: Brc1cc(-c2ccccc2)ccc1-c1ccc(-c2ccccc2)cc1] | 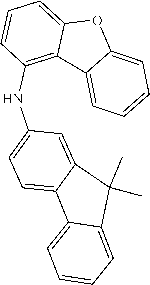 [SMILES: CC1(C)c2ccccc2-c2ccc(Nc3cccc4oc5ccccc5c34)cc21] | 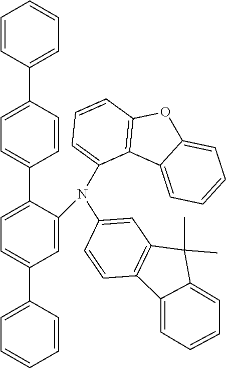 [SMILES: CC1(C)c2ccccc2-c2ccc(N(c3cc(-c4ccccc4)ccc3-c3ccc(-c4ccccc4)cc3)c3cccc4oc5ccccc5c34)cc21] |
| 2j | 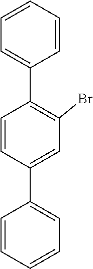 [SMILES: Brc1cc(-c2ccccc2)ccc1-c1ccccc1] | 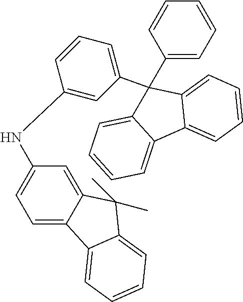 [SMILES: CC1(C)c2ccccc2-c2ccc(Nc3cccc(C4(c5ccccc5)c5ccccc5-c5ccccc54)c3)cc21] | 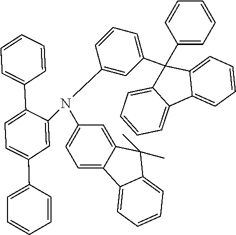 [SMILES: CC1(C)c2ccccc2-c2ccc(N(c3cccc(C4(c5ccccc5)c5ccccc5-c5ccccc54)c3)c3cc(-c4ccccc4)ccc3-c3ccccc3)cc21] |

### 3) Synthesis of N-[4-(4-{[1,1′-biphenyl]-4-yl}-[1,1′-biphenyl]-3-yl)phenyl]-N-(9,9-dimethyl-9H-fluoren-2-yl)-9,9-dimethyl-9H-fluoren-2-amine 3a

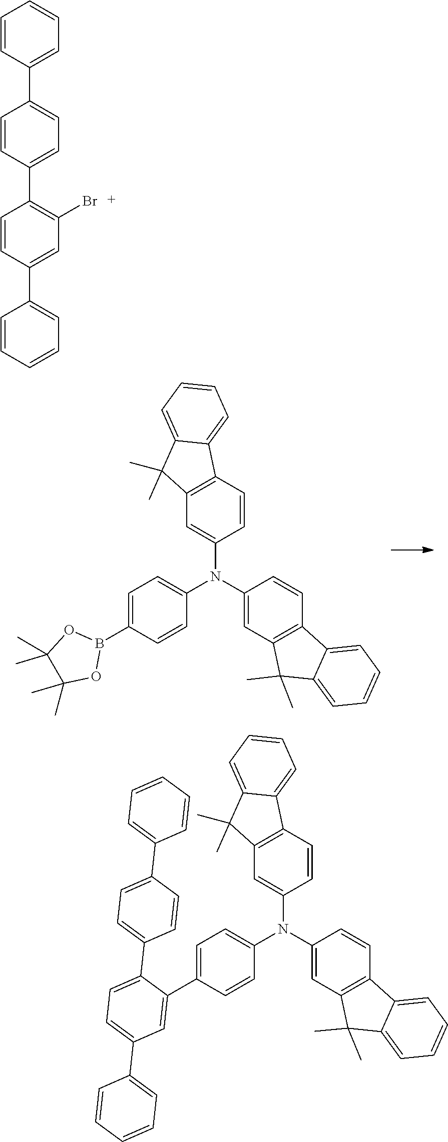

25.9 g (43 mmol) of N-(9,9-dimethyl-9H-fluoren-2-yl)-9,9-dimethyl-N-[4-(4,4,5,5-tetramethyl-1,3,2-dioxaborolan-2-yl)phenyl]-9H-fluoren-2-amine and 16.6 g (43 mmol) of 4-{[1,1′-biphenyl]-4-yl}-3-bromo-1,1′-biphenyl are suspended in 400 ml of dioxane and 13.7 g of caesium fluoride (90 mmol). 4.0 g (5.4 mmol) of bis(tricyclohexylphosphine)palladium dichloride is added to this suspension, and the reaction mixture is heated under reflux for 18 h. After cooling, the organic phase is removed, filtered through silica gel, washed three times with 80 ml of water and then concentrated to dryness. After the crude product has been filtered through silica gel with toluene, the remaining residue is recrystallized from toluene and finally sublimed under high vacuum, purity is 99.9%. The yield is 11 g (33% of theory).

The following compounds are prepared in an analogous manner:

| Ex. | Halide | Amine | Product |
|---|---|---|---|
| 3b | 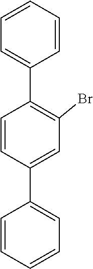 [SMILES: Brc1cc(-c2ccccc2)ccc1-c1ccccc1] | 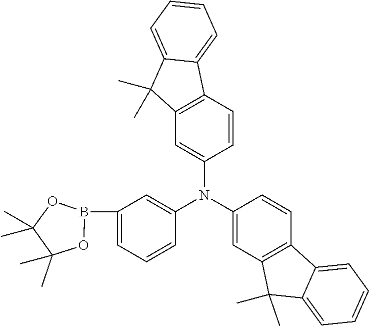 [SMILES: CC1(C)c2ccccc2-c2ccc(N(c3cccc(B4OC(C)(C)C(C)(C)O4)c3)c3ccc4c(c3)C(C)(C)c3ccccc3-4)cc21] | 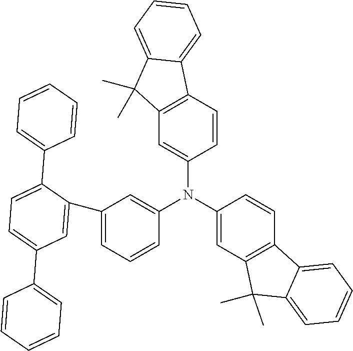 [SMILES: CC1(C)c2ccccc2-c2ccc(N(c3cccc(-c4cc(-c5ccccc5)ccc4-c4ccccc4)c3)c3ccc4c(c3)C(C)(C)c3ccccc3-4)cc21] |
| 3c | 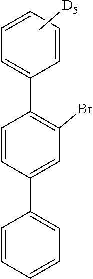 | 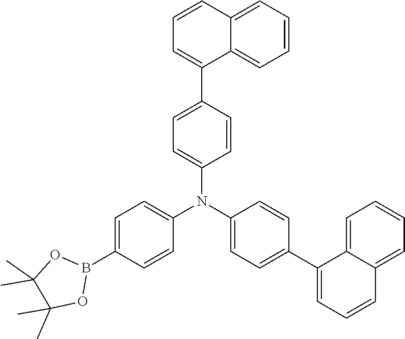 [SMILES: CC1(C)OB(c2ccc(N(c3ccc(-c4cccc5ccccc45)cc3)c3ccc(-c4cccc5ccccc45)cc3)cc2)OC1(C)C] | 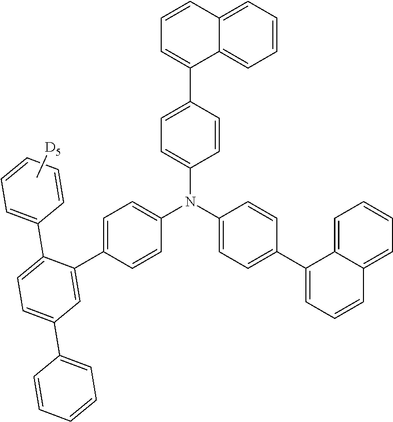 |
| 3d | 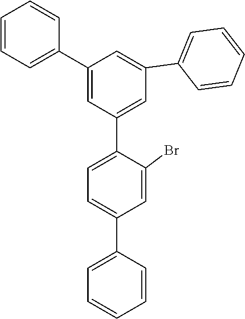 [SMILES: Brc1cc(-c2ccccc2)ccc1-c1cc(-c2ccccc2)cc(-c2ccccc2)c1] | 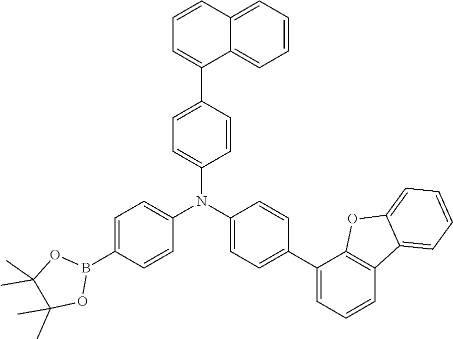 [SMILES: CC1(C)OB(c2ccc(N(c3ccc(-c4cccc5ccccc45)cc3)c3ccc(-c4cccc5c4oc4ccccc45)cc3)cc2)OC1(C)C] | 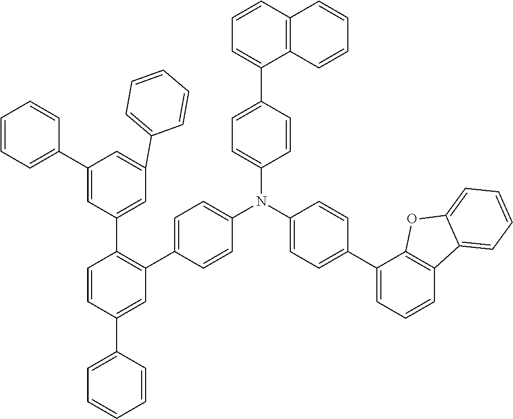 [SMILES: c1ccc(-c2cc(-c3ccccc3)cc(-c3ccc(-c4ccccc4)cc3-c3ccc(N(c4ccc(-c5cccc6ccccc56)cc4)c4ccc(-c5cccc6c5oc5ccccc56)cc4)cc3)c2)cc1] |
| 3e | 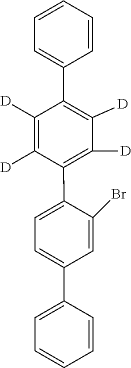 [SMILES: [2H]c1c([2H])c(-c2ccc(-c3ccccc3)cc2Br)c([2H])c([2H])c1-c1ccccc1] | 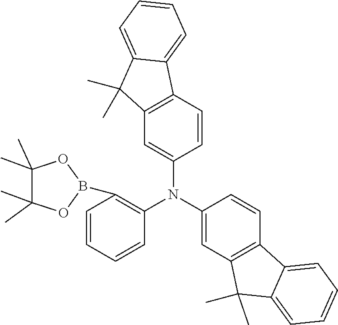 [SMILES: CC1(C)c2ccccc2-c2ccc(N(c3ccc4c(c3)C(C)(C)c3ccccc3-4)c3ccccc3B3OC(C)(C)C(C)(C)O3)cc21] | 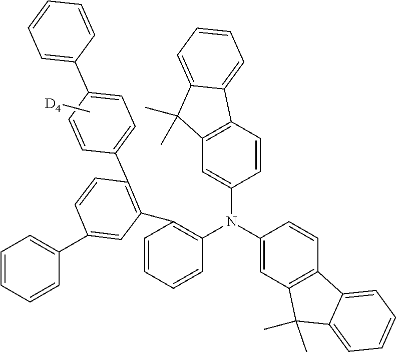 |
| 3f | 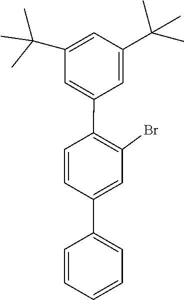 [SMILES: CC(C)(C)c1cc(-c2ccc(-c3ccccc3)cc2Br)cc(C(C)(C)C)c1] | 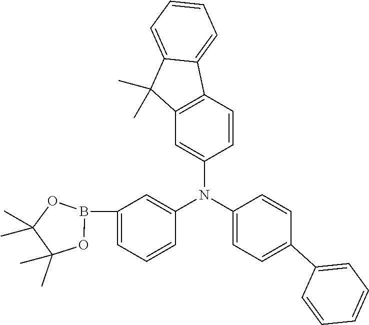 [SMILES: CC1(C)c2ccccc2-c2ccc(N(c3ccc(-c4ccccc4)cc3)c3cccc(B4OC(C)(C)C(C)(C)O4)c3)cc21] | 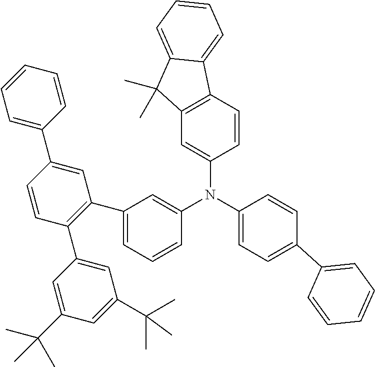 [SMILES: CC(C)(C)c1cc(-c2ccc(-c3ccccc3)cc2-c2cccc(N(c3ccc(-c4ccccc4)cc3)c3ccc4c(c3)C(C)(C)c3ccccc3-4)c2)cc(C(C)(C)C)c1] |
| 3g | 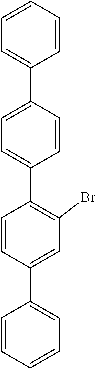 [SMILES: Brc1cc(-c2ccccc2)ccc1-c1ccc(-c2ccccc2)cc1] | 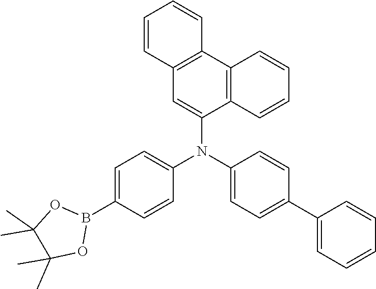 [SMILES: CC1(C)OB(c2ccc(N(c3ccc(-c4ccccc4)cc3)c3cc4ccccc4c4ccccc34)cc2)OC1(C)C] | 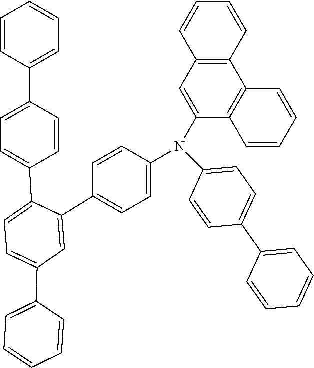 [SMILES: c1ccc(-c2ccc(-c3ccc(-c4ccccc4)cc3-c3ccc(N(c4ccc(-c5ccccc5)cc4)c4cc5ccccc5c5ccccc45)cc3)cc2)cc1] |
| 3h | 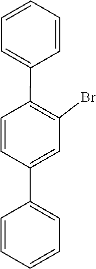 [SMILES: Brc1cc(-c2ccccc2)ccc1-c1ccccc1] | 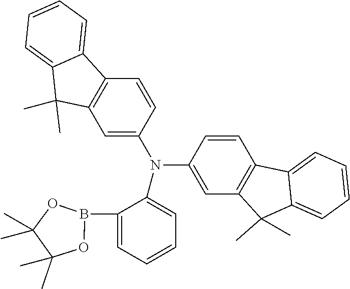 [SMILES: CC1(C)c2ccccc2-c2ccc(N(c3ccc4c(c3)C(C)(C)c3ccccc3-4)c3ccccc3B3OC(C)(C)C(C)(C)O3)cc21] | 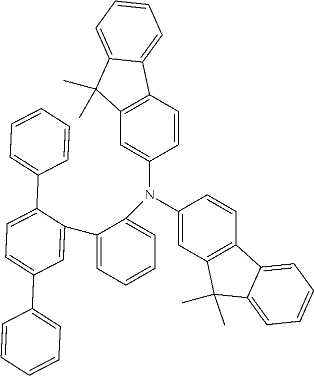 [SMILES: CC1(C)c2ccccc2-c2ccc(N(c3ccc4c(c3)C(C)(C)c3ccccc3-4)c3ccccc3-c3cc(-c4ccccc4)ccc3-c3ccccc3)cc21] |

### B) Device Examples

### 1) General Production Process for the OLEDs and Characterization of the OLEDs

Glass plates which have been coated with structured ITO (indium tin oxide) in a thickness of 50 nm form the substrates to which the OLEDs are applied.

The OLEDs basically have the following layer structure: substrate/hole injection layer (HIL)/hole transport layer (HTL1)/optional second hole transport layer (HTL2)/electron blocker layer (EBL)/emission layer (EML)/optional hole blocker layer (HBL)/electron transport layer (ETL1)/optional second electron transport layer (ETL2)/electron injection layer (EIL) and finally a cathode. The cathode is formed by an aluminium layer of thickness 100 nm. The exact structure of the OLEDs can be found in the tables which follow. The materials required for production of the OLEDs are shown in table 7. The “HTM-a” material used in the HIL and the HTL is a fluorene derivative. The p-dopant A used is NDP-9 from Novaled AG, Dresden.

All materials are applied by thermal vapour deposition in a vacuum chamber. In this case, the emission layer consists of at least one matrix material (host material) and an emitting dopant (emitter) which is added to the matrix material(s) in a particular proportion by volume by co-evaporation. Details given in such a form as H:SEB (95%:5%) mean here that the material H is present in the layer in a proportion by volume of 95% and SEB in a proportion of 5%. Analogously, the electron transport layer and the hole injection layer also consist of a mixture of two materials.

The OLEDs are characterized in a standard manner. For this purpose, the electroluminescence spectra, the external quantum efficiency (EQE, measured in %) as a function of the luminance, calculated from current-voltage-luminance characteristics assuming Lambertian radiation characteristics, and the lifetime are determined. The parameter EQE @ 10 mA/cm² refers to the external quantum efficiency which is attained at 10 mA/cm². The lifetime LT is defined as the time after which the luminance drops from the starting luminance to a certain proportion in the course of operation with constant current density. An LT90 figure means here that the lifetime reported corresponds to the time after which the luminance has dropped to 90% of its starting value. The figure @60 mA/cm², for example, means here that the lifetime in question is measured at 60 mA/cm².

### 2) Examples of Use of the Compounds in the EBL of Blue-Fluorescing OLEDs

OLEDs are produced with the following structure:

TABLE 1
OLED structure

| Ex. | HIL | HTL1 | HTL2 | EBL | EML | ETL1 | EIL |
|---|---|---|---|---|---|---|---|
| | Thickness/ | Thickness/ | Thickness/ | Thickness/ | Thickness/ | Thickness/ | Thickness/ |
| | nm | nm | nm | nm | nm | nm | nm |
| 1 | HTM: p- | HTM | HTM-1: p- | HTM-1 | H:SEB | ETM:LiQ | LiQ |
| | dopant (5%) | 160 nm | dopant | 10 nm | (95%:5%) | (50:50%) | 1 nm |
| | 20 nm | | (5%) | | 20 nm | 30 nm | |
| | | | 20 nm | | | | |
| 2 | HTM: p- | HTM | HTM-2: p- | HTM-2 | H:SEB | ETM:LiQ | LiQ |
| | dopant (5%) | 160 nm | dopant | 10 nm | (95%:5%) | (50:50%) | 1 nm |
| | 20 nm | | (5%) | | 20 nm | 30 nm | |
| | | | 20 nm | | | | |
| 3 | HTM: p- | HTM | HTM-3: p- | HTM-3 | H:SEB | ETM:LiQ | LiQ |
| | dopant (5%) | 160 nm | dopant | 10 nm | (95%:5%) | (50:50%) | 1 nm |
| | 20 nm | | (5%) | | 20 nm | 30 nm | |
| | | | 20 nm | | | | |
| 4 | HTM: p- | HTM | HTM-4: p- | HTM-4 | H:SEB | ETM:LiQ | LiQ |
| | dopant (5%) | 160 nm | dopant | 10 nm | (95%:5%) | (50:50%) | 1 nm |
| | 20 nm | | (5%) | | 20 nm | 30 nm | |
| | | | 20 nm | | | | |

OLEDs 1 to 4 show that the compounds according to the present application are of good suitability for use in the electron blocker layer of blue-fluorescing OLEDs.

The OLEDs have good results for lifetime, efficiency and operating voltage, as shown in the following table:

TABLE 2
OLED data

| | | | LT90 |
|---|---|---|---|
| | V (at | EQE @ | (at |
| | 10 mA/ | 10 mA/ | 60 mA/ |
| Ex. | cm²) | cm² | cm²) |
| 1 | 3.60 | 7.56 | 176 |
| 2 | 3.79 | 8.39 | 173 |
| 3 | 3.47 | 8.16 | 124 |
| 4 | 3.67 | 8.44 | 184 |

### 3) Examples of Use of the Compounds in the EBL of Green-Phosphorescing OLEDs

OLEDs are produced with the following structure:

TABLE 3
OLED structure

| Ex. | HIL | HTL1 | EBL | EML | ETL1 | ETL2 | EIL |
|---|---|---|---|---|---|---|---|
| | Thickness/ | Thickness/ | Thickness/ | Thickness/ | Thickness/ | Thickness/ | Thickness/ |
| | nm | nm | nm | nm | nm | nm | nm |
| 5 | HTM: p- | HTM | HTM-1 | TMM-1 (59%): | ETM | ETM:LiQ | LiQ |
| | dopant (5%) | 220 nm | 10 nm | TMM- | 10 nm | (50:50%) | 1 nm |
| | 20 nm | | | 2(29%):TEG(12%) | | 30 nm | |
| | | | | 30 nm | | | |
| 6 | HTM: p- | HTM | HTM-2 | TMM-1 (59%): | ETM | ETM:LiQ | LiQ |
| | dopant (5%) | 220 nm | 10 nm | TMM- | 10 nm | (50:50%) | 1 nm |
| | 20 nm | | | 2(29%):TEG(12%) | | 30 nm | |
| | | | | 30 nm | | | |
| 7 | HTM: p- | HTM | HTM-3 | TMM-1 (59%): | ETM | ETM:LiQ | LiQ |
| | dopant (5%) | 220 nm | 10 nm | TMM- | 10 nm | (50:50%) | 1 nm |
| | 20 nm | | | 2(29%):TEG(12%) | | 30 nm | |
| | | | | 30 nm | | | |
| 8 | HTM: p- | HTM | HTM-4 | TMM-1 (59%): | ETM | ETM:LiQ | LiQ |
| | dopant (5%) | 220 nm | 10 nm | TMM- | 10 nm | (50:50%) | 1 nm |
| | 20 nm | | | 2(29%):TEG(12%) | | 30 nm | |
| | | | | 30 nm | | | |

OLEDs 5 to 8 show that the compounds according to the present application are of good suitability for use in the electron blocker layer of green-phosphorescing OLEDs.

The OLEDs have good results for lifetime, efficiency and operating voltage, as shown in the following table:

TABLE 4
OLED data

| | | | LT90 |
|---|---|---|---|
| | V (at | EQE @ | (at |
| | 2 mA/ | 10 mA/ | 40 mA/ |
| Ex. | cm²) | cm² | cm²) |
| 5 | 3.99 | 16.76 | 131 |
| 6 | 3.92 | 18.36 | 141 |
| 7 | 3.69 | 16.86 | 182 |
| 8 | 3.80 | 18.79 | 121 |

### 4) Example of Use of the Compounds in the HIL of Blue-Fluorescing OLEDs

OLEDs are produced with the following structure:

TABLE 5
OLED structure

| Ex. | HIL | HTL1 | EBL | EML | ETL1 | EIL |
|---|---|---|---|---|---|---|
| | Thickness/ | Thickness/ | Thickness/ | Thickness/ | Thickness/ | Thickness/ |
| | nm | nm | nm | nm | nm | nm |
| 9 | HTM-1: p- | HTM-1 | EBM | H:SEB | ETM:LiQ | LiQ |
| | dopant (5%) | 180 nm | 10 nm | (95%:5%) | (50:50%) | 1 nm |
| | 20 nm | | | 20 nm | 30 nm | |
| 10 | HTM-1: p- | HTM-3 | EBM | H:SEB | ETM:LiQ | LiQ |
| | dopant (5%) | 180 nm | 10 nm | (95%:5%) | (50:50%) | 1 nm |
| | 20 nm | | | 20 nm | 30 nm | |

OLEDs 9 and 10 show that the compounds according to the present application are of good suitability for use in the hole injection layer of blue-fluorescing OLEDs.

The OLEDs have good results for lifetime, efficiency and operating voltage, as shown in the following table:

TABLE 6
OLED data

| | V (at | EQE @ | LT90 (at |
|---|---|---|---|
| | 10 mA/ | 10 mA/ | 60 mA/ |
| Ex. | cm²) | cm² | cm²) |
| 9 | 3.85 | 9.26 | 167 |
| 10 | 3.84 | 9.76 | 110 |

HTM-2 and HTM-4 can likewise be used as HIL in blue-fluorescing OLEDs, in a correspondingly suitable OLED stack.

### 5) Use of the Compounds in the EBL of Green-Phosphorescing OLEDs

OLEDs are produced with the following structure:

| Ex. | HIL | HTL | EBL | EML | HBL | ETL | EIL |
|---|---|---|---|---|---|---|---|
| | Thickness/ | Thickness/ | Thickness/ | Thickness/ | Thickness/ | Thickness/ | Thickness/ |
| | nm | nm | nm | nm | nm | nm | nm |
| 11 | HTM-a: p- | HTM-a | HTM-5 | TMM-1a (32%) | HBM 5nm | ETM-a: | LiQ |
| | dopant A | 50 nm | 30 nm | TMM-2a (60%) | | LiQ(50%) | 1 nm |
| | (5%) | | | TEG-a (8%) | | 30 nm | |
| | 10 nm | | | 35 nm | | | |

Data of the OLEDs

| | EQE @ | V (at | LT90 (at |
|---|---|---|---|
| | 10 mA/ | 10 mA/ | 60 mA/ |
| Ex. | cm² | cm²) | cm²) |
| 11 | 23.17 | 4.1 | 110 |

HTM-1 to HTM-4 may be used in place of HTM-5 in the stack shown above.

TABLE 7
Materials used

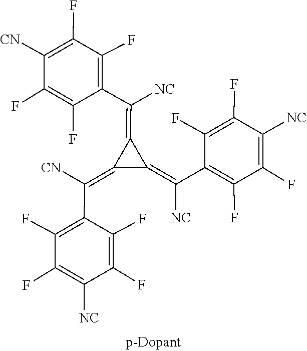

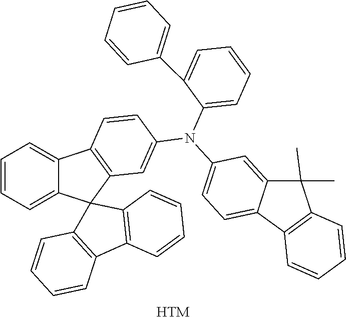

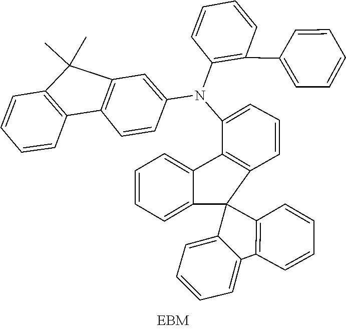 [SMILES: CC1(C)c2ccccc2-c2ccc(N(c3ccccc3-c3ccccc3)c3cccc4c3-c3ccccc3C43c4ccccc4-c4ccccc43)cc21]

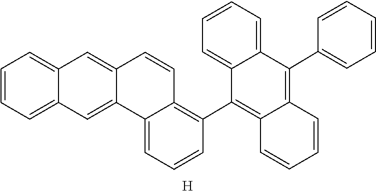

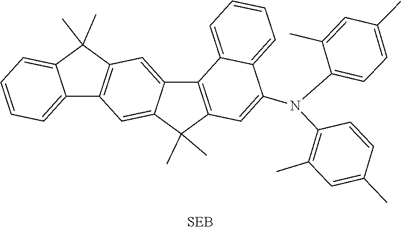 [SMILES: Cc1ccc(N(c2ccc(C)cc2C)c2cc3c(c4ccccc24)-c2cc4c(cc2C3(C)C)-c2ccccc2C4(C)C)c(C)c1]

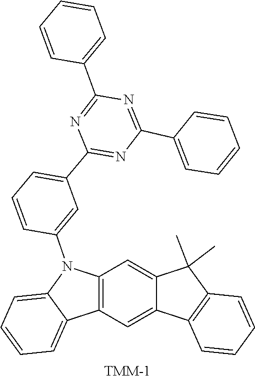

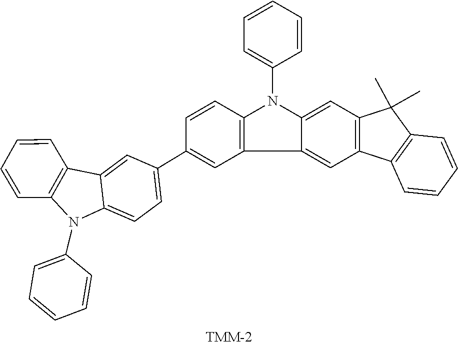

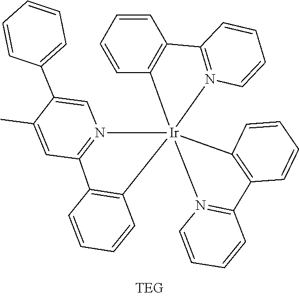

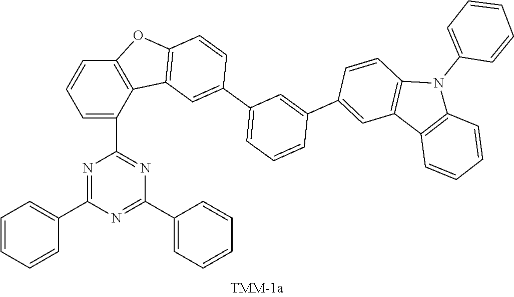 [SMILES: c1ccc(-c2nc(-c3ccccc3)nc(-c3cccc4oc5ccc(-c6cccc(-c7ccc8c(c7)c7ccccc7n8-c7ccccc7)c6)cc5c34)n2)cc1]

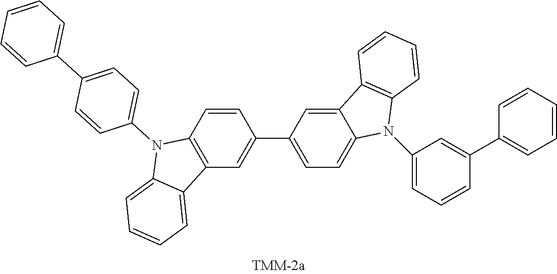 [SMILES: c1ccc(-c2ccc(-n3c4ccccc4c4cc(-c5ccc6c(c5)c5ccccc5n6-c5cccc(-c6ccccc6)c5)ccc43)cc2)cc1]

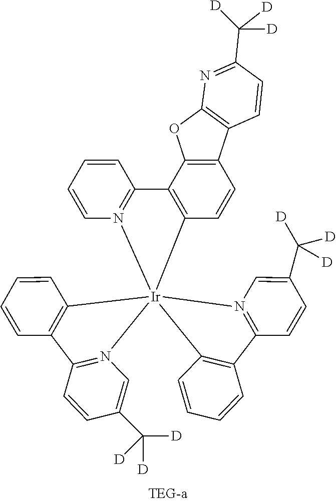

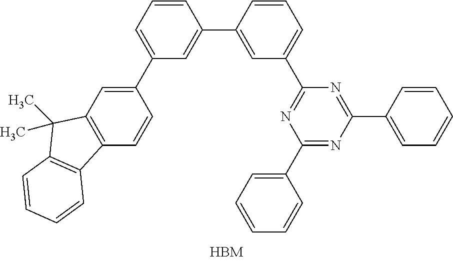 [SMILES: CC1(C)c2ccccc2-c2ccc(-c3cccc(-c4cccc(-c5nc(-c6ccccc6)nc(-c6ccccc6)n5)c4)c3)cc21]

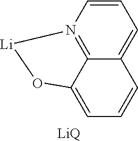

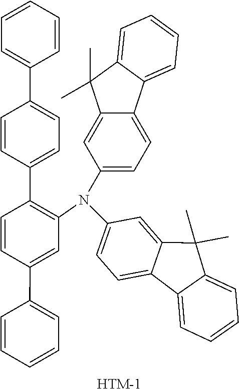

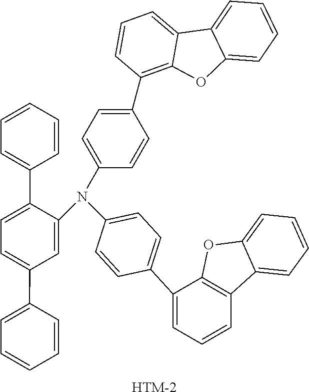

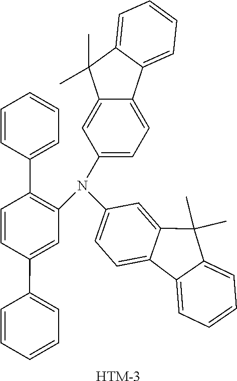

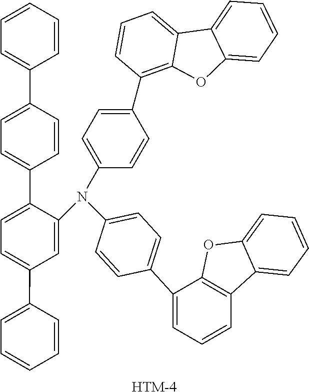

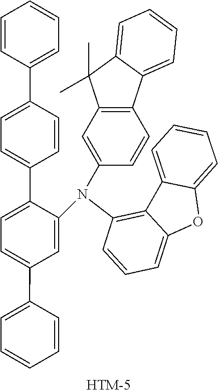

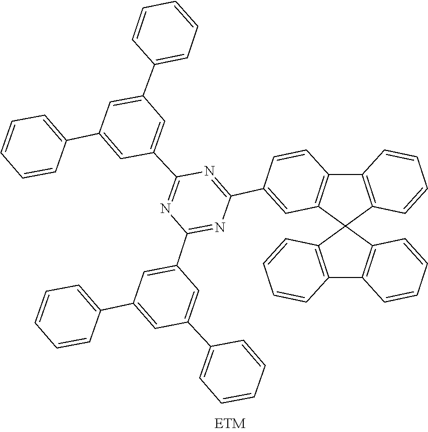 [SMILES: c1ccc(-c2cc(-c3ccccc3)cc(-c3nc(-c4cc(-c5ccccc5)cc(-c5ccccc5)c4)nc(-c4ccc5c(c4)C4(c6ccccc6-c6ccccc64)c4ccccc4-5)n3)c2)cc1]

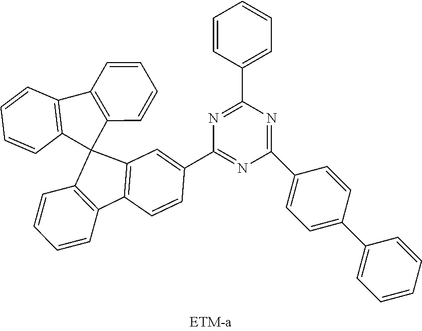 [SMILES: c1ccc(-c2ccc(-c3nc(-c4ccccc4)nc(-c4ccc5c(c4)C4(c6ccccc6-c6ccccc64)c4ccccc4-5)n3)cc2)cc1]

1.-19. (canceled)

20. A compound of one of the following formulae: 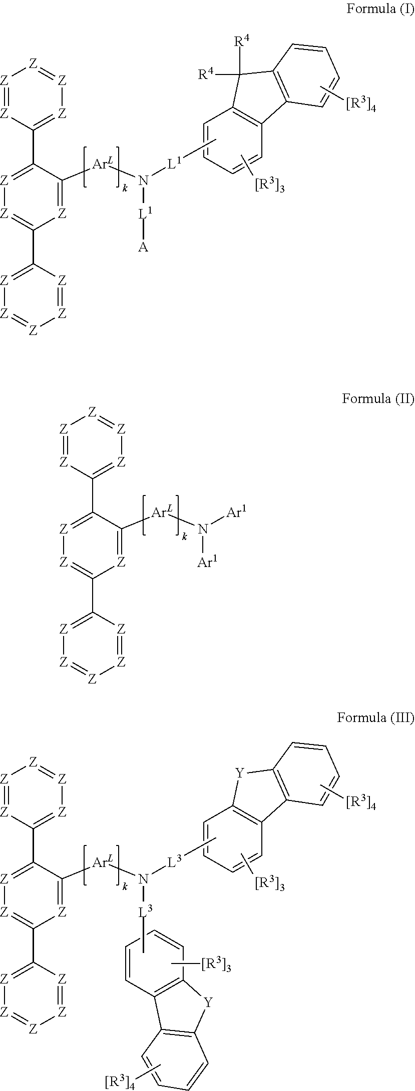
where:
A is a group selected from the following formulae: 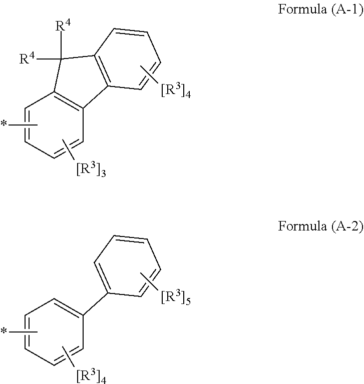
which is bonded to L¹ via the bond marked *;
Z is the same or different at each instance and is selected from CR¹ and N;
Ar^{L} is the same or different at each instance and is selected from aromatic ring systems which have 6 to 40 aromatic ring atoms and are substituted by R² radicals, and heteroaromatic ring systems which have 5 to 40 aromatic ring atoms and are substituted by R² radicals;
k is 0, 1, 2 or 3, where, when k=0, the Ar^{L} group is absent and the two groups that bind to Ar^{L} in formula (I), (II) and (III) are bonded directly to one another, where, when k=2, two Ar^{L} groups are bonded successively in a chain, and where, when k=3, three Ar^{L} groups are bonded successively in a chain;
in the case that k=0, Ar¹ is the same or different at each instance and is selected from aromatic ring systems which have 6 to 40 aromatic ring atoms and are substituted by R³ radicals, and heteroaromatic ring systems which have 5 to 40 aromatic ring atoms and are substituted by R³ radicals, where at least one Ar¹ group is selected from the following groups in which the bond to the nitrogen atom in formula (II) is labelled *: 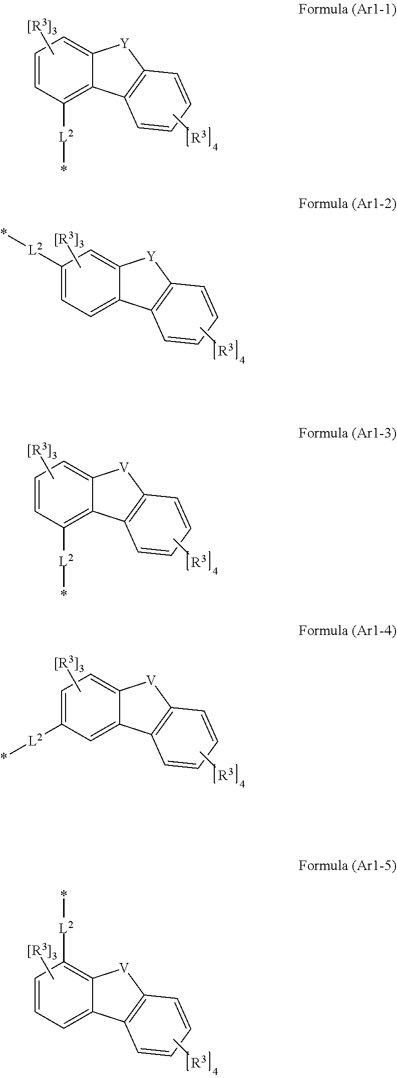 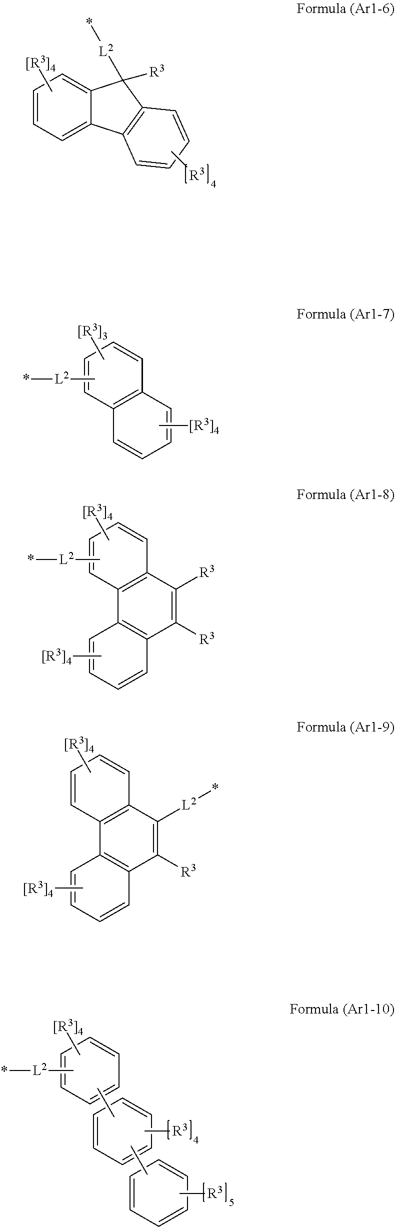
when k=1, 2 or 3, Ar¹ is the same or different at each instance and is selected from aromatic ring systems which have 6 to 40 aromatic ring atoms and are substituted by R³ radicals, and heteroaromatic ring systems which have 5 to 40 aromatic ring atoms and are substituted by R³ radicals;
L¹ is the same or different at each instance and is a single bond, an aromatic ring system which has 6 to 40 aromatic ring atoms and is substituted by R⁵ radicals, or a heteroaromatic ring system which has 5 to 40 aromatic ring atoms and is substituted by R⁵ radicals;
L² is the same or different at each instance and is a single bond, an aromatic ring system which has 6 to 40 aromatic ring atoms and is substituted by R⁵ radicals, or a heteroaromatic ring system which has 5 to 40 aromatic ring atoms and is substituted by R⁵ radicals;
L³ is the same or different at each instance and is an aromatic ring system which has 6 to 40 aromatic ring atoms and is substituted by R⁵ radicals, or a heteroaromatic ring system which has 5 to 40 aromatic ring atoms and is substituted by R⁵ radicals;
Y is the same or different at each instance and is selected from O and S,
V is the same or different at each instance and is selected from Si(R³)₂, C(R³)₂ and a group 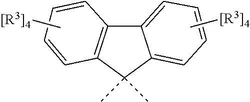
where the dotted bonds are the bonds to the radical of the formula Ar¹-3, Ar¹-4 or Ar¹-5;
R¹ is the same or different at each instance and is selected from H, D, F, Cl, Br, I, C(═O)R⁶, CN, Si(R⁶)₃, N(R⁶)₂, P(═O)(R⁶)₂, OR⁶, S(═O)R⁶, S(═O)₂R⁶, straight-chain alkyl or alkoxy groups having 1 to 20 carbon atoms, branched or cyclic alkyl or alkoxy groups having 3 to 20 carbon atoms, alkenyl or alkynyl groups having 2 to 20 carbon atoms, aromatic ring systems having 6 to 40 aromatic ring atoms excluding fluorenyl, and heteroaromatic ring systems having 5 to 40 aromatic ring atoms; where two or more R¹ radicals may be joined to one another and may form a ring; where the alkyl, alkoxy, alkenyl and alkynyl groups mentioned and the aromatic ring systems and heteroaromatic ring systems mentioned are each substituted by R⁶ radicals; and where one or more CH₂ groups in the alkyl, alkoxy, alkenyl and alkynyl groups mentioned may be replaced by —R⁶C═CR⁶—, —C≡C—, Si(R⁶)₂, C═O, C═NR⁶, —C(═O)O—, —C(═O)NR⁶—, NR⁶, P(═O)(R⁶), —O—, —S—, SO or SO₂;
R² is the same or different at each instance and is selected from H, D, F, Cl, Br, I, C(═O)R⁶, CN, Si(R⁶)₃, N(R⁶)₂, P(═O)(R⁶)₂, OR⁶, S(═O)R⁶, S(═O)₂R⁶, straight-chain alkyl or alkoxy groups having 1 to 20 carbon atoms, branched or cyclic alkyl or alkoxy groups having 3 to 20 carbon atoms, alkenyl or alkynyl groups having 2 to 20 carbon atoms, aromatic ring systems having 6 to 40 aromatic ring atoms, and heteroaromatic ring systems having 5 to 40 aromatic ring atoms; where two or more R² radicals may be joined to one another and may form a ring; where the alkyl, alkoxy, alkenyl and alkynyl groups mentioned and the aromatic ring systems and heteroaromatic ring systems mentioned are each substituted by R⁶ radicals; and where one or more CH₂ groups in the alkyl, alkoxy, alkenyl and alkynyl groups mentioned may be replaced by —R⁶C═CR⁶—, —C≡C—, Si(R⁶)₂, C═O, C═NR⁶, —C(═O)O—, —C(═O)NR⁶—, NR⁶, P(═O)(R⁶), —O—, —S—, SO or SO₂;
R³ is the same or different at each instance and is selected from H, D, F, Cl, Br, I, C(═O)R⁶, CN, Si(R⁶)₃, N(R⁶)₂, P(═O)(R⁶)₂, OR⁶, S(═O)R⁶, S(═O)₂R⁶, straight-chain alkyl or alkoxy groups having 1 to 20 carbon atoms, branched or cyclic alkyl or alkoxy groups having 3 to 20 carbon atoms, alkenyl or alkynyl groups having 2 to 20 carbon atoms, aromatic ring systems having 6 to 40 aromatic ring atoms, and heteroaromatic ring systems having 5 to 40 aromatic ring atoms; where two or more R³ radicals may be joined to one another and may form a ring; where the alkyl, alkoxy, alkenyl and alkynyl groups mentioned and the aromatic ring systems and heteroaromatic ring systems mentioned are each substituted by R⁶ radicals; and where one or more CH₂ groups in the alkyl, alkoxy, alkenyl and alkynyl groups mentioned may be replaced by —R⁶C═CR⁶—, —C≡C—, Si(R⁶)₂, C═O, C═NR⁶, —C(═O)O—, —C(═O)NR⁶—, NR⁶, P(═O)(R⁶), —O—, —S—, SO or SO₂;
R⁴ is the same or different at each instance and is selected from straight-chain alkyl or alkoxy groups having 1 to 20 carbon atoms, branched or cyclic alkyl or alkoxy groups having 3 to 20 carbon atoms, alkenyl or alkynyl groups having 2 to 20 carbon atoms, aromatic ring systems having 6 to 40 aromatic ring atoms, and heteroaromatic ring systems having 5 to 40 aromatic ring atoms; where any two R⁴ radicals may be joined to one another and may form a ring; where the alkyl, alkoxy, alkenyl and alkynyl groups mentioned and the aromatic ring systems and heteroaromatic ring systems mentioned are each substituted by R⁶ radicals; and where one or more CH₂ groups in the alkyl, alkoxy, alkenyl and alkynyl groups mentioned may be replaced by —R⁶C═CR⁶—, —C≡C—, Si(R⁶)₂, C═O, C═NR⁶, —C(═O)O—, —C(═O)NR⁶—, NR⁶, P(═O)(R⁶), —O—, —S—, SO or SO₂; with the proviso that no two R⁴ radicals bonded to the same carbon atom may both be aromatic ring systems;
R⁵ is the same or different at each instance and is selected from H, D, F, Cl, Br, I, C(═O)R⁶, CN, Si(R⁶)₃, N(R⁶)₂, P(═O)(R⁶)₂, OR⁶, S(═O)R⁶, S(═O)₂R⁶, straight-chain alkyl or alkoxy groups having 1 to 20 carbon atoms, branched or cyclic alkyl or alkoxy groups having 3 to 20 carbon atoms, alkenyl or alkynyl groups having 2 to 20 carbon atoms, aromatic ring systems having 6 to 40 aromatic ring atoms, and heteroaromatic ring systems having 5 to 40 aromatic ring atoms; where two or more R⁵ radicals may be joined to one another and may form a ring; where the alkyl, alkoxy, alkenyl and alkynyl groups mentioned and the aromatic ring systems and heteroaromatic ring systems mentioned are each substituted by R⁶ radicals; and where one or more CH₂ groups in the alkyl, alkoxy, alkenyl and alkynyl groups mentioned may be replaced by —R⁶C═CR⁶—, —C≡C—, Si(R⁶)₂, C═O, C═NR⁶, —C(═O)O—, —C(═O)NR⁶—, NR⁶, P(═O)(R⁶), —O—, —S—, So or SO₂;
R³ is the same or different at each instance and is selected from H, D, F, Cl, Br, I, C(═O)R⁷, CN, Si(R⁷)₃, N(R⁷)₂, P(═O)(R⁷)₂, OR⁷, S(═O)R⁷, S(═O)₂R⁷, straight-chain alkyl or alkoxy groups having 1 to 20 carbon atoms, branched or cyclic alkyl or alkoxy groups having 3 to 20 carbon atoms, alkenyl or alkynyl groups having 2 to 20 carbon atoms, aromatic ring systems having 6 to 40 aromatic ring atoms, and heteroaromatic ring systems having 5 to 40 aromatic ring atoms; where two or more R⁶ radicals may be joined to one another and may form a ring; where the alkyl, alkoxy, alkenyl and alkynyl groups mentioned and the aromatic ring systems and heteroaromatic ring systems mentioned are each substituted by R⁷ radicals; and where one or more CH₂ groups in the alkyl, alkoxy, alkenyl and alkynyl groups mentioned may be replaced by —R⁷C═CR⁷—, —C≡C—, Si(R⁷)₂, C═O, C═NR⁷, —C(═O)O—, —C(═O)NR⁷—, NR⁷, P(═O)(R⁷), —O—, —S—, SO or SO₂;
R⁷ is the same or different at each instance and is selected from H, D, F, Cl, Br, I, CN, alkyl or alkoxy groups having 1 to 20 carbon atoms, alkenyl or alkynyl groups having 2 to 20 carbon atoms, aromatic ring systems having 6 to 40 aromatic ring atoms and heteroaromatic ring systems having 5 to 40 aromatic ring atoms; where two or more R⁷ radicals may be joined to one another and may form a ring; and where the alkyl, alkoxy, alkenyl and alkynyl groups, aromatic ring systems and heteroaromatic ring systems mentioned may be substituted by one or more radicals selected from F and CN.

21. The compound according to claim 20, wherein Z is CR¹.

22. The compound according to claim 20, wherein Ar^{L} is the same or different at each instance and is selected from phenyl, biphenyl, naphthyl and fluorenyl, each substituted by R² radicals.

23. The compound according to claim 20, wherein the L¹ is the same or different at each instance and is selected from single bond, phenylene substituted by R⁵ radicals, naphthylene substituted by R⁵ radicals, fluorenylene substituted by R⁵ radicals, and biphenylene substituted by R⁵ radicals.

24. The compound according to claim 20, wherein L² is the same or different at each instance and is selected from single bond, phenylene substituted by R⁵ radicals, naphthylene substituted by R⁵ radicals, fluorenylene substituted by R⁵ radicals, and biphenylene substituted by R⁵ radicals.

25. The compound according to claim 20, wherein L³ is the same or different at each instance and is selected from phenylene substituted by R⁵ radicals, naphthylene substituted by R⁵ radicals, fluorenylene substituted by R⁵ radicals, and biphenylene substituted by R⁵ radicals.

26. The compound according to claim 20, wherein k=1, 2 or 3, and Ar¹ is the same or different at each instance and is selected from monovalent groups derived from benzene, biphenyl, terphenyl, quaterphenyl, naphthalene, fluorene, benzofluorene, spirobifluorene, indenofluorene, indenocarbazole, dibenzofuran, dibenzothiophene, benzocarbazole, carbazole, benzofuran, benzothiophene, indole, quinoline, pyridine, pyrimidine, pyrazine, pyridazine and triazine, where each of the monovalent groups is substituted by R³ radicals.

27. The compound according to claim 20, wherein, in the compounds of one of the formulae (I) to (III), zero, one, two or three R¹ groups per formula are not H or D, and in that these groups that are not H or D are the same or different at each instance and are selected from F, CN, Si(R⁶)₃, straight-chain alkyl groups having 1 to 20 carbon atoms, branched or cyclic alkyl groups having 3 to 20 carbon atoms, aryl groups having 6 to 25, aromatic ring atoms, and heteroaryl groups having 5 to 40 aromatic ring atoms, where the alkyl groups mentioned, the aryl groups mentioned and the heteroaryl groups mentioned are each substituted by R⁶ radicals.

28. The compound according to claim 20, wherein the compounds of one of the formulae (I) to (III) have at least one R¹ group that is a phenyl groups substituted by R⁶ radicals.

29. The compound according to claim 20, wherein all R¹ radicals in formulae (I) to (III) are H or D.

30. The compound according to claim 20, wherein R⁴ is the same or different at each instance and is selected from straight-chain alkyl groups which have 1 to 20 carbon atoms and are each substituted by R⁶ radicals, and branched or cyclic alkyl groups which have 3 to 20 carbon atoms and are each substituted by R⁶ radicals; where any two R⁴ radicals may be joined to one another and may form a ring.

31. The compound according to claim 20, wherein the compound conforms to one of the following formulae: 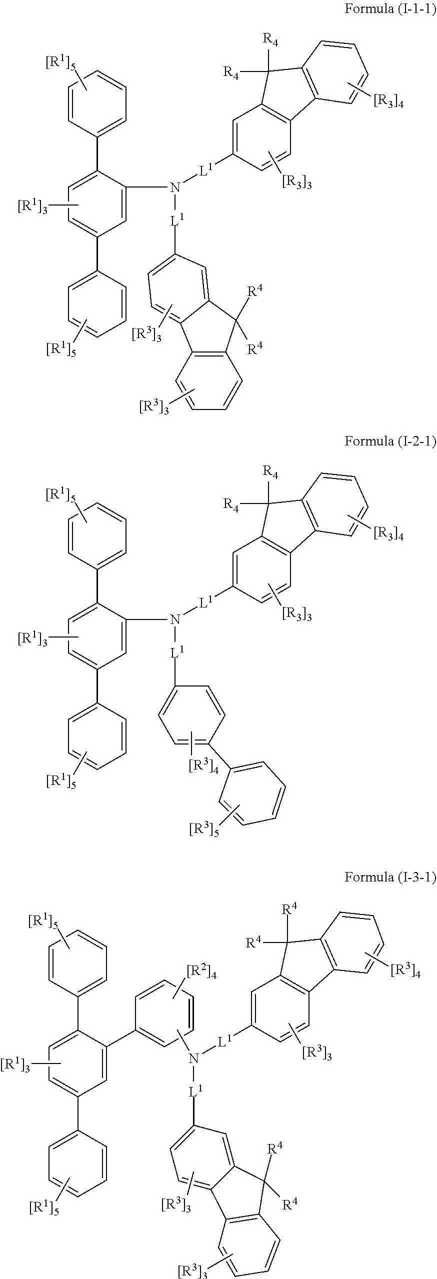 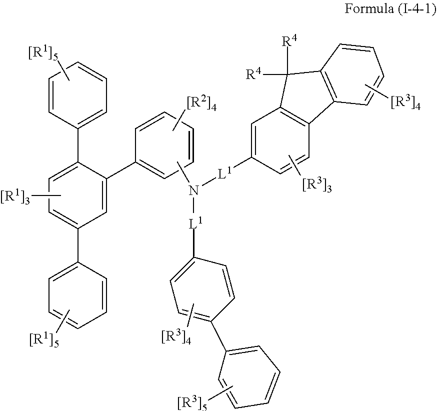
where the groups that occur are as defined in claim 20; or in that the compound conforms to one of the following formulae: 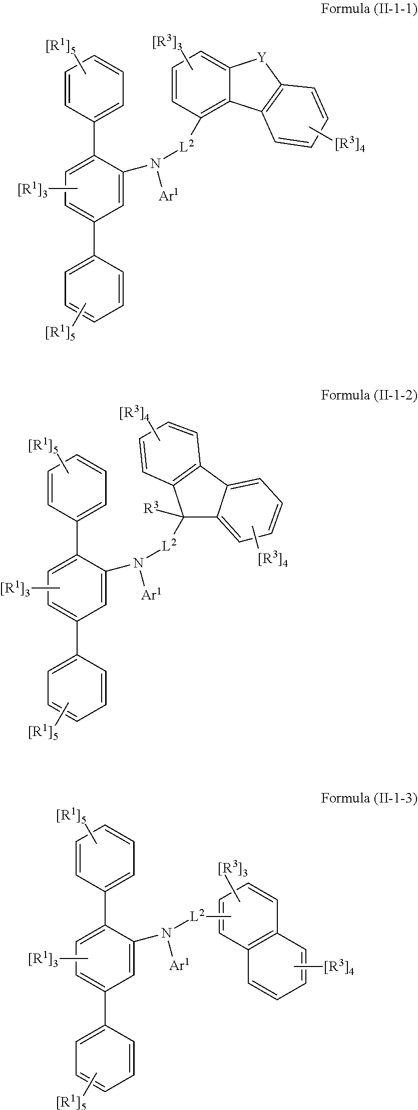 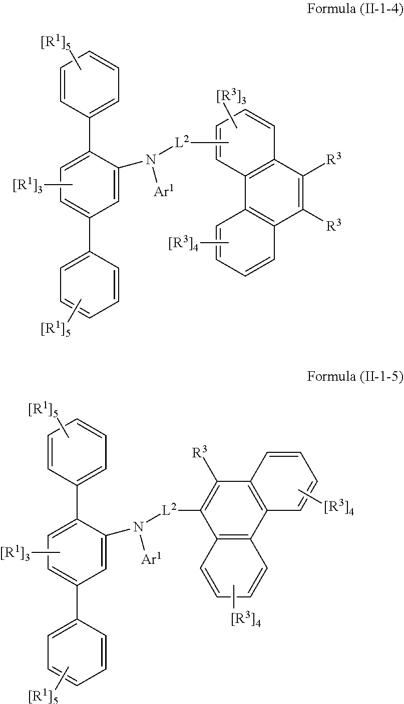
where the groups that occur are as defined in claim 20, and where Ar¹ is selected from aromatic ring systems which have 6 to 40 aromatic ring atoms and are substituted by R³ radicals, and heteroaromatic ring systems which have 5 to 40 aromatic ring atoms and are substituted by R³ radicals; or in that the compound conforms to one of the following formulae: 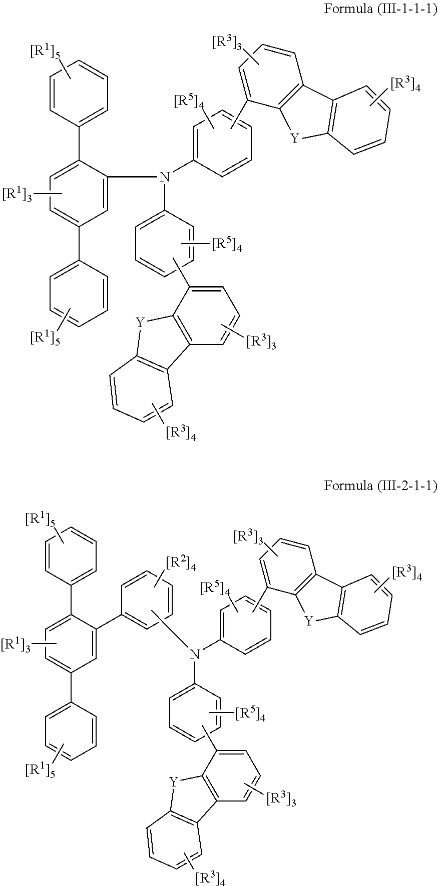
where the groups that occur are defined in claim 20.

32. The compound according to claim 20, wherein the unit 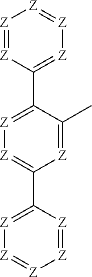
in formulae (I), (II) and (III) has one of the following structures: 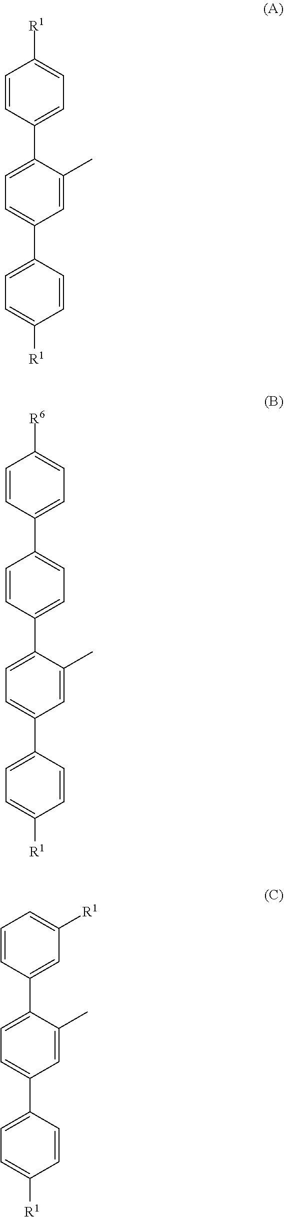 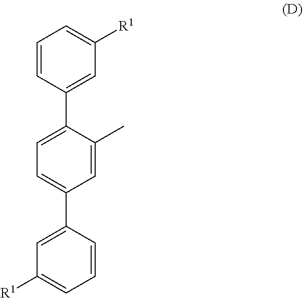
where R¹ and R⁶ are as defined in claim 20.

33. A process for preparing the compound according to claim 20, comprising reacting a terphenyl derivative a) substituted by a reactive group in a coupling reaction with a secondary amine, or b) reacting in a coupling reaction with an aromatic or heteroaromatic species bearing a boron-containing group.

34. An oligomer, polymer or dendrimer containing one or more compounds according to claim 20, wherein the bond(s) to the polymer, oligomer or dendrimer may be localized at any desired positions substituted by R¹, R¹, R³, R⁴ or R⁵ in formula (I), (II) and (III).

35. A formulation comprising at least one compound according to claim 20 and at least one solvent.

36. An electronic device comprising at least one compound according to claim 20.

37. The electronic device according to claim 36, wherein the device is an organic electroluminescent device and comprises an anode, cathode and at least one emitting layer, and wherein the compound is present in a hole-transporting layer or in an emitting layer of the device.

38. A method comprising proving the compound according to claim 20 and incorporating the compound in an electronic device.
